SEQUENCE LISTING (176 sequences)

SEQ ID NO: 1 - protein, 11 aa, unidentified
  VAR_SEQ 2: note = X is any naturally occurring amino acid
  VAR_SEQ 4: note = X is D or E
  VAR_SEQ 7: note = X is any naturally occurring amino acid
  VAR_SEQ 8: note = X is any naturally occurring amino acid
  VAR_SEQ 9: note = X is any naturally occurring amino acid
  VAR_SEQ 10: note = X is any naturally occurrring amino acid
CXIXGRXXXXC

SEQ ID NO: 2 - protein, 17 aa, unidentified
CHKAIDGRSLYNKTLDC

SEQ ID NO: 3 - protein, 17 aa, unidentified
CHKAIEGRSLYNKTLDC

SEQ ID NO: 4 - protein, 4 aa, unidentified
CHKA

SEQ ID NO: 5 - protein, 5 aa, unidentified
CHKAI

SEQ ID NO: 6 - protein, 6 aa, unidentified
CHKAID

SEQ ID NO: 7 - protein, 4 aa, unidentified
IDGR

SEQ ID NO: 8 - protein, 4 aa, unidentified
IEGR

SEQ ID NO: 9 - protein, 8 aa, unidentified
CHKAIDGR

SEQ ID NO: 10 - protein, 8 aa, unidentified
CHKAIEGR

SEQ ID NO: 11 - protein, 4 aa, unidentified
TLDC

SEQ ID NO: 12 - protein, 5 aa, unidentified
KTLDC

SEQ ID NO: 13 - protein, 7 aa, unidentified
YNKTLDC

SEQ ID NO: 14 - protein, 8 aa, unidentified
LYNKTLDC

SEQ ID NO: 15 - protein, 8 aa, unidentified
LTPRGVRL

SEQ ID NO: 16 - protein, 6 aa, unidentified
LVPRGS

SEQ ID NO: 17 - protein, 10 aa, unidentified
ENKSLVPRGS

SEQ ID NO: 18-22 - skipped (5 entries)

SEQ ID NO: 23 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 24 - skipped

SEQ ID NO: 25 - protein, 5 aa, unidentified
SGRSA

SEQ ID NO: 26 - protein, 8 aa, unidentified
PGSGRSAG

SEQ ID NO: 27 - protein, 14 aa, unidentified
PGSGRSASGTTGTG

SEQ ID NO: 28-29 - skipped (2 entries)

SEQ ID NO: 30 - protein, 8 aa, unidentified
  VAR_SEQ 1: note = X is L, R or V
  VAR_SEQ 6: note = X is L, R or V
  VAR_SEQ 8: note = X is N or G
XSGRGXXX

SEQ ID NO: 31 - protein, 432 aa, Rattus norvegicus
MRVWLASLFLCALVANSEGGSELEASDESNCGCQNGGVCVSYKYFSSIRRCSCPKKFKGEHCEIDTSKTCYHGNGQSYRGKANTDTKGRPCLAWNSPAVLQQTYNAHRSDALSLGLGKHNYCRNPDNQRRPWCYVQIGLKQFVQECMVQDCSLSKKPSSTVDQQGFQCGQKALRPRFKIVGGEFTVVENQPWFAAIYLKNKGGSPPSFKCGGSLISPCWVASATHCFVNQPKKEEYVVYLGQSKRNSYNPGEMKFEVEQLILHEDFSDETLAFHNDIALLKIRTSTGQCAQPSRTIQTICLPPRFGDAPFGSDCEITGFGQESATDYFYPKDLKMSVVKIISHEQCKQPHYYGSEINYKMLCAADPEWKTDSCSGDSGGPLICNIDGRPTLSGIVSWGSGCAEKNKPGVYTRVSYFLNWIQSHIGEENGLAF

SEQ ID NO: 32 - protein, 9 aa, unidentified
VVPRVELVA

SEQ ID NO: 33 - protein, 8 aa, unidentified
PPFGRSAG

SEQ ID NO: 34 - skipped

SEQ ID NO: 35 - protein, 8 aa, unidentified
  VAR_SEQ 4: note = X is K or R
GPXXXGGX

SEQ ID NO: 36-38 - skipped (3 entries)

SEQ ID NO: 39 - protein, 8 aa, unidentified
  VAR_SEQ 4: note = X is K or R
GPXXXGGX

SEQ ID NO: 40 - protein, 8 aa, unidentified
  VAR_SEQ 4: note = X is F or Y
GPYXXGGX

SEQ ID NO: 41 - protein, 8 aa, unidentified
  VAR_SEQ 4: note = X is K or R
GPXXKGGX

SEQ ID NO: 42 - protein, 8 aa, unidentified
  VAR_SEQ 4: note = X is K or R
  VAR_SEQ 8: note = X is any naturally occurring amino acid
GPYXKGGX

SEQ ID NO: 43 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 44-46 - skipped (3 entries)

SEQ ID NO: 47 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 48 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 49 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 50 - protein, 7 aa, unidentified
PGRVVGG

SEQ ID NO: 51-52 - skipped (2 entries)

SEQ ID NO: 53 - protein, 17 aa, synthetic construct
CHKAIDKRVLYNKTLDC

SEQ ID NO: 54 - protein, 17 aa, synthetic construct
CHKALVPRGSYNKTLDC

SEQ ID NO: 55 - protein, 17 aa, synthetic construct
CHKAIDGRILYNKTLDC

SEQ ID NO: 56 - protein, 21 aa, synthetic construct
CHKAENKSLVPRGSYNKTLDC

SEQ ID NO: 57 - protein, 17 aa, synthetic construct
CHKALTPRGVRLKTLDC

SEQ ID NO: 58 - protein, 9 aa, synthetic construct
CPGRVVGGC

SEQ ID NO: 59 - protein, 17 aa, synthetic construct
CGGGGSPGRVVGGGGSC

SEQ ID NO: 60 - protein, 9 aa, synthetic construct
CSGRSAGGC

SEQ ID NO: 61 - protein, 16 aa, synthetic construct
CGGGGSGRSAGGGGSC

SEQ ID NO: 62 - protein, 17 aa, synthetic construct
CHKAVVPRVELVATLDC

SEQ ID NO: 63 - protein, 20 aa, synthetic construct
CHKAIPPFGRSAGYNKTLDC

SEQ ID NO: 64 - protein, 20 aa, synthetic construct
CHKAIPGSGRSAGYNKTLDC

SEQ ID NO: 65 - protein, 17 aa, synthetic construct
CHKAISGRSAYNKTLDC

SEQ ID NO: 66 - protein, 26 aa, synthetic construct
CHKAIPGSGRSASGTTGTGYNKTLDC

SEQ ID NO: 67 - protein, 14 aa, Clostridium botulinum
CLRLTKNSRDDSTC

SEQ ID NO: 68 - protein, 14 aa, Clostridium botulinum
CLRLTRNSRDDSTC

SEQ ID NO: 69 - protein, 17 aa, Clostridium botulinum
CHKAIDGRSLYNKTLDC

SEQ ID NO: 70 - protein, 25 aa, Clostridium botulinum
CVRGIITSKTKSLDEGYNKALNELC

SEQ ID NO: 71 - protein, 25 aa, Clostridium botulinum
CVRGIITSKTKSLDEGYNKALNDLC

SEQ ID NO: 72 - protein, 25 aa, Clostridium botulinum
CVRGIITSKTKSLDKGYNKALNDLC

SEQ ID NO: 73 - protein, 25 aa, Clostridium botulinum
CVRGIIPFKTKSLDEGYNKALNYLC

SEQ ID NO: 74 - protein, 25 aa, Clostridium botulinum
CVRGIIPFKTKSLDEGYNKALNDLC

SEQ ID NO: 75 - protein, 11 aa, Clostridium botulinum
CSNSNTKNSLC

SEQ ID NO: 76 - protein, 15 aa, Clostridium botulinum
CKNIVSVKGIRKSIC

SEQ ID NO: 77 - protein, 15 aa, Clostridium botulinum
CTNIFSPKGIRKSIC

SEQ ID NO: 78 - protein, 15 aa, Clostridium botulinum
CKNIVFSKGITKSIC

SEQ ID NO: 79 - protein, 15 aa, Clostridium botulinum
CKNIVFSKGIRKSIC

SEQ ID NO: 80 - protein, 15 aa, Clostridium botulinum
CKSIVSKKGTKNSLC

SEQ ID NO: 81 - protein, 15 aa, Clostridium botulinum
CLNSSFKKNTKKPLC

SEQ ID NO: 82 - protein, 17 aa, Clostridium botulinum
CKSVIPRKGTKAPPRLC

SEQ ID NO: 83 - protein, 17 aa, Clostridium botulinum
CKSIIPRKGTKAPPRLC

SEQ ID NO: 84 - protein, 17 aa, Clostridium botulinum
CKSIIPRKGTKQSPSLC

SEQ ID NO: 85 - protein, 29 aa, Clostridium tetani
CKKIIPPTNIRENLYNRTASLTDLGGELC

SEQ ID NO: 86 - protein, 15 aa, Clostridium botulinum
CKPVMYKNTGKSEQC

SEQ ID NO: 87 - protein, 10 aa, Clostridium botulinum
CKSVKVPGIC

SEQ ID NO: 88 - protein, 10 aa, Clostridium botulinum
CKSVRAPGIC

SEQ ID NO: 89 - protein, 10 aa, Clostridium botulinum
CKSVKAPGIC

SEQ ID NO: 90 - protein, 45 aa, Clostridium botulinum
CPRNGLLYNAIYRNSKNYLNNIDLEDKKTTSKTNVSYPCSLLNGC

SEQ ID NO: 91 - protein, 1296 aa, Clostridium botulinum [sequence omitted]

SEQ ID NO: 92 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 93 - protein, 1292 aa, Clostridium botulinum
MPFVNKPFNYRDPGNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNYLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 94 - protein, 1296 aa, Clostridium botulinum
MPLVNQQINYYDPVNGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTFQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 95 - protein, 1296 aa, Clostridium botulinum
MLFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTELGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGEHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIVLTNSVNEALLNPSSVYTFFSSDYVRKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWGEVYKYIVTNWLAKVNTQIDLIRKKMKEALENQAEATKAIINYQYNQYTEEEKNNINFNIGDLSSKLNDSINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASEINIGSKVNFDPIDKNQIQLFNLESSKIEIILKNAIVYNSMYENFSTSFWIKIPKYFSKINLNNEYTIINCIENNSGWKVSLNYGEIIWTLQDNKQNIQRVVFKYSQMVAISDYINRWIFITITNNRLNNSKIYINGRLIDQKPISNLGNIHASNNIMFKLDGCRDPHRYIWIKYFNLFDKELNEKEIKDLYDNQSNSGILKDFWGNYLQYDKPYYMLNLYDPNKYVDVNNVGIRGYMYLKGPRGSIVTTNIYLNSSLYMGTKFIIKKYASGNKDNIVRNNDRVYINVVVKNKEYRLATNASQAGVEKILSVLEIPDVGNLSQVVVMKSKNDQGIRNKCKMNLQDNNGNDIGFIGFHQFNNIDKLVASNWYNRQIERSSRTFGCSWEFIPVDDGWGESPL

SEQ ID NO: 96 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFAKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLSAQEFEHGKSRIDLTNSVNEALLNPSHVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVDRLKDKVNNTLSTDIPFQLSKYVDNQRLLSTFTEYIKNIINTSILSLRYENNHLIDLSRYASKINIGSRVNFDPIDKNQIQLFNLESSKIEVILKNAIVYNSMYENFSTSFWIKIPKYFSEISLNNEYTIINCIENNSGWKVSLNYGEIIWTLQDNKQNIQRVVFKYSQMVAISDYINRWIFITITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNVGIRGYMYLKGSRSTLLTTNIYLNSGLYMGTKFIIKKYASGNKDNIVRNNDRVYINVVVNNKEYRLATNASQAGVEKILSALEIPDIGNLSQVVVMKSKNDQGIRNKCKMNLQDNNGNDIGFIGFHKFNDIYKLVASNWYNRQIEISSRTFGCSWEFIPVDDGWGEKPL

SEQ ID NO: 97 - protein, 1296 aa, Clostridium botulinum
MPFVNKQFNYKDPVNGVDIAYIKIPNAGQMQPVKAFKIHNKIWVIPERDIFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIINFECKSFGHDVLNLTRNGYGSTQYIRFSPDFTFGFEESLEVDTNPLLGAGKFAIDPAVTLAHELIHAGHRLYGIAINPNRVFKVNTNAYYEMSGLEVSFEELRTFGGHDAKFIDSLQENEFRLYYYNKFKEVASILNKAKSIIGTTASLQYMKNVFKEKYLLSEDTSGKFSVDKLRFDKLYKMLTEIYTEDNFVKFFKVLNRKTYLNFDKAVFKMNIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISSDLIQQYYLTFNFDNEPENISIENLSSDIIGQLELMPNIERFPNGKKYELDKYTMFHYLRAQEFEYGNSRIVLINSVNEALLNPSSVYTFFSSDYVKKANEATEAAMFLGWVEQLVYDFTDETSEVSTMDKIADITIIVPYIGPALNIGNMVYKKKFEEALIFSGAVILLEFVPEIVLPILGTFALVSYTSNKVLTVRTIDNALSKRNEKWEEVYKYIVTNWLAKVNTQINLIRKKMKEALENQAEATKAIINYQYNQYTEEEKNNINFNIGDLSSKLNDSINKAMININKFLDQCSVSYLMNSMIPQGVKQLKDFDTSLRDSLLKYIYDNRGTLIGQVDRLKDKVNNTLSTDIPFQLSKYADNQRLLSTFTEYIKNIINTSILNLRYESNHLIDLSRYASKINIGSRVNFDPIDKNQIQLFNLESSKIEVILKNAIVYNSMYENFSTSFWIKIPKYFSKINLNNEYTIINCIENNSGWKVSLNYGEIIWTLQDNEQNIQRVVFKYSQMVNISDYINRWIFVTITNNRLTKSKIYINGRLIDQKPISNLGNIHASNKIMFKLDGCRDPHRYILIKYFNLFDKELNEKEIKDLYDNQSNSGILKDFWGDYLQYDKPYYMLNLYDPNKYIDVNNIGIRGYMYLKGPRGSVTTTNIYLNSMLYMGTKFIIKKHASGNKDNIVRNNDRVYINVLVKNKEYRLATNASQAGGEKILSAVEIPDVGNLSQVVVMKSKNDQGIRNKCKMNLQDNNGNDIGFIGFHQFNNIAKLVASNWYNRQIGKTSVTLGCSWELIPVDYGWGESSL

SEQ ID NO: 98 - protein, 1297 aa, Clostridium botulinum
MPFVNKQFNYKDTVNGIDIAYIKIPNAGQMQPVKAFKIHNKIWVIPERDTFTNPKEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHNAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPDENYTIKDGFNLKNTNLAANFNGQNTEINSRNFTKLKNFTGLFEFYKLLCVRGIIPFKTKSLDEGYNKALNDLCIKVNNWDLFFSPSEDNFTNDLDKVEEITSDTNIEAAEENISLDLIQQYYLTFDFDNEPENISIENLSSDIIGQLEPMPNIERFPNGKKYELDKYTMFHYLRAQEFEHSKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLVRKKMKEALENQAEATKAIINYQYNQYTEEEKNNINFNIDDLSSKLNESINSAMTNINKFLDQCSVSYLMNSMIPYAVKRLKDFDASVREVLLKYIYDNRGTLILQVDRLKDKVNNTLSADIPFQLSKYVDNKKLLSTFTEYIKNITNTSILSIVVDKDGRLIDLSRYGAEIYNGDKVSYNSIDKNQIKLINLESSAIEVILKNAIVYNSMYENFSTSFWIKIPKYFSKINLNNEYTIINCIENNSGWKVSLNYGEIIWTLQDNQQNIQRVVFKYSQMVNISDYINRWIFVTITNNRLDKSKIYINGRLIDQKPISNLGNIHASNNIMFKLDGCRDPRRYIVIKYFNLFDKELNEKEIKDLYDNQSNSGILKDFWGDYLQYDKPYYMLNLYDPNKYVDVNNIGIRGYMYLKGPRGSVVTTNIYLNSTLYMGTKFIIKKYASGNKDNIVRNNDRVYINVVVKNKEYRLATNALQAGVEKILSALEIPDVGNLSQVVVMKSKNDQGIRNKCKMNLQDNNGNDIGLIGFHQFNNIAKLVASNWYNRQVGKASRTFGCSWEFIPVDDGWGESSQ

SEQ ID NO: 99 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 100 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAVDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNSNREWRVYAYKDFKEEEKKLFLANIYDSNEFYKTIQIKEYDEQPTYSCQLLFKKDEESTDEIGLIGIHRFYESGIVLKDYKNYFCISKWYLKEVKRKPYNPNLGCNWQFIPKDEGWIE

SEQ ID NO: 101 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGAKVEVYNGVELNDKNQFKLTSSANSKIRVTQNQDIIFNSMFLDFSVSFWIRIPKYKNDGIQNYIHNEYTIINCIKNNSGWKISIRGNKIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNLNQEWRVYAYKDFKKKEEKLFLANIYDSNEFYNTIQIKEYDEQPTYSCQLLFKKDEESTDEIGLIGIHRFYESGIVFKDYKDYFCISKWYLKEVKRKPYNPNLGCNWQFIPKDEGWIE

SEQ ID NO: 102 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYTNNEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRKSNSQSINDDIVRKEDYIHLDFVNSNEEWRVYAYKNFKEQEQKLFLSIIYDSNEFYKTIQIKEYDEQPTYSCQLLFKKDEESTDDIGLIGIHRFYESGVLRKKYKDYFCISKWYLKEVKRKPYKSNLGCNWQFIPKDEGWTE

SEQ ID NO: 103 - protein, 1291 aa, Clostridium botulinum
MPVTINNFNYNDPIDNNNIIMMEPPFARGMGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVNDLPIVPNEKKFFMQSTDAIQAEELYTFGGQDPSIISPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVKAPGICIDVDNEDLFFIADKNSFSDDLSKNERIAYNTQNNYIENDFSINELILDTDLISKIELPSENTESLTDFNVYVPVYKKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIETINSALTKRDEKWIDMYGLIVAQWLSTVNTQFYTIKEGMYKALNYQAQALEEIIKYKYNIYSEKERSNINIDFNDVNSKLNEGINQAIDNINNFINECSVSYLMKKMIPLAVEKLLDFDNTLRKNLLNYIDENKLYLIGSAEYEKSKVDKYLKTSIPFDLSTYTNNTILIEIFNKYNSDILNNIILNLRYRDNKLIDLSGYGAKVEVYDGVKLNDKNQFKLTSSANSKIRVIQNQNIIFNSMFLDFSVSFWIRIPKYKNDGIQNYIHNEYTIINCMKNNSGWKISIRGNMIIWTLIDINGKIKSVFFEYSIKEDISEYINRWFFVTITNNSDNAKIYINGKLESHIDIRDIREVIANDEIIFKLDGNIDRTQFIWMKYFSIFNTELSQSNIEEIYKIQSYSEYLKDFWGNPLMYNKEYYMFNAGNKNSYIKLKKDSSVGEILTRSKYNQNSKYINYRDLYIGEKFIIRRKSNSQSINDDIVRKEDYIYLDFFNLNQEWRVYMYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 104 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKFFTDENTIFQYLYSQTFPLDIRDISLTSSFDDALLFSNKVYSFFSMDYIKTANKVVEAGLFAGWVKQIVDDFVIEANKSNTMDKLADISLIVPYIGLALNVGNETAKGNFENAFEIAGASILLEFIPELLIPVVGAFLLESYIDNKNKIIKTIDNALTKRDEKWRDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFKKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSPVGEILTRSKYNQNSNYINYRNLYIGEKFIIRRKSNSQSINDDIVRKEDYIYLDFFNLNQEWRVYALKNFKKKEEKLFLAPISDSDEFYNTIQIKEYDEQPTYSCQLLFKKDEESTDEIGLIGIHRFYESGIVFKDYKYYFCISKWYLKEVKRKPYNPNLGCNWQFIPKDEGWIE

SEQ ID NO: 105 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTKNIYIENYFSINELILDTDLISGIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIIDDFVIEANKSSTMDKIADISLIVPYIGLALNVGNETAKGNFENAFEIAGASILLEFIPELLIPVVGAFLLESYIDNKNKIIKTIDNALTKRVEKWIDMYGLIVAQWLSTVNTQFYTIKEGMYKALNYQAQALEEIIKYKYNIYSEKEKLNINIDFNDINSKLNEGINQAIDNINNFINECSVSYLMKKMIPLAIEKLLDFDNALKKNLLNYIDENKLYLIGSVEEEKSKVDKFFKTIIPFDLSMYTNNTILIEMVNKYNSEILNNIILNLRYRDNNLIDSSGYGAKVEVYNGVELNDKNQFKLTSSANSKIKVTQNQNITFNSMFLDFSVSFWIRIPKYKNDGIQNYIHNEYTIINCMKNNSGWKISIRGNRIIWTLTDINGKTKSVFFEYSIREDISDYINRWFFVTITNNLDNAKIYINGKLESNIDIRDIREVIVNGEIIFKLDGEIDRTQFIWMKYFSIFNTELSQSNVKEIYKIQSYSKYLKDFWGNPLMYNKEYYMFNAGNKNSYIKLVKDSSVGEILTRSKYNQNSNYINYRNLYIGEKFIIRRKSSSQSISDDIVRKEDYIYLDFFNSNREWRVYAYKNFKGQEEKLFLANIYDSNEFYKTIQIKEYDEQPTYSCQLLFKKDEESTDEIGLIGIHNFYESGILFKDYKDYFCISKWYLKEVKKKPYSSNLGCNWQFIPKDEGWTE

SEQ ID NO: 106 - protein, 1292 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGGEERKEGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNGKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDDEIYTIEEGFNISDKNMGKEYRGQNKAINKQAYEEISKEHLAVYKIQMCKSVRAPGICIDVDNEDLFFIADKNSFSDDLSKNERIEYNTQSNYIENDFSINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSNTMDKIADISLIVPYIGLALNVGNETAKGNFENAFEIAGSSILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNISIDFNDINSKLNEGINQAIDNINDFINECSVSYLMKKMIPLAVEKLLDFDNTLKKNLLNYIDENKLYLIGSAEYEKSKVDKHLKTIMTFDLSMYTNNTILIKMVNKYNSEILNNIILNLRYRDNNLIDLSGYGANVEVYDGVELNDKNQFKLTSSTNSEIRVTQNQNIIVNSMFLDFSVSFWIRIPKYKNDGIQNYIHNEYTIINCMKNNSGWKISIRGNRIIWTLIDINGKIKSVFFEYSIRKDVSEYINRWFFVTITNNLDNAKIYINGKLESNMDIRDIREVIANGEIIFKLDGDIDRTQFIWMKYFSIFNTELSQSNIEEIYKIQSYSEYLKDFWGNPLMYNKEYYMFNAGSKNSYIKLKKDSSVGEILTRSKYNQNSQYINYRDLYIGEKFIIKRKSNSQSINDDIVRKEDYIYLDFFNLNQEWRVYAYKDFKGQKEQKLFLANIHDSNEFYKTIQIKEYDEQPTYSCQLLFKKDEESTDEIGLIGIHRFYESGFVFQEYKYYFCISKWYLKEVKKKPYNPDLGCNWQFIPKDEGWTE

SEQ ID NO: 107 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 108 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 109 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHAMHNLYGIAIPNDQRISSVTSNIFYSQYNVKLEYAEIYAFGGPTIDLIPKSARKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINEETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRGNFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKINESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLDEVKLDKTIVFGIDENIDENQMLWIRDFNIFSKELSNEDINIVYEGQILRNVIKDYWGNPLKFDTEYYMINYNYIDRYIAPKNNILVLVQYSDISKLYTKNPITIKSAANKNPYSRILNGDDIMFHMLYDSREYMIIRDTDTIYATQGGQCSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 110 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIIGNTNDTRVRGENVLYFNTTIDNKQYSLGMYKPSRNLGTDLVPLGALDQPMDEIRKYGSFIIQPCNTFDYYASQLFLSSNATTNRIGILSIGSYSFKLGDDYWFNHEYLIPVIKIEHYASLLESTSTHWVFVPASE

SEQ ID NO: 111 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 112 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 113 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 114 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 115 - protein, 1252 aa, Clostridium botulinum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 116 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNININGEIFIYPTNKNQFTIFNSKPSEVNISQNDYIIYDNKYKNFSISFWVRIPNYDNKIVNINNEYTIINCMRDNNSGWKVSLNHNEIIWTLQDNARINQKLVFKYGNANGISDYINKWIFVTITNDRLGDSKLYINGHLIDQKSILNLGNIHVSDNILFKIVNCSYTRYIGIRYFNIFDKELDETEIQTLYSNEPNTNILKDFWGNYLLYDKGYYLLNVLKPNNFIDRRKDSTLSINNIRSTILLANRLYSGIKVKIQRVNDSSTNDRFVRKNDQVYINYISNSSSYSLYADTNTTDKEKTIKSSSSGNRFNQVVVMNSVGNNCTMNFKNNNGNNIGLLGFKADTVVASTWYYTHMRDHTNSNGCFWNFISEEHGWQEK

SEQ ID NO: 117 - protein, 1252 aa, Clostridium botulinum
MPTINSFNYNDPVNDRTILYIKPGGCQEFYKSFNIMKNIWIIPERNVIGTTPQDFHPPTSLKNGDSSYYDPNYLQSYEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFKDNSMNEFIQDPALTLMHELIHSLHGLYGAKGITTQYTITQQQNPLITNIKGTNIEEFLTFGGTDLNIITSAQYNDIYTNLLADYKKIASKLSKVQVSNPLLNPYKDVFEKKYGLDKDASGIYSVNINKFNDIFKKLYSFTEFDLATKFQVKCRQTYIGQYKYFKLSNLLNNSIYNISEGYNINTLKVNFRGQNTNLNPRIITPLTGRGLVKKIIRFCKNIVFSKGIRKSICIEINNGELFFVASDNSYNDDNINTPKEIDDTVTSNNNYENDLDQVILNFNSESAPGLSDEKLNLTIQNDAYIPKYDSNGTSDIEQHDVNELNVFFYLDAQKVPEGENNIDFTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNNKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVDSKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 118 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 119 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSYIEQHDVNELNVFFYLDAQKVPEGENNVNLTSSIDTALLEQPKIYTFFSSEFINNVNKTVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 120 - protein, 1251 aa, Clostridium botulinum
MPKINSFNYNDPVNDNTILYIKPGGCQQFYKSFNIMKNIWIIPERNVIGTIPQNFLPPTSLKNGDSSYYDPNYLQNDQEKDRFLKIVTKVFNRINDNLSGRILLEELSKANPYLGNDNTRDDDFIINDGSAVPIQFSNGSQSILLPTVIIMGAEPDLFETNSSNVSLINNYSPSNHGFGSIAIVTFSPEYSFRFNDNSMNEFIQDPALTLIHELIHSLHGLYGAKGITTKYTITQQQNPLITNIRGINIEEFLTFGGNNLNIITSSQLNDIYTNLLDDYKKIASKLSKVQVSNPQLNPYKDVFQEKYGLDKNASGIYSVNINKFNDIFKKLYSFTEFDLATKFQVKCRETYIGQYKYFKLSNLLNDSIYNISEGYNINTLNVNFRGQNPNLNPRIITPITDRGLVKKIIRFCKNIVSVKGIRKSICIEVNNGDLFFVASEKSYNNDSINIPKEIDDTVTLNNNYENDLDQVILNFNSESAPGLSDKKLNISIQDDVYIPKYDSNGTSDIEQYDVSELNVFFYLDAQKVPEGENNVNLTSSIDTALLEQSKIYTFFSSEFINNVNKPVQAALFVGWIQQVLVDFTTEATQKSTVDKIADISIVVPYIGLALNIGNESQKGNFKDALELLGAGILLEFVPELLIPTILVFTIKSFLGSSDNKNKVIKAINNALKERDEKWKEVYSFIVSNWITKINTQFNKRKEQMYQALQNQVNALKTIIESKYNSYTLEEKNELTNKYDIEQIENELNQKVSIAMNNIDRFLTESSISYLMKLINEVKINKLREYDENVKTYLLDYITKHGSILGESQQELNSMIIDTLNNSIPFKLSSYTDDKILISYFNKFFKTIKSSSVLSMRYKNDKYIDTSGYDSNININGDVFIYPTNKNQFGIYNSKLSEVNISQNDYIIYDNKYKNFSISFWVRIPNYNNKIVNVNNEYTIINCMRDNNSGWKISLNHNEIIWTLQDNAGINQKLVFKYGNANGISDYINKWIFVTITNDRLGYSKLYINGHLIDQKSILNLGNIHVSDNILFKIVNCSYTRYIGMRYFNIFDKELDETEIQTLYNNEPNANVLKDFWGNYLLYNKEYYLLNMLKPSKTISHNRDLTFSIYNNRNIVNGLYRLYSGIKVKIQKINDSDTRDNIVRDNDQVYVNYINGNVYYSLYADTNATNKEKTIKSSTSGNRFNQVVVMNSVRNNCTMNFKNNNGHDIGLLGFKSNALVASTWYYTNMRDHTNSNGCFWSFIPEENGWQEH

SEQ ID NO: 121 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLADYKKIASKLSQVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANKLYLGIKVKIQRVNNSSTNDNLVRKNDQVYINFVPIKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 122 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLDDYKKIASKLSQVQVSNPQLNPYKDVFQEKYGLDKDANGIYSVNINKFNDIFKKLYSFTEFDLATKFQVKCRKTYIGHHKYFRLSDLLNDSIYNISDGYNINTLKVNFRGQNTNLNTRIITPITGRGVVRKIIRFCTNIFSPKGIRKSICIEVNNGELFFVASENSYNDDNINTSKEIDDTVTSNNNYENDLDQVILNFNSESAPGLSDEKLNLTIQDDAYIPKYDSNGTSDIEQYDVSELNVFFYLDAQKVPEGENNVDFTSSIDTALLEQPKIYTFFSSKFISNLNKTMQAALFVSWIQQVLVDFTTEATQKSTVDKIADISIVVPYIGLALNIGNEAQKGNFKDALELLGAGILLEFEPELLIPIILVFTIKSFLGSSDNKNKVIKAINNALKERDENWKEVYSFIVSNWMTKINTQFNKRKEQMYQALQNQVNAIKTIIESKYNSYTLEEKNELKNKYDIEQIENELNQTVSIAMNNIEIFLTESSISYLMKLINEVKINKLKEYDENVKTYLLDYIIKHGSILGESQQELNSMVIDTLNNSIPFKLSSYTDDKILISYFNKFFKTIKSSSVLNMRYKNDKYIDTSGYDSNINIKGDVFIYPTNKNQFGIYNNKLSEVNISQNDYIIYDNKYKNFSISFWVRIPNYDNKIVNVNNEYTIINCMRDNNSGWKVSLNHNEIIWTLQDNAGINQKLVFKYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 123 - protein, 1254 aa, Clostridium botulinum
MLYMPKINSFNYNDPVNDRTILYIKPGGCQQFYKSFNIMKNIWIIPERNVIGTIPQDFQPPTSLKNGDSSYYDPNYLQSNEEKDRFLKIVTKIFNRINDNLSGGILLEELSKANPYLGNDNTPDGDFIINDASAVPIQFSNGSQSILLPNVIIMGAEPDLFETNSSNISLINNYRPSNHGFGSIAIVTFSPEYSFRFNDNSMNEFIQDPALTLMHELIHSLHGLYGAKGITTKYTITQQQNSLITNIRGINIEEFLTFGGNDLNIITSSQFNDIYTNLLDDYKKIASKLSQVRVSNPQLNPYKDVFQEKYGLDKDASGIYSVNINKFNDIFKKLYSFTEFDLATKFQVKCRETYIGQYKYFQLSNLLNDSIYNISEGYNINNLKVNFRGQNANLNPRIITPITGRGLVKKIIRFCKNIVSVKGIRKSICIEVNNGELFFVASENSYNDDNINTPKEIDDTVTSNNNYENDLDQVILNFNSESAPGLSDEKLNLTIQDDAYIPKYDSNGTSDIEQHDVNELNVFFYLDAQKVPEGENNVNLTSSIDTALLEQPKIYTFFSSEFINNVNKPVQAVLFVSWIQQVLVDFTTEATQKSTVDKIADISIVVPYIGLALNIGNEAQKGNFKDALELLGAGILLEFVPELLIPTILVFTIKSFLGSSDNKNKIIKAINNALKERDEKWKEVYSFIVSNWITKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSTLGESQQELNSMVINTLNNSIPFKLSSYTDDKILISYFNKFFKRIKSSSVLNMRYKNDKYVDTSGYDSNININGEIFIYPTNKNQFSIFNSKPSEVNISQNDYIIYDNKYKNFSISFWVRIPNYDNKIVNVNNEYTIINCMRDNNSGWKVSLNHNEIIWTLQDNAGINQKLAFNYGNSNGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNSIISHRRDLTFSFYNHRYIVNGLYRLYSGIKVKIQRVNDSSTNDQFVRKNDQVYINYIYNNLSYSLYADTNIKDKEKTIKSSLSGNIFNQVVVMNSVGNNCTMNFKNNNGNNIGLLGFKDNTLVASTWYYTHMRDNTNSNGCFWNFISEEHGWQEK

SEQ ID NO: 124 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 125 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVRIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 126 - protein, 1279 aa, Clostridium botulinum
MPVVINSFNYNDPVNDETILYMQKPYEERSRKYYKAFEIMPNVWIMPERDTIGTKPDDFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGDHNSTESFIADPAISLAHELIHALHGLYGAKAVTHKETIEVKRGPLMIAEKPIRLEEFLTFGGEDLNIIPSAMKEKIYNDLLANYEKIATRLREVNTAPPEYDINEYKDYFQWKYGLDRNADGSYTVNRNKFNGIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEKSKVYTFFSSEFIDTINESVNAALFIDWINKVIRDFTTEATQKSTVDKIADISLIVPYVGLALNIVIDAEKGNFQEAFELLGAGILLEFVPELTIPVILVFTIKSYIDSYENKNKAIKAINNALIEREAKWKEIYSWIVSNWLTKINTQFNKRKEQMYQALQNQVDAIKTAIEYKYNNYTSDEKNRLESEYNINNIEEELNKKVSLAMKNIERFMTESSISYLMKLINEAEVGKLKKYDRHVKSDLLDYILYHKLILGDQTKELIDLVTSTLNSSIPFELSSYTNDKILIIYFNRLYKKIKDSSILDMRYENNKFIDISGYGSNISINGNVYIYSTNRNQFGIYSDRLSEVNIAQNNDIIYNSRYQNFSISFWVRIPKHYGPMNRNREYTIINCMGNNNSGWKISLRNIRDCEIIWTLQDTSGNKEKLIFRYEELANISDYINKWIFVTITNNRLGNSRIYINGNLIVEKSISNLGDIHVSDNILFKIVGCDDKTYVGIRYFKVFNTELDKTEIETLYSNEPDPSILKDYWGNYLLYNKKYYLFNLLRKDKYITRNSGILNINQQRGVTEGSVFLNYKLYEGVEVIIRKNGPIDISNTDNFVRKNDLAYINVVYHDVEYRLYADISITKPEKIIKLIRTSNPNDSLGQIIVMDSIGNNCTMNFQNNNGGNIGLLGFHSDNLVASSWYYNNIRRNTSSNGCFWSFISKEHGWQE

SEQ ID NO: 127 - protein, 1277 aa, Clostridium botulinum
MPVVINSFNYDDPVNDDTILYMQIPYEEKSKKYYKAFEIMRNVWIMPERNTIGTNPSDFDPPASLKNGSSAYYDPNYLTTDAEKDRYLKTTIKLFKRINSNPAGEVLLQEISYAKPYLGNDHTPINEFHPVTRTTSVNIKSSTNVESSIILNLLVLGAGPNIFENSSYPVRKLMNSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNDLLANYEKIATRLSEVNSAPPEYDINEYKNYFQWKYGLDKNADGSYTVNENKFNEIYKKLYSFTEIDLANKFKVKCRNTYFIKYGFLKVPNLLDDDIYTVSEGFNIGNLAVNNRGQNINLNPKIIDSIPDKGLVEKIVKLCKSIIPRKGTKAPPRLCIRVNNRELFFVASESSYNENDINTPKEIDDTTNLNNNYRNNLDEVILDYNSETIPQISSQTLNTLVQDDSYVPRYDSNGTSEIEEHNVVDLNAFFYLHAQKVPEGETNISLTSSIDTALSEESKVYTFFSSEFINNINKPVHAALFIGWISQVIRDFTTESTQKSTVDKIADISLIVPYVGLALNIGNDARKGNFKEAFELLGAAILLEVVPELLIPVILVFTIKSFIDSSKNEDKIIKAINNSLIEREAKWKEVYSWIVSNWLTRINTQFNKRKEQMYQALQNQVDAIKTVIEYKYNSYTSDEKNRLESEYNINNIEEELNKKVSLAMKNIERFIAESSISYLMKLINEAKVSELREYDEGVKEYLLDYILKNGSILGDHVQELNDLVTSTLNSSIPFELSSYTNDKILIIYFNKLYKKIKDNCILDMRYENNKFIDISGYGSNISINGELYIYTTNRNQFTIYSGKLSEVNIAQNNDIIYNSRYQNFSISFWVRIPRYSNIVNLNNEYTIINCMGNNNSGWKISLNYNKIIWTLQDTAGNNEKLVFNYTQMISISDYINKWIFVTITNNRLGNSRIYINGNLIDQKSISNLGDIHVSDNILFKIVGCNDTRYVGIRYFKVFDTELDKTEIETLYSDEPDPSILKDFWGNYLLYNKRYYLLNLLRKDNAITQSSTFLSISRARGVDRKANIFSNKRLYKGVEVIIRKNEPIDISNTDNFVRKGDLAYINVVDRDVEYRLYANTSNAQPEKTIKLIRTSNSNDSLDQIIVMDSIGNNCTMNFQNNNGGNIGLLGFHSNTLVASSWYYNNIRRNTSSNGCFWSFISKEHGWQE

SEQ ID NO: 128 - protein, 1277 aa, Clostridium botulinum
MPVEINSFNYDDLVNDNTILYIRPPYYERSNTYFKAFNIMENVWIIPERYRLGIEASKFDPPDSLKAGSDGYFDPNYLSTNTEKNRYLQIMIKLFKRINSNEAGKILLNQIKDAIPYLGNSYTAEDQFTTNNRTISFNVRLANGTIEQEMANLIIWGPGPDLTTNRTGGTTYTPAQSLEAIPYKEGFGSIMTIEFSPEYATAFNDISLTSHAPSLFIKDPALILMHELIHVLHGLYGTYTTGFKIKPNITEPYMEVTKPITSGEFLTFGGNDVNKIPQLIQSQLRSKVLDDYEKIASRLNKVNRATAEINIDKFKYSYQLKYQFVKDSNGVYSVDLDKFNKLYDKIYSFTEFNLAHEFKIKTRNSYLAKNFGPFYLPNLLDNSIYNEADGFNIGDLSVNYKGQVIGSDIDSIKKLEGQGVVSRVVRLCLNSSFKKNTKKPLCITVNNGDLFFIASEDSYGEDTINTPKEIDDTTTLVPSFKNILDKVILDFNKQVTPQIPNRRIRTDIQEDNYIPEYDSNGTSEIEEYNVVDLNAFFYLHAQKVPEGETNISLTSSIDTALSEESKVYTFFSSEFIDTINEPVNAALFIDWISKVIRDFTTEATQKSTVDKIADISLIVPYVGLALNIVNETEKGNFKEAFELLGAGILLEFVPELAIPVILVFTIKSYIDSYENKNKIIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFITESSISYLMKLINEAEVGKLKEYDKRVKRHLLEYIFDYRLILGEQGGELIDLVTSTLNTSIPFELSSYTNDKILIIYFNRLYKKIKDSSILDMRYENNKFIDISGYGSNISINGNVYIYSTNRNQFGIYDDRLSEVNIAQNNDIIYNSRYQNFSISFWVRIPKHYRPMNHNREYTIINCMGNNNSGWKISLRTTGDCEIIWTLQDTSGNKKKLIFRYSQLGGISDYINKWIFVTITNNRLGNSRIYINGNLIVEKSISNLGDIHVSDNILFKIVGCDDKMYVGIRYFKVFNTELDKTEIEILYSNEPDPSILKDYWGNYLLYNKKYYLLNLLRNDKYITRNSDILNISHQRGVTKDLFIFSNYKLYEGVEVIIRKNGPIDISNTDNFVRKNDLAYINVVDHGVEYRLYADISITKPEKIIKLIRRSNPDDSLGQIIVMDSIGNNCTMNFQNNNGGNIGLLGFHSDNLVASSWYYNNIRRNTSSNGCFWSFISKEHGWQE

SEQ ID NO: 129 - protein, 1274 aa, Clostridium botulinum
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 130 - protein, 1268 aa, Clostridium botulinum
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 131 - protein, 1297 aa, Clostridium botulinum
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 132 - protein, 1288 aa, Clostridium botulinum
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSL

SEQ ID NO: 133 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 134 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 135 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 136 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 137 - protein, 5 aa, unidentified
  source 1..5: note = motif unit repeated n times (n>0)
GGGGS

SEQ ID NO: 138 - protein, 5 aa, unidentified
GGGGS

SEQ ID NO: 139 - protein, 10 aa, unidentified
GGGGSGGGGS

SEQ ID NO: 140 - protein, 15 aa, unidentified
GGGGSGGGGSGGGGS

SEQ ID NO: 141 - protein, 20 aa, unidentified
GGGGSGGGGSGGGGSGGGGS

SEQ ID NO: 142 - protein, 25 aa, unidentified
GGGGSGGGGSGGGGSGGGGSGGGGS

SEQ ID NO: 143-147 - skipped (5 entries)

SEQ ID NO: 148 - protein, 7 aa, unidentified
ENLYFQG

SEQ ID NO: 149 - protein, 8 aa, unidentified
LEVLFQGP

SEQ ID NO: 150 - protein, 864 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAIDGRSLYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKN

SEQ ID NO: 151 - protein, 847 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKN

SEQ ID NO: 152 - protein, 863 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIDGRSLYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNE

SEQ ID NO: 153 - protein, 846 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKIIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNE

SEQ ID NO: 154 - DNA, 3897 bp, synthetic construct
  source 1..3897: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgtcataaagccattccgccgtttggtcgtagcgcaggttataacaaaaccctggattgtattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 155 - protein, 1299 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAIPPFGRSAGYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 156 - DNA, 3888 bp, synthetic construct
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgtcataaagccattagcggtcgtagcgcatataacaaaaccctggattgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 157 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAISGRSAYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 158 - DNA, 3888 bp, synthetic construct
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgtcataaagccctgacaccgcgtggtgttcgtctgaaaaccctggattgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacattctgaacaatattatcctgaacctgcgttacaaagacaacaatctgatcgatctgagcggctatggtgcaaaagttgaagtctacgacggtgtcgaactgaacgataaaaaccagttcaaactgacctcatcggctaactcaaaaattcgtgtgacgcagaaccaaaacatcatcttcaactcggtctttctggacttcagcgtgtctttctggattcgcatcccgaaatataaaaatgatggcatccagaactacatccataacgaatacaccatcatcaactgtatgaaaaacaacagtggttggaaaatttccatccgtggcaaccgcattatctggaccctgattgatatcaatggtaaaacgaaaagcgtgtttttcgaatacaacatccgtgaagatatctctgaatacatcaatcgctggtttttcgtgaccattacgaacaatctgaacaatgcgaaaatctatatcaacggcaaactggaaagtaataccgacatcaaagatattcgtgaagttatcgccaacggtgaaatcatcttcaaactggatggcgacatcgatcgcacccagttcatttggatgaaatacttctccatcttcaacacggaactgagtcagtccaatatcgaagaacgctacaaaatccaatcatactcggaatacctgaaagatttctggggtaacccgctgatgtacaacaaagaatactacatgttcaacgcgggcaacaaaaactcatacatcaaactgaaaaaagattcgccggtgggtgaaatcctgacccgtagcaaatacaaccagaactctaaatacatcaactatcgcgatctgtacattggcgaaaaatttattatccgtcgcaaaagcaactctcagagtattaatgatgacatcgtgcgtaaagaagactacatctatctggatttctttaatctgaaccaagaatggcgcgtttatacctacaaatacttcaaaaaagaagaaatgaaactgttcctggccccgatttacgacagcgatgaattttacaacaccatccagatcaaagaatacgatgaacagccgacgtatagttgccaactgctgttcaaaaaagacgaagaatccaccgatgaaattggcctgattggtatccaccgtttctatgaaagcggtatcgttttcgaagaatacaaagattacttctgtatctctaaatggtatctgaaagaagtcaaacgcaaaccgtacaacctgaaactgggctgcaactggcaatttatcccgaaagacgaaggctggaccgaa

SEQ ID NO: 159 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKALTPRGVRLKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 160 - DNA, 2589 bp, synthetic construct
  source 1..2589: mol_type = other DNA
atgaagctggaaatcaacaagttcaactataacgatccgatcgatggcattaacgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcaccaactggtttatgttacccataatctgtatggcattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccctgacaccgcgtggtgttcgtctgaaaaccctggattgcattgaggtggaaaacaaagacctgtttctgatcagcaataaagatagcctgaacgatattaacctgagcgaggaaaaaatcaaacctgaaaccaccgtgttcttcaaggataaactgcctccgcaggatattaccctgtccaattatgattttaccgaagccaatagcatcccgagcattagccagcaaaatattctggaacgtaacgaggaactgtatgaaccgattcgtaatagcctgtttgagatcaaaaccatctatgtggacaaactgaccacctttcattttctggaagcgcagaatattgatgagagcatcgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaacgggcaaaatcgacgtgattgataaaagcagcgataccctggcaattgttccgtatattggtccgctgctgaatattggtaatgatattcgtcatggcgattttgtgggtgcaattgaactggcaggcattacagccctgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaatcctgctgaacaaaagtgttgaacaggccatgaaaaacaccgagaagtttatgattaaactgagcaacagctacctgaccaaagaaatgattccgaaagttcaggacaacctgaaaaactttgatctggaaaccaaaaagaccctggacaagttcatcaaagagaaagaagatattctgggcaccaatctgagcagcagcctgcgtcgtaaagttagcattcgtctgaataagaacattgccttcgatatcaacgatatcccgtttagcgaattcgatgatctgatcaaccagtacaaaaatgaa

SEQ ID NO: 161 - protein, 863 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKALTPRGVRLKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNE

SEQ ID NO: 162 - DNA, 2589 bp, synthetic construct
  source 1..2589: mol_type = other DNA
atgaagctggaaatcaacaagttcaactataacgatccgatcgatggcattaacgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcaccaactggtttatgttacccataatctgtatggcattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccattagcggtcgtagcgcatataacaaaaccctggattgcattgaggtggaaaacaaagacctgtttctgatcagcaataaagatagcctgaacgatattaacctgagcgaggaaaaaatcaaacctgaaaccaccgtgttcttcaaggataaactgcctccgcaggatattaccctgtccaattatgattttaccgaagccaatagcatcccgagcattagccagcaaaatattctggaacgtaacgaggaactgtatgaaccgattcgtaatagcctgtttgagatcaaaaccatctatgtggacaaactgaccacctttcattttctggaagcgcagaatattgatgagagcatcgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaacgggcaaaatcgacgtgattgataaaagcagcgataccctggcaattgttccgtatattggtccgctgctgaatattggtaatgatattcgtcatggcgattttgtgggtgcaattgaactggcaggcattacagccctgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaatcctgctgaacaaaagtgttgaacaggccatgaaaaacaccgagaagtttatgattaaactgagcaacagctacctgaccaaagaaatgattccgaaagttcaggacaacctgaaaaactttgatctggaaaccaaaaagaccctggacaagttcatcaaagagaaagaagatattctgggcaccaatctgagcagcagcctgcgtcgtaaagttagcattcgtctgaataagaacattgccttcgatatcaacgatatcccgtttagcgaattcgatgatctgatcaaccagtacaaaaatgaa

SEQ ID NO: 163 - protein, 863 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAISGRSAYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNE

SEQ ID NO: 164 - DNA, 2598 bp, synthetic construct
  source 1..2598: mol_type = other DNA
atgaagctggaaatcaacaagttcaactataacgatccgatcgatggcattaacgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcaccaactggtttatgttacccataatctgtatggcattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccattccgccgtttggtcgtagcgcaggttataacaaaaccctggattgtattgaggtggaaaacaaagacctgtttctgatcagcaataaagatagcctgaacgatattaacctgagcgaggaaaaaatcaaacctgaaaccaccgtgttcttcaaggataaactgcctccgcaggatattaccctgtccaattatgattttaccgaagccaatagcatcccgagcattagccagcaaaatattctggaacgtaacgaggaactgtatgaaccgattcgtaatagcctgtttgagatcaaaaccatctatgtggacaaactgaccacctttcattttctggaagcgcagaatattgatgagagcatcgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaacgggcaaaatcgacgtgattgataaaagcagcgataccctggcaattgttccgtatattggtccgctgctgaatattggtaatgatattcgtcatggcgattttgtgggtgcaattgaactggcaggcattacagccctgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaatcctgctgaacaaaagtgttgaacaggccatgaaaaacaccgagaagtttatgattaaactgagcaacagctacctgaccaaagaaatgattccgaaagttcaggacaacctgaaaaactttgatctggaaaccaaaaagaccctggacaagttcatcaaagagaaagaagatattctgggcaccaatctgagcagcagcctgcgtcgtaaagttagcattcgtctgaataagaacattgccttcgatatcaacgatatcccgtttagcgaattcgatgatctgatcaaccagtacaaaaatgaa

SEQ ID NO: 165 - protein, 866 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHQLVYVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICHKAIPPFGRSAGYNKTLDCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNE

SEQ ID NO: 166 - protein, 864 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKALTPRGVRLKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKN

SEQ ID NO: 167 - protein, 867 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAIPPFGRSAGYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKN

SEQ ID NO: 168 - protein, 864 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCHKAISGRSAYNKTLDCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKN

SEQ ID NO: 169-172 - skipped (4 entries)

SEQ ID NO: 173 - protein, 7 aa, unidentified
  VAR_SEQ 2: note = X is G, P, A or L
  VAR_SEQ 3: note = X is S, G or F
PXXGRSA

SEQ ID NO: 174 - protein, 4 aa, unidentified
GRSA

SEQ ID NO: 175 - DNA, 2589 bp, synthetic construct
  source 1..2589: mol_type = other DNA
atgaagctggaaatcaacaagttcaactataacgatccgatcgatggcattaacgttattaccatgcgtccgcctcgtcatagcgataaaatcaataaaggtaaaggtccgttcaaagcctttcaggtgattaaaaacatttggattgtgccggaacgctacaactttaccaataataccaacgatctgaacattccgagcgaaccgattatggaagcagatgccatttataacccgaactatctgaataccccgagcgaaaaagatgaatttctgcagggtgttatcaaagtgctggaacgcattaaaagcaaaccggaaggtgaaaaactgctggaactgattagcagcagcattccgctgccgctggttagcaatggtgcactgaccctgagcgataatgaaaccattgcatatcaagagaacaacaacattgtgagcaatctgcaggcaaacctggttatttatggtccgggtcctgatattgcaaataatgcaacctatggtctgtatagcaccccgattagtaatggtgaaggtacactgagcgaagttagctttagcccgttttatctgaaaccgtttgatgaaagctatggcaattatcgtagcctggtgaatatcgtgaacaaattcgtgaaacgtgaatttgcacctgatccggcaagcaccctgatgcacgagctggttcatgttacccataatctgtatggcattagcaaccgcaacttctactataactttgacaccggcaaaattgaaaccagccgtcagcagaatagcctgatttttgaagaactgctgacctttggtggcattgatagcaaagcaattagcagcctgatcatcaagaaaattatcgaaaccgccaagaacaactataccacgctgattagcgaacgcctgaataccgttaccgttgaaaatgatctgctgaaatatatcaaaaacaaaatcccggttcagggtcgtctgggtaactttaaactggataccgcagaattcgagaaaaagctgaataccattctgtttgtgctgaacgaaagcaatctggcacagcgttttagcattctggttcgtaaacattacctgaaagaacgtccgattgatccgatttatgtgaacattctggatgacaatagctacagcaccctggaaggttttaacattagcagtcagggtagcaatgattttcagggccagctgctggaaagcagctattttgaaaaaattgaatccaatgcgctgcgtgcctttatcaaaatttgtcataaagccattgatggtcgcagcctgtataacaaaaccctggattgtattgaggtggaaaacaaagacctgtttctgatcagcaataaagatagcctgaacgatattaacctgagcgaggaaaaaatcaaacctgaaaccaccgtgttcttcaaggataaactgcctccgcaggatattaccctgtccaattatgattttaccgaagccaatagcatcccgagcattagccagcaaaatattctggaacgtaacgaggaactgtatgaaccgattcgtaatagcctgtttgagatcaaaaccatctatgtggacaaactgaccacctttcattttctggaagcgcagaatattgatgagagcatcgatagcagcaaaattcgtgttgaactgaccgatagcgttgatgaagcactgagcaatccgaataaagtttatagcccgttcaagaacatgagcaacaccattaatagcattgaaaccggtattaccagcacctacatcttttatcagtggctgcgtagcatcgtgaaagattttagtgatgaaacgggcaaaatcgacgtgattgataaaagcagcgataccctggcaattgttccgtatattggtccgctgctgaatattggtaatgatattcgtcatggcgattttgtgggtgcaattgaactggcaggcattacagccctgctggaatatgttccggaatttaccattccgattctggttggtctggaagttattggtggcgaactggcacgtgaacaggttgaagcaattgttaataatgccctggataaacgcgatcagaaatgggcagaagtttacaatattaccaaagcacagtggtggggcaccattcatttacagattaatacccgtctggcccatacctataaagccctgagccgtcaggcaaatgccattaaaatgaatatggaatttcagctggccaactacaaaggcaacattgatgataaagccaagatcaaaaacgccatcagcgaaaccgaaatcctgctgaacaaaagtgttgaacaggccatgaaaaacaccgagaagtttatgattaaactgagcaacagctacctgaccaaagaaatgattccgaaagttcaggacaacctgaaaaactttgatctggaaaccaaaaagaccctggacaagttcatcaaagagaaagaagatattctgggcaccaatctgagcagcagcctgcgtcgtaaagttagcattcgtctgaataagaacattgccttcgatatcaacgatatcccgtttagcgaattcgatgatctgatcaaccagtacaaaaatgaa

SEQ ID NO: 176 - DNA, 48 bp, synthetic construct
  source 1..48: mol_type = other DNA
tgtcataaagccattgatggtcgcagcctgtataacaaaaccctggat

(19) United States
(12) Patent Application Publication
(10) Pub. No.: US 2026/0317748 A1
(43) Pub. Date: Oct. 15, 2026

(54) Clostridial Neurotoxins Comprising an Activating Exogenous Protease Cleavage Site

(71) Applicant: Ipsen Biopharm Limited, Wrexham (GB)

(72) Inventors: Kevin MOORE, Wrexham (GB); Kin CHAN, Wrexham (GB)

(21) Appl. No.: 19/114,728

(22) PCT Filed: Sep. 28, 2023
(86) PCT No.: PCT/GB2023/052512

§ 371 Date: Mar. 24, 2025

(30) Foreign Application Priority Data
Sep. 28, 2022 (GB) 2214232.7

(51) Int. Cl. C12N 9/52; A61K 8/66; A61K 38/00; A61Q 19/00

(52) U.S. Cl. CPC C12N 9/52; A61K 8/66; A61Q 19/00; C12Y 304/24069; A61K 38/00

(57) ABSTRACT

The present invention relates to clostridial neurotoxins engineered to comprise an exogenous protease cleavage site within a modified activation loop, wherein cleavage at said site produces an active di-chain clostridial neurotoxin. The invention also relates to methods for manufacturing the same, as well as related pharmaceutical compositions, nucleotide 10 sequences, and therapeutic and cosmetic uses. The invention further relates to a method for proteolytically processing said single-chain clostridial neurotoxins into a corresponding di-chain clostridial neurotoxin.

### CROSS-REFERENCE TO RELATED APPLICATIONS

This Application is a U.S. national stage filing of International Application No. PCT/GB2023/052512, filed Sep. 28, 2023, which claims the benefit of and priority to European Application No. GB2214232.7, filed Sep. 28, 2022, the contents of each which are incorporated by reference herein in their entireties.

### REFERENCE TO SEQUENCE LISTING

The instant application contains a Sequence Listing that has been submitted electronically in .xml format and is hereby incorporated by reference in its entirety. The .xml copy was created on May 20, 2026, is named “IPS-030US1_P71907WO_seqlist” and is 239,368 bytes in size.

### FIELD OF THE INVENTION

The present invention relates to clostridial neurotoxins engineered to comprise an exogenous protease cleavage site within a modified activation loop, wherein cleavage at said site produces an active di-chain clostridial neurotoxin. The invention also relates to methods for manufacturing the same, as well as related pharmaceutical compositions, nucleotide sequences, and therapeutic and cosmetic uses. The invention further relates to a method for proteolytically processing said single-chain clostridial neurotoxins into a corresponding di-chain clostridial neurotoxin.

### BACKGROUND OF THE INVENTION

Bacteria in the genus Clostridia produce highly potent and specific protein toxins, which can poison neurons and other cells to which they are delivered. Examples of such clostridial neurotoxins include the neurotoxins produced by C. tetani(TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

Clostridial neurotoxins are expressed as single-chain polypeptides in Clostridium. Each clostridial neurotoxin has a catalytic light chain separated from the heavy chain (encompassing the N-terminal translocation domain and the C-terminal receptor binding domain) by an exposed region called the activation loop. During protein maturation proteolytic cleavage of the activation loop separates the light and heavy chain of the clostridial neurotoxin, which are held together by a disulphide bridge, to create fully active di-chain toxin.

This activation process must be reproduced during standard production of recombinant toxin production. Exogenous proteases such as trypsin or Lys-C with well-defined cleavage motifs are used for proteolytically activating single-chain clostridial neurotoxins in conventional production methods. However, for some clostridial neurotoxins, incubation with Lys-C or trypsin results in partial or improper cleavage of the single-chain polypeptide resulting in the production of contaminating single-chain and/or inactive cleavage/degradation products (e.g. in the case of BoNT/E). For instance, for Botulinum neurotoxin serotype X (BoNT/X, see WO 2018/009903 A2), activation is problematic, with cleavage using trypsin or Lys-C completely degrading the polypeptide. Thus, at present there is no universal exogenous protease for activation of clostridial neurotoxins. This is particularly problematic upon identification of a new clostridial neurotoxin or production of a modified (e.g. chimeric or hybrid) neurotoxin, which requires screening of multiple proteases to determine correct activation. For re-targeted clostridial neurotoxins, some standard proteases used for activation can also cleave within the exogenous targeting moieties, resulting in incorrectly processed proteins with reduced targeting to the desired cell type. To avoid such off-target cleavage, either alternative targeting moieties must be identified (which may not always be possible), or the targeting moieties must be designed to remove the cleavage site for the standard protease, which may negatively impact the structure of the targeting moiety, and/or add to design and production costs.

Furthermore, in vitro activation of clostridial neurotoxins is associated with numerous disadvantages. There is a cost associated with the use of an exogenous protease (particularly GMP-grade protease), and its removal following activation of the clostridial neurotoxin.

Dependence on a single or limited number of suppliers for GMP-grade protease can also create weakness in the supply/production chain. Purification of the activated clostridial neurotoxin from the activating exogenous protease can also affect production efficiency and yield. In addition, production of active di-chain clostridial neurotoxins according to conventional production methods necessitates strict safety and control procedures, also adding to production costs and time. Strict safety precautions are also required for practitioners working with active di-chain clostridial neurotoxins.

The present invention overcomes one or more of the above-mentioned problems.

### SUMMARY OF THE INVENTION

The present inventors have previously demonstrated that insertion of a furin cleavage site into the activation loop of a clostridial neurotoxin allows for the in vivo activation of clostridial neurotoxins (see PCT Application No. PCT/GB2022/050756; which is herein incorporated by reference in its entirety), and have also demonstrated that endosomal proteases can be used to cleave clostridial neurotoxins, opening up a new paradigm allowing for both in vivo activation of clostridial neurotoxins, and also the provision of alternative means for in vitro activation. The present inventors have also previously shown that the BoNT/C1 activation loop can be used as a universal activation loop for clostridial neurotoxins (see WO2020/065336; which is herein incorporated by reference in its entirety). However, the use of the BoNT/C1 activation loop can in some circumstances be undesirable. In particular, the native BoNT/C1 activation loop sequence is sensitive to cleavage by Factor Xa, Lys-C, trypsin and enterokinase. Some clostridial neurotoxins are sensitive to off-target cleavage by one or more of these enzymes. Further, there can be costs and difficulties associated with sourcing these proteases at a GMP-compliant grade. Therefore, providing an expanded tool kit of engineered clostridial neurotoxins and activating proteases would offer further benefits in this technical space. In particular, it would be advantageous to provide means to activate clostridial neurotoxins use proteases which are not associated with issues such as (i) sourcing GMP-compliant proteases; (ii) proteases that can work with desirable re-targeted clostridial neurotoxin architectures; (iii) reducing non-specific cleavage and hence undesirable truncation, which requires additional purification and/or impacts on yield; and/or (iv) increasing utility across clostridial neurotoxin serotypes.

The present inventors have now surprisingly demonstrated that specific cleavage sites for other exogenous proteases, particularly thrombin, tissue plasminogen activator (t-PA) and urokinase (u-PA) can be introduced into a modified BoNT/C1 activation loop, and that this modified BoNT/C1 activation loop can be used to engineer clostridial neurotoxins, which may then be cleaved and activated by these proteases. Thus, the introduction of one or more exogenous protease cleavage site into a modified BoNT/C activation loop does not disrupt the functioning of the modified BoNT/C activation loop, and that a modified BoNT/C activation loop may still be used as a universal activation loop for clostridial neurotoxins. Furthermore, the present inventors have even more surprisingly shown that clostridial neurotoxins engineered in this manner can be activated by such proteases with improved cleavage and specificity and the potential for reduced off-target protease activity, with the potential to provide improved rates of activation, reduced production of truncation products and allowing lower concentrations of activating protease to be used. Further, such exogenous cleavage sites can potentially be introduced whilst advantageously maintaining the clostridial activation loop architecture.

In addition, the exogenous protease-activated engineered clostridial neurotoxins of the invention offer several potential benefits compared with conventionally activated clostridial neurotoxins, such as improving yield and/or reducing manufacturing burden/costs.

Accordingly, the invention provides an engineered clostridial neurotoxin, wherein the endogenous activation loop is replaced by a modified BoNT/C activation loop, in which the endogenous activation site of the BoNT/C activation loop, or part thereof, has been replaced by an exogenous protease cleavage site, wherein cleavage at said cleavage site results in the production of an (active) di-chain form of the engineered clostridial neurotoxin.

The exogenous protease cleavage site is a cleavage site specific for an exogenous proteases may be selected from: (a) thrombin; (b) tissue plasminogen activator (t-PA); and/or (c) urokinase (u-PA); wherein optionally: (i) the exogenous protease is a human protease; (ii) the exogenous protease is comprised in a drug product; and/or (iii) the exogenous protease is a recombinant human protease.

The exogenous protease cleavage site may comprise or consist of: (a) a thrombin consensus sequence of SEQ ID NO: 169 or 170; and/or (b) a uPA and/or t-PA consensus sequence of SEQ ID NOs: 172 or 173. The exogenous protease cleavage site may comprise or consist of one or more of LTPRGVRL (SEQ ID NO: 15), LVPRGS (SEQ ID NO: 16), ENKSLVPRGS (SEQ ID NO: 17), SGRSA (SEQ ID NO: 25), PPFGRSAG (SEQ ID NO: 33), PGSGRSAG (SEQ ID NO: 26), PGSGRSASGTTGTG (SEQ ID NO: 27), KRV (SEQ ID NO: 24), GRI (SEQ ID NO: 44) or PGRVVGG (SEQ ID NO: 50). The exogenous protease cleavage site may be between 3 and 10 amino acids in length, preferably between 3 and 8 amino acids in length. The exogenous protease cleavage site may consist of LTPRGVRL (SEQ ID NO: 15), LVPRGS (SEQ ID NO: 16), ENKSLVPRGS (SEQ ID NO: 17), SGRSA (SEQ ID NO: 25), PPFGRSAG (SEQ ID NO: 33), PGSGRSAG (SEQ ID NO: 26), PGSGRSASGTTGTG (SEQ ID NO: 27), KRV (SEQ ID NO: 24), GRI (SEQ ID NO: 44) or PGRVVGG (SEQ ID NO: 50).

The exogenous protease cleavage site may comprise or consist of LTPRGVRL (SEQ ID NO: 15), LVPRGS (SEQ ID NO: 16) or ENKSLVPRGS (SEQ ID NO: 17), preferably LTPRGVRL (SEQ ID NO: 15), and the exogenous protease cleavage site may be specific for thrombin, wherein optionally the exogenous protease cleavage site consists of LTPRGVRL (SEQ ID NO: 15), LVPRGS (SEQ ID NO: 16) or ENKSLVPRGS (SEQ ID NO: 17), preferably LTPRGVRL (SEQ ID NO: 15).

The exogenous protease cleavage site may comprise or consist of KRV (SEQ ID NO: 24), SGRSA (SEQ ID NO: 25), PPFGRSAG (SEQ ID NO: 33), PGSGRSAG (SEQ ID NO: 26) or PGSGRSASGTTGTG (SEQ ID NO: 27) and the exogenous protease cleavage site may be specific for t-PA and/or u-PA, wherein optionally the exogenous protease cleavage site consists of KRV (SEQ ID NO: 24), SGRSA (SEQ ID NO: 25), PPFGRSAG (SEQ ID NO: 33), PGSGRSAG (SEQ ID NO: 26) or PGSGRSASGTTGTG (SEQ ID NO: 27).

The endogenous neurotoxin activation loop may be one or more selected from SEQ ID NO: 67 to 90.

The exogenous protease cleavage site within the modified BoNT/C activation loop may be the only cleavage site for the exogenous protease within the engineered clostridial neurotoxin; no off-site cleavage may occur on contacting the engineered clostridial neurotoxin with the exogenous protease; and/or no light chain truncation of the engineered clostridial neurotoxin may occur on contacting the engineered clostridial neurotoxin with the exogenous protease.

The clostridial neurotoxin may be (a) a Botulinum Neurotoxin (BoNT) serotype A, serotype B, serotype C, serotype D, serotype E, serotype F, serotype G or serotype X, or a Tetanus Neurotoxin (TeNT); or (b) a chimeric BoNT or a hybrid BoNT. The engineered clostridial neurotoxin may be a re-targeted clostridial neurotoxin in which an endogenous H_{C} or H_{CC} of a clostridial neurotoxin is replaced by an exogenous targeting moiety (TM). In particularly preferred embodiments the clostridial neurotoxin is BoNT/X.

The engineered clostridial neurotoxin may be a single-chain clostridial neurotoxin which (a) (i) is encoded by a nucleotide sequence having at least 70% sequence identity to SEQ ID NO: 154, 156, 158, 160, 162 or 164; or (ii) is encoded by a nucleotide sequence having at least 70% sequence identity to SEQ ID NO: 175, wherein SEQ ID NO: 176 within SEQ ID NO: 175 is replaced by a nucleotide sequence encoding at least one exogenous protease consensus sequence, cleavage site or a modified BoNT/C activation loop as defined in any one of claims 3 to 9; and/or (b) comprises a polypeptide sequence having at least 70% sequence identity to one or more of SEQ ID NOs:155, 157, 159, 161 163, 165, 166, 167 or 168; and/or (c) comprises a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as defined in any one of claims 3 to 9, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as defined in any one of claims 3 to 9, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

The invention also provides a method for proteolytically processing an engineered clostridial neurotoxin of the invention into a corresponding di-chain clostridial neurotoxin, the method comprising contacting the engineered clostridial neurotoxin with an exogenous protease specific for the exogenous protease cleavage site, thereby producing a di-chain clostridial neurotoxin. The invention further provides a di-chain clostridial neurotoxin obtainable said method, wherein optionally (a) the C-terminus of the clostridial neurotoxin light chain ends with the amino acid sequence LVPR, preferably ALVPR, and the N-terminus of the clostridial neurotoxin heavy chain begins with the amino acid sequence GSK or GSY; (b) the C-terminus of the clostridial neurotoxin light chain ends with the amino acid sequence SLVPR, and the N-terminus of the clostridial neurotoxin heavy chain begins with the amino acid sequence GSY; (c) the C-terminus of the clostridial neurotoxin light chain ends with the amino acid sequence LTPR, preferably ALTPR, and the N-terminus of the clostridial neurotoxin heavy chain begins with the amino acid sequence GVR, preferably GVRL; (d) the C-terminus of the clostridial neurotoxin light chain ends with the amino acid sequence PGR, and the N-terminus of the clostridial neurotoxin heavy chain begins with the amino acid sequence VVG; (e) the C-terminus of the clostridial neurotoxin light chain ends with the amino acid sequence DKR, preferably AIDKR, and the N-terminus of the clostridial neurotoxin heavy chain begins with the amino acid sequence VLY; (f) the C-terminus of the clostridial neurotoxin light chain ends with the amino acid sequence DKR, preferably AIDK, and the N-terminus of the clostridial neurotoxin heavy chain begins with the amino acid sequence RVLY; (g) the C-terminus of the clostridial neurotoxin light chain ends with the amino acid sequence SGR, preferably PGSGR, and the N-terminus of the clostridial neurotoxin heavy chain begins with the amino acid sequence SA, preferably SAY, SAG or SAS; (h) the C-terminus of the cleaved engineered clostridial neurotoxin light chain may end with the amino acid sequence SGR, preferably PGSGR, and the N-terminus of the cleaved engineered clostridial neurotoxin heavy chain may begin with the amino acid sequence TL, preferably TLD or TLDC; or (i) the C-terminus of the clostridial neurotoxin light chain ends with the amino acid sequence FGR, and the N-terminus of the clostridial neurotoxin heavy chain begins with the amino acid sequence SA, preferably SAG.

The invention also provides a polynucleotide encoding an engineered clostridial neurotoxin of the invention.

The invention further provides an expression vector comprising a polynucleotide of the invention, which is operably linked to a promoter.

Said polynucleotide or expression vector may: (a) comprise a nucleotide sequence having (i) at least 70% sequence identity to SEQ ID NO: 154, 156, 158, 160, 162 or 164; or (ii) at least 70% sequence identity to SEQ ID NO: 175, wherein SEQ ID NO: 176 within SEQ ID NO: 175 is replaced by a nucleotide sequence encoding at least one exogenous protease consensus sequence, cleavage site or a modified BoNT/C activation loop as defined in any one of claims 3 to 9; (b) encode a polypeptide sequence having at least 70% sequence identity to one or more of SEQ ID NOs: 155, 157, 159, 161 163, 165, 166, 167 or 168; and/or (c) encode a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as defined in any one of claims 3 to 9, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as defined in any one of claims 3 to 9, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

The invention also provides a method of producing an engineered clostridial neurotoxin of the invention comprising the step of expressing a polynucleotide of the invention or an expression vector of the invention in a cell, and recovering the expressed engineered clostridial neurotoxin. Said method may further comprise a step of introducing a polynucleotide of the invention or an expression vector of the invention into the cell.

The invention further provides a cell expressing an engineered clostridial neurotoxin of the invention. Said cell may comprise a polynucleotide of the invention, or an expression vector of the invention.

The invention also provides a pharmaceutical composition comprising an engineered clostridial neurotoxin of the invention, or a di-chain clostridial neurotoxin of the invention, and a pharmaceutically acceptable carrier, excipient, diluent, adjuvant, propellant and/or salt.

The invention further provides an engineered clostridial neurotoxin of the invention, a di-chain clostridial neurotoxin of the invention, or a pharmaceutical composition of the invention, for use in a method of preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder. Preferably, a composition of the invention may be used in the prevention or treatment of a disease or condition selected from: limb spasticity (upper or lower); cervical dystonia; headache disorders (preferably migraine); blepharospasm; hemifacial spasm; and lower urinary tract disorders (e.g. bladder pain syndrome (preferably interstitial cystitis); overactive bladder; and detrusor overactivity (e.g. neurogenic detrusor overactivity.

The invention also provides the use of an engineered clostridial neurotoxin of the invention, a di-chain clostridial neurotoxin of the invention, or a pharmaceutical composition of the invention, in the manufacture of a medicament for preventing or treating a disease or disorder for which a therapy with a botulinum neurotoxin is indicated, wherein optionally said disease or disorder is selected from a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder. Preferably, a composition of the invention may be used in the prevention or treatment of a disease or condition selected from: limb spasticity (upper or lower); cervical dystonia; headache disorders (preferably migraine); blepharospasm; hemifacial spasm; and lower urinary tract disorders (e.g. bladder pain syndrome (preferably interstitial cystitis); overactive bladder; and detrusor overactivity (e.g. neurogenic detrusor overactivity.

In such therapies, the clostridial neurotoxin may be administered to a subject in single-chain form. The clostridial neurotoxin or pharmaceutical composition may be substantially free of a di-chain form of the clostridial neurotoxin. The clostridial neurotoxin or pharmaceutical composition may comprise less than 400 pg di-chain clostridial neurotoxin per 100 ng single-chain clostridial neurotoxin, or less than 300 pg di-chain clostridial neurotoxin per 100 ng single-chain clostridial neurotoxin, or less than 200 pg di-chain clostridial neurotoxin per 100 ng single-chain clostridial neurotoxin, or less than 100 pg di-chain clostridial neurotoxin per 100 ng single-chain clostridial neurotoxin, or less than 50 pg di-chain clostridial neurotoxin per 100 ng single-chain clostridial neurotoxin.

Alternatively in such therapies, the clostridial neurotoxin may be administered to a subject in di-chain form. The clostridial neurotoxin or pharmaceutical composition may be substantially free of a single-chain form of the clostridial neurotoxin. The clostridial neurotoxin or pharmaceutical composition may comprise less than 400 pg single-chain clostridial neurotoxin per 100 ng di-chain clostridial neurotoxin, or less than 300 pg single-chain clostridial neurotoxin per 100 ng di-chain clostridial neurotoxin, or less than 200 pg single-chain clostridial neurotoxin per 100 ng di-chain clostridial neurotoxin, or less than 100 pg single-chain clostridial neurotoxin per 100 ng di-chain clostridial neurotoxin, or less than 50 pg single-chain clostridial neurotoxin per 100 ng di-chain clostridial neurotoxin.

The invention also provides a cosmetic composition comprising an engineered clostridial neurotoxin of the invention, or a di-chain clostridial neurotoxin of the invention, and a cosmetically acceptable carrier, excipient, diluent, adjuvant, propellant and/or salt.

The invention further provides the use of a cosmetic composition of the invention, for preventing or alleviating a cosmetic indication for which the application of a botulinum neurotoxin is indicated.

In such cosmetic indications, the clostridial neurotoxin may be for administration to a subject in single-chain form. The clostridial neurotoxin or cosmetic composition maybe substantially free of a di-chain form of the clostridial neurotoxin. The clostridial neurotoxin or cosmetic composition may comprise less than 400 pg di-chain clostridial neurotoxin per 100 ng single-chain clostridial neurotoxin, or less than 300 pg di-chain clostridial neurotoxin per 100 ng single-chain clostridial neurotoxin, or less than 200 pg di-chain clostridial neurotoxin per 100 ng single-chain clostridial neurotoxin, or less than 100 pg di-chain clostridial neurotoxin per 100 ng single-chain clostridial neurotoxin, or less than 50 pg di-chain clostridial neurotoxin per 100 ng single-chain clostridial neurotoxin.

Alternatively in such cosmetic indications, the clostridial neurotoxin may be for administration to a subject in single-chain form. The clostridial neurotoxin or cosmetic composition may be substantially free of a single-chain form of the clostridial neurotoxin. The clostridial neurotoxin or cosmetic composition may comprise less than 400 pg single-chain clostridial neurotoxin per 100 ng di-chain clostridial neurotoxin, or less than 300 pg single-chain clostridial neurotoxin per 100 ng di-chain clostridial neurotoxin, or less than 200 pg single-chain clostridial neurotoxin per 100 ng di-chain clostridial neurotoxin, or less than 100 pg single-chain clostridial neurotoxin per 100 ng di-chain clostridial neurotoxin, or less than 50 pg single-chain clostridial neurotoxin per 100 ng di-chain clostridial neurotoxin.

The invention also provides a method for proteolytically processing a single-chain clostridial neurotoxin into a corresponding di-chain clostridial neurotoxin, the method comprising: (a) providing a single-chain clostridial neurotoxin; and (b) contacting the single-chain clostridial neurotoxin with an exogenous protease; wherein the single-chain clostridial neurotoxin has exogenous protease cleavage site and/or modified BoNT/C activation loop as defined herein, and wherein the exogenous protease hydrolyses a peptide bond of the exogenous protease cleavage site and/or modified BoNT/C activation loop, thereby producing a di-chain clostridial neurotoxin, wherein optionally the exogenous protease is selected from thrombin, t-PA, u-PA, FIX and FVIIa. The single-chain clostridial neurotoxin may be: (a) an engineered clostridial neurotoxin of the invention; (b) encoded by a nucleic acid sequence comprising or consisting of a nucleotide sequence having at least 70% sequence identity to SEQ ID NO: 154, 156, 158, 160, 162 or 164; or (ii) at least 70% sequence identity to SEQ ID NO: 175, wherein SEQ ID NO: 176 within SEQ ID NO: 175 is replaced by a nucleotide sequence encoding at least one exogenous protease consensus sequence, cleavage site or a modified BoNT/C activation loop of the invention; (c) comprise or consist of a polypeptide sequence having at least 70% sequence identity to one or more of SEQ ID NOs: 155, 157, 159, 161 163, 165, 166, 167 or 168; and/or (d) comprise or consist of a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop of the invention, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop of the invention, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

### BRIEF DESCRIPTION OF THE DRAWINGS

FIG. 1: SDS PAGE gels (+/−DTT) A4952 cleavage by different concentrations of u-PA over time.

FIG. 2: SDS PAGE gels (+/−DTT) A4953 cleavage by different concentrations of FIX over time.

FIG. 3: A SDS PAGE gels (+/−DTT) A4954 cleavage by different concentrations of thrombin over time. B Mass spectrogram of A4954 cleaved by thrombin. C SDS PAGE gel showing thrombin can cleave the PGR cleavage site without truncation.

FIG. 4: A SDS PAGE gel (−/+DTT) showing cleavage of engineered BoNT with an ENKSLVPRGS modified C-loop with thrombin or engineered BoNT with an SGRSA modified C-loop with u-PA or 2 t-PA variants (full-length (t-PAi) and a functional fragment (t-PA_{frag})) after 2 hours. Lane 1=ENKSLVPRGS loop negative control; Lane 2=SGRSA loop negative control; Lane 3=ENKSLVPRGS loop with 0.39 μg thrombin; Lane 4=ENKSLVPRGS loop with 1.95 μg thrombin; Lane 5=ENKSLVPRGS loop with 3.90 μg thrombin; Lane 6=SGRSA loop with 0.39 μg u-PA; Lane 7=SGRSA loop with 1.95 μg u-PA; Lane 8=SGRSA loop with 3.90 μg u-PA; Lane 9=SGRSA loop with 0.39 μg t-PAi; Lane 10=SGRSA loop with 1.95 μg t-PA_{fl}; Lane 11=SGRSA loop with 3.90 μg t-PA_{fl}; Lane 12=SGRSA loop with 0.39 μg t-PA_{frag}; Lane 13=SGRSA loop with 1.95 μg t-PA_{frag}; Lane 14=SGRSA loop with 3.90 μg t-PA_{frag}. B Mass spectrogram of A5045 cleaved by u-PA.

FIG. 5: A SDS PAGE gels (−/+DTT) showing cleavage of engineered BoNT with an LTPRGVRL modified C-loop with increasing concentrations of thrombin after 2 hours (top) or 20 hours (bottom). Possible truncation products indicated with black arrow. B SDS PAGE gels (−/+DTT) showing cleavage of engineered BoNT with a PGSGRSAG modified C-loop with increasing concentrations of u-PA, t-PA_{fl} or t-PA_{frag} after 2 hours (left), 4 hours (middle) or 20 hours (right). Lanes 1-9 are engineered BoNT with a PGSGRSAG modified C-loop, lane 10 is a comparator lane with engineered BoNT with a SGRSA modified C-loop. Possible truncation products indicated with black arrow. C SDS PAGE gels (−/+DTT) showing cleavage of engineered BoNT with a PGSGRSASGTTGTG modified C-loop with increasing concentrations of u-PA, t-PA_{frag} or t-PA_{fl} after 2 hours (left), 4 hours (middle) or 20 hours (right). Possible truncation products indicated with black arrow. D SDS PAGE gels (−/+DTT) showing cleavage of engineered BoNT with a PPFGRSAG modified C-loop with increasing concentrations of u-PA, t-PA_{frag} or t-PA_{fl} after 2 hours (left), 4 hours (middle) or 20 hours (right). Reteplase is visible in some gels (black arrow). E SDS PAGE gels (−/+DTT) showing cleavage of engineered BoNT with a PPFGRSAG modified C-loop with increasing concentrations of t-PAt_{frag} or t-PA_{fl} after 2 hours (left), 4 hours (middle) or 20 hours (right). t-PA_{frag} (black arrow) and t-PA_{fl} (dotted arrow) are visible in some gels.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides.

As used herein, the term “spacer” refers to a flexible peptide used in an exogenous activation loop or modified BoNT/C activation loop, or with an exogenous protease cleavage site which typically is included to preserve the secondary structure of the exogenous/modified BoNT/C activation loop within an engineered clostridial neurotoxin of the invention. A spacer for use in an engineered clostridial neurotoxin of the invention may comprise an amino acid sequence of from 1 to 30 amino acid residues, e.g. from 5 to 30 amino acid residues, from 10 to 25 amino acid residues or about 5 to about 20 amino acid residues. A spacer may comprise or consist of small amino acid residues, such as glycine, threonine, arginine, serine, asparagine, glutamine, alanine, aspartic acid, proline, glutamic acid, lysine, leucine and/or valine, particularly glycine, serine, alanine, leucine and/or valine. Spacers comprising or consisting of glycine, serine and/or alanine may be preferred, with glycine and serine being particularly preferred. Accordingly, the most commonly used spacers have sequences consisting primarily of stretches of Gly and Ser residues (“GS” linker), which comprise a sequence of (Gly-Gly-Gly-Gly-Ser)n (SEQ ID NO: 137). Non-limiting examples of GS linkers include GS5 or (GGGGS)₁ (SEQ ID NO: 138); GS10 or (GGGGS)₂ (SEQ ID NO: 139); GS15 or (GGGGS)₃ (SEQ ID NO: 140); GS20 or (GGGGS)₄ (SEQ ID NO: 141); and GS25 or(GGGGS)₅ (SEQ ID NO: 142).

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more.

As used herein, “reduction” or “inhibition” encompasses a complete inhibition or reduction as compared to a reference level. “Complete inhibition” is a 100% inhibition (i.e. abrogation) as compared to a reference level.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues. Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto.

### Engineered Clostridial Neurotoxins

The present invention provides an engineered clostridial neurotoxin, comprising a exogenous protease cleavage site. Typically cleavage at said exogenous protease cleavage site results in the production of a di-chain form of the engineered clostridial neurotoxin. In other words, cleavage at the exogenous protease cleavage site results in activation of an engineered clostridial neurotoxin.

Typically according to the invention, an engineered clostridial neurotoxin lacks the endogenous activation loop (or a part thereof) of the pre-engineering clostridial neurotoxin from which the engineered neurotoxin is derived. The term “endogenous activation loop” is as described herein. In these engineered clostridial neurotoxins of the invention, the endogenous activation loop (or a part thereof) of the pre-engineering clostridial neurotoxin is replaced by a modified BoNT/C activation loop, particularly a modified BoNT/C1 activation loop.

The endogenous BoNT/C1 activation loop has a consensus sequence of Cyst-(Xaa)ₐ-Ile-Asp/Glu-Gly-Arg-(Yaa)_{b}-Cys (SEQ ID NO: 1), wherein a=1-10 and b=4-15, typically wherein a=2-4 and/or b=6-10, preferably where a=3 and/or b=8. Specific BoNT/C1 activation loops comprise or consist of CHKAIDGRSLYNKTLDC (SEQ ID NO: 2) or CHKAIEGRSLYNKTLDC (SEQ ID NO: 3). Within the endogenous BoNT/C1 activation loop, enterokinase and Factor Xa are both capable of cleaving immediately C-terminal to the IDGR or IEGR sequence, and the presence of lysine and arginine residues allows cleavage by trypsin and Lys-C.

In a modified BoNT/C activation loop of the invention, one or more of these endogenous activation sites within the endogenous activation loop have been replaced by one or more exogenous protease cleavage site. Typically a modified BoNT/C activation loop of the invention lacks a Factor Xa/enterokinase cleavage site, such that the modified BoNT/C activation loop is not sensitive to cleavage by Factor Xa and/or enterokinase. Thus, a modified BoNT/C (BoNT/C1) activation loop of the invention may lack an IDGR or IEGR sequence.

Further, a modified BoNT/C (BoNT/C1) activation loop of the invention typically comprises one or more exogenous protease cleavage site. In other words, a modified BoNT/C (BoNT/C1) activation loop may have the endogenous BoNT/C activation site, or part thereof, replaced by one or more exogenous protease cleavage site, as described herein.

Thus, the invention provides an engineered clostridial neurotoxin, in which the endogenous activation loop is replaced by a modified BoNT/C (BoNT/C1) activation loop, in which the endogenous activation site of the BoNT/C (BoNT/C1) activation loop, or part thereof, has been replaced by one or more exogenous protease cleavage site, and wherein cleavage at said cleavage site results in the production of a di-chain form of the engineered clostridial neurotoxin.

A modified BoNT/C (BoNT/C1) activation loop according to the invention may begin with the N-terminal residues CHKA (SEQ ID NO: 4), such as CHKAI (SEQ ID NO: 5) or CHKAID (SEQ ID NO: 6). Typically, such modified BoNT/C (BoNT/C1) activation loops lack an IDGR (SEQ ID NO: 7) or IEGR (SEQ ID NO: 8) sequence, such that the sequence of the modified BoNT/C (BoNT/C1) activation loop does not comprise the sequence CHKAIDGR (SEQ ID NO: 9) or CHKAIEGR (SEQ ID NO: 10) at the N-terminus. Alternatively or additionally, modified BoNT/C (BoNT/C1) activation loop according to the invention may end with the C-terminal residues TLDC (SEQ ID NO: 11), such as KTLDC (SEQ ID NO: 12), YNKTLDC (SEQ ID NO: 13) or LYNKTLDC (SEQ ID NO: 14). A modified BoNT/C (BoNT/C1) activation loop according to the invention may be between about 10 to about 30 (e.g. 10, 11, 12, 13, 14, 15, 16, 17, 18, 19, 20, 21, 22, 23, 24, 25, 26, 27, 28, 29 or 30) amino acids in length, such as between about 15 to about 25 (e.g. 15, 16, 17, 18, 19, 20, 21, 22, 23, 24 or 25) amino acids in length, preferably between about 17 to about 24 (e.g. 17, 18, 19, 20, 21, 22, 23 or 24) amino acids in length. Particularly preferred are modified BoNT/C (BoNT/C1) activation loops which are 17 amino acids in length, as such modified BoNT/C (BoNT/C1) activation loops are the same length as the endogenous BoNT/C (BoNT/C1) activation loop.

A modified BoNT/C (BoNT/C1) activation loop may comprise one or more exogenous protease cleavage site of LTPRGVRL (SEQ ID NO: 15), ENKSLVPRGS (SEQ ID NO: 17), LVPRGS (SEQ ID NO: 16), KRV (SEQ ID NO: 24), SGRSA (SEQ ID NO: 25), PGSGRSAG (SEQ ID NO: 26), PGSGRSASGTTGTG (SEQ ID NO: 27), PPFGRSAG (SEQ ID NO: 33), VVPRVELVA (SEQ ID NO: 32), or GRI (SEQ ID NO: 44) into the endogenous activation loop of the clostridial neurotoxin, wherein cleavage at said cleavage site results in the production of a di-chain form of the engineered clostridial neurotoxin. In particular, a modified BoNT/C (BoNT/C1) activation loop may comprise one or more exogenous protease cleavage site of LTPRGVRL (SEQ ID NO: 15), KRV (SEQ ID NO: 24), SGRSA (SEQ ID NO: 25), VVPRVELVA (SEQ ID NO: 32), PPFGRSAG (SEQ ID NO: 33) or GRI (SEQ ID NO: 44), preferably LTPRGVRL (SEQ ID NO: 15), KRV (SEQ ID NO: 24), SGRSA (SEQ ID NO: 25) or PPFGRSAG (SEQ ID NO: 33). A modified BoNT/C (BoNT/C1) activation loop may comprise one or more exogenous protease cleavage site of LTPRGVRL (SEQ ID NO: 15), ENKSLVPRGS (SEQ ID NO: 17), SGRSA (SEQ ID NO: 25), PGSGRSAG (SEQ ID NO: 26), PGSGRSASGTTGTG (SEQ ID NO: 27), or PPFGRSAG (SEQ ID NO: 33).

The invention also provides an engineered clostridial neurotoxin, comprising an exogenous protease cleavage site formed by introducing one or more amino acid sequence comprising or consisting of LTPRGVRL (SEQ ID NO: 15), ENKSLVPRGS (SEQ ID NO: 17), LVPRGS (SEQ ID NO: 16), KRV (SEQ ID NO: 24), SGRSA (SEQ ID NO: 25), PGSGRSAG (SEQ ID NO: 26), PGSGRSASGTTGTG (SEQ ID NO: 27), PPFGRSAG (SEQ ID NO: 33), VVPRVELVA (SEQ ID NO: 32), or GRI (SEQ ID NO: 44) into the endogenous activation loop of the clostridial neurotoxin, wherein cleavage at said cleavage site results in the production of a di-chain form of the engineered clostridial neurotoxin. In particular, the one or more amino acid sequence may comprise or consist of LTPRGVRL (SEQ ID NO: 15), KRV (SEQ ID NO: 24), SGRSA (SEQ ID NO: 25), VVPRVELVA (SEQ ID NO: 32), PPFGRSAG (SEQ ID NO: 33) or GRI (SEQ ID NO: 44), preferably LTPRGVRL (SEQ ID NO: 15), KRV (SEQ ID NO: 24), SGRSA (SEQ ID NO: 25) or PPFGRSAG (SEQ ID NO: 33). The invention provides an engineered clostridial neurotoxin, comprising an exogenous protease cleavage site formed by introducing one or more amino acid sequence comprising or consisting of LTPRGVRL (SEQ ID NO: 15), ENKSLVPRGS (SEQ ID NO: 17), SGRSA (SEQ ID NO: 25), PGSGRSAG (SEQ ID NO: 26), PGSGRSASGTTGTG (SEQ ID NO: 27), or PPFGRSAG (SEQ ID NO: 33) into the endogenous activation loop of the clostridial neurotoxin, wherein cleavage at said cleavage site results in the production of a di-chain form of the engineered clostridial neurotoxin. The endogenous (native) activation loop of a clostridial neurotoxin may be replaced (or partially replaced) by said exogenous protease cleavage site. Thus, an engineered clostridial neurotoxin of the invention may comprise an exogenous activation loop.

The term “exogenous protease cleavage site” may be used interchangeably with the term “exogenous protease activation site”. An exogenous activation loop as defined herein will typically comprise or consist of one or more exogenous protease cleavage site.

The engineered clostridial neurotoxins of the invention, whether comprising one or more exogenous protease cleavage site within a modified BoNT/C (BoNT/C1) activation loop or within an exogenous activation loop, may be activated in vivo. Thus, the engineered clostridial neurotoxins open up a new field of processing and therapeutic use for clostridial neurotoxins, enabling toxins to be produced and administered as single-chain clostridial neurotoxins, which are then cleaved to produce the active di-chain form in vivo.

The engineered clostridial neurotoxins of the invention, whether comprising one or more exogenous protease cleavage site within a modified BoNT/C (BoNT/C1) activation loop or within an exogenous activation loop, may be activated in vitro. The introduction of an exogenous protease cleavage site, whether comprising one or more exogenous protease cleavage site within a modified BoNT/C (BoNT/C1) activation loop or within an exogenous activation loop, into an engineered clostridial neurotoxin of the invention typically provides advantages in the manufacturing and/or processing of engineered clostridial neurotoxins of the invention, such as (i) sourcing GMP-compliant proteases; (ii) proteases that can work with desirable re-targeted clostridial neurotoxin architectures; (iii) reducing non-specific cleavage and hence undesirable truncation, which requires additional purification and/or impacts on yield; (iv) increasing utility across clostridial neurotoxin serotypes, or any combination thereof, as described herein; and/or (v) improving the overall rate of activation, which has the potential to enable the production of activated di-chain compositions, reducing the amount of protease required and/or reducing the complexity/number of steps during purification.

The clostridial neurotoxin (pre-engineering) is typically characterised in that the endogenous activation loop is inefficiently proteolytically processed by one or more exogenous protease. In contrast to the clostridial neurotoxin (pre-engineering), an engineered clostridial neurotoxin of the invention is not inefficiently proteolytically processed by the one or more exogenous protease for which a cleavage site has been introduced and/or a peptide bond outside of the exogenous activation loop of the engineered clostridial neurotoxin is not hydrolysed by said one or more exogenous protease. Thus, the clostridial neurotoxin (pre-engineering) is typically resistant to proteolytic processing by one or more exogenous protease. The terms “inefficiently proteolytically processed by one or more exogenous protease”, “resistant to proteolytic processing by one or more exogenous protease”, “not substantially hydrolysed by one or more exogenous protease” “inefficiently activated by one or more exogenous protease”, “resistant to activation by one or more exogenous protease” and “not substantially activated by one or more exogenous protease” are used interchangeably herein. Typically, the clostridial neurotoxin (pre-engineering) is typically resistant to proteolytic processing by the one or more exogenous protease for which cleavage site(s) have been introduced according to the present invention. The clostridial neurotoxin (pre-engineering) may further be resistant to proteolytic processing by one or more exogenous protease for which cleavage site(s) have not been introduced according to the invention.

A clostridial neurotoxin (pre-engineering) is typically one in which a peptide bond (either within or outside of the activation loop) is not, or is not substantially, hydrolysed by one or more exogenous protease. The term “not substantially hydrolysed” means that less than 10%, 5%, 4%, 3%, 2% or 1% of the clostridial neurotoxin present in a reaction contains a peptide bond that has been hydrolysed by one or more exogenous protease in a method of the invention.

Accordingly, a clostridial neurotoxin (pre-engineering) typically does not contain one or more exogenous protease consensus sequence (e.g. any one or more of SEQ ID NOs: 169, 170, 172 or 173) and/or one or more exogenous protease cleavage site (e.g. as defined herein, such as any one of SEQ ID NOs: 15-22, 24-30, 32-42, 44-46 and/or 50-52, particularly any one of SEQ ID NOs: 15-22, 24-30 and/or 32-42, preferably any one of SEQ ID NOs: 15, 17, 16, 26, 25, 27, 24, 33 and/or 32) within its endogenous activation loop.

A clostridial neurotoxin (pre-engineering) may not contain one or more exogenous protease consensus sequence (e.g. any one or more of SEQ ID NOs: 169, 170, 172 or 173) and/or one or more exogenous protease cleavage site (e.g. as defined herein, such as any one of SEQ ID NOs: 15-22, 24-30, 32-42, 44-46 and/or 50-52, particularly any one of SEQ ID NOs: 15-22, 24-30 and/or 32-42, preferably any one of SEQ ID NOs: 15, 17, 16, 25, 26, 27, 24, 33 and/or 32, for an exogenous protease that is intended for use in activating an engineered clostridial neurotoxin. By way of non-limiting example, if an engineered clostridial neurotoxin contains a thrombin cleavage site, then the corresponding pre-engineering clostridial neurotoxin may not comprise a thrombin cleavage site within its endogenous activation loop.

The invention may comprise replacing an endogenous activation loop (or part thereof) of any clostridial neurotoxin with a modified BoNT/C activation loop (or part thereof) comprising one or more exogenous protease cleavage site as described herein. The invention may comprise replacing an endogenous activation loop (or part thereof) of any clostridial neurotoxin with one or more exogenous protease cleavage site or an exogenous activation loop comprising one or more exogenous protease cleavage site as described herein. The clostridial neurotoxin may be a botulinum neurotoxin (BoNT) or a tetanus neurotoxin (TeNT).

Preferably the clostridial neurotoxin is a botulinum neurotoxin, such as BoNT/A, BoNT/B, BoNT/C₁, BoNT/D, BoNT/E, BoNT/F, BoNT/G or BoNT/X, or a chimeric or hybrid thereof. In some particularly preferred embodiments the clostridial neurotoxin is BoNT/X or a chimera or hybrid thereof.

The term “endogenous activation loop” as used herein means an activation loop present in a subject clostridial neurotoxin, e.g. a subject clostridial neurotoxin of the indicated serotype. For example, BoNT/A1 includes a BoNT/A1 heavy chain and light chain, thus the endogenous activation loop of BoNT/A1 is an A1 activation loop. For clostridial neurotoxin chimeras or hybrids, the person skilled in the art can identify the “endogenous activation loop”, for example by determining the serotype(s) from which the L-chain and H_{N} domain are derived.

In some embodiments, a chimera or hybrid clostridial neurotoxin may have an endogenous activation loop that is a fusion of an activation loop from two different serotypes. By way of example, a chimeric clostridial neurotoxin such as BoNT/A1C₁ has a BoNT/A₁ light chain and translocation domain, thus the endogenous BoNT/AlCl activation loop is an A1 activation loop. The endogenous activation loop, is typically bounded by cysteine residues that form a disulphide bridge and covalently link the light and heavy chains of a (pre-engineering) clostridial neurotoxin. Thus, an endogenous activation loop sequence may be recited including the bounding cysteine residues (as described herein), or without the bounding cysteine residues. One of ordinary skill in the art would understand that the definitions may be used interchangeably, and would readily be able to identify an endogenous activation loop, either including or excluding the bounding cysteine residues.

Typically an “endogenous activation loop” is any activation loop that is does not comprise or consist of one or more exogenous protease consensus sequence (e.g. any one or more of SEQ ID NOs: 169, 170, 172 or 173) and/or one or more exogenous protease cleavage sites as described herein (e.g. SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 25, 26, 27, 24, 33 and/or 32, more preferably, SEQ ID NOs: 15, 17, 16, 26, 25 and/or 33).

By contrast, an “exogenous activation loop” as used herein means an activation loop that is different to the endogenous activation loop present in a subject clostridial neurotoxin, e.g., a subject clostridial neurotoxin of the indicated serotype, and wherein the exogenous activation loop comprises one or more exogenous protease cleavage site. For example, a BoNT/A activation loop has a different polypeptide sequence to a wild-type BoNT/X activation loop, therefore the BoNT/A activation loop is exogenous to BoNT/X. By way of further example, a BoNT/C1 activation loop has a different polypeptide sequence to a wild-type BoNT/A1 activation loop, therefore the BoNT/C1 activation loop is exogenous to BoNT/A1.

Thus, the term “exogenous activation loop” as used herein may refer to a modified BoNT/C (BoNT/C1) activation loop of the invention unless expressly stated otherwise. For clostridial neurotoxin chimeras or hybrids, the person skilled in the art can determine whether an activation loop is an “exogenous activation loop”, for example by determining the serotype(s) from which the L-chain and H_{N} domain are derived. For example, where the L-chain is a BoNT/B L-chain and the H_{N} domain is from BoNT/D, the endogenous activation loop may have a portion of a BoNT/B sequence and a portion of a BoNT/D sequence, and if an activation loop (e.g. a C1 activation loop) is different thereto, and comprises one or more exogenous protease cleavage site, it is considered an “exogenous activation loop”.

Determination of whether an activation loop is an “endogenous activation loop” may be made by aligning the sequence of a subject clostridial neurotoxin with the activation loop, and seeing if the activation loop is present in the subject clostridial neurotoxin sequence. If it is present, then the activation loop can be identified as an endogenous activation loop. As described herein, the endogenous activation loop of a clostridial neurotoxin is replaced by an exogenous cleavage site which is one or more exogenous protease cleavage site, by an exogenous activation loop which comprises one or more exogenous protease cleavage site, or by a modified BoNT/C (BoNT/C1) activation loop in which the endogenous BoNT/C (BoNT/C1) activation site (or part thereof) has been replaced by one or more exogenous protease cleavage site.

According to the invention, one or more exogenous protease cleavage site may be inserted between the two cysteine residues that bound the endogenous activation loop of a pre-engineering clostridial neurotoxin, although the precise position of the one or more exogenous protease cleavage site within the endogenous activation loop is not limited, provided that the conformation of the resultant engineered clostridial neurotoxin is not disrupted and/or the engineered clostridial neurotoxin rendered non-functional. In a modified BoNT/C (BoNT/C1) activation loop of the invention, the one or more exogenous protease cleavage site may be inserted between the two cysteine residues that bound the endogenous BoNT/C (BoNT/C1) activation loop.

The entire endogenous activation loop may be replaced by one or more exogenous protease cleavage site or an exogenous activation loop comprising one or more exogenous protease cleavage site as described herein. Alternatively, a part or portion of the endogenous activation loop may be replaced (also referred to herein as partial replacement of the endogenous activation loop), such as at least 5, 10, 15, 20, 25, 30, 35 or 40 amino acid residues of the endogenous activation loop are replaced. Preferably 5 to 20, more preferably 5 to 15 amino acid residues of the endogenous activation loop are replaced. Typically partial replacement involves the replacement of consecutive amino acids within the endogenous activation loop. Thus, in partial replacement of the endogenous activation loop, at least one amino acid residue of the endogenous activation loop is retained. Preferably between about 5 to about 15 (e.g. 5, 6, 7, 8, 9, 10, 11, 12, 13, 14 or 15), such as between about 5 to about 12 (e.g. 5, 6, 7, 8, 9, 10, 11 or 12) of the (e.g. 5, 6, 7, 8, 9, 10, 11, 12, 13, 14 or 15) of the endogenous activation loop is retained. The retained amino acids residues may be at the N-terminal and/or C-terminal of the endogenous activation loop.

The entire (unmodified) BoNT/C (BoNT/C1) activation loop may be replaced by one or more exogenous protease cleavage site as described herein. Preferably, a part or portion of the (unmodified) BoNT/C (BoNT/C1) activation loop may be replaced (also referred to herein as partial replacement of the (unmodified) BoNT/C (BoNT/C1) activation loop), such as at least 5, 10, 11, 12, 13, 14, 15, 16 or 17 amino acid residues of the (unmodified) BoNT/C (BoNT/C1) activation loop are replaced. Preferably 3 to 15, more preferably 5 to 10 amino acid residues of the (unmodified) BoNT/C (BoNT/C1) activation loop are replaced. Typically partial replacement involves the replacement of consecutive amino acids within the (unmodified) BoNT/C (BoNT/C1) activation loop. Thus, in partial replacement of the (unmodified) BoNT/C (BoNT/C1) activation loop, at least one amino acid residue of the (unmodified) BoNT/C (BoNT/C1) activation loop is retained. Preferably between about 5 to about 15 (e.g. 5, 6, 7, 8, 9, 10, 11, 12, 13, 14 or 15), such as between about 5 to about 12 (e.g. 5, 6, 7, 8, 9, 10, 11 or 12) of the (e.g. 5, 6, 7, 8, 9, 10, 11, 12, 13, 14 or 15) of the (unmodified) BoNT/C (BoNT/C1) activation loop is retained. The retained amino acids residues may be at the N-terminal and/or C-terminal of the (unmodified) BoNT/C (BoNT/C1) activation loop.

In some preferred embodiments, the endogenous activation loop is completely replaced in an engineered clostridial neurotoxin of the invention. Typically this means that all the amino acid residues of the activation loop (between the cysteine residues which form the disulphide bond in the active di-chain molecule) are replaced by an exogenous activation loop, modified BoNT/C (BoNT/C1) activation loop or exogenous protease cleavage site according to the invention. Complete replacement of the endogenous activation loop may comprise the introduction of a exogenous activation loop or modified BoNT/C (BoNT/C1) activation loop which consists entirely of one or more exogenous protease cleavage site as described herein.

Alternatively, complete replacement of the endogenous activation loop may comprise the introduction of a exogenous activation loop or modified BoNT/C (BoNT/C1) activation loop which comprises one or more exogenous protease cleavage site as described herein, together with one or more spacer sequence. Each one or more spacer sequence is typically a short peptide (e.g. between about 5 to about 25 amino acids, such as between about 5 to about 20 amino acids, between about 5 to about 15 amino acids, or between about 5 to about 10 amino acids). Such spacers may be present when the one or more exogenous protease cleavage site is a short motif (e.g. typically less than 15, preferably less than 10 or less than 9 amino acids in length). One or more spacer may be present N-terminal and/or C-terminal to each of said exogenous protease cleavage sites. Preferably, a spacer may be a GS spacer as defined herein.

Replacement (including partial replacement) of an endogenous activation loop or an (unmodified) BoNT/C (BoNT/C1) activation loop with one or more exogenous protease cleavage site may be achieved by any method known in the art. For example, replacement might be achieved by way of an amino acid modification. An endogenous activation loop may be replaced by deleting one or more amino acid residues of the endogenous activation loop. An endogenous activation loop may be replaced by substituting one or more amino acid residues of the endogenous activation loop with amino acid residues of an exogenous activation loop. An endogenous activation loop (or a portion thereof) may be deleted, and one or more exogenous protease cleavage site or an exogenous activation loop comprising one or more exogenous protease cleavage site inserted, preferably at the position formally occupied by the endogenous activation loop. Alternatively, the endogenous activation loop may be retained in an engineered clostridial neurotoxin of the invention, and preferably inactivated (e.g. by way of mutation). It is preferred that the endogenous activation loop (a portion thereof or the entire endogenous activation loop) is not present in the engineered clostridial neurotoxin of the invention. It is preferred that the one or more exogenous protease cleavage site or the exogenous activation loop comprising the one or more exogenous protease cleavage site occupies the position in the clostridial neurotoxin formally occupied by the endogenous activation loop. For the avoidance of doubt, when an endogenous activation loop is modified to comprise one or more exogenous protease cleavage site (e.g. by substitution of residues within the endogenous activation loop or by the addition of one or more amino acids to form one or more exogenous protease cleavage site within the endogenous activation loop), the modified activation loop is an exogenous activation loop according to the invention. Therefore, potentially an engineered clostridial neurotoxin can comprise both its endogenous activation/cleavage site and one or more exogenous protease cleavage site, and as such may be activated either by the native activating protease (or equivalents used in conventional recombinant BoNT production, e.g. trypsin or Lys-C), or by one or more exogenous protease.

For example, replacement (including partial replacement) of the unmodified BoNT/C (BoNT/C1) activation loop, and/or replacement (including partial replacement) of the endogenous BoNT/C (BoNT/C) activation site might be achieved by way of an amino acid modification. An unmodified BoNT/C (BoNT/C1) activation loop, and/or endogenous BoNT/C (BoNT/C) activation site may be replaced by deleting one or more amino acid residues of the unmodified BoNT/C (BoNT/C1) activation loop, and/or endogenous BoNT/C (BoNT/C) activation site. An unmodified BoNT/C (BoNT/C1) activation loop, and/or endogenous BoNT/C (BoNT/C) activation site may be replaced by substituting one or more amino acid residues of the unmodified BoNT/C (BoNT/C1) activation loop, and/or endogenous BoNT/C (BoNT/C) activation site with amino acid residues of an exogenous protease cleavage site. An unmodified BoNT/C (BoNT/C1) activation loop, and/or endogenous BoNT/C (BoNT/C) activation site (or a portion thereof) may be deleted, and one or more exogenous protease cleavage site inserted, preferably at the position formally occupied by the unmodified BoNT/C (BoNT/C1) activation loop, and/or endogenous BoNT/C (BoNT/C) activation site. Alternatively, the unmodified BoNT/C (BoNT/C1) activation loop, and/or endogenous BoNT/C (BoNT/C) activation site may be retained in an engineered clostridial neurotoxin of the invention, and preferably inactivated (e.g. by way of mutation). It is preferred that the unmodified BoNT/C (BoNT/C1) activation loop, and/or endogenous BoNT/C (BoNT/C) activation site (or a portion thereof) is not present in the engineered clostridial neurotoxin of the invention. It is preferred that the one or more exogenous protease cleavage site occupies the position in the clostridial neurotoxin formally occupied by the unmodified BoNT/C (BoNT/C1) activation loop, and/or endogenous BoNT/C (BoNT/C) activation site. For the avoidance of doubt, when an unmodified BoNT/C (BoNT/C1) activation loop, and/or endogenous BoNT/C (BoNT/C) activation site is modified to comprise one or more exogenous protease cleavage site (e.g. by substitution of residues within the unmodified BoNT/C (BoNT/C1) activation loop, and/or endogenous BoNT/C (BoNT/C) activation site or by the addition of one or more amino acids to form one or more exogenous protease cleavage site within the BoNT/C (BoNT/C1) activation loop, and/or endogenous BoNT/C (BoNT/C) activation site), the modified BoNT/C (BoNT/C1) activation loop, and/or modified BoNT/C (BoNT/C) activation site is an exogenous activation loop according to the invention. Therefore, potentially an engineered clostridial neurotoxin can comprise both its endogenous activation/cleavage site and one or more exogenous protease cleavage site, and as such may be activated either by the native activating protease (or equivalents used in conventional recombinant BoNT production, e.g. trypsin or Lys-C), or by one or more exogenous protease.

In particular, a BoNT/C (BoNT/C1) activation loop, and/or BoNT/C (BoNT/C) activation site may be modified by (i) substitution of one or more amino acid present within the unmodified BoNT/C (BoNT/C1) activation loop, and/or unmodified/endogenous BoNT/C (BoNT/C) activation site; and/or (ii) insertion of one or more amino acid into the unmodified BoNT/C (BoNT/C1) activation loop, and/or unmodified/endogenous BoNT/C (BoNT/C) activation site. Preferably, a BoNT/C (BoNT/C1) activation loop, and/or BoNT/C (BoNT/C) activation site may be modified by (i) substitution of one or more amino acid present within the unmodified BoNT/C (BoNT/C1) activation loop, and/or unmodified/endogenous BoNT/C (BoNT/C) activation site; or (ii) substitution of one or more amino acid present within the unmodified BoNT/C (BoNT/C1) activation loop, and/or unmodified/endogenous BoNT/C (BoNT/C) activation site and insertion of one or more amino acid into the unmodified BoNT/C (BoNT/C1) activation loop, and/or unmodified/endogenous BoNT/C (BoNT/C) activation site. Between 1-10 (e.g. 1, 2, 3, 4, 5, 6, 7, 8, 9, or 10) amino acids of the unmodified BoNT/C (BoNT/C1) activation loop, and/or unmodified/endogenous BoNT/C (BoNT/C) activation site may be substituted to form a modified BoNT/C (BoNT/C1) activation loop and/or modified BoNT/C (BoNT/C) activation site, such as between about 3-10, 3-9, 5-9, or 5-8 amino acids may be substituted. Alternatively or additionally, between 1-10 (e.g. 1, 2, 3, 4, 5, 6, 7, 8, 9, or 10) amino acids may be inserted into the unmodified BoNT/C (BoNT/C1) activation loop, and/or unmodified/endogenous BoNT/C (BoNT/C) activation site to form a modified BoNT/C (BoNT/C1) activation loop and/or modified BoNT/C (BoNT/C) activation site, such as between about 3-10, 3-9, 4-8, or 4-7 amino acids may be inserted.

Methods for modifying proteins by substitution, insertion or deletion of amino acid residues are known in the art and may be employed in the practice of the present invention. By way of example, amino acid modifications may be introduced by modification of a DNA sequence encoding a clostridial neurotoxin. This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively a modified gene sequence can be chemically synthesised. Any other method known in the art for modifying polypeptides, such as polypeptide synthesis and polypeptide conjugation may also be used to engineer a clostridial neurotoxin according to the invention.

An endogenous activation loop replaced according to the invention may comprise or consist of a polypeptide sequence having at least 70% (e.g. at least 80% or 90%) sequence identity to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 SEQ ID NO: 84, SEQ ID NO: 85, SEQ ID NO: 86, SEQ ID NO: 87, SEQ ID NO: 88, SEQ ID NO: 89 or SEQ ID NO: 90. An endogenous activation loop replaced according to the invention may comprise or consist of a polypeptide sequence having at least 70% (e.g. at least 80% or 90%) sequence identity to SEQ ID NO: 90. In particular, an endogenous activation loop may comprise or consist of a polypeptide sequence having at least 95% sequence identity to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 SEQ ID NO: 84, SEQ ID NO: 85, SEQ ID NO: 86, SEQ ID NO: 87, SEQ ID NO: 88, SEQ ID NO: 89 or SEQ ID NO: 90. An endogenous activation loop replaced according to the invention may comprise or consist of a polypeptide sequence having at least 95% sequence identity to SEQ ID NO: 90. Preferably, an endogenous activation loop comprises or consists of a polypeptide sequence shown as SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83 SEQ ID NO: 84, SEQ ID NO: 85, SEQ ID NO: 86, SEQ ID NO: 87, SEQ ID NO: 88, SEQ ID NO: 89 or SEQ ID NO: 90. An endogenous activation loop replaced according to the invention may comprise or consist of a polypeptide sequence of SEQ ID NO: 90.

An endogenous activation loop replaced according to the invention may comprise or consist of a polypeptide sequence having at least 70% (e.g. at least 80% or 90%) sequence identity to SEQ ID NO: 70, SEQ ID NO: 71, SEQ ID NO: 72, SEQ ID NO: 73, SEQ ID NO: 74 or SEQ ID NO: 90. An endogenous activation loop may comprise or consist of a polypeptide sequence having at least 95% sequence identity to SEQ ID NO: 70, SEQ ID NO: 71, SEQ ID NO: 72, SEQ ID NO: 73, SEQ ID NO: 74, or SEQ ID NO: 90. Preferably, an endogenous activation loop comprises or consists of a polypeptide sequence shown as SEQ ID NO: 70, SEQ ID NO: 71, SEQ ID NO: 72, SEQ ID NO: 73, SEQ ID NO: 74, or SEQ ID NO: 90.

Preferably an endogenous activation loop replaced according to the invention comprises or consists of a polypeptide sequence having at least 70% (e.g. at least 80% or 90%) sequence identity to SEQ ID NO: 72 or SEQ ID NO: 90. An endogenous activation loop may comprise or consist of a polypeptide sequence having at least 95% sequence identity to SEQ ID NO: 72 or SEQ ID NO: 90. More preferably, an endogenous activation loop comprises or consists of a polypeptide sequence shown as SEQ ID NO: 72 or SEQ ID NO: 90.

The present invention encompasses methods and engineered clostridial neurotoxins in which an endogenous activation loop has been replaced by an exogenous cleavage site, which is one or more exogenous protease cleavage site, or an exogenous activation loop which comprises one or more exogenous protease cleavage site, as described herein.

The present invention encompasses methods and engineered clostridial neurotoxins in which an endogenous activation loop has been replaced by a modified BoNT/C (BoNT/C1) activation loop, wherein the endogenous activation site of the BoNT/C (BoNT/C1) activation loop (or part thereof) has been replaced by one or more exogenous protease cleavage site as described herein.

The engineered clostridial neurotoxins of the invention may comprise an exogenous activation loop comprising one or more of any exogenous protease cleavage site as described herein. An exogenous activation loop may be produced by replacing one or more amino acids of an endogenous activation loop of a clostridial neurotoxin, as described herein. In some preferred embodiments, the replaced amino acids of the endogenous activation loop are replaced by one or more exogenous protease cleavage site or exogenous activation loop having the same number of amino acids. In other words, by way of illustration, if five amino acids are replaced in an endogenous activation loop, the replacement one or more exogenous protease cleavage site or exogenous activation loop comprising said one or more exogenous protease cleavage site has five amino acids. If ten amino acids are replaced in an endogenous activation loop, the replacement one or more exogenous protease cleavage site or exogenous activation loop comprising said one or more exogenous protease cleavage site has ten amino acids.

Non-limiting examples of such exogenous activation loops include, SEQ ID NOs: 53-66.

The engineered clostridial neurotoxins of the invention may comprise a modified BoNT/C (BoNT/C1) activation loop comprising one or more of any exogenous protease cleavage site as described herein. A modified BoNT/C (BoNT/C1) activation loop may be produced by replacing one or more amino acids of the unmodified BoNT/C (BoNT/C1) activation loop of a clostridial neurotoxin, as described herein. In some preferred embodiments, the replaced amino acids of the unmodified BoNT/C (BoNT/C1) activation loop are replaced by one or more exogenous protease cleavage site having the same number of amino acids. In other words, by way of illustration, if five amino acids are replaced in an unmodified BoNT/C (BoNT/C1) activation loop, the replacement one or more exogenous protease cleavage site has five amino acids. If ten amino acids are replaced in an unmodified BoNT/C (BoNT/C1) activation loop, the replacement one or more exogenous protease cleavage site has ten amino acids. Non-limiting examples of such modified BoNT/C (BoNT/C1) activation loops are derived herein.

The invention provides a method for manufacturing an engineered clostridial neurotoxin according to the invention, comprising replacing an endogenous activation loop (or part thereof) of a clostridial neurotoxin by an exogenous activation loop or an exogenous cleavage site or a modified BoNT/C (BoNT/C1) activation loop, thereby providing an exogenous clostridial neurotoxin, wherein the exogenous cleavage site is one or more exogenous protease cleavage site as described herein, or the exogenous activation loop or modified BoNT/C (BoNT/C1) activation loop comprises said one or more exogenous protease cleavage site. Typically said one or more exogenous protease cleavage site is selected from a sequence which comprises or consists of the amino acid sequence of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred, or the exogenous activation loop or modified BoNT/C (BoNT/C1) activation loop comprises said one or more exogenous protease cleavage site.

The invention provides an engineered clostridial neurotoxin (e.g. obtainable by a method of the invention), wherein an endogenous activation loop (or part thereof) of a clostridial neurotoxin has been replaced by an exogenous activation loop, an exogenous cleavage site or a modified BoNT/C (BoNT/C1) activation loop, thereby providing an engineered clostridial neurotoxin, wherein the exogenous cleavage site is one or more exogenous protease cleavage site as described herein, or the exogenous activation loop or modified BoNT/C (BoNT/C1) activation loop comprises said one or more exogenous protease cleavage site. Typically said one or more exogenous protease cleavage site is selected from a sequence which comprises or consists of the amino acid sequence of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred, or the exogenous activation loop comprises said one or more exogenous protease cleavage site.

A clostridial neurotoxin of the present invention (e.g. engineered clostridial neurotoxin) may be encoded by a nucleic acid comprising or consisting of a nucleotide sequence having at least 70% sequence identity to any one of SEQ ID NOs: 154, 156, or 158. A clostridial neurotoxin of the present invention may be encoded by a nucleic acid comprising or consisting of a nucleotide sequence having at least 80% or 90% sequence identity to any one of SEQ ID NOs: 154, 156, or 158. Preferably, a clostridial neurotoxin of the present invention may be encoded by a nucleic acid comprising or consisting of a nucleotide sequence comprising (more preferably consisting of) any one of SEQ ID NOs: 154, 156, or 158.

A clostridial neurotoxin of the present invention (e.g. engineered clostridial neurotoxin) may comprise or consist of a polypeptide sequence having at least 70% sequence identity to one or more of SEQ ID NOs: 155, 157, 159, 166, 167 or 168. A clostridial neurotoxin of the present invention may comprise or consist of a polypeptide sequence having at least 80% or 90% sequence identity to one or more of SEQ ID NOs: 155, 157, 159, 166, 167 or 168.

Preferably, a clostridial neurotoxin of the present invention may comprise or consist of (more preferably consist of) a polypeptide sequence shown as any one of SEQ ID NOs: 155, 157, 159, 166, 167 or 168.

The clostridial neurotoxin of the present invention (e.g. engineered clostridial neurotoxin) is preferably BoNT/X, wherein the clostridial neurotoxin is encoded by a nucleic acid comprising or consisting of a nucleotide sequence having at least 70% sequence identity to SEQ ID NO: 160, 162 or 164. The clostridial neurotoxin may be encoded by a nucleic acid comprising or consisting of a nucleotide sequence having at least 80% or 90% sequence identity to SEQ ID NO: 160, 162 or 164. Preferably the clostridial neurotoxin is encoded by a nucleic acid comprising or consisting of a nucleotide sequence comprising (or consisting of) SEQ ID NO: 160, 162 or 164. The clostridial neurotoxin of the present invention is preferably BoNT/X, wherein the clostridial neurotoxin comprises or consists of a polypeptide sequence having at least 70% sequence identity to SEQ ID NO: 161, 163, 165, 166, 167 or 168. The clostridial neurotoxin may comprise or consist of a polypeptide sequence having at least 80% or 90% sequence identity to SEQ ID NO: 161, 163, 165, 166, 167 or 168. Preferably the clostridial neurotoxin comprises (or consists of) a polypeptide sequence shown as SEQ ID NO: 161, 163, 165, 166, 167 or 168.

The engineered clostridial neurotoxin may be a single-chain clostridial neurotoxin encoded by a nucleic acid comprising or consisting of a nucleotide sequence having at least 70% sequence identity to SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin encoded by a nucleic acid comprising or consisting of a nucleotide sequence having at least 80% or 90% sequence identity to SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin encoded by a nucleic acid comprising or consisting of the nucleotide sequence of SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

Alternatively or in addition, the engineered clostridial neurotoxin may be a single-chain clostridial neurotoxin comprising or consisting of a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin comprising or consisting of a polypeptide sequence having (i) at least 80% or 90% sequence identity to SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 80% or 90% identity to SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin comprising or consisting of (i) the polypeptide sequence of SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) the polypeptide sequence of SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

The polypeptide sequences of the invention (or the nucleotide sequences encoding the same) may include a purification tag, such as a His-tag. It is intended that the present invention also encompasses polypeptide sequences (and nucleotide sequences encoding the same) where the purification tag is removed.

### Exogenous Proteases and Exogenous Protease Cleavage Sites

An exogenous protease according to the invention is a protease which (i) is not responsible for cleaving native clostridial holotoxins (i.e. is not a clostridial hydrolase); and (ii) is typically not used in conventional manufacturing of clostridial neurotoxins (e.g. trypsin and/or Lys-C). The term “exogenous” refers to the relationship between the endogenous clostridial activation loop and the protease. As such, a protease which is present with a patient to be treated may be considered an exogenous protease in the context of activating an engineered clostridial neurotoxin of the invention, although said protease could be described as endogenous to the patient to be treated.

Accordingly, non-limiting examples of exogenous protease according to the present invention may be selected from thrombin; tissue plasminogen activator (t-PA); urokinase (u-PA); Factor IX (FIX); Factor VIIa (FVIIa) and/or plasminogen. These proteases have a variety of different functions, and are typically involved in the formation or degradation of blood clots in vivo. Preferably the exogenous protease is selected from thrombin, t-PA and/or u-PA, with thrombin being particularly preferred. The exogenous protease is typically a human protease, such as a recombinant human protease. Alternatively or in addition, the exogenous protease may be comprised in a drug product.

In other words, engineered clostridial neurotoxins of the invention typically comprises at least one cleavage site for one or more of thrombin, t-PA, u-PA, FIX, FVIIa and/or plasminogen. Preferably an engineered clostridial neurotoxin of the invention comprises at least one cleavage site for one or more of thrombin, t-PA and/or u-PA, with at least one cleavage site for thrombin being particularly preferred.

Thrombin is a serine protease which hydrolyses peptide bonds following arginine or lysine residues, including Xaa₁-Xaa₂-Pro/Ala/Leu/Gly-Arg//Ser/Ala/Gly-Xaa₃-Xaa₄-Xaa₅, (SEQ ID NO: 18) where Xaa₁₋₅ may each be independently selected from any amino acid, and wherein “//” indicates the position of the hydrolysed peptide bond. The term “thrombin” encompasses thrombin as described herein, as well as any protease having structural and/or functional similarity (preferably structural and functional similarity) that is capable of hydrolysing a peptide bond of the consensus Xaa₁-Xaa₂-Pro/Ala/Leu/Gly-Arg//Ser/Ala/Gly-Xaa₃-Xaa₄-Xaa₅. A suitable thrombin is human thrombin, which has UniProt Accession No. P00734 (version 2 of the sequence, deposited 1 Jan. 1990, accessed 4 Sep. 2022), herein SEQ ID NO: 23. This sequence is a propeptide, which is converted to the mature human form by a process involving cleavage of an N-terminal polypeptide of residues 1-271 to form prethrombin 2 (P2), which is then further cleaved at Arg320 to yield thrombin. Human thrombin is commercially available from Merck (#69671).

Tissue plasminogen activator (t-PA or tPA) is a serine protease, cleaving in a range of consensus sequences, including (i) cleaving after the arginine residue in the consensus Arg-Val; (ii) Xaa₁-Phe/Tyr-Ser/Gly/Ala-Arg//Xaa₂-Xaa₃-Xaa₄-Xaa₅, (SEQ ID NO: 34) where Xaa₁₋₅ may each be independently selected from any amino acid, and wherein “//” indicates the position of the hydrolysed peptide bond; and/or (iii) Gly-Pro-Xaa₁-Lys/Arg//Xaa₂-Gly-Gly-Xaa₃ (SEQ ID NO: 35), where Xaa₁₋₃ may each be independently selected from any amino acid, and wherein “//” indicates the position of the hydrolysed peptide bond. The term “t-PA” encompasses t-PA described herein, as well as any protease having structural and/or functional similarity (preferably structural and functional similarity) that is capable of hydrolysing a peptide bond of the consensus Arg-Val, Xaa₁-Phe/Tyr-Ser/Gly/Ala-Arg//Xaa₁-Xaa₂-Xaa₃-Xaa₄, or Gly-Pro-Xaa₁-Lys/Arg//Xaa₂-Gly-Gly-Xaa₃. A suitable t-PA is human t-PA, which has UniProt Accession No. P00750 (version 1 of the sequence, deposited 21 Jul. 1986, accessed 11 Sep. 2022), herein SEQ ID NO: 43. This sequence is a propeptide, which is converted to the mature human form by a process involving cleavage of an N-terminal polypeptide of residues 1-35. Human t-PA is commercially available from Merck (#T0831).

Urokinase (also referred to as urokinase plasminogen activator, u-PA or uPA) is a serine protease, cleaving in a range of consensus sequences, including (i) cleaving after the arginine residue in the consensus Arg-Val; and/or (ii) Xaa₁-Ser-Gly/Ser-Arg/Lys//Gly-Leu/Arg/Val-Xaa₂-Asn/Gly (SEQ ID NO: 28), where Xaa_{1.2} may each be independently selected from any amino acid, and wherein “//” indicates the position of the hydrolysed peptide bond. The term “u-PA” encompasses u-PA described herein, as well as any protease having structural and/or functional similarity (preferably structural and functional similarity) that is capable of hydrolysing a peptide bond of the consensus Arg-Val or Xaa₁-Ser-Gly/Ser-Arg/Lys//Gly-Leu/Arg/Val-Xaa₂-Asn/Gly. A suitable u-PA is human u-PA, which has UniProt Accession No. P29598 (version 1 of the sequence, deposited 1 Apr. 1993, accessed 11 Sep. 2022), herein SEQ ID NO: 31. This sequence is a propeptide, which is converted to the mature human form by a process involving cleavage of an N-terminal polypeptide of residues 1-158. Human u-PA is commercially available from Merck (#672112).

Factor IX (FIX) is a serine protease cleaving in a range of consensus sequences, including (i) cleaving after the arginine residue in the consensus Arg-Ile; and/or (ii) Xaa₁-Xaa₂-Gly-Arg//Xaa₃-Xaa₄-Xaa₅-Xaa₆ (SEQ ID NO: 45), where Xaa_{1.6} may each be independently selected from any amino acid, and wherein “//” indicates the position of the hydrolysed peptide bond. The term “Factor IX” encompasses FIX described herein, as well as any protease having structural and/or functional similarity (preferably structural and functional similarity) that is capable of hydrolysing a peptide bond of the consensus Arg-Ile or Xaa₁-Xaa₂-Gly-Arg//Xaa₃-Xaa₄-Xaa₅-Xaa₆. A suitable FIX is human FIX, which has UniProt Accession No. P00740 (version 2 of the sequence, deposited 7 Jun. 2005, accessed 11 Sep. 2022), herein SEQ ID NO: 47. This sequence is a propeptide, which is converted to the mature human form by a process involving cleavage of an N-terminal polypeptide of residues 1-46. Human FIX is commercially available from Merck (#233279).

Factor VIIa (FVIIa) is a serine protease cleaving in a range of consensus sequences, including cleaving after the arginine residue in the motif Arg-Ile. The term “factor VIIa” encompasses FVIIa described herein, as well as any protease having structural and/or functional similarity (preferably structural and functional similarity) that is capable of hydrolysing a peptide bond of the consensus Arg-Ile. A suitable FVIIa is human FVIIa, which has UniProt Accession No. P08709 (version 1 of the sequence, deposited 1 Jan. 1988, accessed 11 Sep. 2022), herein SEQ ID NO: 48. This sequence is a propeptide, which is converted to the mature human form by a process involving cleavage of an N-terminal polypeptide of residues 1-60. Human FVIIa is commercially available from Merck (#P08709).

Plasminogen is a serine protease cleaving in a range of consensus sequences, including (i) cleaving after the lysine residue in the consensus Lys-Xaa, where Xaa is any amino acid; and/or (ii) after the arginine residue in the consensus Arg-Xaa, where Xaa is any amino acid. The term “plasminogen” encompasses plasminogen described herein, as well as any protease having structural and/or functional similarity (preferably structural and functional similarity) that is capable of hydrolysing a peptide bond of the consensus Lys-Xaa or Arg-Xaa.

A suitable plasminogen is human plasminogen, which has UniProt Accession No. P00747 (version 1 of the sequence, deposited 1 Jul. 1989, accessed 11 Sep. 2022), herein SEQ ID NO: 49. This sequence is a propeptide, which is converted to the mature human form (plasmin) by a process involving cleavage of a peptide bond between Arg580 and Val581. Human AEP is commercially available from Merck (#528185).

For in vitro and ex vivo uses, it is within the routine practice of one of ordinary skill in the art to determine the appropriate concentration/unit amount of any exogenous protease to activate an engineered clostridial neurotoxin of the invention under standard/desired conditions.

In the context of the invention the term “thrombin” encompasses a polypeptide sequence having at least 70% sequence identity to SEQ ID NO: 23, or the mature form thereof.

Thus, “thrombin” may comprise a polypeptide sequence having at least 80% or 90% sequence identity to SEQ ID NO: 23, or the mature form thereof. Preferably a thrombin comprises (more preferably consists of) SEQ ID NO: 23, or the mature form thereof.

In the context of the invention the term “tissue plasminogen activator” encompasses a polypeptide sequence having at least 70% sequence identity to SEQ ID NO: 43, or the mature form thereof. Thus, “t-PA” may comprise a polypeptide sequence having at least 80% or 90% sequence identity to SEQ ID NO: 43, or the mature form thereof. Preferably a u-PA comprises (more preferably consists of) SEQ ID NO: 43, or the mature form thereof.

In the context of the invention the term “urokinase” encompasses a polypeptide sequence having at least 70% sequence identity to SEQ ID NO: 31, or the mature form thereof. Thus, “u-PA” may comprise a polypeptide sequence having at least 80% or 90% sequence identity to SEQ ID NO: 31, or the mature form thereof. Preferably a u-PA comprises (more preferably consists of) SEQ ID NO: 31, or the mature form thereof.

In the context of the invention the term “Factor IX” encompasses a polypeptide sequence having at least 70% sequence identity to SEQ ID NO: 47, or the mature form thereof. Thus, “FIX” may comprise a polypeptide sequence having at least 80% or 90% sequence identity to SEQ ID NO: 47, or the mature form thereof. Preferably a FIX comprises (more preferably consists of) SEQ ID NO: 47, or the mature form thereof.

In the context of the invention the term “Factor VIIa” encompasses a polypeptide sequence having at least 70% sequence identity to SEQ ID NO: 48, or the mature form thereof. Thus, “FVIIa” may comprise a polypeptide sequence having at least 80% or 90% sequence identity to SEQ ID NO: 48, or the mature form thereof. Preferably a FVIIa comprises (more preferably consists of) SEQ ID NO: 48, or the mature form thereof.

In the context of the invention the term “plasminogen” encompasses a polypeptide sequence having at least 70% sequence identity to SEQ ID NO: 49. Thus, “plasminogen” may comprise a polypeptide sequence having at least 80% or 90% sequence identity to SEQ ID NO: 49, or the mature form thereof. Preferably a plasminogen comprises (more preferably consists of) SEQ ID NO: 49.

When describing exogenous protease cleavage sites of the invention, It is not intended that any Xaa (e.g. Xaa₁, Xaa₂, Xaa₃, Xaa₄, Xaa₅ or Xaa₆) be limited to only one type of amino acid. Thus, one or more residues present at any Xaa may be independently selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine.

Alternatively/additionally, one or more residues present at any Xaa (e.g. Xaa₁, Xaa₂, Xaa₃, Xaa₄, Xaa₅ or Xaa₆) may be independently selected from a non-standard amino acid (an amino acid that is not part of the standard set of 20 described above). By way of example, non-standard amino acids may include 4-hydroxyproline, 6-N-methyl lysine, 2-aminoisobutyric acid, isovaline, α-methyl serine, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, L-Ornithine, L-2-amino-3-guanidinopropionic acid, or D-isomers of Lysine, Arginine and/or Ornithine, and 4-fluorophenylalanine. Methods for introducing non-standard amino acids into proteins are known in the art, and include recombinant protein synthesis using E. coli auxotrophic expression hosts.

Properties of the standard amino acids are indicated in the table below:

| AMINO ACID | | | SIDE CHAIN |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

Exemplary (typically consensus) cleavage sites for thrombin, t-PA, u-PA, FIX, FVIIa and plasminogen are described herein. An engineered clostridial neurotoxin of the invention may comprise one or more of these cleavage sites.

A thrombin cleavage site may comprise or consist of a consensus sequence selected from:

| P4 | P3 | P2 | P1 | // | P1′ | P2′ | P3′ | P4′ | |
|---|---|---|---|---|---|---|---|---|---|
| X | X | PALG | RK | // | SAG | X | X | X | (SEQ ID NO: 19) |
| and/or | | | | | | | | | |
| X | X | X | RK | // | X | X | X | X | (SEQ ID NO: 20) |
| preferably | | | | | | | | | |
| X | X | P | R | // | SAG | X | X | X | (SEQ ID NO: 21) |
| and/or | | | | | | | | | |
| X | X | P | K | // | SAG | X | X | X | (SEQ ID NO: 22) |
| most preferably | | | | | | | | | |
| AL | X | PLAG | RK | // | SAG | AVL | GL | X | (SEQ ID NO: 169) |
| and/or | | | | | | | | | |
| AL | X | P | R | // | G | AVL | GL | X | (SEQ ID NO: 170) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, a thrombin cleavage site may comprise or consist of a consensus sequence selected from XX(PALG)(RK)|(SAG)XXX (SEQ ID NO: 19) and/or XXX(RK)|XXXX (SEQ ID NO: 20), preferably XXPR|(SAG)XXX (SEQ ID NO: 21) and/or XXPK|(SAG)XXX (SEQ ID NO: 22), more preferably (AL)X(PLAG)(RK)|(SAG)(AVL)(GL)X (SEQ ID NO: 169) and/or (AL)XPR|G(AVL)(GL)X (SEQ ID NO: 170); wherein each X may be independently selected from any amino acid, the amino acid residues shown in parentheses are alternative possible amino acid residues at each position, wherein the bold and underlined residues are preferred. The “|” shows the site of cleavage (i.e. the peptide bond that is hydrolysed). Thus, a preferred thrombin cleavage site may comprise or consist of a PRG motif (SEQ ID NO: 171).

A t-PA cleavage site may comprise or consist of a consensus sequence selected from:

| P4 | P3 | P2 | P1 | // | P1′ | P2′ | P3′ | P4′ | |
|---|---|---|---|---|---|---|---|---|---|
| X | X | X | R | // | V | X | X | X | (SEQ ID NO: 29) |
| and/or | | | | | | | | | |
| X | FY | SGA | R | // | X | X | X | X | (SEQ ID NO: 34) |
| and/or | | | | | | | | | |
| S | FY | SGA | R | // | X | X | X | X | (SEQ ID NO: 38) |
| and/or | | | | | | | | | |
| G | P | X | KR | // | X | G | G | X | (SEQ ID NO: 35) |
| and/or | | | | | | | | | |
| G | P | Y | KR | // | X | G | G | X | (SEQ ID NO: 40) |
| and/or | | | | | | | | | |
| G | P | X | KR | // | K | G | G | X | (SEQ ID NO: 41) |
| and/or | | | | | | | | | |
| G | P | Y | KR | // | K | G | G | X | (SEQ ID NO: 42) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, a t-PA cleavage site may comprise or consist of a consensus sequence selected from XXXR|VXXX (SEQ ID NO: 29), and/or X(FY)(SGA)R|XXXX (SEQ ID NO: 34), and/or S(FY)(SGA)R|XXXX (SEQ ID NO: 38), and/or GPX(KR)|XGGX (SEQ ID NO: 35), and/or GPY(KR)|XGGX (SEQ ID NO: 40), and/or GPX(KR)|KGGX (SEQ ID NO: 41), and/or GPY(KR)|KGGX (SEQ ID NO: 42); wherein each X may be independently selected from any amino acid, the amino acid residues shown in parentheses are alternative possible amino acid residues at each position, wherein the bold and underlined residues are preferred. The “|” shows the site of cleavage (i.e. the peptide bond that is hydrolysed).

A u-PA cleavage site may comprise or consist of a consensus sequence selected from:

| P4 | P3 | P2 | P1 | // | P1′ | P2′ | P3′ | P4′ | |
|---|---|---|---|---|---|---|---|---|---|
| X | X | X | R | // | V | X | X | X | (SEQ ID NO: 29) |

and/or

| | | | | | | | | | |
|---|---|---|---|---|---|---|---|---|---|
| X | S | GS | RK | // | G | LRV | X | NG | (SEQ ID NO: 28) |

preferably

| | | | | | | | | | |
|---|---|---|---|---|---|---|---|---|---|
| X | X | X | R | // | V | X | X | X | (SEQ ID NO: 29) |

and/or

| | | | | | | | | | |
|---|---|---|---|---|---|---|---|---|---|
| X | S | G | R | // | G | LRV | X | NG | (SEQ ID NO: 30) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, a u-PA cleavage site may comprise or consist of a consensus sequence selected from XXXR|VXXX (SEQ ID NO: 29), and/or XS(GS)(RK)|G(LRV)X(NG) (SEQ ID NO: 28), preferably XXXR|VXXX (SEQ ID NO: 29), and/or XSGR|G(LRV)X(NG) (SEQ ID NO: 30); wherein each X may be independently selected from any amino acid, the amino acid residues shown in parentheses are alternative possible amino acid residues at each position, wherein the bold and underlined residues are preferred. The “|” shows the site of cleavage (i.e. the peptide bond that is hydrolysed).

A u-PA and/or t-PA cleavage site may preferably comprise or consist of a consensus sequence selected from:

| P5 | P4 | P3 | P2 | P1 | // | P1′ | P2′ | |
|---|---|---|---|---|---|---|---|---|
| P | GPAL | SGF | GS | RK | // | SGV | AVLR | (SEQ ID NO: 172) |

and/or

| | | | | | | | | |
|---|---|---|---|---|---|---|---|---|
| P | GPAL | SGF | G | R | // | S | A | (SEQ ID NO: 173) |

Thus, a preferred u-PA and/or t-PA cleavage site may comprise or consist of a consensus sequence selected from P(GPAL)(SGF)(GS)(RK)|(SGV)(AVLR) (SEQ ID NO: 172), and/or P(GPAL)(SGF)GR|SA (SEQ ID NO: 173), wherein each X may be independently selected from any amino acid, the amino acid residues shown in parentheses are alternative possible amino acid residues at each position, wherein the bold and underlined residues are preferred. The “|” shows the site of cleavage (i.e. the peptide bond that is hydrolysed). Thus, a preferred u-PA and/or t-PA cleavage site may comprise or consist of a GRSA motif (SEQ ID NO: 174).

An FIX cleavage site may comprise or consist of a consensus sequence of:

| P4 | P3 | P2 | P1 | // | P1′ | P2′ | P3′ | P4′ | |
|---|---|---|---|---|---|---|---|---|---|
| X | X | X | R | // | I | X | X | X | (SEQ ID NO: 46) |

and/or

| | | | | | | | | | |
|---|---|---|---|---|---|---|---|---|---|
| X | X | G | R | // | X | X | X | X | (SEQ ID NO: 45) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, an FIX cleavage site may comprise or consist of a consensus sequence of XXXR|IXXX (SEQ ID NO: 46), and/or XXGR|XXXX (SEQ ID NO: 45); wherein each X may be independently selected from any amino acid, and the bold and underlined residues are preferred. The “|” shows the site of cleavage (i.e. the peptide bond that is hydrolysed).

An FVIIa cleavage site may comprise or consist of a consensus sequence selected from:

| P4 | P3 | P2 | P1 | // | P1′ | P2′ | P3′ | P4′ | |
|---|---|---|---|---|---|---|---|---|---|
| X | X | X | R | // | I | X | X | X | (SEQ ID NO: 46) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, an FVIIa cleavage site may comprise or consist of a consensus sequence selected from XXXR|IXXX (SEQ ID NO: 46); wherein each X may be independently selected from any amino acid, and the bold and underlined residues are preferred. The “|” shows the site of cleavage (i.e. the peptide bond that is hydrolysed).

A plasminogen cleavage site may comprise or consist of a consensus sequence selected from:

| P4 | P3 | P2 | P1 | // | P1′ | P2′ | P3′ | P4′ | |
|---|---|---|---|---|---|---|---|---|---|
| X | X | X | K | // | X | X | X | X | (SEQ ID NO: 51) |

and/or

| | | | | | | | | | |
|---|---|---|---|---|---|---|---|---|---|
| X | X | X | R | // | X | X | X | X | (SEQ ID NO: 52) |

The amino acid residues shown at each position (P4, P3, P2, P1, P1′, P2′, P3′ and P4′) are alternative possible amino acid residues at each position, wherein the bold and underlined residues are preferred. The “//” shows the site of cleavage (i.e. the peptide bond that is hydrolysed).

Thus, a plasminogen cleavage site may comprise or consist of a consensus sequence selected from XXXR|XXXX (SEQ ID NO: 52), and/or XXXK|XXXX (SEQ ID NO: 51); wherein each X may be independently selected from any amino acid, and the bold and underlined residues are preferred. The “|” shows the site of cleavage (i.e. the peptide bond that is hydrolysed).

An exogenous protease cleavage site of the invention may be formed by the insertion of a peptide that is less than 10 amino acids in length, such as between about 3 to 9 about 9 amino acids in length, or between about 3 to about 8 amino acids in length. Thus, an exogenous protease cleavage site of the invention may be formed by the insertion of a peptide that is typically 3, 4, 5, 6, 7, 8 or 9 amino acids in length.

An exogenous protease cleavage site of the invention may comprise or consist of one or more of: LTPRGVRL (SEQ ID NO: 15), LVPRGS (SEQ ID NO: 16), ENKSLVPRGS (SEQ ID NO: 17), KRV (SEQ ID NO: 24), SGRSA (SEQ ID NO: 25), VVPRVELVA (SEQ ID NO: 32), PPFGRSAG (SEQ ID NO: 33), PGSGRSAG (SEQ ID NO: 26), PGSGRSASGTTGTG (SEQ ID NO: 27), GRI (SEQ ID NO: 44) or PGRVVGG (SEQ ID NO: 50).

An exogenous protease cleavage site of the invention may be a thrombin cleavage site, i.e. the exogenous protease cleavage site may be specific for thrombin. Such a thrombin cleavage site may comprise or consist of LTPRGVRL (SEQ ID NO: 15), ENKSLVPRGS (SEQ ID NO: 17) or LVPRGS (SEQ ID NO: 16), preferably LTPRGVRL (SEQ ID NO: 15) or ENKSLVPRGS (SEQ ID NO: 17). A thrombin cleavage site of LTPRGVRL (SEQ ID NO: 15) is particularly preferred.

An exogenous protease cleavage site of the invention may be a t-PA and/or u-PA cleavage site, i.e. the exogenous protease cleavage site may be specific for t-PA and/or u-PA. Such a t-PA and/or u-PA cleavage site may comprise or consist of KRV (SEQ ID NO: 24), SGRSA (SEQ ID NO: 25), VVPRVELVA (SEQ ID NO: 32) or PPFGRSAG (SEQ ID NO: 33), preferably SGRSA (SEQ ID NO: 25), PGSGRSAG (SEQ ID NO: 26) or PGSGRSASGTTGTG (SEQ ID NO: 27) or PPFGRSAG (SEQ ID NO: 33). A t-PA and/or u-PA cleavage site of PPFGRSAG (SEQ ID NO: 33) and/or PGSGRSAG (SEQ ID NO: 26) may be particularly preferred.

An exogenous protease cleavage site of the invention may be a u-PA cleavage site, i.e. the exogenous protease cleavage site may be specific for u-PA. Such a u-PA cleavage site may comprise or consist of KRV (SEQ ID NO: 24), SGRSA (SEQ ID NO: 25), PGSGRSAG (SEQ ID NO: 26), PGSGRSASGTTGTG (SEQ ID NO: 27) or PPFGRSAG (SEQ ID NO: 33), preferably SGRSA (SEQ ID NO: 25), PGSGRSAG (SEQ ID NO: 26) or PGSGRSASGTTGTG (SEQ ID NO: 27). A u-PA cleavage site of PGSGRSAG (SEQ ID NO: 26) may be particularly preferred.

An exogenous protease cleavage site of the invention may be a t-PA cleavage site, i.e. the exogenous protease cleavage site may be specific for t-PA. Such a u-PA cleavage site may comprise or consist of SGRSA (SEQ ID NO: 25), VVPRVELVA (SEQ ID NO: 32) or PPFGRSAG (SEQ ID NO: 33), preferably PPFGRSAG (SEQ ID NO: 33) or SGRSA (SEQ ID NO: 25). A t-PA cleavage site of PPFGRSAG (SEQ ID NO: 33) may be particularly preferred.

An exogenous protease cleavage site of the invention may be an FIX and/or FVIIa cleavage site, i.e. the exogenous protease cleavage site may be specific for FIX and/or FVIIa.

Such a FIX and/or FVIIa cleavage site may comprise or consist of GRI (SEQ ID NO: 44).

An exogenous protease cleavage site of the invention may be a plasminogen cleavage site, i.e. the exogenous protease cleavage site may be specific for plasminogen. Such a plasminogen cleavage site may comprise or consist of PGRVVGG (SEQ ID NO: 50).

An engineered clostridial neurotoxin of the invention may comprise one or more exogenous protease cleavage site as defined herein.

In some embodiments an exogenous protease cleavage site of the invention has at least 70% sequence identity to any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred. An exogenous protease cleavage site may have at least 80%, 85% or 90% sequence identity to any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred. [. Preferably an exogenous protease cleavage site has at least 95% sequence identity to any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred. More preferably, an exogenous protease cleavage site has at least 99% sequence identity to any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred. Particularly preferred is an exogenous protease cleavage site comprising or consisting of any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred.

Typically said exogenous protease cleavage site comprises or consists of one or more of the amino acid sequence of any one of SEQ ID NOs: 15-22, 24-30, 32-42, 44-46 and/or 50-52, particularly SEQ ID NOs: 15-22, 24-30 and/or 32-42, preferably SEQ ID NOs: 15, 17, 16, 26, 25, 27, 24, 33 and/or 32, with SEQ ID NOs: 15, 17, 16, 26, 25 and/or 33 being particularly preferred, or the exogenous activation loop comprises said one or more exogenous protease cleavage site.

An engineered clostridial neurotoxin of the invention may comprise one or more exogenous protease cleavage site. In other words, an engineered clostridial neurotoxin of the invention may comprise one exogenous protease cleavage site as described herein, or multiple exogenous protease cleavage sites. An engineered clostridial neurotoxin of the invention may comprise two, three, four, five, six, seven, eight, nine, ten or more exogenous protease cleavage sites. By way of non-limiting example, an engineered clostridial neurotoxin of the invention may comprise 2 to 7 (2, 3, 4, 5, 6 or 7) exogenous protease cleavage site.

When an engineered clostridial neurotoxin of the invention comprises multiple exogenous protease cleavage sites, these may each be selected independently. Typically, when an engineered clostridial neurotoxin of the invention comprises multiple exogenous protease cleavage sites, each exogenous protease cleavage site may independently be selected from the exogenous protease cleavage sites described herein. Thus, an engineered clostridial neurotoxin of the invention may comprise multiple exogenous protease cleavage sites that are each different from each other, or an engineered clostridial neurotoxin of the invention may comprise two or more copies of a specific exogenous protease cleavage sites, or any combination thereof.

Multiple exogenous protease cleavage sites may be introduced at a single location within a clostridial neurotoxin. Alternatively, multiple exogenous protease cleavage sites may be introduced at multiple locations within a clostridial neurotoxin. By way of non-limiting example, one exogenous protease cleavage site may be introduced within the activation loop or modified BoNT/C (BoNT/C1) activation loop within a clostridial neurotoxin and another (same or different) exogenous protease cleavage site may be introduced within the LH_{N} domain. Typically, where multiple exogenous protease cleavage sites are introduced into an engineered clostridial neurotoxin according to the invention, they are introduced at a single location within a clostridial neurotoxin. Preferably, where multiple exogenous protease cleavage sites are introduced into an engineered clostridial neurotoxin according to the invention, they are each introduced within the activation loop or modified BoNT/C (BoNT/C1) activation loop of the clostridial neurotoxin. The relative positioning of individual exogenous protease cleavage sites within an exogenous activation loop or modified BoNT/C (BoNT/C1) activation loop of the invention may be determined by the structure-function relationship for the exogenous protease in question. It is within the routine practice of one of ordinary skill in the art to appropriately position individual exogenous protease cleavage sites within an exogenous activation loop or modified BoNT/C (BoNT/C1) activation loop comprising multiple exogenous protease cleavage site based on this structure-function relationship, without undue burden.

The exogenous protease cleavage site may be comprises within a modified clostridial toxin activation loop. Said modified clostridial toxin activation loop may be from the same serotype as the engineered clostridial neurotoxin, or a different serotype to the engineered clostridial neurotoxin. Typically said modified clostridial toxin activation loop is from a different serotype as the engineered clostridial neurotoxin. Preferably, said modified clostridial activation loop is from BoNT/C or BoNT/A, with a modified clostridial activation loop from BoNT/C being particularly preferred.

A modified clostridial activation loop is one in which the endogenous activation site, or part thereof, has been replaced by an exogenous protease cleavage site as described herein.

Preferably, a modified clostridial activation loop may be (i) a BoNT/C activation loop in which the endogenous BoNT/C activation site, or part thereof, has been replaced by an exogenous protease cleavage site as defined herein; or (ii) a BoNT/A activation loop in which the endogenous BoNT/A activation site, or part thereof, has been replaced by an exogenous protease cleavage site as defined herein. A modified clostridial activation loop which is a BoNT/C activation loop in which the endogenous BoNT/C activation site, or part thereof, has been replaced by an exogenous protease cleavage site as defined herein, is particularly preferred.

As described herein, one or more exogenous protease cleavage site may be comprised in an exogenous activation loop or modified BoNT/C activation loop together with one or more spacer sequence as defined herein. Spacer sequences may typically be present when the one or more exogenous protease cleavage site is a short motif (e.g. typically less than 15, preferably less than 10 or less than 9 amino acids in length). One or more spacer may be present N-terminal and/or C-terminal to each of said exogenous protease cleavage sites. Preferably, a spacer may be a GS spacer as defined herein.

An exogenous activation loop or modified BoNT/C activation loop may comprise one or more exogenous protease cleavage site (e.g. two, three, four or five exogenous protease cleavage sites, typically two or three exogenous protease cleavage sites). Said one or more exogenous protease cleavage sites may be cleavable by different exogenous proteases, By way of non-limiting example, an exogenous activation loop or modified BoNT/C activation loop may comprise a thrombin cleavage site and a t-PA cleavage site. Inclusion of one or more exogenous protease cleavage sites within an exogenous activation loop, or modified BoNT/C activation loop wherein said one or more exogenous protease sites are cleavable by different exogenous proteases may increase processing of an engineered clostridial neurotoxin comprising said exogenous activation loop or modified BoNT/C activation loop, particularly in vivo processing of said engineered clostridial neurotoxin. Alternatively, multiple copies of the same exogenous protease cleavage site may be comprised within an exogenous activation loop or modified BoNT/C activation loop. Whether an exogenous activation loop or modified BoNT/C activation loop comprises multiple different exogenous protease cleavage sites or multiple copies of the same exogenous protease cleavage site, said cleavage sites may be directly linked, or may be separated by one or more spacer as described herein.

Non-limiting examples of exogenous activation loops, including modified BoNT/C activation loops according to the invention include:

| SEQ ID | | | |
|---|---|---|---|
| NO: | Sequence | Description of cleavage specificity | Length |
| 53 | CHKAIDKRVLYNKTLDC | minimal uPA/tPA | 17 |
| 54 | CHKALVPRGSYNKTLDC | minimal Thrombin | 17 |
| 55 | CHKAIDGRILYNKTLDC | minimal FIX/FVII | 17 |
| 56 | CHKAENKSLVPRGSYNKTLDC | Thrombin full | 21 |
| 57 | CHKALTPRGVRLKTLDC | Thrombin alternative | 17 |
| 58 | CPGRVVGGC | native plasminogen | 9 |
| 59 | CGGGGSPGRVVGGGGSC | native plasminogen loop with spacers | 17 |
| | | (G4S) | |
| 60 | CSGRSAGGC | optimal uPA in plasminogen loop | 9 |
| 61 | CGGGGSGRSAGGGGSC | optimal uPA with spacers (G4S) | 16 |
| 62 | CHKAVVPRVELVATLDC | tPA alternative substrate | 17 |
| 63 | CHKAIPPFGRSAGYNKTLDC | tPA Alternative from phage display | 17 |
| | | library | |
| 64 | CHKAIPGSGRSAGYNKTLDC | uPA Alternative from phage display | 17 |
| | | library | |
| 65 | CHKAISGRSAYNKTLDC | uPA Alternative from phage display | 17 |
| | | library | |
| 66 | CHKAIPGSGRSASGTTGTGYNKTLDC | uPA Alternative from phage display | 24 |
| | | library | |

wherein substituted residues are shown in bold; inserted residues are shown in italics; plasminogen residues are double-underlined; and the remaining residues are present in the corresponding unmodified clostridial activation loop.

An exogenous activation loop or modified BoNT/C activation loop comprising one or more exogenous protease cleavage site may be any length, provided that the architecture of the exogenous activation loop or modified BoNT/C activation loop, and typically the clostridial neurotoxin, is preserved and cleavage at the one or more exogenous protease cleavage site results in the formation of an active di-chain form of the engineered clostridial neurotoxin. The exogenous activation loop or modified BoNT/C activation loop may be between about 10 to about 80, such as between about 10 to about 50, between about 10 to about 40, or between about 10 to about 30 (e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50) amino acids in length, such as between about 15 to about 35 (e.g. 15, 16, 17, 18, 19, 20, 21, 22, 23, 24, 25, 26, 27, 28, 29, 30, 31, 32, 33, 34 or 35) amino acids in length, preferably between about 15 to about 30 (e.g. 15, 16, 17, 18, 19, 20, 21, 22, 23, 24, 25, 26, 27, 28, 29 or 30) amino acids in length. Particularly preferred are exogenous activation loops or modified BoNT/C activation loops which are 17 amino acids in length, as such exogenous activation loops or modified BoNT/C activation loops are the same length as the endogenous BoNT/C (BoNT/C1) activation loop.

### Clostridial Neurotoxins

The term “neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. It is this di-chain form that is the active form of the toxin. The two chains are termed the heavy chain (H-chain), which has a molecular mass of approximately 100 kDa, and the light chain (L-chain), which has a molecular mass of approximately 50 kDa.

A clostridial neurotoxin of the invention may be catalytically active also referred to as active) or catalytically inactive. Preferably a clostridial neurotoxin of the invention is catalytically active.

The terms “catalytically active” or “active” as used interchangeably herein refer to a clostridial neurotoxin L-chain (or a clostridial neurotoxin comprising such an L-chain) having non-cytotoxic protease activity. Specifically, active clostridial neurotoxin L-chain has endopeptidase activity and is capable of cleaving a protein of the exocytic fusion apparatus in a target cell. A protein of the exocytic fusion apparatus is preferably a SNARE protein, such as SNAP25, synaptobrevin/VAMP, or syntaxin.

The term “catalytically inactive” as used herein in respect of a clostridial neurotoxin L-chain means that said L-chain exhibits substantially no non-cytotoxic protease activity, preferably the term “catalytically inactive” as used herein in respect of a clostridial neurotoxin L-chain means that said L-chain exhibits no non-cytotoxic protease activity. In one embodiment, a catalytically inactive clostridial neurotoxin L-chain is one that does not cleave a protein of the exocytic fusion apparatus in a target cell. The term “substantially no non-cytotoxic protease activity” means that the clostridial neurotoxin L-chain has less than 5% of the non-cytotoxic protease activity of a catalytically active clostridial neurotoxin L-chain, for example less than 2%, 1% or preferably less than 0.1% of the non-cytotoxic protease activity of a catalytically active clostridial neurotoxin L-chain. Non-cytotoxic protease activity can be determined in vitro by incubating a test clostridial neurotoxin L-chain with a SNARE protein and comparing the amount of SNARE protein cleaved by the test clostridial neurotoxin L-chain when compared to the amount of SNARE protein cleaved by a catalytically active clostridial neurotoxin L-chain under the same conditions. Routine techniques, such as SDS-PAGE and Western blotting can be used to quantify the amount of SNARE protein cleaved. Suitable in vitro assays are described in WO 2019/145577 A1, which is incorporated herein by reference.

The clostridial neurotoxin (e.g. pre-engineering) may be BoNT/A. Exemplary reference BoNT/A sequences are shown as SEQ ID NOs: 91-98 and 135, particularly SEQ ID NOs: 91 and 135.

The clostridial neurotoxin (e.g. pre-engineering) may be BoNT/B. Exemplary reference BoNT/B sequences are shown as SEQ ID NOs: 99-106, particularly SEQ ID NO: 99.

The clostridial neurotoxin (e.g. pre-engineering) may be BoNT/C. An exemplary reference BoNT/C₁ sequence is shown as SEQ ID NO: 107.

The clostridial neurotoxin (e.g. pre-engineering) may be BoNT/D. An exemplary reference BoNT/D sequence is shown as SEQ ID NOs: 108.

The clostridial neurotoxin (e.g. pre-engineering) may be BoNT/E. Exemplary reference BoNT/E sequences are shown as SEQ ID NO: 111-123, particularly SEQ ID NO: 111.

The clostridial neurotoxin (e.g. pre-engineering) may be BoNT/F. Exemplary reference BoNT/F sequences are shown as SEQ ID NO: 124-130, particularly SEQ ID NO: 124.

The clostridial neurotoxin (e.g. pre-engineering) may be BoNT/G. An exemplary reference BoNT/G sequence is shown as SEQ ID NO: 131.

The clostridial neurotoxin (e.g. pre-engineering) may be BoNT/X. An exemplary reference BoNT/X sequence is shown as SEQ ID NO: 133.

The clostridial neurotoxin (e.g. pre-engineering) may be TeNT. An exemplary reference TeNT sequence is shown as SEQ ID NO: 134.

In some preferred embodiments, clostridial neurotoxin (e.g. pre-engineering) is a BoNT/A, BoNT/B or BoNT/X, or a chimera thereof (e.g. BoNT/AB) as described herein.

As discussed above, activated clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Examples of light chain reference sequences include:
Botulinum type A neurotoxin: amino acid residues 1-448
Botulinum type B neurotoxin: amino acid residues 1-440
Botulinum type C₁ neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464). Preferably the L-chain in an engineered clostridial neurotoxin of the invention is a BoNT/X L-chain.

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C₁ neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

Alternatively, clostridial neurotoxin L-chains may be defined as the first amino acid (including or excluding an initial methionine residue) through to the first cysteine residue of the endogenous activation loop. In addition or alternatively, a clostridial neurotoxin L-chain may be defined as the amino acid sequence N-terminal to the cleavage site within the endogenous activation loop.

Clostridial neurotoxin L-chains may be defined as a clostridial neurotoxin domain which comprises the metal coordinating HExxH motif (SEQ ID NO: 143), which typically functions to cleave a SNARE protein substrate.

The term “light-chain” (or “L-chain”) encompasses variants and fragments thereof, provided said variants and fragments still demonstrate non-cytotoxic protease activity (which can be determined using standard assays known in the art, examples of which are described herein). By way of example, a variant may have at least 70%, preferably at least 80%, more preferably at least 90%, and most preferably at least 95% or at least 98% amino acid sequence homology with a reference L-chain. The term fragment, when used in relation to a L-chain, means a peptide having at least 200, preferably at least 250, more preferably at least 300, even more preferably at least 350, and most preferably at least 400 amino acid residues of the reference L-chain. In the case of a clostridial L-chain, the fragment preferably at least 300, more preferably at least 350, and most preferably at least 400 amino acid residues of the reference L-chain. L-chain ‘fragments’ of the present invention embrace fragments of variant L-chains based on the reference sequences.

A clostridial neurotoxin H-chains may be defined as the second cysteine of the endogenous activation loop through to the final amino acid. In addition or alternatively, a clostridial neurotoxin H-chain may be defined as starting from the amino acid sequence C-terminal to the cleavage site within the endogenous activation loop. In addition or alternatively, a clostridial neurotoxin H-chain may be defined as starting from the amino acid C-terminal to the cysteine residue (typically the second cysteine residue) that forms a disulphide bond between the L- and H-chain and so defines the C-terminal of the endogenous activation loop.

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored (see Shone C. (1987) Eur. J. Biochem; vol. 167(1): pp. 175-180).

A further example is provided by Blaustein R. (1987), which describes a simple in vitro assay employing planar phospholipid bilayer membranes. The membranes are challenged with a test molecule and the requisite translocation function is confirmed by an increase in conductance across said membranes (see Blaustein (1987) FEBS Letts; vol. 226, no. 1: pp. 115-120).

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variants and/or fragments of translocation domains, so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (e.g. H_{N} domain). Translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, the Translocation Domain may be a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. The H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, the H-chain may be incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Botulinum type X neurotoxin—amino acid residues (461-890)
Tetanus neurotoxin—amino acid residues (458-879)

For recently-identified BoNT/X, the translocation domain has been reported as corresponding to amino acids 460-890 thereof, with the L-chain and H_{C} boundaries potentially varying by approximately 10 amino acids (e.g. 461-889 or 454-891). Preferably, the translocation domain of an engineered clostridial neurotoxin of the invention is a BoNT/X translocation domain.

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of clostridial neurotoxin H_{N} regions comprising a translocation domain can be useful in aspects of the present invention with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, in the context of the present invention a translocation domain can include clostridial neurotoxin H_{N} regions comprising a translocation domain having a length of, for example, at least 350 amino acids, at least 375 amino acids, at least 400 amino acids and at least 425 amino acids. Also encompassed are clostridial neurotoxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin (O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51), the translocation domain of Pseudomonas exotoxin type A (Prior et al. Biochemistry (1992) 31, 3555-3559), the translocation domains of anthrax toxin (Blanke et al. Proc. Natl. Acad. Sci. USA (1996) 93, 8437-8442), a variety of fusogenic or hydrophobic peptides of translocating function (Plank et al. J. Biol. Chem. (1994) 269, 12918-12924; and Wagner et al (1992) PNAS, 89, pp. 7934-7938), and amphiphilic peptides (Murata et al(1992) Biochem., 31, pp. 1986-1992). The Translocation Domain may mirror the Translocation Domain present in a naturally-occurring protein, or may include amino acid variations so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded Aspike proteins have particular application in the context of the present invention, for example, the E1 protein of SFV and the G protein of the G protein of VSV.

Use of the (reference) Translocation Domains listed in Table (below) includes use of sequence variants thereof. A variant may comprise one or more conservative nucleic acid substitutions and/or nucleic acid deletions or insertions, with the proviso that the variant possesses the requisite translocating function. A variant may also comprise one or more amino acid substitutions and/or amino acid deletions, insertions or insertion-deletions (indels), so long as the variant possesses the requisite translocating function.

| Translocation | | |
|---|---|---|
| Domain source | Amino acid residues | References |
| Diphtheria toxin | 194-380 | Silverman et al., 1994, J. Biol. Chem. |
| | | 269, 22524-22532 |
| | | London E., 1992, Biochem. Biophys. |
| | | Acta., 1113, 25-51 |
| Domain II of | 405-613 | Prior et al., 1992, Biochemistry 31, |
| pseudomonas | | 3555-3559 |
| exotoxin | | Kihara & Pastan, 1994, Bioconj Chem. 5, |
| | | 532-538 |
| Influenza virus | GLFGAIAGFIENGWE | Plank et al., 1994, J. Biol. Chem. 269, |
| haemagglutinin | GMIDGWYG (SEQ ID | 12918-12924 |
| | NO: 73), and | Wagner et al., 1992, PNAS, 89, 7934-7938 |
| | Variants thereof | Murata et al., 1992, Biochemistry 31, |
| | | 1986-1992 |
| Semliki Forest virus | Translocation domain | Kielian et al., 1996, J Cell Biol. 134(4), |
| fusogenic protein | | 863-872 |
| Vesicular Stomatitis | 118-139 | Yao et al., 2003, Virology 310(2), 319-332 |
| virus glycoprotein | | |
| G | | |
| SER virus F protein | Translocation domain | Seth et al., 2003, J Virol 77(11) |
| | | 6520-6527 |
| Foamy virus | Translocation domain | Picard-Maureau et al., 2003, J Virol. |
| envelope | | 77(8), 4722-4730 |
| glycoprotein | | |

Examples of clostridial neurotoxin H_{C} domain reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—1880-D1315

For recently-identified BoNT/X, the H_{C} domain has been reported as corresponding to amino acids 893-1306 thereof, with the domain boundary potentially varying by approximately amino acids (e.g. 868-1306 or 918-1306). Preferably the H_{C} domain of an engineered clostridial neurotoxin of the invention is a BoNT/X H_{C} domain.

The clostridial neurotoxins described herein may further comprise a translocation facilitating domain. Said domain facilitates delivery of the non-cytotoxic protease into the cytosol of the target cell and are described, for example, in WO 08/008803 and WO 08/008805, each of which is herein incorporated by reference thereto.

By way of example, suitable translocation facilitating domains include an enveloped virus fusogenic peptide domain, for example, suitable fusogenic peptide domains include influenzavirus fusogenic peptide domain (eg. influenza A virus fusogenic peptide domain of 23 amino acids), alphavirus fusogenic peptide domain (eg. Semliki Forest virus fusogenic peptide domain of 26 amino acids), vesiculovirus fusogenic peptide domain (eg. vesicular stomatitis virus fusogenic peptide domain of 21 amino acids), respirovirus fusogenic peptide domain (eg. Sendai virus fusogenic peptide domain of 25 amino acids), morbiliivirus fusogenic peptide domain (eg. Canine distemper virus fusogenic peptide domain of 25 amino acids), avulavirus fusogenic peptide domain (eg. Newcastle disease virus fusogenic peptide domain of 25 amino acids), henipavirus fusogenic peptide domain (eg. Hendra virus fusogenic peptide domain of 25 amino acids), metapneumovirus fusogenic peptide domain (eg. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Botulinum type X neurotoxin—amino acid residues (890-1121)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide.

Alternatively, a clostridial neurotoxin H_{CN} translocation facilitating domain may be combined with a non-clostridial translocation domain peptide. Alternatively, a clostridial neurotoxin H_{CN} facilitating domain may be combined or with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

In some embodiments the clostridial neurotoxins of the present invention may lack a functional H_{C} domain of a clostridial neurotoxin. Accordingly, said clostridial neurotoxins are not able to bind rat synaptosomal membranes (via a clostridial H_{C} component) in binding assays as described in Shone et al. (1985) Eur. J. Biochem. 151, 75-82. The clostridial neurotoxins may preferably lack the last 50 C-terminal amino acids of a clostridial neurotoxin holotoxin. The clostridial neurotoxins may preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland TC (1997) Nat. Struct. Biol. 4: 788-792; Herreros J (2000) Biochem. J. 347:199-204; Halpern J (1993) J. Biol. Chem. 268:15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25:90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (Hoc), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking afunctional heavy chain H_{C} peptide (or domain) such that the heavy-chain is incapable of binding to cell surface receptors to which a native clostridial neurotoxin binds means that the clostridial heavy-chain simply lacks a functional H_{C0} peptide. In other words, the H_{C0} peptide region may be either partially or wholly deleted, or otherwise modified (e.g. through conventional chemical or proteolytic treatment) to inactivate its native binding ability for nerve terminals at the neuromuscular junction.

Thus, a clostridial neurotoxin H_{N} peptide of the present invention may be C-terminally extended, i.e. it may be associated with all or part of a clostridial neurotoxin H_{C} domain, e.g. the H_{CN}, H_{CC} or H_{C} domain. References herein to a clostridial neurotoxin H_{N} peptide of the present invention encompass such C-terminally extended H_{N} peptides, which comprise one or more amino acid residues from a clostridial neurotoxin H_{C} domain. Alternatively, a clostridial neurotoxin H_{N} peptide of the present invention may not be associated with (or lack) all or part of a clostridial neurotoxin H_{C} domain, e.g. the H_{CN}, H_{C0} or H_{C} domain.

Typically if a clostridial neurotoxin of the invention or a clostridial neurotoxin H_{N} peptide of the present invention lacks all or part of a C-terminal peptide portion (Hoc) of a clostridial neurotoxin it thus lacks the H_{C} binding function of native clostridial neurotoxin. By way of example, a C-terminally extended clostridial H_{N} peptide may lack the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. Alternatively, the clostridial H_{N} peptide of the present invention may lack the entire C-terminal peptide portion (Hoc) of a clostridial neurotoxin and thus lacks the H_{C} binding function of native clostridial neurotoxin. By way of example, the clostridial H_{N} peptide may lack the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K4252)
Botulinum type F neurotoxin—amino acid residues (Y1106-E1274)
Botulinum type G neurotoxin—amino acid residues (Y1106-E1297)
Botulinum type X neurotoxin—amino acid residues (Y1122-D1306)
Tetanus neurotoxin—amino acid residues (Y1128-D1315).

The above-identified reference sequences should be considered a guide as slight variations may occur according to sub-serotypes.

The present invention is suitable for application to many different varieties of clostridial neurotoxin. Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing. The term “clostridial neurotoxin” also embraces botulinum neurotoxin serotype H. In some preferred embodiments, the clostridial neurotoxin is BoNT/A, more preferably BoNT/A₁. In other preferred embodiments, the clostridial neurotoxin is BoNT/X.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, a clostridial neurotoxin of the invention may be a modified clostridial neurotoxin, or a modified clostridial neurotoxin derivative, or a clostridial neurotoxin derivative. In particular, an engineered clostridial neurotoxin of the invention may be an engineered modified clostridial neurotoxin, or an engineered modified clostridial neurotoxin derivative, or an engineered clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B Hoc subdomain have been disclosed in WO 2013/180799 and in WO 2016/154534 (both herein incorporated by reference).

A suitable amino acid residue substitution, insertion, indel or deletion in the BoNT/B H_{CC} subdomain may include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191 M and S1199Y, E1191 M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191 M and S1199Y. Such modifications are present in chimeric clostridial neurotoxin of SEQ ID NO: 136. E1191 M may correspond to position 1204 of SEQ ID NO: 136 and S1199Y may correspond to position 1212. Thus, SEQ ID NO: 136 may comprise 1204M and 1212Y.

The modification may be a modification when compared to unmodified BoNT/B shown as SEQ ID NO: 99, wherein the amino acid residue numbering is determined by alignment with SEQ ID NO: 99. As the presence of a methionine residue at position 1 of SEQ ID NO: 99 (as well as the SEQ ID NOs corresponding to other clostridial neurotoxin polypeptides described herein, including chimeric clostridial neurotoxin polypeptides) is optional, the skilled person will take the presence/absence of the methionine residue into account when determining amino acid residue numbering. For example, where SEQ ID NO: 99 includes a methionine, the position numbering will be as defined above (e.g. E1191 will be E1191 of SEQ ID NO: 99). Alternatively, where the methionine is absent from SEQ ID NO: 99 the amino acid residue numbering should be modified by −1 (e.g. E1191 will be E1190 of SEQ ID NO: 99). Accordingly, an initial methionine amino acid residue of a polypeptide sequence of the chimeric clostridial neurotoxin may be optional or absent. Similar considerations apply when the methionine at position 1 of the other polypeptide sequences described herein is present/absent, and the skilled person will readily determine the correct amino acid residue numbering using techniques routine in the art. Alignment may be carried out using any of the methods described herein for determining sequence homology and/or % sequence identity.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL (SEQ ID NO: 144), xExxxLL (SEQ ID NO: 145), xExxxIL (SEQ ID NO: 146), and xExxxLM (SEQ ID NO: 147) (wherein x is any amino acid). Suitable tyrosine-based motifs include Y-x-x-Hy (wherein Hy is a hydrophobic amino acid). Examples of modified clostridial neurotoxins comprising leucine- and tyrosine-based motifs are described in WO 2002/008268, which is hereby incorporated by reference in its entirety.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. A hybrid clostridial neurotoxin may contain the entire light chain from one clostridial neurotoxin subtype and the heavy chain from another clostridial neurotoxin subtype. A chimeric clostridial neurotoxin may contain a portion (e.g. the binding domain) of the heavy chain of one clostridial neurotoxin subtype, with another portion of the heavy chain being from another clostridial neurotoxin subtype. A chimeric clostridial neurotoxin, particularly a chimeric BoNT, may be defined in terms of the serotype or sub-serotype of the four main domains of the neurotoxin: L-chain, H_{N}, H_{CN} and Hoc (as defined herein). For example, the (pre-engineering) LH_{N}/A1-H_{C}B1 chimera of SEQ ID NO: 136 may be described as an AABB chimera. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, a clostridial neurotoxin of the invention may be a hybrid clostridial neurotoxin, or a chimeric clostridial neurotoxin. In particular, an engineered clostridial neurotoxin of the invention may be an engineered hybrid clostridial neurotoxin, or an engineered chimeric clostridial neurotoxin.

In some preferred embodiments, a clostridial neurotoxin is BoNT/A comprising at least one domain from a non-BoNT/A clostridial neurotoxin (e.g. a BoNT/A hybrid or chimera). For example, a clostridial neurotoxin of the invention (comprising one or more exogenous protease cleavage site) may comprise:
i. A BoNT/A L-chain and a non-BoNT/A H_{N} and H_{C} domain;
ii. A BoNT/A H_{N} domain and a non-BoNT/A L-chain and H_{C} domain
iii. A BoNT/A H_{C} domain and a non-BoNT/A L-chain and H_{N} domain;
iv. A BoNT/A L-chain and H_{N} domain and a non-BoNT/A H_{C} domain
v. A BoNT/A L-chain and H_{C} domain and a non-BoNT/A H_{N} domain; or
vi. A BoNT/A H_{N} domain and H_{C} domain and a non-BoNT/A L-chain.

By way of non-limiting example, a clostridial neurotoxin of the invention (e.g. an engineered clostridial neurotoxin) comprises a BoNT/A L-chain and H_{N} domain and a BoNT/B H_{C} domain (such as LH_{N}/A1-H_{C}/B1). An exemplary non-engineered LH_{N}/A1-H_{C}B1 chimera that may be modified to comprises one or more exogenous protease cleavage site according to the invention is given in SEQ ID NO: 136. Exemplary engineered forms of the LH_{N}/A1-H_{C}B1 chimera of SEQ ID NO: 136 are given in SEQ ID NOs: 155, 157 and 159. A clostridial neurotoxin of the invention (e.g. an engineered clostridial neurotoxin) may comprise a BoNT/A L-chain and H_{N} domain and a BoNT/C1 H_{C} domain. A clostridial neurotoxin of the invention (e.g. an engineered clostridial neurotoxin) may comprise a BoNT/A L-chain and H_{N} domain and a BoNT/D H_{C} domain. A clostridial neurotoxin of the invention (e.g. an engineered clostridial neurotoxin) may comprise a BoNT/A L-chain and H_{N} domain and a BoNT/E H_{C} domain. A clostridial neurotoxin of the invention (e.g. an engineered clostridial neurotoxin) may comprise a BoNT/A L-chain and H_{N} domain and a BoNT/F H_{C} domain. A clostridial neurotoxin of the invention (e.g. an engineered clostridial neurotoxin) may comprise a BoNT/A L-chain and H_{N} domain and a BoNT/G H_{C} domain. A clostridial neurotoxin of the invention (e.g. an engineered clostridial neurotoxin) may comprise a BoNT/A L-chain and H_{N} domain and a BoNT/X H_{C} domain. A clostridial neurotoxin of the invention (e.g. an engineered clostridial neurotoxin) may comprise a BoNT/A L-chain and H_{N} domain and a TeNT H_{C} domain.

For example, a clostridial neurotoxin of the invention (e.g. an engineered clostridial neurotoxin comprising one or more exogenous protease cleavage site) may comprise:
i. A BoNT/B L-chain and a non-BoNT/B H_{N} and H_{C} domain;
ii. A BoNT/B H_{N} domain and a non-BoNT/B L-chain and H_{C} domain
iii. A BoNT/B H_{C} domain and a non-BoNT/B L-chain and H_{N} domain;
iv. A BoNT/B L-chain and H_{N} domain and a non-BoNT/B H_{C} domain
v. A BoNT/B L-chain and H_{C} domain and a non-BoNT/B H_{N} domain; or
vi. A BoNT/B H_{N} domain and H_{C} domain and a non-BoNT/B L-chain.

For example, a clostridial neurotoxin of the invention (e.g. an engineered clostridial neurotoxin comprising one or more exogenous protease cleavage site) may comprise:
i. A BoNT/C1 L-chain and a non-BoNT/C1 H_{N} and H_{C} domain;
ii. A BoNT/C1 H_{N} domain and a non-BoNT/C1 L-chain and H_{C} domain
iii. A BoNT/C1 H_{C} domain and a non-BoNT/C1 L-chain and H_{N} domain;
iv. A BoNT/C1 L-chain and H_{N} domain and a non-BoNT/C1 H_{C} domain
v. A BoNT/C1 L-chain and H_{C} domain and a non-BoNT/C1 H_{N} domain; or
vi. A BoNT/C1 H_{N} domain and H_{C} domain and a non-BoNT/C1 L-chain.

Non-limiting examples include BoNT/C1 chimeras where the non-BoNT/C1 element is from a BoNT/D (i.e. BoNT/CD chimeras).

For example, a clostridial neurotoxin of the invention (e.g. an engineered clostridial neurotoxin comprising one or more exogenous protease cleavage site) may comprise:
i. A BoNT/D L-chain and a non-BoNT/D H_{N} and H_{C} domain;
ii. A BoNT/D H_{N} domain and a non-BoNT/D L-chain and H_{C} domain
iii. A BoNT/D H_{C} domain and a non-BoNT/D L-chain and H_{N} domain;
iv. A BoNT/D L-chain and H_{N} domain and a non-BoNT/D H_{C} domain
v. A BoNT/D L-chain and H_{C} domain and a non-BoNT/D H_{N} domain; or
vi. A BoNT/D H_{N} domain and H_{C} domain and a non-BoNT/D L-chain.

Non-limiting examples include BoNT/D chimeras where the non-BoNT/D element is from BoNT/C1 (i.e. BoNT/DC1 chimeras).

For example, a clostridial neurotoxin of the invention (e.g. an engineered clostridial neurotoxin comprising one or more exogenous protease cleavage site) may comprise:
i. A BoNT/E L-chain and a non-BoNT/E H_{N} and H_{C} domain;
ii. A BoNT/E H_{N} domain and a non-BoNT/E L-chain and H_{C} domain
iii. A BoNT/E H_{C} domain and a non-BoNT/E L-chain and H_{N} domain;
iv. A BoNT/E L-chain and H_{N} domain and a non-BoNT/E H_{C} domain
v. A BoNT/E L-chain and H_{C} domain and a non-BoNT/E H_{N} domain; or
vi. A BoNT/E H_{N} domain and H_{C} domain and a non-BoNT/E L-chain.

For example, a clostridial neurotoxin of the invention (e.g. an engineered clostridial neurotoxin comprising one or more exogenous protease cleavage site) may comprise:
i. A BoNT/F L-chain and a non-BoNT/F H_{N} and H_{C} domain;
ii. A BoNT/F H_{N} domain and a non-BoNT/F L-chain and H_{C} domain
iii. A BoNT/F H_{C} domain and a non-BoNT/F L-chain and H_{N} domain;
iv. A BoNT/F L-chain and H_{N} domain and a non-BoNT/F H_{C} domain
v. A BoNT/F L-chain and H_{C} domain and a non-BoNT/F H_{N} domain; or
vi. A BoNT/F H_{N} domain and H_{C} domain and a non-BoNT/F L-chain.

For example, a clostridial neurotoxin of the invention (e.g. an engineered clostridial neurotoxin comprising one or more exogenous protease cleavage site) may comprise:
i. A BoNT/G L-chain and a non-BoNT/G H_{N} and H_{C} domain;
ii. A BoNT/G H_{N} domain and a non-BoNT/G L-chain and H_{C} domain
iii. A BoNT/G H_{C} domain and a non-BoNT/G L-chain and H_{N} domain;
iv. A BoNT/G L-chain and H_{N} domain and a non-BoNT/G H_{C} domain
v. A BoNT/G L-chain and H_{C} domain and a non-BoNT/G H_{N} domain; or
vi. A BoNT/G H_{N} domain and H_{C} domain and a non-BoNT/G L-chain.

For example, a clostridial neurotoxin of the invention (e.g. an engineered clostridial neurotoxin comprising one or more exogenous protease cleavage site) may comprise:
i. A BoNT/X L-chain and a non-BoNT/X H_{N} and H_{C} domain;
ii. A BoNT/X H_{N} domain and a non-BoNT/X L-chain and H_{C} domain
iii. A BoNT/X H_{C} domain and a non-BoNT/X L-chain and H_{N} domain;
iv. A BoNT/X L-chain and H_{N} domain and a non-BoNT/X H_{C} domain
v. A BoNT/X L-chain and H_{C} domain and a non-BoNT/X H_{N} domain; or
vi. A BoNT/X H_{N} domain and H_{C} domain and a non-BoNT/X L-chain.

For example, a clostridial neurotoxin of the invention (e.g. an engineered clostridial neurotoxin comprising one or more exogenous protease cleavage site) may comprise:
i. A TeNT L-chain and a non-TeNT H_{N} and H_{C} domain;
ii. A TeNT H_{N} domain and a non-TeNT L-chain and H_{C} domain
iii. A TeNT H_{C} domain and a non-TeNT L-chain and H_{N} domain;
iv. A TeNT L-chain and H_{N} domain and a non-TeNT H_{C} domain
v. A TeNT L-chain and H_{C} domain and a non-TeNT H_{N} domain; or
vi. A TeNT H_{N} domain and H_{C} domain and a non-TeNT L-chain.

The term “clostridial neurotoxin” may also embrace newly discovered botulinum neurotoxin and botulinum neurotoxin-like protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OT022244.1), which cleaves VAMP2 and SNAP25, the Chryseobacterium pipero encoded toxin (NCBI Ref. Seq: WP_034687872.1) and the mosquito BoNT-like protein PMP1 (NCBI Ref. Seq: QEZ70852.1).

The term “clostridial neurotoxin” is intended to embrace re-targeted clostridial neurotoxins. In a re-targeted clostridial neurotoxin, the clostridial neurotoxin is modified to include an exogenous ligand (i.e. not derived from a clostridial neurotoxin) known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety. Thus, a clostridial neurotoxin of the invention may be a re-targeted clostridial neurotoxin. In particular, an engineered clostridial neurotoxin of the invention may be an engineered re-targeted clostridial neurotoxin. The engineered re-targeted clostridial neurotoxins of the invention may comprise TM that are presented at the N- or C-terminus of the single-chain neurotoxin, or the TM may be presented centrally within the single-chain neurotoxin. In some preferred embodiments, the engineered re-targeted clostridial neurotoxins of the invention may comprise TM that are presented at the N- or C-terminus of the single-chain neurotoxin.

Engineering re-targeted clostridial neurotoxins may allow for the use of TM that are susceptible to cleavage by proteases conventionally used to activate recombinantly produced re-targeted clostridial neurotoxins, such as trypsin, Lys-C and/or BoNT hydrolase. Thus, engineering re-targeted clostridial neurotoxins to include one or more exogenous protease activation site according to the invention may allow for improvements in stability compared to a corresponding re-targeted clostridial neurotoxins which is activated by a conventional activating protease such as Lys-C, trypsin and/or BoNT hydrolase.

In some preferred embodiments, an engineered re-targeted clostridial neurotoxin comprises a BoNT/A light chain (LC/A) and/or a BoNT/A translocation domain (H_{N}/A), particularly preferred both a LC/A and H_{N}/A, optionally wherein in the pre-engineered re-targeted clostridial neurotoxin the endogenous BoNT/A activation loop has been replaced with an endogenous BoNT/C activation loop. Non-limiting examples of engineered re-targeted clostridial neurotoxins, may comprise SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

In some particularly preferred embodiments, an engineered re-targeted clostridial neurotoxin comprises a BoNT/X light chain (LC/X) and/or a BoNT/X translocation domain (H_{N}/X), particularly preferred both a LC/X and H_{N}/X, optionally wherein in the pre-engineered re-targeted clostridial neurotoxin the endogenous BoNT/X activation loop has been replaced with an endogenous BoNT/C activation loop. Such engineered re-targeted BoNT/X are particularly preferred. Non-limiting examples of engineered re-targeted clostridial neurotoxins, may comprise SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

The clostridial neurotoxin of the present invention (e.g. an engineered clostridial neurotoxin) may lack a functional H_{C} domain of a clostridial neurotoxin and also lack any functionally equivalent TM. Accordingly, said polypeptides lack the natural binding function of a clostridial neurotoxin and are not able to bind rat synaptosomal membranes (via a clostridial H_{C} component, or via any functionally equivalent TM) in binding assays as described in Shone et al. (1985) Eur. J. Biochem. 151, 75-82. Preferably the TM is not a Wheat Germ Agglutinin (WGA) peptide. Thus, in some preferred embodiments the clostridial neurotoxin is a re-targeted clostridial neurotoxin in which an endogenous H_{C} or H_{CC} of a clostridial neurotoxin is replaced by an exogenous TM. Particularly preferred are embodiments in which the engineered clostridial neurotoxin is a re-targeted clostridial neurotoxin in which an endogenous H_{C} or H_{CC} of a clostridial neurotoxin is replaced by an exogenous TM.

A clostridial neurotoxin of the invention (e.g. an engineered clostridial neurotoxin) may comprise an LH_{N} polypeptide (e.g. an engineered LH_{N} polypeptide), i.e. a polypeptide comprising or consisting of a clostridial L-chain and a clostridial H_{N} domain, as defined herein.

A clostridial neurotoxin (e.g. an engineered clostridial neurotoxin) may comprise an LH_{N} polypeptide (e.g. an engineered LH_{N} polypeptide) and a targeting moiety (TM).

The present invention also embraces clostridial neurotoxins that have an additional non-native protease cleavage site. Such a site will require an exogenous protease for cleavage, which allows for improved control over the timing and location of cleavage events. Additional non-native protease cleavage sites that may be employed in clostridial neurotoxins include:

TEV(Tobacco Etch virus)
(SEQ ID NO: 148)
(ENLYFQIG)
PreScission
(SEQ ID NO: 149)
(LEVLFQIGP).

Additional protease cleavage sites include recognition sequences that are cleaved by a non-cytotoxic protease, for example by the light chain of a clostridial neurotoxin. These include the SNARE (e.g. SNAP-25, syntaxin, VAMP) protein recognition sequences that are cleaved by non-cytotoxic proteases such as the light chain of a clostridial neurotoxin. Clostridial neurotoxins comprising non-native protease cleavage sites are described in U.S. Pat. No. 7,132,259, EP 1206554-B2 and US 2007/0166332, all of which are hereby incorporated by reference in their entirety. Also embraced by the term protease cleavage site is an intein, which is a self-cleaving sequence. The self-splicing reaction is controllable, for example by varying the concentration of reducing agent present.

The present invention also embraces clostridial neurotoxins comprising a “destructive cleavage site”. In said clostridial neurotoxins, a non-native protease cleavage site is incorporated into the clostridial neurotoxin, at a location chosen such that cleavage at said site will decrease the activity of, or inactivate, the clostridial neurotoxin. The destructive protease cleavage site can be susceptible to cleavage by a local protease, in the event that the clostridial neurotoxin, following administration, migrates to a non-target location. Suitable non-native protease cleavage sites include those described above. Clostridial neurotoxins comprising a destructive cleavage site are described in WO 2010/094905 and WO 2002/044199, both of which are hereby incorporated by reference in their entirety.

The clostridial neurotoxins (e.g. engineered clostridial neurotoxins) of the present invention, especially the light chain component thereof, may be PEGylated—this may help to increase stability, for example duration of action of the light chain component. PEGylation is particularly preferred when the light chain comprises a BoNT/A, B or C1 protease. PEGylation preferably includes the addition of PEG to the N-terminus of the light chain component. By way of example, the N-terminus of a light chain may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is hereby incorporated by reference in its entirety.

A chimeric clostridial neurotoxin of the invention may not comprise a therapeutic or diagnostic agent (e.g. a nucleic acid, protein, peptide or small molecule therapeutic or diagnostic agent) additional to the light-chain and heavy-chain. For example, in one embodiment, the chimeric clostridial neurotoxin may not comprise a covalently or non-covalently associated therapeutic or diagnostic agent. Thus, a chimeric clostridial neurotoxin of the invention preferably does not function as a delivery vehicle for a further therapeutic or diagnostic agent.

In embodiments where a chimeric clostridial neurotoxin described herein has a tag for purification (e.g. a His-tag) and/or a linker, said tag and/or linker are optional.

The clostridial neurotoxins (e.g. engineered clostridial neurotoxins) of the present invention may be free from the complexing proteins that are present in a naturally occurring clostridial neurotoxin complex.

The clostridial neurotoxins (e.g. engineered clostridial neurotoxins) of the present invention can be produced using recombinant nucleic acid technologies. Thus, in an engineered clostridial neurotoxin (as described above) may be a recombinant engineered clostridial neurotoxin. A single-chain clostridial neurotoxin (as described herein) may be a recombinant single-chain neurotoxin.

Tolerance (i.e. a reduction in the number and/or severity of any side effects) to an engineered clostridial neurotoxin of the invention may be increased compared with the tolerance to the corresponding (pre-engineering) clostridial neurotoxin. In particular, tolerance to an engineered clostridial neurotoxin of the invention may be increased compared with the tolerance to the corresponding (pre-engineering) clostridial neurotoxin when the pre-engineering clostridial neurotoxin is administered (e.g. in di-chain form).

An engineered clostridial neurotoxin of the invention may have equivalent or increased potency compared with the potency of the corresponding (pre-engineering) clostridial neurotoxin. In particular, potency of an engineered clostridial neurotoxin of the invention may be equivalent to or increased compared with the potency of the corresponding (pre-engineering) clostridial neurotoxin when the pre-engineering clostridial neurotoxin is administered in di-chain form. The term “equivalent potency” as used herein means that an engineered clostridial neurotoxin has a potency of at least about 80%, at least about 85%, at least about 90%, at least about 95%, at least about 99%, up to about 100% of the potency of the corresponding (pre-engineering) clostridial neurotoxin. Preferably “equivalent potency” as used herein means that an engineered clostridial neurotoxin has a potency of at least about 95%, at least about 99%, at least about 100%, at least about 101%, up to about 105% of the potency of the corresponding (pre-engineering) clostridial neurotoxin. The term “increased potency” as used herein means that an engineered clostridial neurotoxin has a potency of at least about 10%, at least about 15%, at least about 20%, at least about 25% greater potency compared with the potency of the corresponding (pre-engineering) clostridial neurotoxin. Potency may be measured using any appropriate assay, conventional examples of which are described herein.

An engineered clostridial neurotoxin of the invention typically has an improved safety profile and/or therapeutic window compared with the safety profile and/or therapeutic window of the corresponding (pre-engineering) clostridial neurotoxin. Without being bound by theory, this may be by virtue of its improved tolerance and/or equivalent or increased potency. In particular, an engineered clostridial neurotoxin of the invention may have an improved safety profile and/or therapeutic window compared with the safety profile and/or therapeutic window of the corresponding (pre-engineering) clostridial neurotoxin when the pre-engineering clostridial neurotoxin is administered (e.g. in di-chain form).

One way in which these advantageous properties (which represent an increase in the therapeutic index) may be defined is in terms of the Safety Ratio (for clinical applications) or Tolerance Index (TI, in animal models, which may be calculated as described below) of the engineered clostridial toxin. In this regard, undesired effects of a clostridial neurotoxin (such as those caused by diffusion of the neurotoxin away from the site of administration) can be assessed experimentally by measuring percentage bodyweight loss in a relevant animal model (e.g. a mouse, where loss of bodyweight is detected within seven days of administration). Desired on-target effects of a clostridial toxin can be assessed experimentally by any appropriate technique, depending on the target cell of interest. Suitable assays are known in the art and it would be routine for one of ordinary skill to select an appropriate assay for a given target cell type. For clostridial neurotoxins of the invention which target motor neurons, a Digital Abduction Score (DAS) assay, a measurement of muscle paralysis, may be used. The DAS assay may be performed by injection of 20 μl of (engineered) clostridial toxin, formulated in Gelatin Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice are suspended briefly by the tail in order to elicit a characteristic startle response in which the mouse extends its hind limbs and abducts its hind digits. Following clostridial toxin injection, the varying degrees of digit abduction are scored on a five-point scale (0=normal to 4=maximal reduction in digit abduction and leg extension). For clostridial neurotoxins of the invention which target other subtypes of neurones, any appropriate assay known in the art may be used. SNARE cleavage assays may also be used to assess the activity of engineered clostridial neurotoxins of the invention, examples of which are well-described in the art (e.g. Western blot). Assays to detect and/or quantify the effect of an engineered clostridial neurotoxin on the release of a maker signalling molecule may also be used. The specific marker signally molecule may be selected depending on the cell type(s) targeted by the engineered clostridial neurotoxins. For example, the signalling molecule may be a hormone, substance P, CGRP, glutamate, glycine, depending on whether cells involved with hormone secretion or pain-sensing neurons are targeted. For the treatment of pain, animal studies may be used to assess if there is a greater tolerance to a noxious stimulus. Typical in vivo assays will measure different types of pain (e.g., mechanical, cold, heat) and the readout could be behavioural (e.g., licking/biting the treated site or withdrawal from the noxious stimulus) or may involve the use of the Von Frey test. Any appropriate nociception test may be used, and examples of such tests are well-known in the art.

The Safety Ratio or TI of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio or TI scores are therefore desired, and indicate a toxin that is able to effectively paralyse a target muscle with little undesired off-target effects. An engineered toxin of the present invention may have a Safety Ratio and/or TI that is higher than the Safety Ratio and/or TI of an equivalent unmodified (pre-engineering) single-chain clostridial neurotoxin. The calculation for TI may vary depending on the experimental model used.

For example, in a DAS mouse model, an engineered clostridial toxin of the present invention has a TI of at least 8 (for example, at least 8, 9, 10, 15, 20, 25, 30, 35, 40, 45 or 50), wherein TI is calculated as: dose of toxin required for −10% bodyweight change (pg/mouse) divided by DAS ED₅₀ (pg/mouse) [ED₅₀=dose required to produce a DAS score of 2].

For clinical use a Safety Ratio may be calculated.

Any clostridial neurotoxin as described herein may be engineered according to the invention to comprise any exogenous protease cleavage site, exogenous activation loop comprising one or more exogenous protease cleavage site or modified BoNT/C (BoNT/C1) activation loop as described herein.

The invention provides a nucleic acid (for example, a DNA or RNA) comprising a nucleic acid sequence encoding a clostridial neurotoxin (e.g. an engineered clostridial neurotoxin) as described herein. The nucleic acid sequence may be prepared as part of an expression vector in which the nucleic acid is operably linked to a promoter. Preferably, the nucleic acid may be prepared as part of a DNA expression vector comprising a promoter and a terminator.

Preferably the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

Alternatively, a promoter may preferably be selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

The nucleic acid molecules and expression vectors of the present invention may be preferably designed in silico, and then synthesised by conventional synthesis techniques, including conventional DNA synthesis techniques.

The above-mentioned nucleic acid sequence information is optionally modified for codon-biasing according to the ultimate host cell (e.g. E. coli) expression system that is to be employed.

The present invention provides a nucleotide sequence encoding an engineered clostridial neurotoxin of the present invention. The nucleotide sequence of the invention encodes a polypeptide comprising one or more exogenous protease cleavage site as described herein.

The nucleotide sequence may comprise a sequence having at least 70% sequence identity to SEQ ID NO: 154, 156, 158, 160, 162 or 164. The nucleotide sequence may comprise a sequence having at least 80% or 90% sequence identity to SEQ ID NO: 154, 156, 158, 160, 162 or 164. Preferably the nucleotide sequence comprises (more preferably consists of) SEQ ID NO: 154, 156, 158, 160, 162 or 164. The nucleotide sequence may encode an engineered clostridial neurotoxin comprising or consisting of an amino acid sequence having at least 70% sequence identity to one or more of SEQ ID NOs: 155, 157, 159, 161, 163, 165, 166, 167 or 168. The nucleotide sequence may encode an engineered clostridial neurotoxin comprising or consisting of an amino acid sequence having at least 80% or 90% sequence identity to one or more of SEQ ID NOs: 155, 157, 159, 161, 163, 165, 166, 167 or 168. Preferably the nucleotide sequence encode an engineered clostridial neurotoxin comprising (more preferably consisting of) any one of SEQ ID NOs: 155, 157, 159, 161, 163, 165, 166, 167 or 168

The engineered clostridial neurotoxin may be encoded by a nucleic acid comprising or consisting of a nucleotide sequence having at least 70% sequence identity to SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be encoded by a nucleic acid comprising or consisting of a nucleotide sequence having at least 80% or 90% sequence identity to SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may encoded by a nucleic acid comprising or consisting of the nucleotide sequence of SEQ ID NO: 175, wherein SEQ ID NO: 176 within SEQ ID NO: 175 is replaced by a nucleotide sequence encoding at least one exogenous protease cleavage site or a modified BoNT/C activation loop as defined herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

Alternatively or in addition, the engineered clostridial neurotoxin may be a single-chain clostridial neurotoxin comprising or consisting of a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin comprising or consisting of a polypeptide sequence having (i) at least 80% or 90% sequence identity to SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) at least 80% or 90% identity to SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65. The engineered clostridial neurotoxin may be a single-chain clostridial neurotoxin comprising or consisting of (i) the polypeptide sequence of SEQ ID NO: 150, wherein SEQ ID NO: 2 within SEQ ID NO: 150 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65; or (ii) the polypeptide sequence of SEQ ID NO: 152, wherein SEQ ID NO: 2 within SEQ ID NO: 152 has been replaced by one or more exogenous protease cleavage site and/or modified BoNT/C activation loop as described herein, wherein the one or more exogenous protease site is preferably selected from one or more SEQ ID NOs: 15, 33 or 25, and/or the modified BoNT/C activation loop is preferably selected from one or more SEQ ID NOs: 57, 63 and/or 65.

The terms “nucleotide sequence” and “nucleic acid” and “polynucleotide” are used synonymously herein. Preferably the nucleotide sequence is a DNA sequence.

The invention provides a method of producing a single-chain (engineered) clostridial neurotoxin protein having a light chain and a heavy chain, the method comprising expressing a polynucleotide or expression vector described herein in a suitable host cell, and recovering the expressed engineered clostridial neurotoxin. Recovering the expressed engineered clostridial neurotoxin may comprise lysing the host cell to provide a host cell homogenate containing the single-chain (engineered) clostridial neurotoxin protein, and/or isolating the single-chain (engineered) clostridial neurotoxin protein. Said method may further comprise a step of introducing the polynucleotide or expression vector described herein into the host cell. Suitable host cells include bacterial cell lines used for the recombinant production of clostridial neurotoxins, particularly Escherichia coli cells.

The present invention provides a method for proteolytically processing an (engineered) clostridial neurotoxin of the present invention into a corresponding di-chain clostridial neurotoxin, the method comprising contacting the (engineered) clostridial neurotoxin with one or more exogenous protease thereby producing a di-chain clostridial neurotoxin (e.g. wherein the light chain and heavy chain are joined together by a disulphide bond).

The present invention therefore provides a di-chain clostridial neurotoxin obtainable by a method of the invention.

The term “obtainable” as used herein also encompasses the term “obtained”. Preferably the term “obtainable” means obtained.

### Activation of an Engineered Clostridial Neurotoxin

The invention provides a method for proteolytically processing an engineered clostridial neurotoxin of the invention into a corresponding di-chain clostridial neurotoxin, the method comprising contacting the engineered clostridial neurotoxin with one or more exogenous protease, thereby producing a di-chain clostridial neurotoxin. Said contacting may be in vitro, ex vivo, or in vivo, preferably in vitro. The therapeutic methods and uses of the invention may comprise the in vivo activation of an engineered clostridial neurotoxin of the invention by cleavage at the one or more exogenous protease activation site by one or more exogenous protease expression within target cells.

Thus, a method of the invention may further comprise contacting an engineered clostridial neurotoxin with one or more exogenous protease thereby producing a corresponding di-chain engineered clostridial neurotoxin. Preferably said contacting occurs in vitro.

The invention also provides a method for proteolytically processing a single-chain clostridial neurotoxin into a corresponding di-chain clostridial neurotoxin, the method comprising: (a) providing a single-chain clostridial neurotoxin; and (b) contacting the single-chain clostridial neurotoxin with one or more exogenous protease; wherein the single-chain clostridial neurotoxin has an activation loop comprising or consisting of one or more exogenous protease consensus sequence (e.g. any one or more of SEQ ID NOs: 169, 170, 172 or 173) or cleavage site as described herein (e.g. any one or more of SEQ ID NOs: 15-22, 24-30, 32-42, 44-46 and/or 50-52, particularly any one of SEQ ID NOs: 15-22, 24-30 and/or 32-42, preferably any one of SEQ ID NOs: 15, 17, 16, 26, 25, 27, 24, 33 and/or 32, with SEQ ID NOs: 15, 17, 16, 26, 25 and/or 33 being particularly preferred); and wherein one or more exogenous protease hydrolyses a peptide bond of the activation loop thereby producing a di-chain clostridial neurotoxin. Preferably said contacting occurs in vitro.

The present invention encompasses contacting a single-chain clostridial neurotoxin (e.g. an engineered clostridial neurotoxin of the invention) with one or more exogenous protease, wherein one or more exogenous protease is capable of hydrolysing a peptide bond in an activation loop of the single-chain clostridial neurotoxin thereby producing a di-chain clostridial neurotoxin. Preferably said contacting occurs in vitro.

The contacting can occur under any suitable conditions that result in the production of greater than 30%, 40%, 50% or 60% (preferably greater than 70%) of single-chain clostridial neurotoxin being proteolytically processed into the corresponding di-chain clostridial neurotoxin without, or without substantial, hydrolysis of a peptide bond outside of the activation loop of said clostridial neurotoxin. “Without substantial hydrolysis” may mean less than 5%, 4%, 3%, 2% or 1% of the clostridial neurotoxins contacted contain a peptide bond outside of the activation loop that has been hydrolysed by one or more exogenous protease in a method of the invention.

The skilled person can select appropriate reaction times, temperatures, buffers, and molar ratios of protease to single-chain clostridial neurotoxin to achieve the above. Optimisation of such conditions can be determined empirically using routine techniques, such as SDS-PAGE (e.g. stained with Coomassie or a dye of similar sensitivity) visual analysis of the reaction products following said contacting or spectrometric techniques (e.g. mass spectrometry).

When assessed by SDS-PAGE (e.g. stained with Coomassie or a dye of similar sensitivity), a method of the invention preferably results in the production of a clostridial neurotoxin L-chain and H-chain only.

The proteolytic processing by one or more exogenous protease in a method of the invention typically results in the production of less than 5 degradation products of a clostridial neurotoxin L-chain or H-chain, more preferably less than 4, 3, 2 or 1 degradation products. Preferably, the L-chain and H-chain produced by a method of the invention are full-length L-chain and H-chain.

Typically, processing by each of the one or more exogenous protease in a method of the invention hydrolyses 5 or fewer, 4 or fewer, 3 or fewer, 2 or fewer or a single peptide bond within the engineered clostridial neurotoxin, preferably hydrolysis of one or two peptide bonds.

Preferably processing by each of the one or more exogenous protease in a method of the invention hydrolyses 5 or fewer, 4 or fewer, 3 or fewer, 2 or fewer or a single peptide bond within the activation loop of the engineered clostridial neurotoxin, preferably hydrolysis of one or two peptide bonds. Where two or more exogenous protease cleavage sites are present within an engineered clostridial neurotoxin of the invention, the total number of peptide bonds that may be hydrolysed by the two or more exogenous proteases is typically 10 or fewer, 9 or fewer, 8 or fewer, 7 or fewer, 6 or fewer, 5 or fewer, 4 or fewer, 3 or fewer, 2 or fewer.

Preferably a single peptide bond is hydrolysed by each of the two or more exogenous proteases. Exemplary exogenous protease cleavage sites are described herein, with the position of the peptide bond that is hydrolysed indicated.

For in vitro activation of engineered clostridial neurotoxins by one or more exogenous protease of the invention, any appropriate conditions for activation may be used. It is within the routine practice of one of ordinary skill in the art to determine suitable conditions. By way of non-limiting example, between about 0.5 μg to about 10 μg of thrombin may be used per 1 mg of engineered clostridial neurotoxin, with activation carried out at room temperature (about 20° C.) and a pH of about 7.2 for 1 hour. By way of a further non-limiting example, between about 0.5 μg to about 10 μg of u-PA may be used per 1 mg of engineered clostridial neurotoxin, with activation carried out at room temperature (about 20° C.) and a pH of about 7.2 for 1 hour. By way of a yet further non-limiting example, between about 0.5 μg to about 100 μg of t-PA may be used per 1 mg of engineered clostridial neurotoxin, with activation carried out at room temperature (about 20° C.) and a pH of about 7.2 for 1 hour.

The step of contacting a clostridial neurotoxin with one or more exogenous protease according to the invention may occur within a patient treated with the clostridial neurotoxin. In other words, contacting a clostridial neurotoxin with one or more exogenous protease according to the invention may involve one or more exogenous protease endogenously present within the patient. Accordingly, contacting a clostridial neurotoxin with one or more exogenous protease according to the invention may occur in vivo following administration of the clostridial neurotoxin to an individual. When the contacting step occurs in vivo, it typically involves one or more exogenous protease endogenously present within the patient treated according to the invention.

Typically the step of contacting a clostridial neurotoxin with one or more exogenous protease according to the invention occurs in vitro, e.g. during manufacture of the engineered clostridial neurotoxin.

The invention also provides a di-chain clostridial neurotoxin that is obtainable by a method of the invention. As activation to the di-chain form occurs by cleavage at one or more exogenous protease cleavage site as described herein, the resulting C- and N-terminal cleaved ends of the di-chain clostridial neurotoxin will differ in sequence compared with the corresponding (pre-engineering) clostridial neurotoxin. By way of non-limiting example: (a) the C-terminal end of the cleaved engineered clostridial neurotoxin light chain may end with the amino acid sequence LVPR, preferably ALVPR, and the N-terminus of the cleaved engineered clostridial neurotoxin heavy chain may begin with the amino acid sequence GSK or GSY; (b) the C-terminus of the cleaved engineered clostridial neurotoxin light chain may end with the amino acid sequence SLVPR, and the N-terminus of the cleaved engineered clostridial neurotoxin heavy chain may begin with the amino acid sequence GSY; (c) the C-terminus of the cleaved engineered clostridial neurotoxin light chain may end with the amino acid sequence LTPR, preferably ALTPR, and the N-terminus of the cleaved engineered clostridial neurotoxin heavy chain may begin with the amino acid sequence GVR, preferably GVRL; (d) the C-terminus of the cleaved engineered clostridial neurotoxin light chain may end with the amino acid sequence PGR, and the N-terminus of the cleaved engineered clostridial neurotoxin heavy chain may begin with the amino acid sequence VVG; (e) the C-terminus of the cleaved engineered clostridial neurotoxin light chain may end with the amino acid sequence DKR, preferably AIDKR, and the N-terminus of the cleaved engineered clostridial neurotoxin heavy chain may begin with the amino acid sequence VLY; (f) the C-terminus of the cleaved engineered clostridial neurotoxin light chain may end with the amino acid sequence DKR, preferably AIDK, and the N-terminus of the cleaved engineered clostridial neurotoxin heavy chain may begin with the amino acid sequence RVLY; (g) the C-terminus of the cleaved engineered clostridial neurotoxin light chain may end with the amino acid sequence SGR, preferably PGSGR, and the N-terminus of the cleaved engineered clostridial neurotoxin heavy chain may begin with the amino acid sequence SA, preferably SAY, SAG or SAS; (h) the C-terminus of the cleaved engineered clostridial neurotoxin light chain may end with the amino acid sequence SGR, preferably PGSGR, and the N-terminus of the cleaved engineered clostridial neurotoxin heavy chain may begin with the amino acid sequence TL, preferably TLD or TLDC; (i) the C-terminus of the cleaved engineered clostridial neurotoxin light chain may end with the amino acid sequence FGR, and the N-terminus of the cleaved engineered clostridial neurotoxin heavy chain may begin with the amino acid sequence SA, preferably SAG; or (j) the C-terminus of the cleaved engineered clostridial neurotoxin light chain may end with the amino acid sequence VVPR, and the N-terminus of the cleaved engineered clostridial neurotoxin heavy chain may begin with the amino acid sequence VELVA. In contrast, conventional trypsin cleavage of the (pre-engineering) BoNT/A will result in a di-chain having a LC with a C-terminus ending with the sequence TSK, and a HC with an N-terminus beginning ALNDLC. These di-chain clostridial neurotoxins may be used in therapy as described herein. All disclosure herein in relation to therapeutic indications and formulations in the context of engineered or single-chain clostridial neurotoxins of the invention applies equally and without reservation to di-chain clostridial neurotoxin that is obtainable by a method of the invention unless otherwise stated.

### Therapy and Formulations

A clostridial neurotoxin of the present invention suitably finds utility in medicine and/or in cosmetics. In use, as the engineered clostridial neurotoxin of the invention may be cleaved in vivo by one or more exogenous protease as described herein, the clostridial neurotoxin may be in a single-chain form for administration. Preferably, the engineered clostridial neurotoxin of the invention is for administration in di-chain form (e.g. having been obtained by a method of the invention).

The (engineered) clostridial neurotoxins of the invention may be used to prevent or treat certain medical or cosmetic diseases and conditions. Thus, in a further aspect, the present invention provides a di-chain (engineered) clostridial neurotoxin as described above, for use in medicine. In addition, as described herein, the invention relates to single-chain clostridial neurotoxins for use to prevent or treat certain medical or cosmetic diseases and conditions, wherein the single-chain clostridial neurotoxin is administered to a subject. In addition, as described herein, the invention relates to a di-chain clostridial neurotoxin that is obtainable by a method of the invention for use to prevent or treat certain medical or cosmetic diseases and conditions, wherein the di-chain clostridial neurotoxin that is obtainable by a method of the invention is administered to a subject. Thus, in a further aspect, the present invention provides an (engineered) clostridial neurotoxin as described above, for use in medicine.

Accordingly, the present invention provides a clostridial neurotoxin (e.g. an engineered clostridial neurotoxin) as described above, for use in the prevention or treatment of a disease or condition selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, tics, tremors, bruxism, anal fissure, achalasia, dysphagia, lacrimation, hyperhydrosis, excessive salivation, excessive gastrointestinal secretions, muscle pain (e.g. pain from muscle spasms), headache pain (e.g. tension headache or migraine), phantom pain (e.g. phantom limb pain), brow furrows, skin wrinkles, cancer, uterine disorders, uro-genital disorders, urogenital-neurological disorders, bladder pain syndrome, interstitial cystitis, chronic neurogenic inflammation, and a smooth muscle disorder. In some instances, the condition may be selected from phantom pain (e.g. phantom limb pain) and bladder pain syndrome. Similarly, the invention also relates to single-chain clostridial neurotoxins and di-chain clostridial neurotoxins that are obtainable by a method of the invention for use in the treatment or prevention of the above-mentioned diseases or conditions. Preferably, a composition of the invention may be used in the prevention or treatment of a disease or condition selected from: limb spasticity (upper or lower); cervical dystonia; headache disorders (preferably migraine); blepharospasm; hemifacial spasm; and lower urinary tract disorders (e.g. bladder pain syndrome (preferably interstitial cystitis); overactive bladder; and detrusor overactivity (e.g. neurogenic detrusor overactivity.

Where a clostridial neurotoxin of the invention (e.g. an engineered clostridial neurotoxin) comprises a BoNT/X sequence (or portion thereof) said clostridial neurotoxin may be able to target other types of secretory cells other than neurons, due to its ability to cleave VAMP4, VAMP5 and/or Ykt6. In some embodiments, the secretory cell targeted is a secretory immune cell. A “secretory immune cell” as used herein, refers to immune cells that secrets cytokines, chemokines, or antibodies. Such secretory immune cells may be innate immune cells including, without limitation, natural killer cells, mast cells, eosinophils, basophils, macrophages, neutrophils, and dendritic cells. Secretory immune cells that secret antibodies (e.g. white blood cells) may also be targeted by the clostridial neurotoxins of the present disclosure. Non-limiting examples of antibody secreting cells include, without limitation, plasma B cells, plasmocytes, plasmacytes, and effector B cells. In some embodiments, the clostridial neurotoxin may modulate an immune response. Thus, further contemplated herein are therapeutic use of a clostridial neurotoxin of the invention to treat a condition associated with unwanted secretion, preferably unwanted immune secretion. Conditions associated with unwanted immune secretion include, without limitation: inflammation, psoriasis, allergy, haemophagocytic lymphohistiocytosis, and alcoholic pancreatic disease.

The invention also provides the use of a clostridial neurotoxin (e.g. an engineered clostridial neurotoxin or a di-chain clostridial neurotoxin that is obtainable by a method of the invention) as described above, in the manufacture of a medicament for use in a method for preventing or treating a disease or disorder as described herein.

The invention also provides a method of treating a disease or disorder as described herein, said method comprising administering a therapeutically effective amount of an clostridial neurotoxin (e.g. an engineered clostridial neurotoxin or a di-chain clostridial neurotoxin that is obtainable by a method of the invention) as described above to a subject in need thereof.

The invention also provides the non-therapeutic use of a composition as described herein for treating an aesthetic or cosmetic condition. For cosmetic or aesthetic use, the individual to be treated is preferably not suffering from a disease or disorder, such as those associated with unwanted neuronal activity and described above. More preferably, said individual is a healthy individual, i.e. an individual which is not suffering from any disease. Preferably, a composition of the invention may be used in the prevention or treatment of upper facial lines—glabellar lines, lateral canthal lines and/or intrathecal lines.

The invention provides a pharmaceutical composition comprising an (engineered) clostridial neurotoxin or a di-chain clostridial neurotoxin of the invention and a pharmaceutically acceptable carrier, excipient, adjuvant, propellant and/or salt. Preferably the (engineered) clostridial neurotoxin is in single-chain form (e.g. engineered to comprise a one or more exogenous protease cleavage site). A pharmaceutical composition of the invention may be a liquid composition (or formulation) or a solid composition (or formulation).

The invention also provides a cosmetic composition comprising an (engineered) clostridial neurotoxin of the invention or a di-chain clostridial neurotoxins of the invention and a cosmetically acceptable carrier, excipient, diluent, adjuvant, propellant and/or salt. The invention also provides the use of a cosmetic composition comprising a clostridial neurotoxin (e.g. an engineered clostridial neurotoxin or a di-chain clostridial neurotoxin that is obtainable by a method of the invention) for preventing or alleviating a cosmetic indication for which the application of a botulinum neurotoxin is indicated. The invention also provides the use of a cosmetic composition comprising a clostridial neurotoxin (e.g. an engineered clostridial neurotoxin or a di-chain clostridial neurotoxin that is obtainable by a method of the invention) for preventing or alleviating a cosmetic indication for which the application of a botulinum neurotoxin is indicated. Preferably the (engineered) clostridial neurotoxin is in di-chain form (e.g. engineered to comprise one or more exogenous protease cleavage site and cleavage by one or more exogenous protease during manufacturing). A cosmetic composition of the invention may be a liquid composition (or formulation) or a solid composition (or formulation).

The clostridial neurotoxins of the present invention (e.g. an engineered clostridial neurotoxin) may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

A liquid composition of the invention may be (i) a pre-lyophilisation solution, (ii) a post-reconstitution solution, or (iii) a solution which is not intended for lyophilisation and/or which has not undergone post-lyophilisation reconstitution. Liquid compositions of class (iii) may also be referred to as a “ready-to-use” compositions or “ready-to-use” solutions, as they are manufactured and formulated as a liquid and sold for use in liquid form. All disclosure herein in relation to liquid formulations applies to any liquid formulation, including pre-lyophilisation solutions, post-reconstitution solutions and ready-to-use compositions, unless expressly stated to the contrary. A liquid composition may be packaged based on the amount (particularly the absolute weight) of the chimeric clostridial neurotoxin of the invention, as described herein. The liquid composition may be packaged to allow for up to 15 injections to be administered from a single container.

A solid composition may be packaged based on the amount (particularly the absolute weight) of the engineered clostridial neurotoxin of the invention, as described herein

In the case of a clostridial neurotoxin (e.g. an engineered clostridial neurotoxin) that is to be delivered locally, the clostridial neurotoxin (e.g. an engineered clostridial neurotoxin) may be formulated as a cream (e.g. for topical application), or for sub-dermal injection.

Local delivery means may include an aerosol, or other spray (e.g. a nebuliser). In this regard, an aerosol formulation of a clostridial neurotoxin (e.g. an engineered clostridial neurotoxin) enables delivery to the lungs and/or other nasal and/or bronchial or airway passages.

Clostridial neurotoxins of the invention (e.g. an engineered clostridial neurotoxin) may be administered to a patient by intrathecal or epidural injection in the spinal column at the level of the spinal segment involved in the innervation of an affected organ.

A preferred route of administration is via laproscopic and/or localised, particularly intramuscular, injection.

The dosage ranges for administration of the compositions of the present invention are those to produce the desired therapeutic effect. A therapeutically effective dose refers to an amount of the chimeric neurotoxin, to be used in a composition of the present invention which prevents, ameliorates or treats the symptoms accompanying a disease or condition referred herein. Therapeutic efficacy and toxicity of the compound are typically determined in the art by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. In general terms, it will be appreciated that the dosage range required for a given pharmaceutical depends on the precise nature of the composition, the route of administration, the nature of the formulation, the age of the patient, the nature, extent or severity of the patient's condition, contraindications, if any, and the judgement of the attending physician.

In relation to the engineered neurotoxin compositions of the invention, suitable single unit doses (also referred to as unit doses), i.e. the dose administered per injection site are described in the art, such as in WO2021/186160, WO2021/186167, WO2023/047127, WO2023/089343 and WO2023/041934, each of which is herein incorporated by reference in its entirety. By way of non-limiting example, a single unit dose is 15,000 μg of engineered neurotoxin, 25,000 μg of engineered neurotoxin or 36,000 μg of engineered neurotoxin.

A treatment may comprise injections at multiple injection sites (typically no more than 20, preferably no more than 15 injection sites), with a single unit dose injected at each injection site.

Fluid dosage forms are typically prepared utilising the clostridial neurotoxin (e.g. an engineered clostridial neurotoxin) and a pyrogen-free sterile vehicle. The clostridial neurotoxin (e.g. an engineered clostridial neurotoxin), depending on the vehicle and concentration used, can be either dissolved or suspended in the vehicle. In preparing solutions the clostridial neurotoxin (e.g. an engineered clostridial neurotoxin) can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, viral delivery systems or high-pressure aerosol impingement.

Disclosure related to the various methods of the invention are intended to be applied equally to other methods, the clostridial neurotoxins, e.g. engineered clostridial neurotoxins (whether in single-chain or di-chain forms), uses or pharmaceutical compositions, as well as medical uses thereof and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{[ \begin{matrix}length of the longer sequence plus the number of gaps \\ introduced into the longer sequence in order \\ to align the two sequences\end{matrix} ]} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

```
SEQUENCE INFORMATION
Where an initial Met amino acid residue or a corresponding initial codon is indi-
cated in any of the following SEQ ID NOs, said residue/codon is optional.
C1 activation loop consensus
SEQ ID NO: 1
C-(X)ₐ-I-D/E-G-R-(Y)b-C
wherein a = 1-10 and b = 4-15
C1 activation loop
SEQ ID NO: 2
CHKAIDGRSLYNKTLDC
C1 activation loop
SEQ ID NO: 3
CHKAIEGRSLYNKTLDC
N-terminal residues of modified C1 activation loop
SEQ ID NO: 4
CHKA
N-terminal residues of modified C1 activation loop
SEQ ID NO: 5
CHKAI
N-terminal residues of modified C1 activation loop
SEQ ID NO: 6
CHKAID
Residues missing from modified C1 activation loop
SEQ ID NO: 7
IDGR
Residues missing from modified C1 activation loop
SEQ ID NO: 8
IEGR
Residues missing from modified C1 activation loop
SEQ ID NO: 9
CHKAIDGR
Residues missing from modified C1 activation loop
SEQ ID NO: 10
CHKAIEGR
C-terminal residues of modified C1 activation loop
SEQ ID NO: 11
TLDC
C-terminal residues of modified C1 activation loop
SEQ ID NO: 12
KTLDC
C-terminal residues of modified C1 activation loop
SEQ ID NO: 13
YNKTLDC
C-terminal residues of modified C1 activation loop
SEQ ID NO: 14
LYNKTLDC
thrombin cleavage site
SEQ ID NO: 15
LTPRGVRL
thrombin cleavage site
SEQ ID NO: 16
LVPRGS
thrombin cleavage site
SEQ ID NO: 17
ENKSLVPRGS
thrombin cleavage site consensus
SEQ ID NO: 18
X₁-X₂-X₃-R-X₄-X₅-X₆-X₇
where: X₃ is P, A, L or G
            X₄ is S, A or G
thrombin cleavage site consensus
SEQ ID NO: 19
X₁-X₂-X₃-X₄-X₅-X₆-X₇-X₈
where: X₃ is P, A, L or G
            X₃ is R or K
            X₅ is S, A or G
  thrombin cleavage site consensus
SEQ ID NO: 20
X₁-X₂-X₃-X₄-X₅-X₆-X₇-X₈
where: X₄ is R or K
thrombin cleavage site consensus
SEQ ID NO: 21
X₁-X₂-P-R-X₃-X₄-X₅-X₆
where: X₃ is S, A or G
thrombin cleavage site consensus
SEQ ID NO: 22
X₁-X₂-P-K-X₃-X₄-X₅-X₆
where: X₃ is S, A or G
thrombin
SEQ ID NO: 23
MAHVRGLQLPGCLALAALCSLVHSQHVFLAPQQARSLLQRVRRANTFLEEVRKGNLERECVEETCSYE
EAFEALESSTATDVFWAKYTACETARTPRDKLAACLEGNCAEGLGTNYRGHVNITRSGIECQLWRSRY
PHKPEINSTTHPGADLQENFCRNPDSSTTGPWCYTTDPTVRRQECSIPVCGQDQVTVAMTPRSEGSSV
NLSPPLEQCVPDRGQQYQGRLAVTTHGLPCLAWASAQAKALSKHQDENSAVOLVENFCRNPDGDEEGV
WCYVAGKPGDFGYCDLNYCEEAVEEETGDGLDEDSDRAIEGRTATSEYQTFFNPRTFGSGEADCGLRP
LFEKKSLEDKTERELLESYIDGRIVEGSDAEIGMSPWQVMLFRKSPQELLCGASLISDRWVLTAAHCL
LYPPWDKNFTENDLLVRIGKHSRTRYERNIEKISMLEKIYIHPRYNWRENLDRDIALMKLKKPVAFSD
YIHPVCLPDRETAASLLQAGYKGRVTGWGNLKETWTANVGKGQPSVLQVVNLPIVERPVCKDSTRIRI
TDNMFCAGYKPDEGKRGDACEGDSGGPFVMKSPFNNRWYQMGIVSWGEGCDRDGKYGFYTHVFRLKKW
IQKVIDQFGE
urokinase (u-PA) cleavage site
SEQ ID NO: 24
KRV
urokinase (u-PA) cleavage site
SEQ ID NO: 25
SGRSA
urokinase (u-PA) cleavage site
SEQ ID NO: 26
PGSGRSAG
urokinase (u-PA) cleavage site
SEQ ID NO: 27
PGSGRSASGTTGTG
u-PA cleavage site consensus
SEQ ID NO: 28
X₁-S-X₂-X₃-G-X₄-X₅-X₆
where: X₂ is G or S
          X₃ is R or K
          X₄ is L, R or V
          X6 is N or G
u-PA cleavage site consensus
SEQ ID NO: 29
X₁-X₂-X₃-R-V-X₄-X₅-X₆
u-PA cleavage site consensus
SEQ ID NO: 30
X₁-S-G-R-G-X₁-X₂-X₃
where: X₁ is L, R or V
            X₃ is N or G
u-PA
SEQ ID NO: 31
MRVWLASLFLCALVANSEGGSELEASDESNCGCONGGVCVSYKYESSIRRCSCPKKFKGEHCEIDTSK
TCYHGNGQSYRGKANTDTKGRPCLAWNSPAVLOQTYNAHRSDALSLGLGKHNYCRNPDNORRPWCYVQ
IGLKQFVQECMVQDCSLSKKPSSTVDQQGFQCGQKALRPRFKIVGGEFTVVENQPWFAAIYLKNKGGS
PPSFKCGGSLISPCWVASATHCFVNQPKKEEYVVYLGQSKRNSYNPGEMKFEVEQLILHEDFSDETLA
FHNDIALLKIRTSTGQCAQPSRTIQTICLPPRFGDAPFGSDCEITGFGQESATDYFYPKDLKMSVVKI
ISHEQCKQPHYYGSEINYKMLCAADPEWKTDSCSGDSGGPLICNIDGRPTLSGIVSWGSGCAEKNKPG
VYTRVSYFLNWIQSHIGEENGLAF
tissue plasminogen activator (t-PA) cleavage site
SEQ ID NO: 32
VVPRVELVA
tissue plasminogen activator (t-PA) cleavage site
SEQ ID NO: 33
PPFGRSAG
t-PA cleavage site consensus
SEQ ID NO: 34
X₁-X₂-X₃-R-X₄-X₅
where: X2 is F or Y
            X₃ is S, G or A
t-PA cleavage site consensus
SEQ ID NO: 35
G-P-X₁-X₂-X₃-G-G-X₄
where: X₂ is K or R
t-PA cleavage site consensus
SEQ ID NO: 36
X₁-X₂-X₃-R-V-X₄-X₅-X₆
t-PA cleavage site consensus
SEQ ID NO: 37
X₁-X₂-X₃-R-X₄-X₅-X₆-X₇
where: X₂ is F or Y
            X₃ is S, G or A
t-PA cleavage site consensus
SEQ ID NO: 38
S-X₁-X₂-R-X₃-X₄-X₅-X₆
where: X₁ is F or Y
          X₂ is S, G or A
t-PA cleavage site consensus
SEQ ID NO: 39
G-P-X₁-X₂-X₃-G-G-X₄
where: X₂ is K or R
t-PA cleavage site consensus
SEQ ID NO: 40
G-P-Y-X₁-X₂-G-G-X₂
where: X₁ is For Y
t-PA cleavage site consensus
SEQ ID NO: 41
G-P-X₁-X₂-K-G-G-X₃
where: X₂ is K or R
  t-PA cleavage site consensus
SEQ ID NO: 42
G-P-Y-X₁-K-G-G-X₂
where: X1 is K or R
t-PA
SEQ ID NO: 43
MDAMKRGLCCVLLLCGAVFVSPSQEIHARFRRGARSYQVICRDEKTOMIYQQHQSWLRPVLRSNRVEY
CWCNSGRAQCHSVPVKSCSEPRCFNGGTCQQALYFSDFVCQCPEGFAGKCCEIDTRATCYEDQGISYR
GTWSTAESGAECTNWNSSALAQKPYSGRRPDAIRLGLGNHNYCRNPDRDSKPWCYVFKAGKYSSEFCS
TPACSEGNSDCYFGNGSAYRGTHSLTESGASCLPWNSMILIGKVYTAQNPSAQALGLGKHNYCRNPDG
DAKPWCHVLKNRRLTWEYCDVPSCSTCGLRQYSQPQFRIKGGLFADIASHPWQAAIFAKHRRSPGERF
LCGGILISSCWILSAAHCFQERFPPHHLTVILGRTYRVVPGEEEQKFEVEKYIVHKEFDDDTYDNDIA
LLQLKSDSSRCAQESSVVRTVCLPPADLOLPDWTECELSGYGKHEALSPFYSERLKEAHVRLYPSSRC
TSQHLLNRTVTDNMLCAGDTRSGGPQANLHDACQGDSGGPLVCLNDGRMTLVGIISWGLGCGQKDVPG
VYTKVINYLDWIRDNMRP
FVII/FIX cleavage site
SEQ ID NO: 44
GRI
FIX cleavage site consensus
SEQ ID NO: 45
X₁-X₂-G-R-X₃-X₄-X₅-X₆
FVII/FIX cleavage site consensus
SEQ ID NO: 46
X₁-X₂-X₃-R-I-X₄-X₅-X₆
FIX
SEQ ID NO: 47
MQRVNMIMAESPGLITICLLGYLLSAECTVELDHENANKILNRPKRYNSGKLEEFVOGNLERECMEEK
CSFEEAREVFENTERTTEFWKQYVDGDQCESNPCLNGGSCKDDINSYECWCPFGFEGKNCELDVTCNI
KNGRCEQFCKNSADNKVVCSCTEGYRLAENQKSCEPAVPFPCGRVSVSQTSKLTRAETVFPDVDYVNS
TEAETILDNITQSTQSFNDFTRVVGGEDAKPGQFPWQVVLNGKVDAFCGGSIVNEKWIVTAAHCVETG
VKITVVAGEHNIEETEHTEQKRNVIRIIPHHNYNAAINKYNHDIALLELDEPLVLNSYVTPICIADKE
YTNIFLKFGSGYVSGWGRVFHKGRSALVLQYLRVPLVDRATCLRSTKFTIYNNMFCAGFHEGGRDSCQ
GDSGGPHVTEVEGTSFLTGIISWGEECAMKGKYGIYTKVSRYVNWIKEKTKLT
FVII
SEQ ID NO: 48
MVSQALRLLCLLLGLQGCLAAGGVAKASGGETRDMPWKPGPHRVFVTQEEAHGVLHRRRRANAFLEEL
RPGSLERECKEEQCSFEEAREIFKDAERTKLFWISYSDGDQCASSPCQNGGSCKDQLQSYICFCLPAF
EGRNCETHKDDQLICVNENGGCEQYCSDHTGTKRSCRCHEGYSLLADGVSCTPTVEYPCGKIPILEKR
NASKPQGRIVGGKVCPKGECPWQVLLLVNGAQLCGGTLINTIWVVSAAHCFDKIKNWRNLIAVLGEHD
LSEHDGDEQSRRVAQVIIPSTYVPGTTNHDIALLRLHQPVVLTDHVVPLCLPERTESERTLAFVRESL
VSGWGQLLDRGATALELMVLNVPRLMTQDCLQQSRKVGDSPNITEYMFCAGYSDGSKDSCKGDSGGPH
ATHYRGTWYLTGIVSWGQGCATVGHFGVYTRVSQYIEWLQKLMRSEPRPGVLLRAPFP
Plasminogen
SEQ ID NO: 49
MEHKEVVLLLLLFLKSGQGEPLDDYVNTQGASLFSVTKKQLGAGSIEECAAKCEEDEEFTCRAFQYHS
KEQQCVIMAENRKSSIIIRMRDVVLFEKKVYLSECKTGNGKNYRGTMSKTKNGITCQKWSSTSPHRPR
FSPATHPSEGLEENYCRNPDNDPQGPWCYTTDPEKRYDYCDILECEEECMHCSGENYDGKISKTMSGL
ECQAWDSQSPHAHGYIPSKFPNKNLKKNYCRNPDRELRPWCFTTDPNKRWELCDIPRCTTPPPSSGPT
YQCLKGTGENYRGNVAVTVSGHTCQHWSAQTPHTHNRTPENFPCKNLDENYCRNPDGKRAPWCHTTNS
QVRWEYCKIPSCDSSPVSTEQLAPTAPPELTPVVQDCYHGDGQSYRGTSSTTTTGKKCQSWSSMTPHR
HQKTPENYPNAGLTMNYCRNPDADKGPWCFTTDPSVRWEYCNLKKCSGTEASVVAPPPVVLLPDVETP
SEEDCMFGNGKGYRGKRATTVTGTPCQDWAAQEPHRHSIFTPETNPRAGLEKNYCRNPDGDVGGPWCY
TTNPRKLYDYCDVPQCAAPSFDCGKPQVEPKKCPGRVVGGCVAHPHSWPWQVSLRTRFGMHFCGGTLI
SPEWVLTAAHCLEKSPRPSSYKVILGAHQEVNLEPHVQEIEVSRLFLEPTRKDIALLKLSSPAVITDK
VIPACLPSPNYVVADRTECFITGWGETQGTFGAGLLKEAQLPVIENKVCNRYEFLNGRVOSTELCAGH
LAGGTDSCQGDSGGPLVCFEKDKYILQGVTSWGLGCARPNKPGVYVRVSRFVTWIEGVMRNN
Plasminogen cleavage site
SEQ ID NO: 50
PGRVVGG
Plasminogen cleavage site consensus
SEQ ID NO: 51
X₁-X₂-X₃-K-X₄-X₅-X₆-X₇
Plasminogen cleavage site consensus
SEQ ID NO: 52
X₁-X₂-X₃-R-X₄-X₅-X₆-X₇
modified C-loop with minimal uPA/tPA cleavage site
SEQ ID NO: 53
CHKAIDKRVLYNKTLDC
modified C-loop with minimal thrombin cleavage site
SEQ ID NO: 54
CHKALVPRGSYNKTLDC
modified C-loop with minimal FIX/FVII cleavage site
SEQ ID NO: 55
CHKAIDGRILYNKTLDC
modified C-loop with full thrombin cleavage site
SEQ ID NO: 56
CHKAENKSLVPRGSYNKTLDC
modified C-loop with alternative thrombin cleavage site
SEQ ID NO: 57
CHKALTPRGVRLKTLDC
modified C-loop with native plasminogen cleavage site
SEQ ID NO: 58
CPGRVVGGC
modified C-loop with native plasminogen cleavage site with
spacers
SEQ ID NO: 59
CGGGGSPGRVVGGGGSC
optimal u-PA cleavage site in plasminogen loop
SEQ ID NO: 60
CSGRSAGGC
optimal u-PA cleavage site with spacers
SEQ ID NO: 61
CGGGGSGRSAGGGGSC
modified C-loop with alternative t-PA cleavage site
SEQ ID NO: 62
CHKAVVPRVELVATLDC
modified C-loop with alternative t-PA cleavage site
SEQ ID NO: 63
CHKAIPPFGRSAGYNKTLDC
modified C-loop with alternative t-PA cleavage site
SEQ ID NO: 64
CHKAIPGSGRSAGYNKTLDC
modified C-loop with alternative u-PA cleavage site
SEQ ID NO: 65
CHKAISGRSAYNKTLDC
modified C-loop with alternative t-PA cleavage site
SEQ ID NO: 66
CHKAIPGSGRSASGTTGTGYNKTLDC
endogenous BoNT/D Activation Loop
SEQ ID NO: 67
CLRLTKNSRDDSTC
endogenous BoNT/DC Activation Loop
SEQ ID NO: 68
CLRLTRNSRDDSTC
endogenous BoNT/C1 and CD Activation Loop
SEQ ID NO: 69
CHKAIDGRSLYNKTLDC
endogenous BoNT/A4 Activation Loop
SEQ ID NO: 70
CVRGIITSKTKSLDEGYNKALNELC
endogenous BoNT/A5 and A7 Activation Loop
SEQ ID NO: 71
CVRGIITSKTKSLDEGYNKALNDLC
endogenous BoNT/A1 and A6 Activation Loop
SEQ ID NO: 72
CVRGIITSKTKSLDKGYNKALNDLC
endogenous BoNT/A3 Activation Loop
SEQ ID NO: 73
CVRGIIPFKTKSLDEGYNKALNYLC
endogenous BoNT/A2 and A8 Activation Loop
SEQ ID NO: 74
CVRGIIPFKTKSLDEGYNKALNDLC
endogenous BoNT/H Activation Loop
SEQ ID NO: 75
CSNSNTKNSLC
endogenous BoNT/E1 to E5, E9 and E12 Activation Loop
SEQ ID NO: 76
CKNIVSVKGIRKSIC
endogenous BoNT/E11 Activation Loop
SEQ ID NO: 77
CTNIFSPKGIRKSIC
endogenous BoNT/E7, E8 and E10 Activation Loop
SEQ ID NO: 78
CKNIVFSKGITKSIC
endogenous BoNT/E6 Activation Loop
SEQ ID NO: 79
CKNIVFSKGIRKSIC
endogenous BoNT/F7 Activation Loop
SEQ ID NO: 80
CKSIVSKKGTKNSLC
endogenous BoNT/F5 Activation Loop
SEQ ID NO: 81
CLNSSFKKNTKKPLC
endogenous BoNT/F1 and F6 Activation Loop
SEQ ID NO: 82
CKSVIPRKGTKAPPRLC
endogenous BoNT/F4 Activation Loop
SEQ ID NO: 83
CKSIIPRKGTKAPPRLC
endogenous BoNT/F2 and F3 Activation Loop
SEQ ID NO: 84
CKSIIPRKGTKQSPSLC
endogenous TeNT Activation Loop
SEQ ID NO: 85
CKKIIPPTNIRENLYNRTASLTDLGGELC
endogenous BoNT/G Activation Loop
SEQ ID NO: 86
CKPVMYKNTGKSEQC
endogenous BoNT/B4 Activation Loop
SEQ ID NO: 87
CKSVKVPGIC
endogenous BoNT/B2, B3, B6 and B8 Activation Loop
SEQ ID NO: 88
CKSVRAPGIC
endogenous BoNT/B1, B5 and B7 Activation Loop
SEQ ID NO: 89
CKSVKAPGIC
endogenous BoNT/X Activation Loop
SEQ ID NO: 90
CPRNGLLYNAIYRNSKNYLNNIDLEDKKTTSKTNVSYPCSLLNGC
(BoNT/A1 - UniProt P10845)
SEQ ID NO: 91
MPFVNKQFNYKDPVNGVDIAYIKIPNVGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNINLAANFNGQNTEINNMNFTKLKNFT

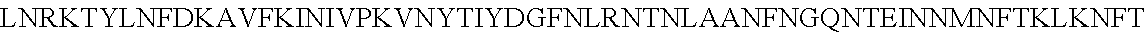

ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLIVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNORLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLEDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
In some embodiments, valine27 may be substituted with alanine, as shown in SEQ ID NO:
135.
The endogenous activation loop is dash-underlined.
(BoNT/A2 - GenBank Accession No. X73423.1)
SEQ ID NO: 92
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADI IQFECKSFGHDVLNLTRNGYGSTQYIRFSPDFTFGFEESLEVDTNPLLGAGKFATDPAVTLAHEL
IHAEHRLYGIAINPNRVFKVNTNAYYEMSGLEVSFEELRTFGGHDAKFIDSLQENEFRLYYYNKFKDVASTLNKA
KSIIGTTASLQYMKNVFKEKYLLSEDTSGKFSVDKLKFDKLYKMLTEIYTEDNFVNFFKVINRKTYLNFDKAVFR

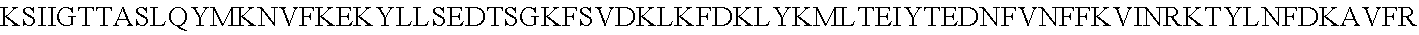

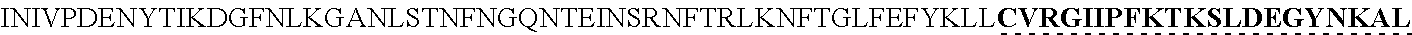


GQLEPMPNIERFPNGKKYELDKYTMFHYLRAQEFEHGDSRIILTNSAEEALLKPNVAYTFFSSKYVKKINKAVEA
FMFLNWAEELVYDFTDETNEVTTMDKIADITIIVPYIGPALNIGNMLSKGEFVEAIIFTGVVAMLEFIPEYALPV
FGTFAIVSYIANKVLTVQTINNALSKRNEKWDEVYKYTVTNWLAKVNTQIDLIREKMKKALENQAEATKAIINYQ
YNQYTEEEKNNINFNIDDLSSKLNESINSAMININKFLDQCSVSYLMNSMIPYAVKRLKDFDASVRDVLLKYIYD
NRGTLVLQVDRLKDEVNNILSADIPFQLSKYVDNKKLLSTFTEYIKNIVNTSILSIVYKKDDLIDLSRYGAKINI
GDRVYYDSIDKNQIKLINLESSTIEVILKNAIVYNSMYENFSTSFWIKIPKYFSKINLNNEYTIINCIENNSGWK
VSLNYGEIIWTLQDNKONIQRVVFKYSQMVNISDYINRWIFVTITNNRLTKSKIYINGRLIDQKPISNLGNIHAS
NKIMFKLDGCRDPRRYIMIKYFNLFDKELNEKEIKDLYDSQSNSGILKDFWGNYLQYDKPYYMLNLFDPNKYVDV
NNIGIRGYMYLKGPRGSVVTTNIYLNSTLYEGTKFIIKKYASGNEDNIVRNNDRVYINVVVKNKEYRLATNASQA
GVEKILSALEIPDVGNLSQVVVMKSKDDQGIRNKCKMNLQDNNGNDIGFIGFHLYDNIAKLVASNWYNRQVGKAS
RTFGCSWEFIPVDDGWGESSL
The endogenous activation loop is dash-underlined.
(BoNT/A3 - GenBank Accession No. DQ185900.1)
SEQ ID NO: 93
MPFVNKPFNYRDPGNGVDIAYIKIPNAGQMQPVKAFKIHEGVWVIPERDTFTNPEEGDLNPPPEAKQVPVSYYDS
TYLSTDNEKDNYLKGVIKLFDRIYSTGLGRMLLSFIVKGIPFWGGSTIDTELKVIDTNCINVIEPGGSYRSEELN
LVITGPSADIIQFECKSFGHDVFNLTRNGYGSTQYIRFSPDFTFGFEESLEVDTNPLLGAGTFATDPAVTLAHEL
IHAAHRLYGIAINPNRVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKVINRKTYLNFDKAVFR

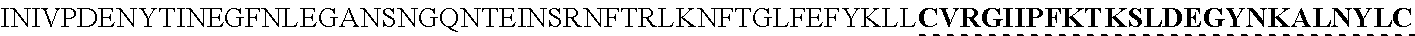

IKVNNWDLFFSPSEDNFTNDLDKVEEITADTNIEAAEENISSDLIQQYYLTFDFDNEPENISIENLSSDIIGQLE
PMPNIERFPNGKKYELDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVALLEFIPEYSLPVFGTF
AIVSYIANKVLTVQTINNALSKRNEKWDEVYKYTVTNWLAKVNTQIDLIREKMKKALENQAEATRAIINYQYNQY
TEEEKNNINFNIDDLSSKLNRSINRAMININKFLDQCSVSYLMNSMIPYAVKRLKDFDASVRDVLLKYIYDNRGT
LILQVDRLKDEVNNILSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTIINCIENNSGWKVSLN
YGEIIWTLQDNKQNIQRVVFKYSQMVNISDYINRWMFVTITNNRLTKSKIYINGRLIDQKPISNLGNIHASNKIM
FKLDGCRDPRRYIMIKYFNLFDKELNEKEIKDLYDSQSNPGILKDFWGNYLQYDKPYYMLNLFDPNKYVDVNNIG
IRGYMYLKGPRGSVMTTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVASNWYNRQVGKASRTFG
CSWEFIPVDDGWGESSL
The endogenous activation loop is dash-underlined.
(BoNT/A4 - GenBank Accession No. EU341307.1)
SEQ ID NO: 94
MPLVNQQINYYDPVNGVDIAYIKIPNAGKMQPVKAFKIHNKVWVIPERDIFTNPEEVDLNPPPEAKQVPISYYDS
AYLSTDNEKDNYLKGVIKLFERIYSTDLGRMLLISIVRGIPFWGGGKIDTELKVIDTNCINIIQLDDSYRSEELN
LAIIGPSANIIESQCSSFRDDVLNLTRNGYGSTQYIRFSPDFTVGFEESLEVDTNPLLGAGKFAQDPAVALAHEL
IHAEHRLYGIAINTNRVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKVLNRKTYLNFDKAVFK

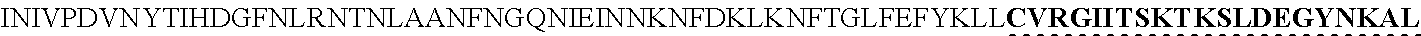

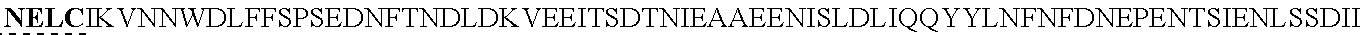


GQLEPMPNIERFPNGKKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSGAVILLEIVPEIALPV
LGTFALVSYVSNKVLTVQTIDNALSKRNEKWDEVYKYIVTNWLAIVNTQINLIREKMKKALENQAEATKAIINYQ
YNQYTEEEKNNINFNIDDLSSKLNESINSAMININKFLDQCSVSYLMNSMIPYAVKRLKDFDASVRDVLLKYIYD
NRGTLIGQVNRLKDKVNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNNEYTIINCMENNSGWK
VSLNYGEIIWTFQDTQEIKQRVVFKYSQMINISDYINRWIFVTITNNRITKSKIYINGRLIDQKPISNLGNIHAS
NKIMFKLDGCRDPHRYIVIKYFNLFDKELSEKEIKDLYDNQSNSGILKDFWGDYLOYDKSYYMLNLYDPNKYVDV
NNVGIRGYMYLKGPRDNVMTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWRERPL
The endogenous activation loop is dash-underlined.
(BoNT/A5 - GenBank Accession No. EU679004.1)
SEQ ID NO: 95
MLFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTELGRMLLTSIVRGIPFWGGSTIDTELKVIDTNCINVIQPDGSYRSEELN
LVIIGPSADIIQFECKSFGHDVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGEHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK

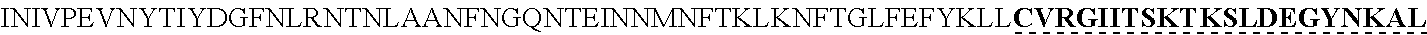

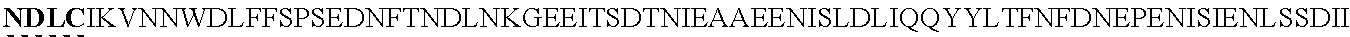


GQLELMPNIERFPNGKKYELDKYTMFHYLRAQEFEHGKSRIVLTNSVNEALLNPSSVYTFFSSDYVRKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWGEVYKYIVTNWLAKVNTQIDLIRKKMKEALENQAEATKAIINYQ
YNQYTEEEKNNINFNIGDLSSKLNDSINKAMININKFLNQCSVSYLMNSMIPYGVKRLEDFDASLKDALLKYIYD
NRGTLIGQVDRLKDKVNNTLSTDIPFQLSKYVDNORLLSTFTEYIKNIINTSILNLRYESNHLIDLSRYASEINI
GSKVNFDPIDKNQIQLFNLESSKIEIILKNAIVYNSMYENFSTSFWIKIPKYFSKINLNNEYTIINCIENNSGWK
VSLNYGEIIWTLQDNKONIQRVVFKYSOMVAISDYINRWIFITITNNRLNNSKIYINGRLIDQKPISNLGNIHAS
NNIMFKLDGCRDPHRYIWIKYFNLFDKELNEKEIKDLYDNQSNSGILKDFWGNYLQYDKPYYMLNLYDPNKYVDV
NNVGIRGYMYLKGPRGSIVTTNIYLNSSLYMGTKFIIKKYASGNKDNIVRNNDRVYINVVVKNKEYRLATNASQA
GVEKILSVLEIPDVGNLSQVVVMKSKNDQGIRNKCKMNLQDNNGNDIGFIGFHQFNNIDKLVASNWYNRQIERSS
RTFGCSWEFIPVDDGWGESPL
The endogenous activation loop is dash-underlined.
(BoNT/A6 - GenBank Accession No. FJ981696.1)
SEQ ID NO: 96
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK

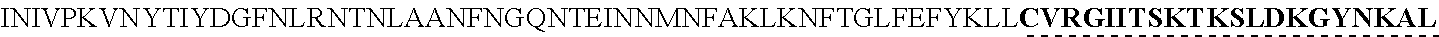

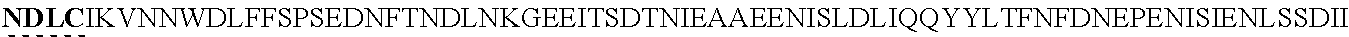


GQLELMPNIERFPNGKKYELDKYTMFHYLSAQEFEHGKSRIDLTNSVNEALLNPSHVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFAIVSYIANKVLTVQTINNALSKRNEKWDEVYKYTVTNWLAKVNTQIDLIREKMKKALENQAEATKAIINYQ
YNQYTEEEKNNINFNIDDLSSKLNESINSAMININKFLDQCSVSYLMNSMIPYAVKRLKDFDASVRDVLLKYIYD
NRGTLIGQVDRLKDKVNNTLSTDIPFQLSKYVDNORLLSTFTEYIKNIINTSILSLRYENNHLIDLSRYASKINI
GSRVNFDPIDKNOIQLFNLESSKIEVILKNAIVYNSMYENFSTSFWIKIPKYFSEISLNNEYTIINCIENNSGWK
VSLNYGEIIWTLQDNKQNIQRVVFKYSQMVAISDYINRWIFITITNNRLTKSKIYINGRLIDQKPISNLGNIHAS
NKIMFKLDGCRDPRRYIMIKYFNLFDKELNEKEIKDLYDSQSNSGILKDFWGNYLQYDKPYYMLNLFDPNKYVDV
NNVGIRGYMYLKGSRSTLLTTNIYLNSGLYMGTKFIIKKYASGNKDNIVRNNDRVYINVVVNNKEYRLATNASQA
GVEKILSALEIPDIGNLSQVVVMKSKNDQGIRNKCKMNLQDNNGNDIGFIGFHKENDIYKLVASNWYNRQIEISS
RTFGCSWEFIPVDDGWGEKPL
The endogenous activation loop is dash-underlined.
(BoNT/A7 - GenBank Accession No. JQ954969.1)
SEQ ID NO: 97
MPFVNKQFNYKDPVNGVDIAYIKIPNAGQMQPVKAFKIHNKIWVIPERDIFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIINFECKSFGHDVLNLTRNGYGSTQYIRFSPDFTFGFEESLEVDTNPLLGAGKFAIDPAVTLAHEL
IHAGHRLYGIAINPNRVFKVNTNAYYEMSGLEVSFEELRTFGGHDAKFIDSLQENEFRLYYYNKFKEVASILNKA
KSIIGTTASLQYMKNVFKEKYLLSEDTSGKFSVDKLRFDKLYKMLTEIYTEDNFVKFFKVLNRKTYLNFDKAVFK

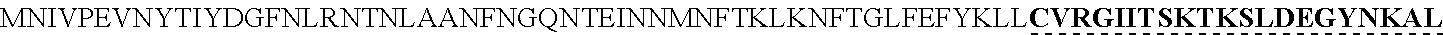

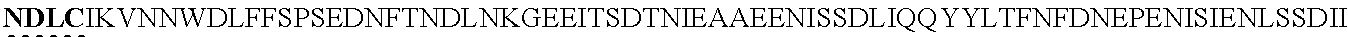


GQLELMPNIERFPNGKKYELDKYTMFHYLRAQEFEYGNSRIVLINSVNEALLNPSSVYTFFSSDYVKKANEATEA
AMFLGWVEQLVYDFTDETSEVSTMDKIADITIIVPYIGPALNIGNMVYKKKFEEALIFSGAVILLEFVPEIVLPI
LGTFALVSYTSNKVLTVRTIDNALSKRNEKWEEVYKYIVTNWLAKVNTQINLIRKKMKEALENQAEATKAIINYQ
YNQYTEEEKNNINFNIGDLSSKLNDSINKAMININKFLDQCSVSYLMNSMIPQGVKQLKDFDTSLRDSLLKYIYD
NRGTLIGQVDRLKDKVNNTLSTDIPFQLSKYADNORLLSTFTEYIKNIINTSILNLRYESNHLIDLSRYASKINI
GSRVNFDPIDKNQIQLFNLESSKIEVILKNAIVYNSMYENFSTSFWIKIPKYFSKINLNNEYTIINCIENNSGWK
VSLNYGEIIWTLQDNEQNIQRVVFKYSQMVNISDYINRWIFVTITNNRLTKSKIYINGRLIDQKPISNLGNIHAS
NKIMFKLDGCRDPHRYILIKYFNLFDKELNEKEIKDLYDNQSNSGILKDFWGDYLQYDKPYYMLNLYDPNKYIDV
NNIGIRGYMYLKGPRGSVTTTNIYLNSMLYMGTKFIIKKHASGNKDNIVRNNDRVYINVLVKNKEYRLATNASQA
GGEKILSAVEIPDVGNLSQVVVMKSKNDQGIRNKCKMNLQDNNGNDIGFIGFHQFNNIAKLVASNWYNRQIGKTS
VTLGCSWELIPVDYGWGESSL
The endogenous activation loop is dash-underlined.
(BoNT/A8 - GenBank Accession No. KM233166.1)
SEQ ID NO: 98
MPFVNKQFNYKDTVNGIDIAYIKIPNAGQMQPVKAFKIHNKIWVIPERDTFTNPKEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHDVLNLTRNGYGSTQYIRFSPDFTFGFEESLEVDTNPLLGAGKFATDPAVTLAHEL
IHAEHRLYGIAINPNRVFKVNTNAYYEMSGLEVSFEELRTFGGHNAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK

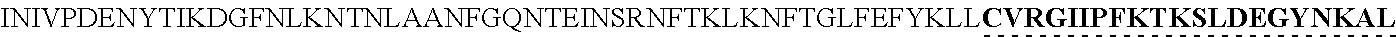

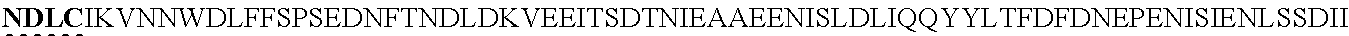


GQLEPMPNIERFPNGKKYELDKYTMFHYLRAQEFEHSKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLVRKKMKEALENQAEATKAIINYQ
YNQYTEEEKNNINFNIDDLSSKLNESINSAMTNINKFLDQCSVSYLMNSMIPYAVKRLKDFDASVREVLLKYIYD
NRGTLILQVDRLKDKVNNTLSADIPFQLSKYVDNKKLLSTFTEYIKNITNTSILSIVVDKDGRLIDLSRYGAEIY
NGDKVSYNSIDKNQIKLINLESSAIEVILKNAIVYNSMYENFSTSFWIKIPKYFSKINLNNEYTIINCIENNSGW
KVSLNYGEIIWTLQDNQQNIQRVVFKYSQMVNISDYINRWIFVTITNNRLDKSKIYINGRLIDQKPISNLGNIHA
SNNIMFKLDGCRDPRRYIVIKYFNLFDKELNEKEIKDLYDNQSNSGILKDFWGDYLQYDKPYYMLNLYDPNKYVD
VNNIGIRGYMYLKGPRGSVVTTNIYLNSTLYMGTKFIIKKYASGNKDNIVRNNDRVYINVVVKNKEYRLATNALQ
AGVEKILSALEIPDVGNLSQVVVMKSKNDQGIRNKCKMNLQDNNGNDIGLIGFHQFNNIAKLVASNWYNRQVGKA
SRTFGCSWEFIPVDDGWGESSQ
The endogenous activation loop is dash-underlined.
(BoNT/B1 - UniProt P10844)
SEQ ID NO: 99
MPVTINNENYNDPIDNNNIIMMEPPFARGTGRYYKAFKITDRIWIIPERYTFGYKPEDEN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMOSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGONKAINKQA

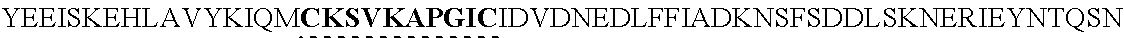

YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDFDNTLKKNLLNYIDENKLYLIGSAEYEKSKVNKYLKTIMPEDL
SIYINDTILIEMENKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTIINNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCOLLFKKDEESTDEIGLIGIHRFYESGIVFEEYKDYFCIS
KWYLKEVKRKPYNLKLGCNWQFIPKDEGWTE
The endogenous activation loop is dash-underlined.
(BoNT/B2 - GenBank Accession No. AB084152.1)
SEQ ID NO: 100
MPVTINNFNYNDPIDNNNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNEKKFFMOSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGFNISDKNMEKEYRGONKAINKQAYEEISKEHLAVYKIQMCKSVRAPGICIDVD
NEDLFFIADKNSFSDDLSKNERIEYDTOSNYIENRSSIDELILDTNLISKIELPSENTESLTDFNVDVPVYEKQP
AIKKIFTDENTIFQYLYSQTFPLDIRDISLTSSFDDALLFSNKVYSFFSMDYIKTANKVVEAGLFAGWVKQIVDD
FVIEANKSSTMDKIADISLIVPYIGLALNVGNETAKGNFENAFEIAGASILLEFIPELLIPVVGAFLLESYIDNK
NKIIKTIDNALTKRDEKWIDMYGLIVAQWLSTVNTQFYTIKEGMYKALNYQAQALEEIIKYKYNIYSEKEKSNIN
IDFNDINSKLNEGINQAVDNINNFINECSVSYLMKKMIPLAVEKLLDFDNTLKKNLLNYIDENKLYLIGSAEYEK
SKVDKHLKTIIPFDLSMYTNNTILIEIFNKYNSEILNNIILNLRYRDNNLIDLSGYGANVEVYDGVELNDKNQFK
LTSSTNSEIRVTQNQNIIFNSMFLDFSVSFWIRIPKYKNDGIQNYIHNEYTIINCIKNNSGWKISIRGNRIIWTL
TDINGKTKSVFFEYSIREDISDYINRWFFVTITNNSDNAKIYINGKLESNIDIKDIGEVIANGEIIFKLDGDIDR
TQFIWMKYFSIFNTELSQSNIKEIYKIQSYSEYLKDFWGNPLMYNKEYYMFNAGNKNSYIKLKKDSSVGEILTRS
KYNQNSNYINYRNLYIGEKFIIRRKSNSQSINDDIVRKEDYIYLDFFNSNREWRVYAYKDFKEEEKKLFLANIYD
SNEFYKTIQIKEYDEQPTYSCOLLFKKDEESTDEIGLIGIHRFYESGIVLKDYKNYFCISKWYLKEVKRKPYNPN
LGCNWQFIPKDEGWIE
The endogenous activation loop is dash-underlined.
(BoNT/B3 - GenBank Accession No. EF028400.1)
SEQ ID NO: 101
MPVTINNFNYNDPIDNDNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNEKKFFMOSTDAIQAEELYTFGGQDPRIITPSTDKSIYDKVLQNFRGIV
DRLNKVLVCISDPNININIYKNKFKDKYKFVEDSEGKYSIDVESFDKLYKSLMFGFTETNIAENYKIKTRASYFS
DSLPPVKIKNLLDNEIYTIEEGFNISDKNMEKEYRGONKAINKQAYEEISKEHLAVYKIQMCKSVRAPGICIDVD
NEDLFFIADKNSFSDDLSKNERIEYDTQSNYIENRSSIDELILDTNLISKIELPSENTESLTDFNVDVPVYEKOP
AIKKIFTDENTIFQYLYSQTFPLDIRDISLTSSFDDALLFSNKVYSFFSMDYIKTANKVVEAGLFAGWVKQIVDD
FVIEANKSSTMDKIADISLIVPYIGLALNVGNETAKGNFENAFEIAGASILLEFIPELLIPVVGAFLLESYIDNK
NKIIKTIDNALTKRDEKWIDMYGLIVAQWLSTVNTQFYTIKEGMYKALNYQAQALEEIIKYKYNIYSEKEKSNIN
IDENDINSKLNEGINQAIDNINNFINECSVSYLMKKMIPLAVEKLLDFDNTLKKNLLNYIDENKLYLIGSAEYEK
SKVDKHLKTIIPFDLSMYINNTILIEIFNKYNSEILNNIILNLRYRDNNLIDLSGYGAKVEVYNGVELNDKNQFK
LTSSANSKIRVTQNQDIIFNSMFLDFSVSFWIRIPKYKNDGIONYIHNEYTIINCIKNNSGWKISIRGNKIIWTL
TDINGKTKSVFFEYSIRKDVSEYINRWFFVTITNNSDNAKIYINGKLESNIDIKDIGEVIANGEIIFKLDGDIDR
TQFIWMKYFSIFNTELSQSNIKEIYKIQSYSEYLKDFWGNPLMYNKEYYMFNAGNKNSYIKLKKDSSVGEILTRS
KYNQNSNYINYRNLYIGEKFIIRRKSNSQSINDDIVRKEDYIYLDFFNLNQEWRVYAYKDFKKKEEKLFLANIYD
SNEFYNTIQIKEYDEQPTYSCQLLFKKDEESTDEIGLIGIHRFYESGIVEKDYKDYFCISKWYLKEVKRKPYNPN
LGCNWQFIPKDEGWIE
The endogenous activation loop is dash-underlined.
(BoNT/B4 - GenBank Accession No. EF051570.1)
SEQ ID NO: 102
MPVTINNFNYNDPIDNDNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVEQ
KKGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNEKKFFMQSTDTIQAEELYTFGGQDPSIISPSTDKSIYDKVLQNFRGIV
DRLNKVLVCISDPNININIYKNKFKDKYKFVEDSEGKYSIDVESFNKLYKSLMFGFTEINIAENYKIKTRASYFS

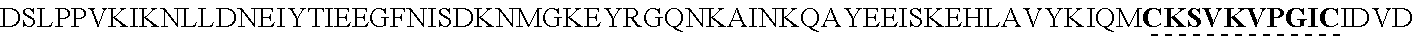

NENLFFIADKNSFSDDLSKNERVEYNTONNYIGNDFPINELILDTDLISKIELPSENTESLTDFNVDVPVYEKOP
AIKKVFTDENTIFQYLYSQTFPLNIRDISLTSSFDDALLVSSKVYSFFSMDYIKTANKVVEAGLFAGWVKQIVDD
FVIEANKSSTMDKIADISLIVPYIGLALNVGDETAKGNFESAFEIAGSSILLEFIPELLIPVVGVFLLESYIDNK
NKIIKTIDNALTKRVEKWIDMYGLIVAQWLSTVNTQFYTIKEGMYKALNYQAQALEEIIKYKYNIYSEEEKSNIN
INFNDINSKLNDGINQAMDNINDFINECSVSYLMKKMIPLAVKKLLDFDNTLKKNLLNYIDENKLYLIGSVEDEK
SKVDKYLKTIIPFDLSTYTNNEILIKIFNKYNSEILNNIILNLRYRDNNLIDLSGYGAKVEVYDGVKLNDKNQFK
LTSSADSKIRVTQNQNIIFNSMFLDFSVSFWIRIPKYRNDDIQNYIHNEYTIINCMKNNSGWKISIRGNRIIWTL
IDINGKTKSVFFEYNIREDISEYINRWFFVTITNNLDNAKIYINGTLESNMDIKDIGEVIVNGEITFKLDGDVDR
TQFIWMKYFSIFNTQLNQSNIKEIYKIQSYSEYLKDFWGNPLMYNKEYYMFNAGNKNSYIKLVKDSSVGEILIRS
KYNQNSNYINYRNLYIGEKFIIRRKSNSQSINDDIVRKEDYIHLDFVNSNEEWRVYAYKNFKEQEQKLFLSIIYD
SNEFYKTIQIKEYDEQPTYSCQLLFKKDEESTDDIGLIGIHRFYESGVLRKKYKDYFCISKWYLKEVKRKPYKSN
LGCNWQFIPKDEGWTE
The endogenous activation loop is dash-underlined.
(BoNT/B5 - GenBank Accession No. EF033130.1)
SEQ ID NO: 103
MPVTINNFNYNDPIDNNNIIMMEPPFARGMGRYYKAFKITDRIWIIPERYTFGYKPEDFNKSSGIFNRDVCEYYD
PDYLNTNDKKNIFLOTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHELIHVLHGLYGIKVNDLPIVPNEKKFFMOSTDAIQAEELYTFGGQDPSIISPSTDKSIYDKVLQNFRGIV
DRLNKVLVCISDPNININIYKNKFKDKYKFVEDSEGKYSIDVESFDKLYKSLMFGFTETNIAENYKIKTRASYFS

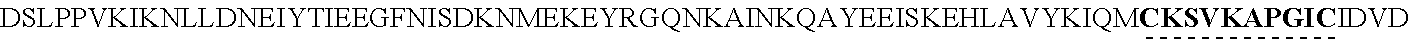

NEDLFFIADKNSFSDDLSKNERIAYNTONNYIENDFSINELILDTDLISKIELPSENTESLTDFNVYVPVYKKQP
AIKKIFTDENTIFQYLYSQTFPLDIRDISLTSSFDDALLFSNKVYSFFSMDYIKTANKVVEAGLFAGWVKQIVDD
FVIEANKSSTMDKIADISLIVPYIGLALNVGNETAKGNFENAFEIAGASILLEFIPELLIPVVGAFLLESYIDNK
NKIIETINSALTKRDEKWIDMYGLIVAQWLSTVNTQFYTIKEGMYKALNYQAQALEEIIKYKYNIYSEKERSNIN
IDFNDVNSKLNEGINQAIDNINNFINECSVSYLMKKMIPLAVEKLLDFDNTLRKNLLNYIDENKLYLIGSAEYEK
SKVDKYLKTSIPFDLSTYTNNTILIEIFNKYNSDILNNIILNLRYRDNKLIDLSGYGAKVEVYDGVKLNDKNQFK
LTSSANSKIRVIQNQNIIFNSMFLDFSVSFWIRIPKYKNDGIQNYIHNEYTIINCMKNNSGWKISIRGNMIIWTL
IDINGKIKSVFFEYSIKEDISEYINRWFFVTITNNSDNAKIYINGKLESHIDIRDIREVIANDEIIFKLDGNIDR
TQFIWMKYFSIFNTELSQSNIEEIYKIQSYSEYLKDFWGNPLMYNKEYYMFNAGNKNSYIKLKKDSSVGEILTRS
KYNQNSKYINYRDLYIGEKFIIRRKSNSQSINDDIVRKEDYIYLDFFNLNQEWRVYMYKYFKKEEEKLFLAPISD
SDEFYNTIQIKEYDEQPTYSCOLLFKKDEESTDEIGLIGIHRFYESGIVFKEYKDYFCISKWYLKEVKRKPYNSK
LGCNWQFI PKDEGWTE
The endogenous activation loop is dash-underlined.
(BoNT/B6 - GenBank Accession No. AB302852.1)
SEQ ID NO: 104
MPVTINNFNYNDPIDNNNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNEKKFFMOSTDAIQAEELYTFGGQDPSIITPSTDKSIYDKVLONFRGIV
DRLNKVLVCISDPNININIYKNKFKDKYKFVEDSEGKYSIDVESFDKLYKSLMFGFTETNIAENYKIKTRASYFS

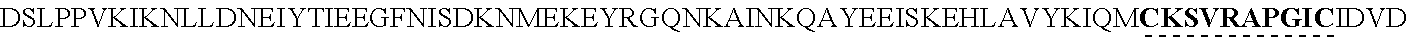

NEDLFFIADKNSFSDDLSKNERIEYDTQSNYIENRSSIDELILDTNLISKIELPSENTESLTDFNVDVPVYEKQP
AIKKFFTDENTIFQYLYSQTFPLDIRDISLTSSFDDALLFSNKVYSFFSMDYIKTANKVVEAGLFAGWVKQIVDD
FVIEANKSNTMDKLADISLIVPYIGLALNVGNETAKGNFENAFEIAGASILLEFIPELLIPVVGAFLLESYIDNK
NKIIKTIDNALTKRDEKWRDMYGLIVAQWLSTVNTQFYTIKEGMYKALNYQAQALEEIIKYKYNIYSEKEKSNIN
IDFNDINSKLNEGINQAIDNINNFINECSVSYLMKKMIPLAVEKLLDFDNTLKKNLLNYIDENKLYLIGSAEYEK
SKVDKHLKTIIPFDLSMYTNNTILIEIFKKYNSEILNNIILNLRYRDNNLIDLSGYGANVEVYDGVELNDKNQFK
LTSSTNSEIRVTQNQNIIFNSMFLDFSVSFWIRIPKYKNDGIQNYIHNEYTIINCIKNNSGWKISIRGNRIIWTL
TDINGKTKSVFFEYSIREDISDYINRWFFVTITNNSDNAKIYINGKLESNIDIKDIGEVIANGEIIFKLDGDIDR
TQFIWMKYFSIFNTELSQSNIKEIYKIQSYSEYLKDFWGNPLMYNKEYYMFNAGNKNSYIKLKKDSPVGEILTRS
KYNQNSNYINYRNLYIGEKFIIRRKSNSQSINDDIVRKEDYIYLDFFNLNQEWRVYALKNFKKKEEKLFLAPISD
SDEFYNTIQIKEYDEQPTYSCQLLFKKDEESTDEIGLIGIHRFYESGIVEKDYKYYFCISKWYLKEVKRKPYNPN
LGCNWQFIPKDEGWIE
The endogenous activation loop is dash-underlined.
(BoNT/B7 - GenBank Accession No. JQ354985.1)
SEQ ID NO: 105
MPVTINNENYNDPIDNNNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNEKKFFMOSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETNIAENYKIKTRASYFS

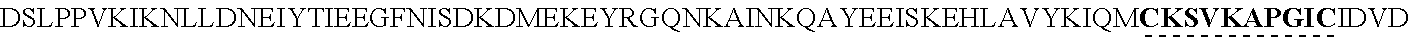

NEDLFFIADKNSFSDDLSKNERIEYNTKNIYIENYFSINELILDTDLISGIELPSENTESLTDFNVDVPVYEKOP
AIKKIFTDENTIFQYLYSQTFPLDIRDISLTSSFDDALLFSNKVYSFFSMDYIKTANKVVEAGLFAGWVKQIIDD
FVIEANKSSTMDKIADISLIVPYIGLALNVGNETAKGNFENAFEIAGASILLEFIPELLIPVVGAFLLESYIDNK
NKIIKTIDNALTKRVEKWIDMYGLIVAQWLSTVNTQFYTIKEGMYKALNYQAQALEEIIKYKYNIYSEKEKLNIN
IDFNDINSKLNEGINQAIDNINNFINECSVSYLMKKMIPLAIEKLLDFDNALKKNLLNYIDENKLYLIGSVEEEK
SKVDKFFKTIIPFDLSMYINNTILIEMVNKYNSEILNNIILNLRYRDNNLIDSSGYGAKVEVYNGVELNDKNQFK
LTSSANSKIKVTQNQNITFNSMFLDFSVSFWIRIPKYKNDGIONYIHNEYTIINCMKNNSGWKISIRGNRIIWTL
TDINGKTKSVFFEYSIREDISDYINRWFFVTITNNLDNAKIYINGKLESNIDIRDIREVIVNGEIIFKLDGEIDR
TQFIWMKYFSIFNTELSQSNVKEIYKIQSYSKYLKDFWGNPLMYNKEYYMFNAGNKNSYIKLVKDSSVGEILTRS
KYNQNSNYINYRNLYIGEKFIIRRKSSSQSISDDIVRKEDYIYLDFFNSNREWRVYAYKNFKGQEEKLFLANIYD
SNEFYKTIQIKEYDEQPTYSCOLLFKKDEESTDEIGLIGIHNFYESGILFKDYKDYFCISKWYLKEVKKKPYSSN
LGCNWQFIPKDEGWTE
The endogenous activation loop is dash-underlined.
(BoNT/B8 - GenBank Accession No. JQ964806.1)
SEQ ID NO: 106
MPVTINNFNYNDPIDNNNIIMMEPPFARGTGRYYKAFKITDRIWIIPERYTFGYKPEDFNKSSGIFNRDVCEYYD
PDYLNTNDKKNIFLQTMIKLFNRIKSKPLGEKLLEMIINGIPYLGDRRVPLEEFNTNIASVTVNKLISNPGGEER
KEGIFANLIIFGPGPVLNENETIDIGIQNHFASREGFGGIMQMKFCPEYVSVENNVQENKGASIFNRRGYFSDPA
LILMHELIHVLHGLYGIKVDDLPIVPNGKKFFMQSTDAIQAEELYTFGGQDPSIITPSTDKSIYDKVLONFRGIV
DRLNKVLVCISDPNININIYKNKFKDKYKFVEDSEGKYSIDVESFDKLYKSLMFGFTETNIAENYKIKTRASYFS

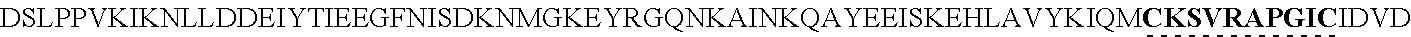

NEDLFFIADKNSFSDDLSKNERIEYNTOSNYIENDFSINELILDTDLISKIELPSENTESLTDFNVDVPVYEKQP
AIKKIFTDENTIFQYLYSQTFPLDIRDISLTSSFDDALLFSNKVYSFFSMDYIKTANKVVEAGLFAGWVKQIVDD
FVIEANKSNTMDKIADISLIVPYIGLALNVGNETAKGNFENAFEIAGSSILLEFIPELLIPVVGAFLLESYIDNK
NKIIKTIDNALTKRDEKWIDMYGLIVAQWLSTVNTQFYTIKEGMYKALNYQAQALEEIIKYKYNIYSEKEKSNIS
IDFNDINSKLNEGINQAIDNINDFINECSVSYLMKKMIPLAVEKLLDFDNTLKKNLLNYIDENKLYLIGSAEYEK
SKVDKHLKTIMTFDLSMYTNNTILIKMVNKYNSEILNNIILNLRYRDNNLIDLSGYGANVEVYDGVELNDKNQFK
LTSSTNSEIRVTQNQNIIVNSMFLDFSVSFWIRIPKYKNDGIQNYIHNEYTIINCMKNNSGWKISIRGNRIIWTL
IDINGKIKSVFFEYSIRKDVSEYINRWFFVTITNNLDNAKIYINGKLESNMDIRDIREVIANGEIIFKLDGDIDR
TQFIWMKYFSIFNTELSQSNIEEIYKIQSYSEYLKDFWGNPLMYNKEYYMFNAGSKNSYIKLKKDSSVGEILTRS
KYNQNSQYINYRDLYIGEKFIIKRKSNSQSINDDIVRKEDYIYLDFFNLNQEWRVYAYKDFKGQKEQKLFLANIH
DSNEFYKTIQIKEYDEQPTYSCOLLFKKDEESTDEIGLIGIHRFYESGFVFQEYKYYFCISKWYLKEVKKKPYNP
DLGCNWQFIPKDEGWTE
The endogenous activation loop is dash-underlined.
(BoNT/C1 - UniProt P18640)
SEQ ID NO: 107
MPITINNFNYSDPVDNKNILYLDTHLNTLANEPEKAFRITGNIWVIPDRFSRNSNPNLNK
PPRVTSPKSGYYDPNYLSTDSDKDTFLKEIIKLFKRINSREIGEELIYRLSTDIPEPGNN
NTPINTFDFDVDFNSVDVKTRQGNNWVKTGSINPSVIITGPRENIIDPETSTFKLINNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSENKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA

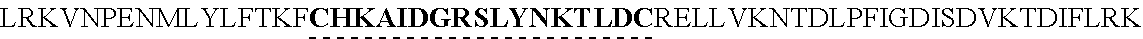

DINEETEVIYYPDNVSVDQVILSKNTSEHGOLDLLYPSIDSESEILPGENQVFYDNRTON
VDYLNSYYYLESQKLSDNVEDFTFTRSIEEALDNSAKVYTYFPTLANKVNAGVQGGLFLM
WANDVVEDFTTNILRKDILDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYENNINDSKILSLONRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVFNTRRNNN
DENEGYKIIIKRIRGNTNDTRVRGGDILYFDMTINNKAYNLFMKNETMYADNHSTEDIYA
IGLREQTKDINDNIIFQIQPMNNTYYYASQIFKSNENGENISGICSIGTYRFRLGGDWYR
HNYLVPTVKQGNYASLLESTSTHWGFVPVSE
The endogenous activation loop is dash-underlined.
(BoNT/D - UniProt P19321)
SEQ ID NO: 108
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNOSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
VVYSSQYNVKNRTHYFSRHYLPVFANILDDNIYTIRDGENLINKGFNIENSGQNIERNPA

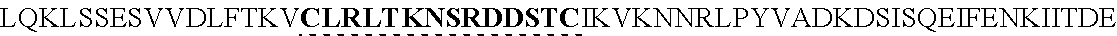

TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNFNQAFATAGVAFLLEGEP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLINSHNEYTIINSIEQNS
GWKLCIRNGNIEWILQDVNRKYKSLIFDYSESLSHTGYTNKWFFVTIINNIMGYMKLYIN
GELKQSQKIEDLDEVKLDKTIVFGIDENIDENQMLWIRDENIFSKELSNEDINIVYEGQI
LRNVIKDYWGNPLKEDTEYYIINDNYIDRYIAPESNVLVLVQYPDRSKLYTGNPITIKSV
SDKNPYSRILNGDNIILHMLYNSRKYMIIRDTDTIYATQGGECSONCVYALKLQSNLGNY
GIGIFSIKNIVSKNKYCSQIFSSFRENTMLLADIYKPWRFSFKNAYTPVAVTNYETKLLS
TSSFWKFISRDPGWVE
The endogenous activation loop is dash-underlined.
(BoNT/CD - GenBank Accession No. AB200360.1)
SEQ ID NO: 109
MPITINNFNYSDPVDNKNILYLDTHLNTLANEPEKAFRIIGNIWVIPDRFSRDSNPNLNKPPRVTSPKSGYYDPN
YLSTDSEKDTFLKEIIKLFKRINSREIGEELIYRLATDIPFPGNNNTPINTFDFDVDFNSVDVKTRQGNNWVKTG
SINPSVIITGPRENIIDPETSTFKLTNNTFAAQEGFGALSIISISPRFMLTYSNATNNVGEGRFSKSEFCMDPIL
ILMHELNHAMHNLYGIAIPNDORISSVTSNIFYSQYNVKLEYAEIYAFGGPTIDLIPKSARKYFEEKALDYYRSI
AKRLNSITTANPSSFNKYIGEYKQKLIRKYRFVVESSGEVAVDRNKFAELYKELTQIFTEFNYAKIYNVQNRKIY

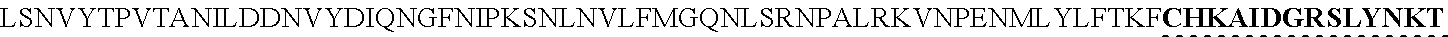

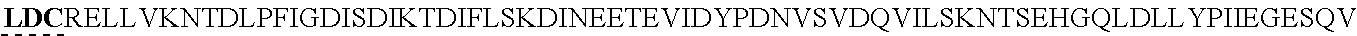


LPGENQVFYDNRTQNVDYLNSYYYLESQKLSDNVEDFTFTTSIEEALDNSGKVYTYFPKLADKVNTGVQGGLFLM
WANDVVEDFTTNILRKDTLDKISDVSAIIPYIGPALNISNSVRRGNFTEAFAVTGVTILLEAFQEFTIPALGAFV
IYSKVQERNEIIKTIDNCLEQRIKRWKDSYEWMIGTWLSRITTQFNNISYQMYDSLNYQADAIKDKIDLEYKKYS
GSDKENIKSQVENLKNSLDIKISEAMNNINKFIRECSVTYLFKNMLPKVIDELNKFDLKTKTELINLIDSHNIIL
VGEVDRLKAKINESFENTIPFNIFSYTNNSLLKDIINEYFNSINDSKILSLONKKNALVDTSGYNAEVRLEGDVQ
VNTIYTNDFKLSSSGDKIIVNLNNNILYSAIYENSSVSFWIKISKDLTNSHNEYTIINSIKQNSGWKLCIRNGNI
EWILQDINRKYKSLIFDYSESLSHTGYTNKWFFVTITNNIMGYMKLYINGELKQSERIEDLDEVKLDKTIVFGID
ENIDENQMLWIRDFNIFSKELSNEDINIVYEGQILRNVIKDYWGNPLKFDTEYYMINYNYIDRYIAPKNNILVLV
QYSDISKLYTKNPITIKSAANKNPYSRILNGDDIMFHMLYDSREYMIIRDTDTIYATQGGQCSKNCVYALKLQSN
LGNYGIGIFSIKNIVSQNKYCSQIFSSFMKNTMLLADIYKPWRFSFENAYTPVAVTNYETKLLSTSSFWKFISRD
PGWVE
The endogenous activation loop is dash-underlined.
(BoNT/DC - GenBank Accession No. AB745660.1)
SEQ ID NO: 110
MTWPVKDFNYSDPVNDNDILYLRIPQNKLITTPVKAFMITQNIWVIPERFSSDTNPSLSKPPRPTSKYQSYYDPS
YLSTDEQKDTFLKGIIKLFKRINERDIGKKLINYLVVGSPFMGDSSTPEDTFDFTRHTTNIAVEKFENGSWKVTN
IITPSVLIFGPLPNILDYTASLTLQGQQSNPSFEGFGTLSILKVAPEFLLTFSDVTSNQSSAVLGKSIFCMDPVI
ALMHELTHSLHQLYGINIPSDKRIRPQVSEGFFSQDGPNVQFEELYTFGGSDVEIIPQIERLQLREKALGHYKDI
AKRLNNINKTIPSSWSSNIDKYKKIFSEKYNFDKDNTGNFVVNIDKFNSLYSDLTNVMSEVVYSSQYNVKNRTHY

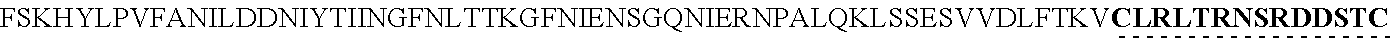

IQVKNNTLPYVADKDSISQEIFESQIITDETNVENYSDNFSLDESILDAKVPTNPEAVDPLLPNVNMEPLNVPGE
EEVFYDDITKDVDYLNSYYYLEAQKLSNNVENITLTTSVEEALGYSNKIYTFLPSLAEKVNKGVQAGLFLNWANE
VVEDFTTNIMKKDTLDKISDVSAIIPYIGPALNIGNSALRGNFKOAFATAGVAFLLEGFPEFTIPALGVFTFYSS
IQEREKIIKTIENCLEQRVKRWKDSYQWMVSNWLSRITTQFNHISYQMYDSLSYQADAIKAKIDLEYKKYSGSDK
ENIKSQVENLKNSLDVKISEAMNNINKFIRECSVTYLFKNMLPKVIDELNKFDLKTKTELINLIDSHNIILVGEV
DRLKAKVNESFENTIPFNIFSYTNNSLLKDMINEYFNSINDSKILSLONKKNTLMDTSGYNAEVRVEGNVQLNPI
FPFDFKLGSSGDDRGKVIVTQNENIVYNAMYESFSISFWIRINKWVSNLPGYTIIDSVKNNSGWSIGIISNFLVF
TLKQNENSEQDINFSYDISKNAAGYNKWFFVTITTNMMGNMMIYINGKLIDTIKVKELTGINFSKTITFQMNKIP
NTGLITSDSDNINMWIRDFYIFAKELDDKDINILFNSLQYTNVVKDYWGNDLRYDKEYYMINVNYMNRYMSKKGN
GIVFNTRKNNNDFNEGYKIIIKRIIGNTNDTRVRGENVLYFNTTIDNKQYSLGMYKPSRNLGTDLVPLGALDQPM
DEIRKYGSFIIQPCNTFDYYASQLFLSSNATTNRIGILSIGSYSFKLGDDYWFNHEYLIPVIKIEHYASLLESTS
THWVFVPASE
The endogenous activation loop is dash-underlined.
(BoNT/E - UniProt Q00496)
SEQ ID NO: 111
MPKINSFNYNDPVNDRTILYIKPGGCQEFYKSFNIMKNIWIIPERNVIGTTPQDFHPPTS
LKNGDSSYYDPNYLOSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRFNDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL

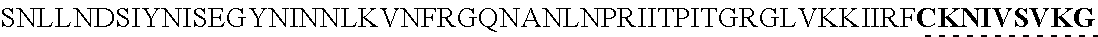

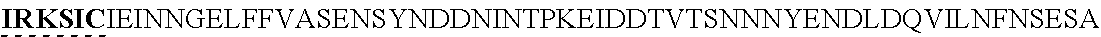


PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDETTEANQKSTVDKIADIS
IVVPYIGLALNIGNEAQKGNEKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SONDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSINDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
The endogenous activation loop is dash-underlined.
(BoNT/E1 - GenBank Accession No. GQ244314.1)
SEQ ID NO: 112
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRFNDNSMNEFIQDPALTLMHELIHSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY

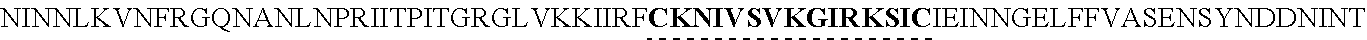

PKEIDDTVTSNNNYENDLDQVILNFNSESAPGLSDEKLNLTIONDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNOFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
The endogenous activation loop is dash-underlined.
(BoNT/E2 - GenBank Accession No. EF028404.1)
SEQ ID NO: 113
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGIQDILLPNVII
MGAEPDLFETNSSNISLRNNYMPSNHGFGSIAIVTFSPEYSFRFNDNSMNEFIQDPALTLMHELIHSLHGLYGAK
GITTKYTITQKONPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY

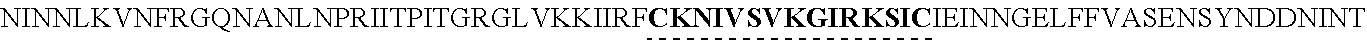

PKEIDDTVTSNNNYENDLDQVILNFNSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYNNEPNANILKDFWGNYLLYDKEYYLLNVLKPNNFIDRRTDSTLSINNIRSTILLANRLYSGI
KVKIQRVNNSSTNDNLVRKNDQVYINFVASKTHLFPLYADTNTTNKEKTIKSSSSGNRFNQVVVMNSVGNNCTMN
FKNNNGNNIGMLGFKDNTLVASTWYYTHMRDNTNSNGCFWNFISEEHGWQEK
The endogenous activation loop is dash-underlined.
(BoNT/E3 - GenBank Accession No. EF028403.1)
SEQ ID NO: 114
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQHILLPNVII
MGAEPDLFETNSSNISLRNNYMPSNHGFGSIAIVTFSPEYSFRFNDNSINEFIQDPALTLMHELIHSLHGLYGAK
GITTTCIITQQQNPLITNRKGINIEEFLTFGGNDLNIITVAQYNDIYTNLLNDYRKIASKLSKVQVSNPQLNPYK
DIFQEKYGLDKDASGIYSVNINKFDDILKKLYSFTEFDLATKFQVKCRETYIGQYKYFKLSNLLNDSIYNISEGY

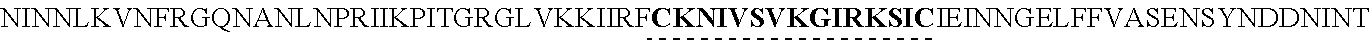

PKEIDDTVTSNNNYENDLDQVILNFNSESAPGLSDEKLNLTIONDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SONDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
The endogenous activation loop is dash-underlined.
(BoNT/E4 - GenBank Accession No. AB088207.1)
SEQ ID NO: 115
MPTINSFNYNDPVNNRTILYIKPGGCQQFYKSFNIMKNIWIIPERNVIGTIPQDFLPPTSLKNGDSSYYDPNYLQ
SDQEKDKFLKIVTKIFNRINDNLSGRILLEELSKANPYLGNDNTPDGDFIINDASAVPIQFSNGSQSILLPNVII
MGAEPDLFETNSSNISLRNNYMPSNHGFGSIAIVTFSPEYSFRFKDNSMNEFIQDPALTLMHELIHSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY

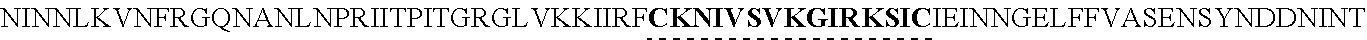

PKEIDDTVTSNNNYENDLDQVILNFNSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVGWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNALKAIIESKYNSYTLEEKNELTNKYDIEQIENELNQK
VSIAMNNIDRFLTESSISYLMKLINEVKINKLREYDENVKTYLLDYIIKHGSILGESQQELNSMVIDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNSGINQKLAF
NYGNANGISDYINKWIFVTITNDRLGDSKLYINGNLIDKKSILNLGNIHVSDNILFKIVNCSYTRYIGIRYFNIF
DKELDETEIQTLYNNEPNANILKDFWGNYLLYDKEYYLLNVLKPNNFINRRTDSTLSINNIRSTILLANRLYSGI
KVKIQRVNNSSTNDNLVRKNDQVYINFVASKTHLLPLYADTATTNKEKTIKISSSGNRFNQVVVMNSVGNNCTMN
FKNNNGNNIGLLGFKADTVVASTWYYTHMRDNTNSNGFFWNFISEEHGWQEK
The endogenous activation loop is dash-underlined.
(BoNT/E5 - GenBank Accession No. AB037711.1)
SEQ ID NO: 116
MPKINSFNYNDPVNDRTILYIKPGGCQEFYKSFNIMKNIWIIPERNVIGTTPQDFHPPTSLKNGDSSYYDPNYLQ
SDEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRFNDNSMNEFIQDPALTLMHELIHSLHGLYGAK
GITTKYTITQKQNPLITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLATKFQVKCRQTYIGQYKYFKLSNLLNDSIYNISEGY

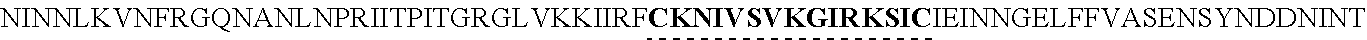

PKEIDDTVTSNNNYENDLDQVILNFNSESAPGLSDEKLNLTIONDAYIPKYDSNGTSDIEQHDVNELNVFFYLDA
QKVPEGENNVNLTSSIDTALLEQPKIYTFFSSEFINNVNKPVQAALFVSWIQQVLVDFTTEANQKSTVDKIADIS
IVVPYIGLALNIGNEAQKGNFKDALELLGAGILLEFVPELLIPTILVFTIKSFLGSSDNKNKVIKAINNALKERD
EKWKEVYSFIVSNWMTKINTQFNKRKEQMYQALQNQVNALKTIIEFKYNSYTLEEKKELKNNYDIEQIENELNQK
VSIAMNNIDRFLTESSISYLMKLINEVKINKLREYDENVKTYLLDYIIQHGSILGESQQELNSMVIDTLNNSIPF
KLSSYTDDKILISYFNKFFKRIKSSSVLNMRYKNDKYVDTSGYDSNININGEIFIYPTNKNQFTIFNSKPSEVNI
SONDYIIYDNKYKNFSISFWVRIPNYDNKIVNINNEYTIINCMRDNNSGWKVSLNHNEIIWTLQDNARINQKLVF
KYGNANGISDYINKWIFVTITNDRLGDSKLYINGHLIDQKSILNLGNIHVSDNILFKIVNCSYTRYIGIRYFNIF
DKELDETEIQTLYSNEPNTNILKDFWGNYLLYDKGYYLLNVLKPNNFIDRRKDSTLSINNIRSTILLANRLYSGI
KVKIQRVNDSSTNDRFVRKNDQVYINYISNSSSYSLYADTNTTDKEKTIKSSSSGNRFNQVVVMNSVGNNCTMNF
KNNNGNNIGLLGFKADTVVASTWYYTHMRDHTNSNGCFWNFISEEHGWQEK
The endogenous activation loop is dash-underlined.
(BoNT/E6 - GenBank Accession No. AM695759.1)
SEQ ID NO: 117
MPTINSFNYNDPVNDRTILYIKPGGCQEFYKSFNIMKNIWIIPERNVIGTTPQDFHPPTSLKNGDSSYYDPNYLQ
SYEEKDRFLKIVTKIFNRINNNLSGGILLEELSKANPYLGNDNTPDNQFHIGDASAVEIKFSNGSQDILLPNVII
MGAEPDLFETNSSNISLRNNYMPSNHGFGSIAIVTFSPEYSFRFKDNSMNEFIQDPALTLMHELIHSLHGLYGAK
GITTQYTITQQQNPLITNIKGTNIEEFLTFGGTDLNIITSAQYNDIYTNLLADYKKIASKLSKVQVSNPLLNPYK
DVFEKKYGLDKDASGIYSVNINKENDIFKKLYSFTEFDLATKFQVKCRQTYIGQYKYFKLSNLLNNSIYNISEGY

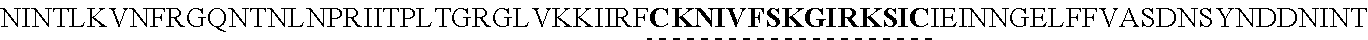

PKEIDDTVTSNNNYENDLDQVILNENSESAPGLSDEKLNLTIQNDAYIPKYDSNGTSDIEQHDVNELNVFFYLDA
QKVPEGENNIDFTSSIDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNALKTIIESKYNSYTLEEKNELINKYNIEQIENELNQK
VSIAMNNIEIFLTESSISYLMKLINEVKINKLREYDENVKTYLLDYIIKHGSILGESQQELNSMVIDTLNNSIPF
KLSSYTDDKILISYFNKFFKRIKSSSVLNMRYKNDKYVDTSGYDSNININGDVYKYPTNKNQFGIYNNKLSEVNI
SQNDYIIYDNKYKNFSISFWVRIPNYDNKIVNVNNEYTIINCMRDNNSGWKVSLNHNEIIWTLQDNSGINQKLAF
NYGNANGISDYINKWIFVTITNDRLGDSKLYINGNLIDKKSILNLGNIHVSDNILFKIVNCSYTRYIGIRYFNIF
DKELDETEIQTLYNNEPNANILKDFWGNYLLYDKEYYLLNVLKPNNFINRRTDSTLSINNIRSTILLANRLYSGI
KVKIQRVNNSSTNDNLVRKNDQVYINFVDSKTHLLPLYADTATTNKEKTIKISSSGNRFNQVVVMNSVGNNCTMN
FKNNNGNNIGLLGFKADTVVASTWYYTHMRDNTNSNGFFWNFISEEHGWQEK
The endogenous activation loop is dash-underlined.
(BoNT/E7 - GenBank Accession No. JN695729.1)
SEQ ID NO: 118
MPKINSFNYNDPVNDKTILYIKPGGCQQFYKSFNIMKNIWIIPERNVIGTIPQDFLPPTSLKNGDSSYYDPNYLQ
SNEEKDRFLKIVTKIFNRINDNLSGRILLEELSKANPYLGNDNTPDNQFHIGDASAVEIKFSNGNQSILLPNVII
MGAEPDLFETNSSNISLRNNYMPSNHGFGSIAIVTFSPEYSFRFNDNSMNEFIQDPALTLMHELIHSLHGLYGAK
RITTKYTITQQQNPLITNIRGTNIEEFLTFGGTDLNIITSAQYNDIYTNLLADYKKIASKLSKVQVSNPQLNPYK
DIFQEKYGLDKNASGIYSVNINKFDDIFKKLYSFTEFDLATKFQVKCRQTYIGQYKYFKLSNLLNNSIYNISEGY

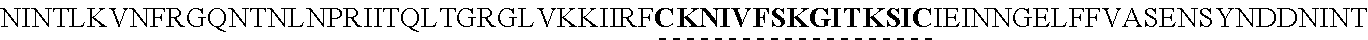

PKEIDDTVTSNNNYENDLDQVILNENSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNAIKTIIESKYNSYTLEEKNELTNKYDIKQIENELNQK
VSIAMNNIDRFLTESSISYLMKLINEVKINKLREYDENVKTYLLNYIIQHGSILGESQQELNSMVTDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLANRLYSGI
KVKIQRVNNSSTNDNLVRKNDQVYINFVASKTHLFPLYADTATTNKEKTIKISSSGNRFNQVVVMNSVGNNCTMN
FKNNNGNNIGLLGFKADTVVASTWYYTHMRDHTNSNGCFWNFISEEHGWQEK
The endogenous activation loop is dash-underlined.
(BoNT/E8 - GenBank Accession No. JN695730.1)
SEQ ID NO: 119
MPKINSFNYNDPVNDKTILYIKPGGCQQFYKSFNIMKNIWIIPERNVIGTIPQDFLPPTSLKNGDSSYYDPNYLQ
SNEEKDRFLKIVTKIFNRINDNLSGRILLEELSKANPYLGNDNTPDNQFHIGDASAVEIKFSNGNQSILLPNVII
MGAEPDLFETNSSNISLRNNYMPSNHGFGSIAIVTFSPEYSFRFNDNSMNEFIQDPALTLMHELIHSLHGLYGAK
RITTKYTITQQQNPLITNIRGTNIEEFLTFGGTDLNIITSAQYNDIYTNLLADYKKIASKLSKVQVSNPQLNPYK
DIFQEKYGLDKNASGIYSVNINKFDDIFKKLYSFTEFDLATKFQVKCRQTYIGQYKYFKLSNLLNNSIYNISEGY

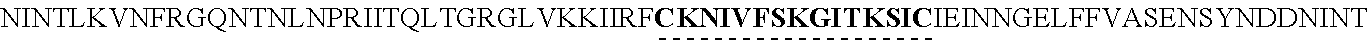

PKEIDDTVTSNNNYENDLDQVILNFNSESAPGLSDEKLNLTIONDAYIPKYDSNGTSYIEQHDVNELNVFFYLDA
QKVPEGENNVNLTSSIDTALLEQPKIYTFFSSEFINNVNKTVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNALKTIIESKYNSYTLEEKNELTNKYNIEQIENELNQK
VSIAMNNIEIFLTESSISYLMKLINEVKINKLREYDENVKTYLLDYIIKHGSILGESQQELNSMVIDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDKKSILNLGNIHVSDNILFKIVNCSYTRYIGIRYFNIF
DKELDETEIQTLYNNEPNANILKDFWGNYLLYDKEYYLLNVLKPNNFIDRRTDSTLSINNIRSTILLANRLYSGI
KVKIQRVNNSSTNDNLVRKNDQVYINFVASKTHLFPLYADTNTTNKEKTIKSSSSGNRFNQVVVMNSVGNNCTMN
FKNNNGNNIGMLGFKDNTLVASTWYYTHMRDNTNSNGCFWNFISEEHGWQEK
The endogenous activation loop is dash-underlined.
(BoNT/E9 - GenBank Accession No. JX424534.1)
SEQ ID NO: 120
MPKINSFNYNDPVNDNTILYIKPGGCQQFYKSFNIMKNIWIIPERNVIGTIPQNFLPPTSLKNGDSSYYDPNYLQ
NDQEKDRFLKIVTKVFNRINDNLSGRILLEELSKANPYLGNDNTRDDDFIINDGSAVPIQFSNGSQSILLPTVII
MGAEPDLFETNSSNVSLINNYSPSNHGFGSIAIVTFSPEYSFRFNDNSMNEFIQDPALTLIHELIHSLHGLYGAK
GITTKYTITQQQNPLITNIRGINIEEFLTFGGNNLNIITSSQLNDIYTNLLDDYKKIASKLSKVQVSNPQLNPYK
DVFQEKYGLDKNASGIYSVNINKENDIFKKLYSFTEFDLATKFQVKCRETYIGQYKYFKLSNLLNDSIYNISEGY

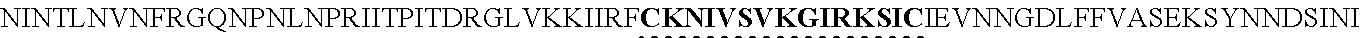

PKEIDDTVTLNNNYENDLDQVILNFNSESAPGLSDKKLNISIQDDVYIPKYDSNGTSDIEQYDVSELNVFFYLDA
QKVPEGENNVNLTSSIDTALLEQSKIYTFFSSEFINNVNKPVQAALFVGWIQQVLVDFTTEATQKSTVDKIADIS
IVVPYIGLALNIGNESQKGNFKDALELLGAGILLEFVPELLIPTILVFTIKSFLGSSDNKNKVIKAINNALKERD
EKWKEVYSFIVSNWITKINTQFNKRKEQMYQALQNQVNALKTIIESKYNSYTLEEKNELTNKYDIEQIENELNQK
VSIAMNNIDRFLTESSISYLMKLINEVKINKLREYDENVKTYLLDYITKHGSILGESQQELNSMIIDTLNNSIPF
KLSSYTDDKILISYFNKFFKTIKSSSVLSMRYKNDKYIDTSGYDSNININGDVFIYPTNKNOFGIYNSKLSEVNI
SQNDYIIYDNKYKNFSISFWVRIPNYNNKIVNVNNEYTIINCMRDNNSGWKISLNHNEIIWTLQDNAGINQKLVF
KYGNANGISDYINKWIFVTITNDRLGYSKLYINGHLIDQKSILNLGNIHVSDNILFKIVNCSYTRYIGMRYFNIF
DKELDETEIQTLYNNEPNANVLKDFWGNYLLYNKEYYLLNMLKPSKTISHNRDLTFSIYNNRNIVNGLYRLYSGI
KVKIQKINDSDTRDNIVRDNDQVYVNYINGNVYYSLYADTNATNKEKTIKSSTSGNRFNQVVVMNSVRNNCTMNF
KNNNGHDIGLLGFKSNALVASTWYYTNMRDHTNSNGCFWSF IPEENGWQEH
The endogenous activation loop is dash-underlined.
(BoNT/E10 - GenBank Accession No. KF861917.1)
SEQ ID NO: 121
MPKINSFNYNDPVNDKTILYIKPGGCQQFYKSFNIMKNIWIIPERNVIGTIPQDFLPPTSLKNGDSSYYDPNYLQ
SNEEKDRFLKIVTKIFNRINDNLSGGILLEELSKANPYLGNDNTPNNQFHIGDASAVEIKFSNGSQSILLPTVII
MGAEPDLFETNSSNISLKNNYMPSNHGFGSIAIVTFSPEYSFRFNDNSMNEFIQDPALTLMHELIHSLHGLYGAK
GITTKYTITQQQNPLITNIRGTNIEEFLTFGGTDLNIITNAQSNDIYTNLLADYKKIASKLSQVQVSNPOLNPYK
DIFQEKYGLDKNASGIYSVNINKFDDIFKKLYSFTEFDLATKFQVKCRQTYIGQYKYFKLSNLLNNSIYNISEGY

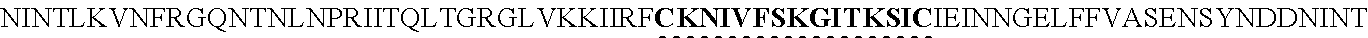

PKEIDDTVTSNNNYENDLDQVILNENSESAPGLSDEKLNLTIQNDAYIPKYDSNGTSDIEQHDVNELNVFFYLDA
QKVPEGENNVNLTSSIDTALLEQPKIYTFFSSEFINNVNKPVQAALFVSWIQQVLVDETTEANQKSTVDKIADIS
IVVPYIGLALNIGNEAQKGNFKDALELLGAGILLEFEPELLIPTILVFTIKSFLGSSDNKNKVIKAINNALKERD
EKWKEVYSFIVSNWMTKINTQFNKRKEQMYQALQNQVNALKTIIESKYNSYTLEEKNELTNKYNIEQIENELNQK
VSIAMNNIEIFLTESSISYLMKLINEVKINKLREYDENVKTYLLDYIIKHGSILGESQQELNSMVIDTLNNSIPF
KLSSYTDDKILISYFNKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEIIWTLQDNAGINQKLAF
NYGNANGISDYINKWIFVTITNDRLGDSKLYINGNLIDKKSILNLGNIHVSDNILFKIVNCSYTRYIGMRYFNIF
DKELDKTEIETLYNNEPNTNILKDFWGNYLLYDKEYYLLNVLKPNNVIDSNRDSTFSIHNIRSTIVLANKLYLGI
KVKIQRVNNSSTNDNLVRKNDQVYINFVPIKTHLFPLYADTNTTNKEKTIKSSSSGNRFNQVVVMNSVGNNCTMN
FKNNNGNNIGMLGFKDNTLVASTWYYTHMRDNTNSNGCFWNFISEEHGWQEK
The endogenous activation loop is dash-underlined.
(BoNT/E11 - GenBank Accession No. KF861875.1)
SEQ ID NO: 122
MPKINSFNYNDPVNDKTILYIKPGGCQQFYKSFNIMKNIWIIPERNVIGTIPQDFLPPTSLKNGDSSYYDPNYLQ
SNEEKDRFLKIVTKIFNRINDNLSGGILLEELSKANPYLGNDNTPNNQFHIGDASAVEIKFSNGSQSILLPTVII
MGAEPDLFETNSSNISLKNNYMPSNHGFGSIAIVTFSPEYSFRFNDNSMNEFIQDPALTLMHELIHSLHGLYGAK
GITTKYTITQQQNPLITNIRGTNIEEFLTFGGTDLNIITNAQSNDIYTNLLDDYKKIASKLSQVQVSNPQLNPYK
DVFQEKYGLDKDANGIYSVNINKENDIFKKLYSFTEFDLATKFQVKCRKTYIGHHKYFRLSDLLNDSIYNISDGY

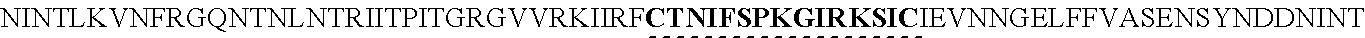

SKEIDDTVTSNNNYENDLDQVILNENSESAPGLSDEKLNLTIQDDAYIPKYDSNGTSDIEQYDVSELNVFFYLDA
QKVPEGENNVDFTSSIDTALLEQPKIYTFFSSKFISNLNKTMQAALFVSWIQQVLVDETTEATQKSTVDKIADIS
IVVPYIGLALNIGNEAQKGNFKDALELLGAGILLEFEPELLIPIILVFTIKSFLGSSDNKNKVIKAINNALKERD
ENWKEVYSFIVSNWMTKINTQFNKRKEQMYQALQNQVNAIKTIIESKYNSYTLEEKNELKNKYDIEQIENELNQT
VSIAMNNIEIFLTESSISYLMKLINEVKINKLKEYDENVKTYLLDYIIKHGSILGESQQELNSMVIDTLNNSIPF
KLSSYTDDKILISYFNKFFKTIKSSSVLNMRYKNDKYIDTSGYDSNINIKGDVFIYPTNKNQFGIYNNKLSEVNI
SONDYIIYDNKYKNFSISFWVRIPNYDNKIVNVNNEYTIINCMRDNNSGWKVSLNHNEIIWTLQDNAGINQKLVF
KYGNANGISDYINKWIFVTITNDRLGDSKLYINGNLIDKKSILNLGNIHVSDNILFKIVNCSYTRYIGMRYFNIF
DKELDKTEIETLYNNEPNTNILKDFWGNYLLYDKEYYLLNVLKPNNVIDSNRDSTFSIHNIRSTIVLANRLYSGI
KVKIQRVNNSSTNDNLVRKNDQVYINFVASKTHLFPLYADTNTTNKEKTIKSSSSGNRFNQVVVMNSVGNNCTMN
FKNNNGNNIGMLGFKDNTLVASTWYYTHMRDNTNSNGCFWNFISEEHGWQEK
The endogenous activation loop is dash-underlined.
(BoNT/E12 - GenBank Accession No. KM370319.1)
SEQ ID NO: 123
MLYMPKINSFNYNDPVNDRTILYIKPGGCQQFYKSFNIMKNIWIIPERNVIGTIPQDFQPPTSLKNGDSSYYDPN
YLQSNEEKDRFLKIVTKIFNRINDNLSGGILLEELSKANPYLGNDNTPDGDFIINDASAVPIQFSNGSQSILLPN
VIIMGAEPDLFETNSSNISLINNYRPSNHGFGSIAIVTFSPEYSFRFNDNSMNEFIQDPALTLMHELIHSLHGLY
GAKGITTKYTITQQQNSLITNIRGINIEEFLTFGGNDLNIITSSQFNDIYTNLLDDYKKIASKLSQVRVSNPQLN
PYKDVFQEKYGLDKDASGIYSVNINKENDIFKKLYSFTEFDLATKFQVKCRETYIGQYKYFQLSNLLNDSIYNIS

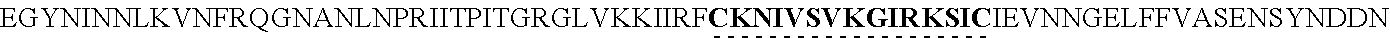

INTPKEIDDTVTSNNNYENDLDQVILNFNSESAPGLSDEKLNLTIQDDAYIPKYDSNGTSDIEQHDVNELNVFFY
LDAQKVPEGENNVNLTSSIDTALLEQPKIYTFFSSEFINNVNKPVQAVLFVSWIQQVLVDETTEATQKSTVDKIA
DISIVVPYIGLALNIGNEAQKGNFKDALELLGAGILLEFVPELLIPTILVFTIKSFLGSSDNKNKIIKAINNALK
ERDEKWKEVYSFIVSNWITKINTQFNKRKEQMYQALQNQVNAIKTIIESKYNSYTLEEKNELTNKYDIKQIENEL
NQKVSIAMNNIDRFLTESSISYLMKLINEVKINKLREYDENVKTYLLNYIIQHGSTLGESQQELNSMVINTLNNS
IPFKLSSYTDDKILISYFNKFFKRIKSSSVLNMRYKNDKYVDTSGYDSNININGEIFIYPTNKNQFSIFNSKPSE
VNISQNDYIIYDNKYKNFSISFWVRIPNYDNKIVNVNNEYTIINCMRDNNSGWKVSLNHNEIIWTLQDNAGINQK
LAFNYGNSNGISDYINKWIFVTITNDRLGDSKLYINGNLIDQKSILNLGNIHVSDNILFKIVNCSYTRYIGIRYF
NIFDKELDETEIQTLYSNEPNTNILKDFWGNYLLYDKEYYLLNVLKPNSIISHRRDLTFSFYNHRYIVNGLYRLY
SGIKVKIQRVNDSSTNDQFVRKNDQVYINYIYNNLSYSLYADTNIKDKEKTIKSSLSGNIFNQVVVMNSVGNNCT
MNFKNNNGNNIGLLGFKDNTLVASTWYYTHMRDNTNSNGCFWNFISEEHGWQEK
The endogenous activation loop is dash-underlined.
(BoNT/F1 - UniProt A7GBG3)
SEQ ID NO: 124
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG

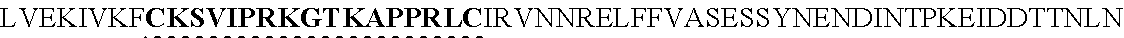

NNYRNNLDEVILDYNSETIPQISNOTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNOFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITONSNFLNINQORGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
The endogenous activation loop is dash-underlined.
(BoNT/F2 - GenBank Accession No. GU213209.1)
SEQ ID NO: 125
MPVVINSFNYNDPVNDETILYMQKPYEERSRKYYKAFEIMPNVWIMPERDTIGTKPDEFQVPDSLKNGSSAYYDP
NYLTTDAEKDRYLKTMIKLFNRINSNPTGKVLLEEVSNARPYLGDDDTLINEFFPVNVTTSVNIKFSTDVESSII
SNLLVLGAGPDIFKAYCTPLVRFNKSDKLIEPSNHGFGSINILTFSPEYEHIFNDISGGNHNSTESFIADPAISL
AHELIHALHGLYGAKAVTHKESLVAERGPLMIAEKPIRLEEFLTFGGEDLNIIPSAMKEKIYNDLLANYEKIATR
LREVNTAPPGYDINEYKDYFQWKYGLDRNADGSYTVNRNKFNEIYKKLYSFTEIDLANKFKVKCRNTYFIKYGFV

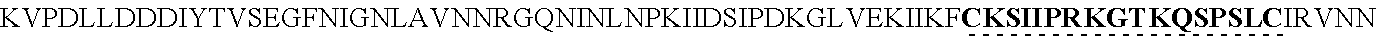

RELFFVASESSYNESDINTPKEIDDTTNLNNNYRNNLDEVILDYNSETIPQISNRTLNTLVQDNSYVPRYDSNGT
SEIEEYDVVDFNVFFYLHAQKVPEGETNISLTSSIDTALLEESKVYTFFSSEFIDTINKPVNAALFIDWISKVIR
DFTTEATQKSTVDKIADISLIVPYVGLALNIVIEAEKGNFEEAFELLGAGILLEFVPELTIPVILVFTIKSYIDS
YENKNKAIKAINNSLIEREAKWKEIYSWIVSNWLTRINTQFNKRKEQMYQALQNQVDAIKTAIEYKYNNYTSDEK
NRLESKYNINNIEEELNKKVSLAMKNIERFMTESSISYLMKLINEAEVGKLKEYDKHVKSDLLDYILYHKLILGE
QTKELIDLVTSTLNSSIPFELSSYTNDKILIIYFNRLYKKIKDSSILDMRYENNKFIDISGYGSNISINGNVYIY
STNRNQFGIYSGRLSEVNIAQNNDIIYNSRYQNFSISFWVRIPKHYRPMNRNREYTIINCMGNNNSGWKISLRTI
RDCEIIWTLQDTSGNKEKLIFRYEELASISDYINKWIFVTITNNRLGNSRIYINGNLIVEKSISNLGDIHVSDNI
LFKIVGCDDETYVGIRYFKVENTELDKTEIETLYSNEPDPSILKDYWGNYLLYNKKYYLFNLLRKDKYITRNSGI
LNINQQRGVTGGISVFLNYKLYEGVEVIIRKNAPIDISNTDNFVRKNDLAYINVVDHGVEYRLYADISITKSEKI
IKLIRTSNPNDSLGQIIVMDSIGNNCTMNFQNNDGSNIGLLGFHSDDLVASSWYYNHIRRNTSSNGCFWSFISKE
HGWKE
The endogenous activation loop is dash-underlined.
(BoNT/F3 - GenBank Accession No. GU213227.1)
SEQ ID NO: 126
MPVVINSFNYNDPVNDETILYMQKPYEERSRKYYKAFEIMPNVWIMPERDTIGTKPDDFQVPDSLKNGSSAYYDP
NYLTTDAEKDRYLKTMIKLFNRINSNPTGKVLLEEVSNARPYLGDDDTLINEFFPVNVTTSVNIKFSTDVESSII
SNLLVLGAGPDIFKAYCTPLVRFNKSDKLIEPSNHGFGSINILTFSPEYEHIFNDISGGDHNSTESFIADPAISL
AHELIHALHGLYGAKAVTHKETIEVKRGPLMIAEKPIRLEEFLTFGGEDLNIIPSAMKEKIYNDLLANYEKIATR
LREVNTAPPEYDINEYKDYFQWKYGLDRNADGSYTVNRNKFNGIYKKLYSFTEIDLANKFKVKCRNTYFIKYGFV

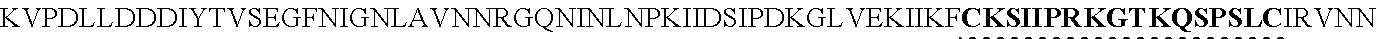

RELFFVASESSYNESDINTPKEIDDTTNLNNNYRNNLDEVILDYNSETIPQISNRTLNTLVQDNSYVPRYDSNGT
SEIEEYDVVDFNVFFYLHAQKVPEGETNISLTSSIDTALLEKSKVYTFFSSEFIDTINESVNAALFIDWINKVIR
DFTTEATQKSTVDKIADISLIVPYVGLALNIVIDAEKGNFQEAFELLGAGILLEFVPELTIPVILVFTIKSYIDS
YENKNKAIKAINNALIEREAKWKEIYSWIVSNWLTKINTQFNKRKEQMYQALQNQVDAIKTAIEYKYNNYTSDEK
NRLESEYNINNIEEELNKKVSLAMKNIERFMTESSISYLMKLINEAEVGKLKKYDRHVKSDLLDYILYHKLILGD
QTKELIDLVTSTLNSSIPFELSSYTNDKILIIYFNRLYKKIKDSSILDMRYENNKFIDISGYGSNISINGNVYIY
STNRNQFGIYSDRLSEVNIAONNDIIYNSRYQNFSISFWVRIPKHYGPMNRNREYTIINCMGNNNSGWKISLRNI
RDCEIIWTLQDTSGNKEKLIFRYEELANISDYINKWIFVTITNNRLGNSRIYINGNLIVEKSISNLGDIHVSDNI
LFKIVGCDDKTYVGIRYFKVENTELDKTEIETLYSNEPDPSILKDYWGNYLLYNKKYYLFNLLRKDKYITRNSGI
LNINQQRGVTEGSVFLNYKLYEGVEVIIRKNGPIDISNTDNFVRKNDLAYINVVYHDVEYRLYADISITKPEKII
KLIRTSNPNDSLGQIIVMDSIGNNCTMNFQNNNGGNIGLLGFHSDNLVASSWYYNNIRRNTSSNGCFWSFISKEH
GWQE
The endogenous activation loop is dash-underlined.
(BoNT/F4 - GenBank Accession No. GU213214.1)
SEQ ID NO: 127
MPVVINSFNYDDPVNDDTILYMQIPYEEKSKKYYKAFEIMRNVWIMPERNTIGTNPSDFDPPASLKNGSSAYYDP
NYLTTDAEKDRYLKTTIKLFKRINSNPAGEVLLQEISYAKPYLGNDHTPINEFHPVTRTTSVNIKSSTNVESSII
LNLLVLGAGPNIFENSSYPVRKLMNSGEVYDPSNDGFGSINIVTFSPEYEYTFNDISGGHNSSTESFIADPAISL
AHELIHALHGLYGARGVTYKETIKVKQAPLMIAEKPIRLEEFLTFGGQDLNIITSAMKEKIYNDLLANYEKIATR
LSEVNSAPPEYDINEYKNYFQWKYGLDKNADGSYTVNENKFNEIYKKLYSFTEIDLANKFKVKCRNTYFIKYGFL

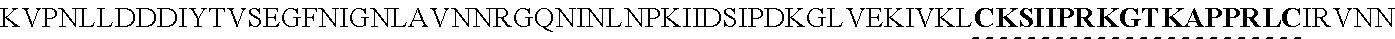

RELFFVASESSYNENDINTPKEIDDTTNLNNNYRNNLDEVILDYNSETIPOISSQTLNTLVQDDSYVPRYDSNGT
SEIEEHNVVDLNAFFYLHAQKVPEGETNISLTSSIDTALSEESKVYTFFSSEFINNINKPVHAALFIGWISQVIR
DFTTESTQKSTVDKIADISLIVPYVGLALNIGNDARKGNFKEAFELLGAAILLEVVPELLIPVILVFTIKSFIDS
SKNEDKIIKAINNSLIEREAKWKEVYSWIVSNWLTRINTQFNKRKEQMYQALQNQVDAIKTVIEYKYNSYTSDEK
NRLESEYNINNIEEELNKKVSLAMKNIERFIAESSISYLMKLINEAKVSELREYDEGVKEYLLDYILKNGSILGD
HVQELNDLVTSTLNSSIPFELSSYTNDKILIIYFNKLYKKIKDNCILDMRYENNKFIDISGYGSNISINGELYIY
TTNRNQFTIYSGKLSEVNIAQNNDIIYNSRYQNFSISFWVRIPRYSNIVNLNNEYTIINCMGNNNSGWKISLNYN
KIIWTLQDTAGNNEKLVFNYTQMISISDYINKWIFVTITNNRLGNSRIYINGNLIDQKSISNLGDIHVSDNILFK
IVGCNDTRYVGIRYFKVFDTELDKTEIETLYSDEPDPSILKDFWGNYLLYNKRYYLLNLLRKDNAITQSSTFLSI
SRARGVDRKANIFSNKRLYKGVEVIIRKNEPIDISNTDNFVRKGDLAYINVVDRDVEYRLYANTSNAQPEKTIKL
IRTSNSNDSLDQIIVMDSIGNNCTMNFQNNNGGNIGLLGFHSNTLVASSWYYNNIRRNTSSNGCFWSFISKEHGW
QE
The endogenous activation loop is dash-underlined.
(BoNT/F5 - GenBank Accession No. GU213211.1)
SEQ ID NO: 128
MPVEINSFNYDDLVNDNTILYIRPPYYERSNTYFKAFNIMENVWIIPERYRLGIEASKFDPPDSLKAGSDGYFDP
NYLSTNTEKNRYLQIMIKLFKRINSNEAGKILLNQIKDAIPYLGNSYTAEDQFTTNNRTISFNVRLANGTIEQEM
ANLIIWGPGPDLTTNRTGGTTYTPAQSLEAIPYKEGFGSIMTIEFSPEYATAFNDISLTSHAPSLFIKDPALILM
HELIHVLHGLYGTYTTGFKIKPNITEPYMEVTKPITSGEFLTFGGNDVNKIPQLIQSQLRSKVLDDYEKIASRLN
KVNRATAEINIDKFKYSYQLKYQFVKDSNGVYSVDLDKFNKLYDKIYSFTEFNLAHEFKIKTRNSYLAKNFGPFY

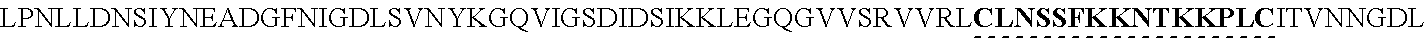

FFIASEDSYGEDTINTPKEIDDTTTLVPSFKNILDKVILDENKQVTPQIPNRRIRTDIQEDNYIPEYDSNGTSEI
EEYNVVDLNAFFYLHAQKVPEGETNISLTSSIDTALSEESKVYTFFSSEFIDTINEPVNAALFIDWISKVIRDFT
TEATQKSTVDKIADISLIVPYVGLALNIVNETEKGNFKEAFELLGAGILLEFVPELAIPVILVFTIKSYIDSYEN
KNKIIKAINNSLIEREAKWKEIYSWIVSNWLTRINTQFNKRKEQMYQALQNQVDAIKTAIEYKYNNYTSDEKNRL
ESEYNINNIEEELNKKVSLAMKNIERFITESSISYLMKLINEAEVGKLKEYDKRVKRHLLEYIFDYRLILGEQGG
ELIDLVTSTLNTSIPFELSSYTNDKILIIYFNRLYKKIKDSSILDMRYENNKFIDISGYGSNISINGNVYIYSTN
RNQFGIYDDRLSEVNIAQNNDIIYNSRYQNFSISFWVRIPKHYRPMNHNREYTIINCMGNNNSGWKISLRTTGDC
EIIWTLQDTSGNKKKLIFRYSQLGGISDYINKWIFVTITNNRLGNSRIYINGNLIVEKSISNLGDIHVSDNILFK
IVGCDDKMYVGIRYFKVENTELDKTEIEILYSNEPDPSILKDYWGNYLLYNKKYYLLNLLRNDKYITRNSDILNI
SHQRGVTKDLFIFSNYKLYEGVEVIIRKNGPIDISNTDNFVRKNDLAYINVVDHGVEYRLYADISITKPEKIIKL
IRRSNPDDSLGQIIVMDSIGNNCTMNFQNNNGGNIGLLGFHSDNLVASSWYYNNIRRNTSSNGCFWSFISKEHGW
QE
The endogenous activation loop is dash-underlined.
(BoNT/F6 - GenBank Accession No. M92906.1)
SEQ ID NO: 129
MPVAINSFNYNDPVNDDTILYMQIPYEEKSKKYYKAFEIMRNVWIIPERNTIGTNPSDFDPPASLKNGSSAYYDP
NYLTTDAEKDRYLKTTIKLFKRINSNPAGKVLLQEISYAKPYLGNDHTPIDEFSPVTRTTSVNIKLSTNVESSML
LNLLVLGAGPDIFESCCYPVRKLIDPDVVYDPSNYGFGSINIVTFSPEYEYTFNDISGGHNSSTESFIADPAISL
AHELIHALHGLYGARGVTYEETIEVKOAPLMIAEKPIRLEEFLTFGGQDLNIITSAMKEKIYNNLLANYEKIATR
LSEVNSAPPEYDINEYKDYFQWKYGLDKNADGSYTVNENKFNEIYKKLYSFTESDLANKFKVKCRNTYFIKYEFL

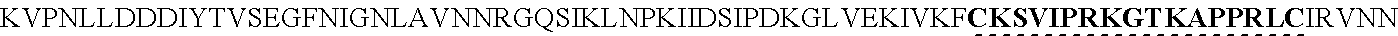

SELFFVASESSYNENDINTPKEIDDTTNLNNNYRNNLDEVILDYNSQTIPQISNRTLNTLVQDNSYVPRYDSNGT
SEIEEYDVVDFNVFFYLHAQKVPEGETNISLTSSIDTALLEESKDIFFSSEFIDTINKPVNAALFIDWISKVIRD
FTTEATQKSTVDKIADISLIVPYVGLALNIIIEAEKGNFEEAFELLGVGILLEFVPELTIPVILVFTIKSYIDSY
ENKNKAIKAINNSLIEREAKWKEIYSWIVSNWLTRINTQFNKRKEQMYQALQNQVDAIKTAIEYKYNNYTSDEKN
RLESEYNINNIEEELNKKVSLAMKNIERFMTESSISYLMKLINEAKVGKLKKYDNHVKSDLLNYILDHRSILGEQ
TNELSDLVTSTLNSSIPFELSSYTNDKILIIYFNRLYKKIKDSSILDMRYENNKFIDISGYGSNISINGNVYIYS
TNRNQFGIYNSRLSEVNIAQNNDIIYNSRYQNFSISFWVRIPKHYKPMNHNREYTIINCMGNNNSGWKISLRTVR
DCEIIWTLQDTSGNKENLIFRYEELNRISNYINKWIFVTITNNRLGNSRIYINGNLIVEKSISNLGDIHVSDNIL
FKIVGCDDETYVGIRYFKVENTELDKTEIETLYSNEPDPSILKNYWGNYLLYNKKYYLFNLLRKDKYITLNSGIL
NINQQRGVTEGSVFLNYKLYEGVEVIIRKNGPIDISNTDNFVRKNDLAYINVVDRGVEYRLYADTKSEKEKIIRT
SNLNDSLGQIIVMDSIGNNCTMNFQNNNGSNIGLLGFHSNNLVASSWYYNNIRRNTSSNGCFWSSISKENGWKE
The endogenous activation loop is dash-underlined.
(BoNT/F7 - GenBank Accession No. GU213233.1)
SEQ ID NO: 130
MPVNINNFNYNDPINNTTILYMKMPYYEDSNKYYKAFEIMDNVWIIPERNIIGKKPSDFYPPISLDSGSSAYYDP
NYLTTDAEKDRFLKTVIKLFNRINSNPAGQVLLEEIKNGKPYLGNDHTAVNEFCANNRSTSVEIKESNGTTDSML
LNLVILGPGPNILECSTFPVRIFPNNIAYDPSEKGFGSIQLMSFSTEYEYAFNDNTDLFIADPAISLAHELIHVL
HGLYGAKGVTNKKVIEVDQGALMAAEKDIKIEEFITFGGQDLNIIINSTNQKIYDNLLSNYTAIASRLSQVNINN
SALNTTYYKNFFQWKYGLDQDSNGNYTVNISKFNAIYKKLFSFTECDLAQKFQVKNRSNYLFHFKPFRLLDLLDD

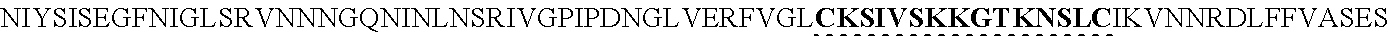

SYNENGINSPKEIDDTTITNNNYKKNLDEVILDYNSDAIPNLSSRLLNTTAQNDSYVPKYDSNGTSEIKEYTVDK
LNVFFYLYAQKAPEGESAISLTSSVNTALLDASKVYTFFSSDFINTVNKPVQAALFISWIQQVINDFTTEATQKS
TIDKIADISLVVPYVGLALNIGNEVQKGNFKEAIELLGAGILLEFVPELLIPTILVFTIKSFINSDDSKNKIIKA
INNALRERELKWKEVYSWIVSNWLTRINTQFNKRKEQMYQALQNQVDGIKKIIEYKYNNYTLDEKNRLKAEYNIY
SIKEELNKKVSLAMONIDRFLTESSISYLMKLINEAKINKLSEYDKRVNOYLLNYILENSSTLGTSSVQELNNLV
SNTLNNSIPFELSEYTNDKILISYFNRFYKRIIDSSILNMKYENNRFIDSSGYGSNISINGDIYIYSTNRNOFGI
YSSRLSEVNITONNTIIYNSRYQNFSVSFWVRIPKYNNLKNLNNEYTIINCMRNNNSGWKISLNYNNIWTLQDT
TGNNQKLVFNYTQMIDISDYINKWTFVTITNNRLGHSKLYINGNLTDQKSILNLGNIHVDDNILFKIVGCNDTRY
VGIRYFKIFNMELDKTEIETLYHSEPDSTILKDFWGNYLLYNKKYYLLNLLKPNMSVTKNSDILNINRQRGIYSK
TNIFSNARLYTGVEVIIRKVGSTDTSNTDNFVRKNDTVYINVVDGNSEYQLYADVSTSAVEKTIKLRRISNSNYN
SNQMIIMDSIGDNCTMNFKTNNGNDIGLLGF HLNNLVASSWYYKNIRNNTRNNGCFWSFISKEHGWQE
The endogenous activation loop is dash-underlined.
(BoNT/G - UniProt Q60393)
SEQ ID NO: 131
MPVNIKXFNYNDPINNDDIIMMEPENDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVENNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGENIASKNLKTEFNGONKAVNKEAY

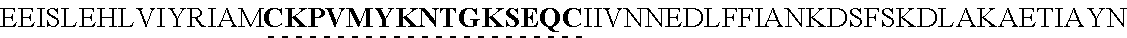

TQNNTIENNFSIDQLILDNDLSSGIDLPNENTEPFTNEDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDENDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVENNYISNISSNAILSLSYRGGRLIDSSGYGATMNVGSDVIEND
IGNGQFKLNNSENSNITAHQSKFVVYDSMFDNESINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMQNIYIKYFSKASMGETAPRTNENNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
The endogenous activation loop is dash-underlined.
(BoNT/FA - GenBank Accession No. KGO15617.1)
SEQ ID NO: 132
MPVVINSFNYDDPVNDNTIIYIRPPYYETSNTYFKAFQIMDNVWIIPERYRLGIDPSLFNPPVSLKAGSDGYFDP
NYLSTNTEKNKYLQIMIKLFKRINSKPAGQILLEEIKNAIPYLGNSYTQEEQFTTNNRTVSFNVKLANGNIVQQM
ANLIIWGPGPDLTTNKTGGIIYSPYQSMEATPYKDGFGSIMTVEFSPEYATAFNDISIASHSPSLFIKDPALILM
HELIHVLHGLYGTYITEYKITPNVVQSYMKVTKPITSAEFLTFGGRDRNIVPQSIQSQLYNKVLSDYKRIASRLN
KVNTATALINIDEFKNLYEWKYQFAKDSNGVYSVDLNKFEQLYKKIYSFTEFNLAYEFKIKTRLGYLAENFGPFY

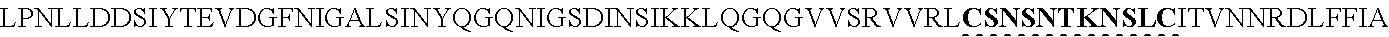

SQESYGENTINTYKEIDDTTTLDPSFEDILDKVILNFNEQVIPQMPNRNVSTDIQKDNYIPKYDYNRTDIIDSYE
VGRNYNTFFYLNAQKFSPNESNITLTSSFDTGLLEGSKVYTFFSSDFINNINKPVQALLFIEWVKQVIRDETTEA
TKTSTVDKLKDISLVVPYIGLALNIGDEIYKQHFAEAVELVGAGLLLEFSPEFLIPTLLIFTIKGYLTGSIRDKD
KIIKTLDNALNVRDQKWKELYRWVVSKWLTTINTQFNKRKEQMYKALKNQATAIKKIIENKYNNYTTDEKSKIDS
SYNINEIERTLNEKINLAMKNIEQFITESSIAYLINIINNETIQKLKSYDDLVRRYLLGYIRNHSSILGNSVEEL
NSKVNNHLDNGIPFELSSYTNDSLLIRYFNKNYGELKYNCILNIKYEMDRDKLVDSSGYRSRINIGTGVKFSEID
KNQVQLSNLESSKIEVILNNGVIYNSMYENFSTSFWIRIPKYFRNINNEYKIISCMQNNSGWEVSLNFSNMNSKI
IWTLQDTEGIKKTVVFQYTQNINISDYINRWIFVTITNNRLSNSKIYINGRLINEESISDLGNIHASNNIMFKLD
GCRDPHRYIWIKYFNLFDKELNKKEIKDLYDNQSNSGILKDFWGDYLQYDKPYYMLNLYDPNKYLDVNNVGIRGY
MYLKGPRGRIVTTNIYLNSTLYMGTKFIIKKYASGNKDNIVRNNDRVYINVVVKNKEYRLATNASQAGVEKILSA
VEIPDVGNLSQVVVMKSENDQGIRNKCKMNLQDNNGNDIGFIGFHQFNNIAKLVASNWYNRQIGKASRTFGCSWE
FIPVDDGWGESSL
The endogenous activation loop is dash-underlined.
(Polypeptide Sequence of BoNT/X)
SEQ ID NO: 133
MKLEINKFNYNDPIDGINVITMRPPRHSDKINKGKGPFKAFQVIKNIWIVPERYNFINNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI

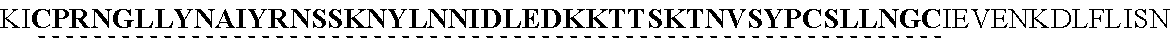

KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKINWYFIPKDEGWDED
The endogenous activation loop is dash-underlined.
(TeNT - UniProt P04958)
SEQ ID NO: 134
MPITINNFRYSDPVNNDTIIMMEPPYCKGLDIYYKAFKITDRIWIVPERYEFGTKPEDEN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT

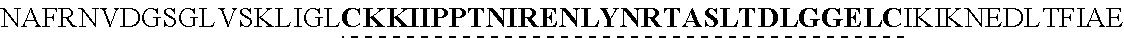

KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLOWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKOLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMENNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWIND
The endogenous activation loop is dash-underlined.
(BoNT/A GenBank Accession No. AF488749.1)
SEQ ID NO: 135
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDINCINVIQPDGSYRSEELNLVIIGPSADIIQFECKSFGHEVLNLTRNGY
GSTQYIRFSPDFTFGFEESLEVDINPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGENLRNTNLAANFNGQNTEINNMNFTKLKNFT

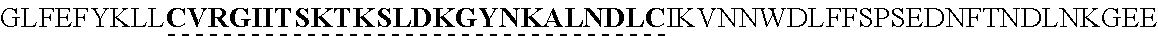

ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLIVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNN
EYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLFDKELN
EKEIKDLYDNQSNSGILKDFWGDYLQYDKPYYMLNLYDPNKYVDVNNVGIRGYMYLKGPR
GSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
The endogenous activation loop is dash-underlined.
non-engineered BoNT/AB chimera
SEQ ID NO: 136
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK

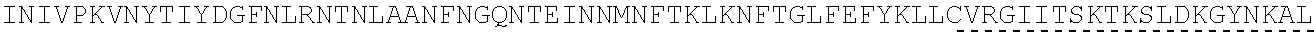

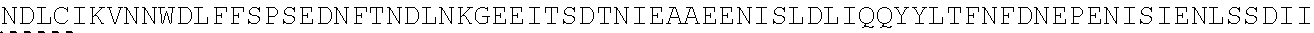


GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNORLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIONYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
Sytll-binding mutations E1191M and S1199Y are bold and underlined.
The endogenous activation loop is dash-underlined.
GS linker consensus
SEQ ID NO: 137
(GGGGS)ₙ
GS5 linker
SEQ ID NO: 138
GGGGS
GS10 linker
SEQ ID NO: 139
GGGGSGGGGS
GS15 linker
SEQ ID NO: 140
GGGGSGGGGSGGGGS
GS20 linker
GGGGSGGGGSGGGGSGGGGS
GS25 linker
SEQ ID NO: 142
GGGGSGGGGSGGGGSGGGGSGGGGS
metal coordinating SNARE cleavage motif
SEQ ID NO: 143
HEXXH
leucine-based motif
SEQ ID NO: 144
xDxxxLL
leucine-based motif
SEQ ID NO: 145
xExxxLL
leucine-based motif
SEQ ID NO: 146
xExxxIL
leucine-based motif
SEQ ID NO: 147
xExxxLM
TEV(Tobacco Etch virus) cleavage motif
SEQ ID NO: 148
ENLYFQG
PreScission cleavage motif
SEQ ID NO: 149
LEVLFQGP
LC/A1-Cloop-H_{N}/A1
SEQ ID NO: 150
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCHKAIDGRSLYNKTLDCIKVN
NWDLFFSPSEDNFINDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVS
YIANKVLTVQTIDNALSKRNEKWDEVYKYIVTNWLAKVNTQIDLIRKKMKEALENQAEATKAIINYQYNQYTEEE
KNNINFNIDDLSSKLNESINKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKN
LC/A1 is italicised
BoNT/C activation loop is bold and underlined
H_{N}A1 is C-terminal to the C activation loop and is neither underlined nor italicised
LC/A1- Exogenous Activation Loop -H_{N}/A1
SEQ ID NO: 151
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDINCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGONTEINNMNFTKLKNFTGLFEFYKLL[EXOGENOUS PROTEASE
CLEAVAGE SITE AS DEFINED HEREIN]
IKVNNWDLFFSPSEDNFTNDLNKGEEITSDTNIEAAEENISLDLIQQYYLTFNFDNEPENISIENLSSDIIGQLE
LMPNIERFPNGKKYELDKYTMFHYLRAQEFEHGKSRIALTNSVNEALLNPSRVYTFFSSDYVKKVNKATEAAMFL
GWVEQLVYDFTDETSEVSTTDKIADITIIIPYIGPALNIGNMLYKDDFVGALIFSGAVILLEFIPEIAIPVLGTF
ALVSYIANKVLTVQTIDNALSKRNEKWDEVYKYIVTNWLAKVNTQIDLIRKKMKEALENQAEATKAIINYQYNQY
TEEEKNNINFNIDDLSSKLNESINKAMININKFLNQCSVSYLMNSMIPYGVKRLEDFDASLKDALLKYIYDNRGT
LIGQVDRLKDKVNNTLSTDIPFQLSKYVDNORLLSTFTEYIKN
LC/A1 is italicised
Exogenous activation loop is bold and underlined
H_{N}A1 is C-terminal to the C activation loop and is neither underlined nor italicised
LC/X-Cloop-HN/X
SEQ ID NO: 152:
MKLEINKFNYNDPIDGINVITMRPPRHSDKINKGKGPFKAFQVIKNIWIVPERYNFINNINDLNIPSEPIMEADA
IYNPNYLNTPSEKDEFLQGVIKVLERIKSKPEGEKLLELISSSIPLPLVSNGALTLSDNETIAYQENNNIVSNLQ
ANLVIYGPGPDIANNATYGLYSTPISNGEGTLSEVSFSPFYLKPFDESYGNYRSLVNIVNKFVKREFAPDPASTL
MHELVHVTHNLYGISNRNFYYNFDTGKIETSROONSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVRKHYLKERPIDPIYVN
ILDDNSYSTLEGFNISSQGSNDFQGQLLESSYFEKIESNALRAFIKICHKAIDGRSLYNKTLDCIEVENKDLFLI
SNKDSLNDINLSEEKIKPETTVFFKDKLPPQDITLSNYDFTEANSIPSISQQNILERNEELYEPIRNSLFEIKTI
YVDKLTTFHFLEAQNIDESIDSSKIRVELTDSVDEALSNPNKVYSPFKNMSNTINSIETGITSTYIFYQWLRSIV
KDFSDETGKIDVIDKSSDTLAIVPYIGPLLNIGNDIRHGDFVGAIELAGITALLEYVPEFTIPILVGLEVIGGEL
AREQVEAIVNNALDKRDQKWAEVYNITKAQWWGTIHLQINTRLAHTYKALSRQANAIKMNMEFQLANYKGNIDDK
AKIKNAISETEILLNKSVEQAMKNTEKFMIKLSNSYLTKEMIPKVQDNLKNEDLETKKTLDKFIKEKEDILGTNL
SSSLRRKVSIRLNKNIAFDINDIPFSEFDDLINQYKNE
LC/X is italicised
BoNT/C activation loop is bold and underlined
H_{N}X is C-terminal to the C activation loop and is neither underlined nor italicised
LC/X-Exogenous Activation Loop-HN/X
SEQ ID NO: 153:
MKLEINKFNYNDPIDGINVITMRPPRHSDKINKGKGPFKAFQVIKNIWIVPERYNFINNINDLNIPSEPIMEADA
IYNPNYLNTPSEKDEFLOGVIKVLERIKSKPEGEKLLELISSSIPLPLVSNGALTLSDNETIAYQENNNIVSNLQ
ANLVIYGPGPDIANNATYGLYSTPISNGEGTLSEVSFSPFYLKPFDESYGNYRSLVNIVNKFVKREFAPDPASTL
MHELVHVTHNLYGISNRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVRKHYLKERPIDPIYVN
ILDDNSYSTLEGFNISSQGSNDFQGQLLESSYFEKIESNALRAFIKI[EXOGENOUS PROTEASE CLEAVAGE
SITE AS DEFINED HEREIN]
IEVENKDLFLISNKDSLNDINLSEEKIKPETTVFFKDKLPPQDITLSNYDFTEANSIPSISQQNILERNEELYEP
IRNSLFEIKTIYVDKLTTFHFLEAQNIDESIDSSKIRVELTDSVDEALSNPNKVYSPFKNMSNTINSIETGITST
YIFYQWLRSIVKDESDETGKIDVIDKSSDTLAIVPYIGPLLNIGNDIRHGDFVGAIELAGITALLEYVPEFTIPI
LVGLEVIGGELAREQVEAIVNNALDKRDQKWAEVYNITKAQWWGTIHLQINTRLAHTYKALSRQANAIKMNMEFQ
LANYKGNIDDKAKIKNAISETEILLNKSVEQAMKNTEKFMIKLSNSYLTKEMIPKVQDNLKNEDLETKKTLDKFI
KEKEDILGTNLSSSLRRKVSIRLNKNIAFDINDIPFSEFDDLINQYKNE
LC/X is italicised
Exogenous activation loop is bold and underlined
H_{N}X is C-terminal to the C activation loop and is neither underlined nor italicised
Nucleic acid encoding exemplary engineered BoNT/A1B1 chimera
with modified Cloop/PPFGRSAG of SEQ ID NO: 63
SEQ ID NO: 154:
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGTCATAAAGCCATTCCGCCGTTTGGTCGTAGCGCAGGTTATAACAAAACCCTGGATTGTATT
AAGGTAAACAATTGGGATCTGTTCTTTTCGCCATCCGAAGATAATTTTACCAACGACCTGAACAAGGGTGAAGAA
ATCACCAGCGATACGAATATTGAAGCAGCGGAAGAGAATATCAGCCTGGATCTGATCCAGCAGTACTATCTGACC
TTTAACTTCGACAATGAACCGGAGAACATTAGCATTGAGAATCTGAGCAGCGACATTATCGGTCAGCTGGAACTG
ATGCCGAATATCGAACGTTTCCCGAACGGCAAAAAGTACGAGCTGGACAAGTACACTATGTTCCATTACCTGCGT
GCACAGGAGTTTGAACACGGTAAAAGCCGTATCGCGCTGACCAACAGCGTTAACGAGGCCCTGCTGAACCCGAGC
CGTGTCTATACCTTCTTCAGCAGCGACTATGTTAAGAAAGTGAACAAAGCCACTGAGGCCGCGATGTTCCTGGGC
TGGGTGGAACAGCTGGTATATGACTTCACGGACGAGACGAGCGAAGTGAGCACTACCGACAAAATTGCTGATATT
ACCATCATTATCCCGTATATTGGTCCGGCACTGAACATTGGCAACATGCTGTACAAAGACGATTTTGTGGGTGCC
CTGATCTTCTCCGGTGCCGTGATTCTGCTGGAGTTCATTCCGGAGATTGCGATCCCGGTGTTGGGTACCTTCGCG
CTGGTGTCCTACATCGCGAATAAGGTTCTGACGGTTCAGACCATCGATAACGCGCTGTCGAAACGTAATGAAAAA
TGGGACGAGGTTTACAAATACATTGTTACGAATTGGCTGGCGAAAGTCAATACCCAGATCGACCTGATCCGTAAG
AAAATGAAAGAGGCGCTGGAGAATCAGGCGGAGGCCACCAAAGCAATTATCAACTACCAATACAACCAGTACACG
GAAGAAGAGAAGAATAACATTAACTTCAATATCGATGATTTGAGCAGCAAGCTGAATGAATCTATCAACAAAGCG
ATGATCAATATCAACAAGTTTTTGAATCAGTGTAGCGTTTCGTACCTGATGAATAGCATGATTCCGTATGGCGTC
AAACGTCTGGAGGACTTCGACGCCAGCCTGAAAGATGCGTTGCTGAAATACATTTACGACAATCGTGGTACGCTG
ATTGGCCAAGTTGACCGCTTGAAAGACAAAGTTAACAATACCCTGAGCACCGACATCCCATTTCAACTGAGCAAG
TATGTTGATAATCAACGTCTGTTGAGCACTTTCACCGAGTATATCAAAAACATTCTGAACAATATTATCCTGAAC
CTGCGTTACAAAGACAACAATCTGATCGATCTGAGCGGCTATGGTGCAAAAGTTGAAGTCTACGACGGTGTCGAA
CTGAACGATAAAAACCAGTTCAAACTGACCTCATCGGCTAACTCAAAAATTCGTGTGACGCAGAACCAAAACATC
ATCTTCAACTCGGTCTTTCTGGACTTCAGCGTGTCTTTCTGGATTCGCATCCCGAAATATAAAAATGATGGCATC
CAGAACTACATCCATAACGAATACACCATCATCAACTGTATGAAAAACAACAGTGGTTGGAAAATTTCCATCCGT
GGCAACCGCATTATCTGGACCCTGATTGATATCAATGGTAAAACGAAAAGCGTGTTTTTCGAATACAACATCCGT
GAAGATATCTCTGAATACATCAATCGCTGGTTTTTCGTGACCATTACGAACAATCTGAACAATGCGAAAATCTAT
ATCAACGGCAAACTGGAAAGTAATACCGACATCAAAGATATTCGTGAAGTTATCGCCAACGGTGAAATCATCTTC
AAACTGGATGGCGACATCGATCGCACCCAGTTCATTTGGATGAAATACTTCTCCATCTTCAACACGGAACTGAGT
CAGTCCAATATCGAAGAACGCTACAAAATCCAATCATACTCGGAATACCTGAAAGATTTCTGGGGTAACCCGCTG
ATGTACAACAAAGAATACTACATGTTCAACGCGGGCAACAAAAACTCATACATCAAACTGAAAAAAGATTCGCCG
GTGGGTGAAATCCTGACCCGTAGCAAATACAACCAGAACTCTAAATACATCAACTATCGCGATCTGTACATTGGC
GAAAAATTTATTATCCGTCGCAAAAGCAACTCTCAGAGTATTAATGATGACATCGTGCGTAAAGAAGACTACATC
TATCTGGATTTCTTTAATCTGAACCAAGAATGGCGCGTTTATACCTACAAATACTTCAAAAAAGAAGAAATGAAA
CTGTTCCTGGCCCCGATTTACGACAGCGATGAATTTTACAACACCATCCAGATCAAAGAATACGATGAACAGCCG
ACGTATAGTTGCCAACTGCTGTTCAAAAAAGACGAAGAATCCACCGATGAAATTGGCCTGATTGGTATCCACCGT
TTCTATGAAAGCGGTATCGTTTTCGAAGAATACAAAGATTACTTCTGTATCTCTAAATGGTATCTGAAAGAAGTC
AAACGCAAACCGTACAACCTGAAACTGGGCTGCAACTGGCAATTTATCCCGAAAGACGAAGGCTGGACCGAA
Exemplary engineered BoNT/A1B1 chimera with modified Cloop/
PPFGRSAG of SEQ ID NO: 63
SEQ ID NO: 155:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGONTEINNMNFTKLKNFTGLFEFYKLLCHKAIPPFGRSAGYNKTLDCI
KVNNWDLFFSPSEDNFTNDLNKGEEITSDTNIEAAEENISLDLIQQYYLTFNFDNEPENISIENLSSDIIGQLEL
MPNIERFPNGKKYELDKYTMFHYLRAQEFEHGKSRIALTNSVNEALLNPSRVYTFFSSDYVKKVNKATEAAMFLG
WVEQLVYDFTDETSEVSTTDKIADITIIIPYIGPALNIGNMLYKDDFVGALIFSGAVILLEFIPEIAIPVLGTFA
LVSYIANKVLTVQTIDNALSKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKNILNNIILNLRYKDNNLIDLSGYGAKVEVYDGVE
LNDKNQFKLTSSANSKIRVTQNQNIIFNSVFLDFSVSFWIRIPKYKNDGIQNYIHNEYTIINCMKNNSGWKISIR
GNRIIWTLIDINGKTKSVFFEYNIREDISEYINRWFFVTITNNLNNAKIYINGKLESNTDIKDIREVIANGEIIF
KLDGDIDRTQFIWMKYFSIFNTELSQSNIEERYKIQSYSEYLKDFWGNPLMYNKEYYMFNAGNKNSYIKLKKDSP
VGEILTRSKYNQNSKYINYRDLYIGEKFIIRRKSNSQSINDDIVRKEDYIYLDFFNLNQEWRVYTYKYFKKEEMK
LFLAPIYDSDEFYNTIQIKEYDEQPTYSCOLLFKKDEESTDEIGLIGIHRFYESGIVFEEYKDYFCISKWYLKEV
KRKPYNLKLGCNWQF IPKDEGWTE
LC/A1 is italicised
Exogenous activation loop of SEQ ID NO: 63 is bold and underlined
HNB1 is C-terminal to the activation loop and is neither underlined nor italicised
SEQ ID NO: 156:
Nucleic acid encoding exemplary engineered BoNT/A1B1 chimera
with modified Cloop/ SGRSA of SEQ ID NO: 65
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGTCATAAAGCCATTagcggtcgtagcgcaTATAACAAAACCCTGGATTGCATTAAGGTAAAC
AATTGGGATCTGTTCTTTTCGCCATCCGAAGATAATTTTACCAACGACCTGAACAAGGGTGAAGAAATCACCAGC
GATACGAATATTGAAGCAGCGGAAGAGAATATCAGCCTGGATCTGATCCAGCAGTACTATCTGACCTTTAACTTC
GACAATGAACCGGAGAACATTAGCATTGAGAATCTGAGCAGCGACATTATCGGTCAGCTGGAACTGATGCCGAAT
ATCGAACGTTTCCCGAACGGCAAAAAGTACGAGCTGGACAAGTACACTATGTTCCATTACCTGCGTGCACAGGAG
TTTGAACACGGTAAAAGCCGTATCGCGCTGACCAACAGCGTTAACGAGGCCCTGCTGAACCCGAGCCGTGTCTAT
ACCTTCTTCAGCAGCGACTATGTTAAGAAAGTGAACAAAGCCACTGAGGCCGCGATGTTCCTGGGCTGGGTGGAA
CAGCTGGTATATGACTTCACGGACGAGACGAGCGAAGTGAGCACTACCGACAAAATTGCTGATATTACCATCATT
ATCCCGTATATTGGTCCGGCACTGAACATTGGCAACATGCTGTACAAAGACGATTTTGTGGGTGCCCTGATCTTC
TCCGGTGCCGTGATTCTGCTGGAGTTCATTCCGGAGATTGCGATCCCGGTGTTGGGTACCTTCGCGCTGGTGTCC
TACATCGCGAATAAGGTTCTGACGGTTCAGACCATCGATAACGCGCTGTCGAAACGTAATGAAAAATGGGACGAG
GTTTACAAATACATTGTTACGAATTGGCTGGCGAAAGTCAATACCCAGATCGACCTGATCCGTAAGAAAATGAAA
GAGGCGCTGGAGAATCAGGCGGAGGCCACCAAAGCAATTATCAACTACCAATACAACCAGTACACGGAAGAAGAG
AAGAATAACATTAACTTCAATATCGATGATTTGAGCAGCAAGCTGAATGAATCTATCAACAAAGCGATGATCAAT
ATCAACAAGTTTTTGAATCAGTGTAGCGTTTCGTACCTGATGAATAGCATGATTCCGTATGGCGTCAAACGTCTG
GAGGACTTCGACGCCAGCCTGAAAGATGCGTTGCTGAAATACATTTACGACAATCGTGGTACGCTGATTGGCCAA
GTTGACCGCTTGAAAGACAAAGTTAACAATACCCTGAGCACCGACATCCCATTTCAACTGAGCAAGTATGTTGAT
AATCAACGTCTGTTGAGCACTTTCACCGAGTATATCAAAAACATTCTGAACAATATTATCCTGAACCTGCGTTAC
AAAGACAACAATCTGATCGATCTGAGCGGCTATGGTGCAAAAGTTGAAGTCTACGACGGTGTCGAACTGAACGAT
AAAAACCAGTTCAAACTGACCTCATCGGCTAACTCAAAAATTCGTGTGACGCAGAACCAAAACATCATCTTCAAC
TCGGTCTTTCTGGACTTCAGCGTGTCTTTCTGGATTCGCATCCCGAAATATAAAAATGATGGCATCCAGAACTAC
ATCCATAACGAATACACCATCATCAACTGTATGAAAAACAACAGTGGTTGGAAAATTTCCATCCGTGGCAACCGC
ATTATCTGGACCCTGATTGATATCAATGGTAAAACGAAAAGCGTGTTTTTCGAATACAACATCCGTGAAGATATC
TCTGAATACATCAATCGCTGGTTTTTCGTGACCATTACGAACAATCTGAACAATGCGAAAATCTATATCAACGGC
AAACTGGAAAGTAATACCGACATCAAAGATATTCGTGAAGTTATCGCCAACGGTGAAATCATCTTCAAACTGGAT
GGCGACATCGATCGCACCCAGTTCATTTGGATGAAATACTTCTCCATCTTCAACACGGAACTGAGTCAGTCCAAT
ATCGAAGAACGCTACAAAATCCAATCATACTCGGAATACCTGAAAGATTTCTGGGGTAACCCGCTGATGTACAAC
AAAGAATACTACATGTTCAACGCGGGCAACAAAAACTCATACATCAAACTGAAAAAAGATTCGCCGGTGGGTGAA
ATCCTGACCCGTAGCAAATACAACCAGAACTCTAAATACATCAACTATCGCGATCTGTACATTGGCGAAAAATTT
ATTATCCGTCGCAAAAGCAACTCTCAGAGTATTAATGATGACATCGTGCGTAAAGAAGACTACATCTATCTGGAT
TTCTTTAATCTGAACCAAGAATGGCGCGTTTATACCTACAAATACTTCAAAAAAGAAGAAATGAAACTGTTCCTG
GCCCCGATTTACGACAGCGATGAATTTTACAACACCATCCAGATCAAAGAATACGATGAACAGCCGACGTATAGT
TGCCAACTGCTGTTCAAAAAAGACGAAGAATCCACCGATGAAATTGGCCTGATTGGTATCCACCGTTTCTATGAA
AGCGGTATCGTTTTCGAAGAATACAAAGATTACTTCTGTATCTCTAAATGGTATCTGAAAGAAGTCAAACGCAAA
CCGTACAACCTGAAACTGGGCTGCAACTGGCAATTTATCCCGAAAGACGAAGGCTGGACCGAA
Exemplary engineered BoNT/A1B1 chimera with modified Cloop/
SGRSA of SEQ ID NO: 65
SEQ ID NO: 157:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGONTEINNMNFTKLKNFTGLFEFYKLLCHKAISGRSAYNKTLDCIKVN
NWDLFFSPSEDNFTNDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVS
YIANKVLTVQTIDNALSKRNEKWDEVYKYIVTNWLAKVNTQIDLIRKKMKEALENQAEATKAIINYQYNQYTEEE
KNNINFNIDDLSSKLNESINKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKNILNNIILNLRYKDNNLIDLSGYGAKVEVYDGVELND
KNQFKLTSSANSKIRVTQNQNIIFNSVFLDFSVSFWIRIPKYKNDGIQNYIHNEYTIINCMKNNSGWKISIRGNR
IIWTLIDINGKTKSVFFEYNIREDISEYINRWFFVTITNNLNNAKIYINGKLESNTDIKDIREVIANGEIIFKLD
GDIDRTQFIWMKYFSIFNTELSQSNIEERYKIQSYSEYLKDFWGNPLMYNKEYYMFNAGNKNSYIKLKKDSPVGE
ILTRSKYNQNSKYINYRDLYIGEKFIIRRKSNSQSINDDIVRKEDYIYLDFFNLNQEWRVYTYKYFKKEEMKLFL
APIYDSDEFYNTIQIKEYDEQPTYSCOLLFKKDEESTDEIGLIGIHRFYESGIVFEEYKDYFCISKWYLKEVKRK
PYNLKLGCNWQFIPKDEGWTE
LC/A1 is italicised
Exogenous activation loop of SEQ ID NO: 65 is bold and underlined
H_{N}B1 is C-terminal to the activation loop and is neither underlined nor italicised
Nucleic acid encoding exemplary engineered BoNT/A1B1 chimera
with modified Cloop/ LTPRGVRL of SEQ ID NO: 57
SEQ ID NO: 158:
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGtgtcATAAAGCCctgacaccgcgtggtgttcgtctgAAAACCCTGGATTGCATTAAGGTAAAC
AATTGGGATCTGTTCTTTTCGCCATCCGAAGATAATTTTACCAACGACCTGAACAAGGGTGAAGAAATCACCAGC
GATACGAATATTGAAGCAGCGGAAGAGAATATCAGCCTGGATCTGATCCAGCAGTACTATCTGACCTTTAACTTC
GACAATGAACCGGAGAACATTAGCATTGAGAATCTGAGCAGCGACATTATCGGTCAGCTGGAACTGATGCCGAAT
ATCGAACGTTTCCCGAACGGCAAAAAGTACGAGCTGGACAAGTACACTATGTTCCATTACCTGCGTGCACAGGAG
TTTGAACACGGTAAAAGCCGTATCGCGCTGACCAACAGCGTTAACGAGGCCCTGCTGAACCCGAGCCGTGTCTAT
ACCTTCTTCAGCAGCGACTATGTTAAGAAAGTGAACAAAGCCACTGAGGCCGCGATGTTCCTGGGCTGGGTGGAA
CAGCTGGTATATGACTTCACGGACGAGACGAGCGAAGTGAGCACTACCGACAAAATTGCTGATATTACCATCATT
ATCCCGTATATTGGTCCGGCACTGAACATTGGCAACATGCTGTACAAAGACGATTTTGTGGGTGCCCTGATCTTC
TCCGGTGCCGTGATTCTGCTGGAGTTCATTCCGGAGATTGCGATCCCGGTGTTGGGTACCTTCGCGCTGGTGTCC
TACATCGCGAATAAGGTTCTGACGGTTCAGACCATCGATAACGCGCTGTCGAAACGTAATGAAAAATGGGACGAG
GTTTACAAATACATTGTTACGAATTGGCTGGCGAAAGTCAATACCCAGATCGACCTGATCCGTAAGAAAATGAAA
GAGGCGCTGGAGAATCAGGCGGAGGCCACCAAAGCAATTATCAACTACCAATACAACCAGTACACGGAAGAAGAG
AAGAATAACATTAACTTCAATATCGATGATTTGAGCAGCAAGCTGAATGAATCTATCAACAAAGCGATGATCAAT
ATCAACAAGTTTTTGAATCAGTGTAGCGTTTCGTACCTGATGAATAGCATGATTCCGTATGGCGTCAAACGTCTG
GAGGACTTCGACGCCAGCCTGAAAGATGCGTTGCTGAAATACATTTACGACAATCGTGGTACGCTGATTGGCCAA
GTTGACCGCTTGAAAGACAAAGTTAACAATACCCTGAGCACCGACATCCCATTTCAACTGAGCAAGTATGTTGAT
AATCAACGTCTGTTGAGCACTTTCACCGAGTATATCAAAAACATTCTGAACAATATTATCCTGAACCTGCGTTAC
AAAGACAACAATCTGATCGATCTGAGCGGCTATGGTGCAAAAGTTGAAGTCTACGACGGTGTCGAACTGAACGAT
AAAAACCAGTTCAAACTGACCTCATCGGCTAACTCAAAAATTCGTGTGACGCAGAACCAAAACATCATCTTCAAC
TCGGTCTTTCTGGACTTCAGCGTGTCTTTCTGGATTCGCATCCCGAAATATAAAAATGATGGCATCCAGAACTAC
ATCCATAACGAATACACCATCATCAACTGTATGAAAAACAACAGTGGTTGGAAAATTTCCATCCGTGGCAACCGC
ATTATCTGGACCCTGATTGATATCAATGGTAAAACGAAAAGCGTGTTTTTCGAATACAACATCCGTGAAGATATC
TCTGAATACATCAATCGCTGGTTTTTCGTGACCATTACGAACAATCTGAACAATGCGAAAATCTATATCAACGGC
AAACTGGAAAGTAATACCGACATCAAAGATATTCGTGAAGTTATCGCCAACGGTGAAATCATCTTCAAACTGGAT
GGCGACATCGATCGCACCCAGTTCATTTGGATGAAATACTTCTCCATCTTCAACACGGAACTGAGTCAGTCCAAT
ATCGAAGAACGCTACAAAATCCAATCATACTCGGAATACCTGAAAGATTTCTGGGGTAACCCGCTGATGTACAAC
AAAGAATACTACATGTTCAACGCGGGCAACAAAAACTCATACATCAAACTGAAAAAAGATTCGCCGGTGGGTGAA
ATCCTGACCCGTAGCAAATACAACCAGAACTCTAAATACATCAACTATCGCGATCTGTACATTGGCGAAAAATTT
ATTATCCGTCGCAAAAGCAACTCTCAGAGTATTAATGATGACATCGTGCGTAAAGAAGACTACATCTATCTGGAT
TTCTTTAATCTGAACCAAGAATGGCGCGTTTATACCTACAAATACTTCAAAAAAGAAGAAATGAAACTGTTCCTG
GCCCCGATTTACGACAGCGATGAATTTTACAACACCATCCAGATCAAAGAATACGATGAACAGCCGACGTATAGT
TGCCAACTGCTGTTCAAAAAAGACGAAGAATCCACCGATGAAATTGGCCTGATTGGTATCCACCGTTTCTATGAA
AGCGGTATCGTTTTCGAAGAATACAAAGATTACTTCTGTATCTCTAAATGGTATCTGAAAGAAGTCAAACGCAAA
CCGTACAACCTGAAACTGGGCTGCAACTGGCAATTTATCCCGAAAGACGAAGGCTGGACCGAA
Exemplary engineered BoNT/A1B1 chimera with modified Cloop/
LTPRGVRL of SEQ ID NO: 57
SEQ ID NO: 159:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDINCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGONTEINNMNFTKLKNFTGLFEFYKLLCHKALTPRGVRLKTLDCIKVN
NWDLFFSPSEDNFTNDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVS
YIANKVLTVQTIDNALSKRNEKWDEVYKYIVTNWLAKVNTQIDLIRKKMKEALENQAEATKAIINYQYNQYTEEE
KNNINFNIDDLSSKLNESINKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKNILNNIILNLRYKDNNLIDLSGYGAKVEVYDGVELND
KNQFKLTSSANSKIRVTQNQNIIFNSVFLDFSVSFWIRIPKYKNDGIQNYIHNEYTIINCMKNNSGWKISIRGNR
IIWTLIDINGKTKSVFFEYNIREDISEYINRWFFVTITNNLNNAKIYINGKLESNTDIKDIREVIANGEIIFKLD
GDIDRTQFIWMKYFSIFNTELSQSNIEERYKIQSYSEYLKDFWGNPLMYNKEYYMFNAGNKNSYIKLKKDSPVGE
ILTRSKYNQNSKYINYRDLYIGEKFIIRRKSNSQSINDDIVRKEDYIYLDFFNLNQEWRVYTYKYFKKEEMKLFL
APIYDSDEFYNTIQIKEYDEQPTYSCQLLFKKDEESTDEIGLIGIHRFYESGIVFEEYKDYFCISKWYLKEVKRK
PYNLKLGCNWQFIPKDEGWTE
LC/A1 is italicised
Exogenous activation loop of SEQ ID NO: 57 is bold and underlined
H_{N}B1 is C-terminal to the activation loop and is neither underlined nor italicised
Nucleic acid encoding exemplary engineered LC/X- modified
Cloop(LTPRGVRL) of SEQ ID NO: 57-H_{N}/X
SEQ ID NO: 160:
ATGAAGCTGGAAATCAACAAGTTCAACTATAACGATCCGATCGATGGCATTAACGTTATTACCATGCGTCCGCCT
CGTCATAGCGATAAAATCAATAAAGGTAAAGGTCCGTTCAAAGCCTTTCAGGTGATTAAAAACATTTGGATTGTG
CCGGAACGCTACAACTTTACCAATAATACCAACGATCTGAACATTCCGAGCGAACCGATTATGGAAGCAGATGCC
ATTTATAACCCGAACTATCTGAATACCCCGAGCGAAAAAGATGAATTTCTGCAGGGTGTTATCAAAGTGCTGGAA
CGCATTAAAAGCAAACCGGAAGGTGAAAAACTGCTGGAACTGATTAGCAGCAGCATTCCGCTGCCGCTGGTTAGC
AATGGTGCACTGACCCTGAGCGATAATGAAACCATTGCATATCAAGAGAACAACAACATTGTGAGCAATCTGCAG
GCAAACCTGGTTATTTATGGTCCGGGTCCTGATATTGCAAATAATGCAACCTATGGTCTGTATAGCACCCCGATT
AGTAATGGTGAAGGTACACTGAGCGAAGTTAGCTTTAGCCCGTTTTATCTGAAACCGTTTGATGAAAGCTATGGC
AATTATCGTAGCCTGGTGAATATCGTGAACAAATTCGTGAAACGTGAATTTGCACCTGATCCGGCAAGCACCCTG
ATGCACCAACTGGTTTATGTTACCCATAATCTGTATGGCATTAGCAACCGCAACTTCTACTATAACTTTGACACC
GGCAAAATTGAAACCAGCCGTCAGCAGAATAGCCTGATTTTTGAAGAACTGCTGACCTTTGGTGGCATTGATAGC
AAAGCAATTAGCAGCCTGATCATCAAGAAAATTATCGAAACCGCCAAGAACAACTATACCACGCTGATTAGCGAA
CGCCTGAATACCGTTACCGTTGAAAATGATCTGCTGAAATATATCAAAAACAAAATCCCGGTTCAGGGTCGTCTG
GGTAACTTTAAACTGGATACCGCAGAATTCGAGAAAAAGCTGAATACCATTCTGTTTGTGCTGAACGAAAGCAAT
CTGGCACAGCGTTTTAGCATTCTGGTTCGTAAACATTACCTGAAAGAACGTCCGATTGATCCGATTTATGTGAAC
ATTCTGGATGACAATAGCTACAGCACCCTGGAAGGTTTTAACATTAGCAGTCAGGGTAGCAATGATTTTCAGGGC
CAGCTGCTGGAAAGCAGCTATTTTGAAAAAATTGAATCCAATGCGCTGCGTGCCTTTATCAAAATTtgtcATAAA
GCCctgacaccgcgtggtgttcgtctgAAAACCCTGGATTGCATTGAGGTGGAAAACAAAGACCTGTTTCTGATC
AGCAATAAAGATAGCCTGAACGATATTAACCTGAGCGAGGAAAAAATCAAACCTGAAACCACCGTGTTCTTCAAG
GATAAACTGCCTCCGCAGGATATTACCCTGTCCAATTATGATTTTACCGAAGCCAATAGCATCCCGAGCATTAGC
CAGCAAAATATTCTGGAACGTAACGAGGAACTGTATGAACCGATTCGTAATAGCCTGTTTGAGATCAAAACCATC
TATGTGGACAAACTGACCACCTTTCATTTTCTGGAAGCGCAGAATATTGATGAGAGCATCGATAGCAGCAAAATT
CGTGTTGAACTGACCGATAGCGTTGATGAAGCACTGAGCAATCCGAATAAAGTTTATAGCCCGTTCAAGAACATG
AGCAACACCATTAATAGCATTGAAACCGGTATTACCAGCACCTACATCTTTTATCAGTGGCTGCGTAGCATCGTG
AAAGATTTTAGTGATGAAACGGGCAAAATCGACGTGATTGATAAAAGCAGCGATACCCTGGCAATTGTTCCGTAT
ATTGGTCCGCTGCTGAATATTGGTAATGATATTCGTCATGGCGATTTTGTGGGTGCAATTGAACTGGCAGGCATT
ACAGCCCTGCTGGAATATGTTCCGGAATTTACCATTCCGATTCTGGTTGGTCTGGAAGTTATTGGTGGCGAACTG
GCACGTGAACAGGTTGAAGCAATTGTTAATAATGCCCTGGATAAACGCGATCAGAAATGGGCAGAAGTTTACAAT
ATTACCAAAGCACAGTGGTGGGGCACCATTCATTTACAGATTAATACCCGTCTGGCCCATACCTATAAAGCCCTG
AGCCGTCAGGCAAATGCCATTAAAATGAATATGGAATTTCAGCTGGCCAACTACAAAGGCAACATTGATGATAAA
GCCAAGATCAAAAACGCCATCAGCGAAACCGAAATCCTGCTGAACAAAAGTGTTGAACAGGCCATGAAAAACACC
GAGAAGTTTATGATTAAACTGAGCAACAGCTACCTGACCAAAGAAATGATTCCGAAAGTTCAGGACAACCTGAAA
AACTTTGATCTGGAAACCAAAAAGACCCTGGACAAGTTCATCAAAGAGAAAGAAGATATTCTGGGCACCAATCTG
AGCAGCAGCCTGCGTCGTAAAGTTAGCATTCGTCTGAATAAGAACATTGCCTTCGATATCAACGATATCCCGTTT
AGCGAATTCGATGATCTGATCAACCAGTACAAAAATGAA
Exemplary engineered LC/X- modified Cloop(LTPRGVRL) of SEQ ID
NO: 57-H_{N}/X
SEQ ID NO: 161:
MKLEINKFNYNDPIDGINVITMRPPRHSDKINKGKGPFKAFQVIKNIWIVPERYNFINNINDLNIPSEPIMEADA
IYNPNYLNTPSEKDEFLOGVIKVLERIKSKPEGEKLLELISSSIPLPLVSNGALTLSDNETIAYQENNNIVSNLQ
ANLVIYGPGPDIANNATYGLYSTPISNGEGTLSEVSFSPFYLKPFDESYGNYRSLVNIVNKFVKREFAPDPASTL
MHQLVYVTHNLYGISNRNFYYNFDTGKIETSROONSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVRKHYLKERPIDPIYVN
ILDDNSYSTLEGFNISSQGSNDFQGQLLESSYFEKIESNALRAFIKICHKALTPRGVRLKTLDCIEVENKDLFLI
SNKDSLNDINLSEEKIKPETTVFFKDKLPPQDITLSNYDFTEANSIPSISQQNILERNEELYEPIRNSLFEIKTI
YVDKLTTFHFLEAQNIDESIDSSKIRVELTDSVDEALSNPNKVYSPFKNMSNTINSIETGITSTYIFYQWLRSIV
KDFSDETGKIDVIDKSSDTLAIVPYIGPLLNIGNDIRHGDFVGAIELAGITALLEYVPEFTIPILVGLEVIGGEL
AREQVEAIVNNALDKRDQKWAEVYNITKAQWWGTIHLQINTRLAHTYKALSRQANAIKMNMEFQLANYKGNIDDK
AKIKNAISETEILLNKSVEQAMKNTEKFMIKLSNSYLTKEMIPKVQDNLKNFDLETKKTLDKFIKEKEDILGTNL
SSSLRRKVSIRLNKNIAFDINDIPFSEFDDLINQYKNE
LC/X is italicised
Exogenous activation loop of SEQ ID NO: 57 is bold and underlined
H_{N}X is C-terminal to the activation loop and is neither underlined nor italicised
Nucleic acid encoding exemplary engineered LC/X- modified
Cloop(SGRSA) of SEQ ID NO: 65-H_{N}/X
SEQ ID NO: 162:
ATGAAGCTGGAAATCAACAAGTTCAACTATAACGATCCGATCGATGGCATTAACGTTATTACCATGCGTCCGCCT
CGTCATAGCGATAAAATCAATAAAGGTAAAGGTCCGTTCAAAGCCTTTCAGGTGATTAAAAACATTTGGATTGTG
CCGGAACGCTACAACTTTACCAATAATACCAACGATCTGAACATTCCGAGCGAACCGATTATGGAAGCAGATGCC
ATTTATAACCCGAACTATCTGAATACCCCGAGCGAAAAAGATGAATTTCTGCAGGGTGTTATCAAAGTGCTGGAA
CGCATTAAAAGCAAACCGGAAGGTGAAAAACTGCTGGAACTGATTAGCAGCAGCATTCCGCTGCCGCTGGTTAGC
AATGGTGCACTGACCCTGAGCGATAATGAAACCATTGCATATCAAGAGAACAACAACATTGTGAGCAATCTGCAG
GCAAACCTGGTTATTTATGGTCCGGGTCCTGATATTGCAAATAATGCAACCTATGGTCTGTATAGCACCCCGATT
AGTAATGGTGAAGGTACACTGAGCGAAGTTAGCTTTAGCCCGTTTTATCTGAAACCGTTTGATGAAAGCTATGGC
AATTATCGTAGCCTGGTGAATATCGTGAACAAATTCGTGAAACGTGAATTTGCACCTGATCCGGCAAGCACCCTG
ATGCACCAACTGGTTTATGTTACCCATAATCTGTATGGCATTAGCAACCGCAACTTCTACTATAACTTTGACACC
GGCAAAATTGAAACCAGCCGTCAGCAGAATAGCCTGATTTTTGAAGAACTGCTGACCTTTGGTGGCATTGATAGC
AAAGCAATTAGCAGCCTGATCATCAAGAAAATTATCGAAACCGCCAAGAACAACTATACCACGCTGATTAGCGAA
CGCCTGAATACCGTTACCGTTGAAAATGATCTGCTGAAATATATCAAAAACAAAATCCCGGTTCAGGGTCGTCTG
GGTAACTTTAAACTGGATACCGCAGAATTCGAGAAAAAGCTGAATACCATTCTGTTTGTGCTGAACGAAAGCAAT
CTGGCACAGCGTTTTAGCATTCTGGTTCGTAAACATTACCTGAAAGAACGTCCGATTGATCCGATTTATGTGAAC
ATTCTGGATGACAATAGCTACAGCACCCTGGAAGGTTTTAACATTAGCAGTCAGGGTAGCAATGATTTTCAGGGC
CAGCTGCTGGAAAGCAGCTATTTTGAAAAAATTGAATCCAATGCGCTGCGTGCCTTTATCAAAATTTGTCATAAA
GCCATTagcggtcgtagcgcaTATAACAAAACCCTGGATTGCATTGAGGTGGAAAACAAAGACCTGTTTCTGATC
AGCAATAAAGATAGCCTGAACGATATTAACCTGAGCGAGGAAAAAATCAAACCTGAAACCACCGTGTTCTTCAAG
GATAAACTGCCTCCGCAGGATATTACCCTGTCCAATTATGATTTTACCGAAGCCAATAGCATCCCGAGCATTAGC
CAGCAAAATATTCTGGAACGTAACGAGGAACTGTATGAACCGATTCGTAATAGCCTGTTTGAGATCAAAACCATC
TATGTGGACAAACTGACCACCTTTCATTTTCTGGAAGCGCAGAATATTGATGAGAGCATCGATAGCAGCAAAATT
CGTGTTGAACTGACCGATAGCGTTGATGAAGCACTGAGCAATCCGAATAAAGTTTATAGCCCGTTCAAGAACATG
AGCAACACCATTAATAGCATTGAAACCGGTATTACCAGCACCTACATCTTTTATCAGTGGCTGCGTAGCATCGTG
AAAGATTTTAGTGATGAAACGGGCAAAATCGACGTGATTGATAAAAGCAGCGATACCCTGGCAATTGTTCCGTAT
ATTGGTCCGCTGCTGAATATTGGTAATGATATTCGTCATGGCGATTTTGTGGGTGCAATTGAACTGGCAGGCATT
ACAGCCCTGCTGGAATATGTTCCGGAATTTACCATTCCGATTCTGGTTGGTCTGGAAGTTATTGGTGGCGAACTG
GCACGTGAACAGGTTGAAGCAATTGTTAATAATGCCCTGGATAAACGCGATCAGAAATGGGCAGAAGTTTACAAT
ATTACCAAAGCACAGTGGTGGGGCACCATTCATTTACAGATTAATACCCGTCTGGCCCATACCTATAAAGCCCTG
AGCCGTCAGGCAAATGCCATTAAAATGAATATGGAATTTCAGCTGGCCAACTACAAAGGCAACATTGATGATAAA
GCCAAGATCAAAAACGCCATCAGCGAAACCGAAATCCTGCTGAACAAAAGTGTTGAACAGGCCATGAAAAACACC
GAGAAGTTTATGATTAAACTGAGCAACAGCTACCTGACCAAAGAAATGATTCCGAAAGTTCAGGACAACCTGAAA
AACTTTGATCTGGAAACCAAAAAGACCCTGGACAAGTTCATCAAAGAGAAAGAAGATATTCTGGGCACCAATCTG
AGCAGCAGCCTGCGTCGTAAAGTTAGCATTCGTCTGAATAAGAACATTGCCTTCGATATCAACGATATCCCGTTT
AGCGAATTCGATGATCTGATCAACCAGTACAAAAATGAA
Exemplary engineered LC/X- modified Cloop(SGRSA) of SEQ ID NO:
65-H_{N}/X
SEQ ID NO: 163:
MKLEINKFNYNDPIDGINVITMRPPRHSDKINKGKGPFKAFQVIKNIWIVPERYNFINNINDLNIPSEPIMEADA
IYNPNYLNTPSEKDEFLQGVIKVLERIKSKPEGEKLLELISSSIPLPLVSNGALTLSDNETIAYQENNNIVSNLQ
ANLVIYGPGPDIANNATYGLYSTPISNGEGTLSEVSFSPFYLKPFDESYGNYRSLVNIVNKFVKREFAPDPASTL
MHQLVYVTHNLYGISNRNFYYNFDIGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVRKHYLKERPIDPIYVN
ILDDNSYSTLEGFNISSQGSNDFQGQLLESSYFEKIESNALRAFIKICHKAISGRSAYNKTLDCIEVENKDLFLI
SNKDSLNDINLSEEKIKPETTVFFKDKLPPQDITLSNYDFTEANSIPSISQQNILERNEELYEPIRNSLFEIKTI
YVDKLTTFHFLEAQNIDESIDSSKIRVELTDSVDEALSNPNKVYSPFKNMSNTINSIETGITSTYIFYQWLRSIV
KDFSDETGKIDVIDKSSDTLAIVPYIGPLLNIGNDIRHGDFVGAIELAGITALLEYVPEFTIPILVGLEVIGGEL
AREQVEAIVNNALDKRDQKWAEVYNITKAQWWGTIHLQINTRLAHTYKALSRQANAIKMNMEFQLANYKGNIDDK
AKIKNAISETEILLNKSVEQAMKNTEKFMIKLSNSYLTKEMIPKVQDNLKNFDLETKKTLDKFIKEKEDILGTNL
SSSLRRKVSIRLNKNIAFDINDIPFSEFDDLINQYKNE
LC/X is italicised
Exogenous activation loop of SEQ ID NO: 65 is bold and underlined
HNX is C-terminal to the activation loop and is neither underlined nor italicised
Nucleic acid encoding exemplary engineered LC/X- modified
Cloop(PPFGRSAG) of SEQ ID NO: 63-H_{N}/X
SEQ ID NO: 164:
ATGAAGCTGGAAATCAACAAGTTCAACTATAACGATCCGATCGATGGCATTAACGTTATTACCATGCGTCCGCCT
CGTCATAGCGATAAAATCAATAAAGGTAAAGGTCCGTTCAAAGCCTTTCAGGTGATTAAAAACATTTGGATTGTG
CCGGAACGCTACAACTTTACCAATAATACCAACGATCTGAACATTCCGAGCGAACCGATTATGGAAGCAGATGCC
ATTTATAACCCGAACTATCTGAATACCCCGAGCGAAAAAGATGAATTTCTGCAGGGTGTTATCAAAGTGCTGGAA
CGCATTAAAAGCAAACCGGAAGGTGAAAAACTGCTGGAACTGATTAGCAGCAGCATTCCGCTGCCGCTGGTTAGC
AATGGTGCACTGACCCTGAGCGATAATGAAACCATTGCATATCAAGAGAACAACAACATTGTGAGCAATCTGCAG
GCAAACCTGGTTATTTATGGTCCGGGTCCTGATATTGCAAATAATGCAACCTATGGTCTGTATAGCACCCCGATT
AGTAATGGTGAAGGTACACTGAGCGAAGTTAGCTTTAGCCCGTTTTATCTGAAACCGTTTGATGAAAGCTATGGC
AATTATCGTAGCCTGGTGAATATCGTGAACAAATTCGTGAAACGTGAATTTGCACCTGATCCGGCAAGCACCCTG
ATGCACCAACTGGTTTATGTTACCCATAATCTGTATGGCATTAGCAACCGCAACTTCTACTATAACTTTGACACC
GGCAAAATTGAAACCAGCCGTCAGCAGAATAGCCTGATTTTTGAAGAACTGCTGACCTTTGGTGGCATTGATAGC
AAAGCAATTAGCAGCCTGATCATCAAGAAAATTATCGAAACCGCCAAGAACAACTATACCACGCTGATTAGCGAA
CGCCTGAATACCGTTACCGTTGAAAATGATCTGCTGAAATATATCAAAAACAAAATCCCGGTTCAGGGTCGTCTG
GGTAACTTTAAACTGGATACCGCAGAATTCGAGAAAAAGCTGAATACCATTCTGTTTGTGCTGAACGAAAGCAAT
CTGGCACAGCGTTTTAGCATTCTGGTTCGTAAACATTACCTGAAAGAACGTCCGATTGATCCGATTTATGTGAAC
ATTCTGGATGACAATAGCTACAGCACCCTGGAAGGTTTTAACATTAGCAGTCAGGGTAGCAATGATTTTCAGGGC
CAGCTGCTGGAAAGCAGCTATTTTGAAAAAATTGAATCCAATGCGCTGCGTGCCTTTATCAAAATTTGTCATAAA
GCCATTCCGCCGTTTGGTCGTAGCGCAGGTTATAACAAAACCCTGGATTGTATTGAGGTGGAAAACAAAGACCTG
TTTCTGATCAGCAATAAAGATAGCCTGAACGATATTAACCTGAGCGAGGAAAAAATCAAACCTGAAACCACCGTG
TTCTTCAAGGATAAACTGCCTCCGCAGGATATTACCCTGTCCAATTATGATTTTACCGAAGCCAATAGCATCCCG
AGCATTAGCCAGCAAAATATTCTGGAACGTAACGAGGAACTGTATGAACCGATTCGTAATAGCCTGTTTGAGATC
AAAACCATCTATGTGGACAAACTGACCACCTTTCATTTTCTGGAAGCGCAGAATATTGATGAGAGCATCGATAGC
AGCAAAATTCGTGTTGAACTGACCGATAGCGTTGATGAAGCACTGAGCAATCCGAATAAAGTTTATAGCCCGTTC
AAGAACATGAGCAACACCATTAATAGCATTGAAACCGGTATTACCAGCACCTACATCTTTTATCAGTGGCTGCGT
AGCATCGTGAAAGATTTTAGTGATGAAACGGGCAAAATCGACGTGATTGATAAAAGCAGCGATACCCTGGCAATT
GTTCCGTATATTGGTCCGCTGCTGAATATTGGTAATGATATTCGTCATGGCGATTTTGTGGGTGCAATTGAACTG
GCAGGCATTACAGCCCTGCTGGAATATGTTCCGGAATTTACCATTCCGATTCTGGTTGGTCTGGAAGTTATTGGT
GGCGAACTGGCACGTGAACAGGTTGAAGCAATTGTTAATAATGCCCTGGATAAACGCGATCAGAAATGGGCAGAA
GTTTACAATATTACCAAAGCACAGTGGTGGGGCACCATTCATTTACAGATTAATACCCGTCTGGCCCATACCTAT
AAAGCCCTGAGCCGTCAGGCAAATGCCATTAAAATGAATATGGAATTTCAGCTGGCCAACTACAAAGGCAACATT
GATGATAAAGCCAAGATCAAAAACGCCATCAGCGAAACCGAAATCCTGCTGAACAAAAGTGTTGAACAGGCCATG
AAAAACACCGAGAAGTTTATGATTAAACTGAGCAACAGCTACCTGACCAAAGAAATGATTCCGAAAGTTCAGGAC
AACCTGAAAAACTTTGATCTGGAAACCAAAAAGACCCTGGACAAGTTCATCAAAGAGAAAGAAGATATTCTGGGC
ACCAATCTGAGCAGCAGCCTGCGTCGTAAAGTTAGCATTCGTCTGAATAAGAACATTGCCTTCGATATCAACGAT
ATCCCGTTTAGCGAATTCGATGATCTGATCAACCAGTACAAAAATGAA
Exemplary engineered LC/X- modified Cloop(PPFGRSAG) of SEQ ID
NO: 63-H_{N}/X
SEQ ID NO: 165:
MKLEINKFNYNDPIDGINVITMRPPRHSDKINKGKGPFKAFQVIKNIWIVPERYNFINNINDLNIPSEPIMEADA
IYNPNYLNTPSEKDEFLQGVIKVLERIKSKPEGEKLLELISSSIPLPLVSNGALTLSDNETIAYQENNNIVSNLQ
ANLVIYGPGPDIANNATYGLYSTPISNGEGTLSEVSFSPFYLKPFDESYGNYRSLVNIVNKFVKREFAPDPASTL
MHQLVYVTHNLYGISNRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVRKHYLKERPIDPIYVN
ILDDNSYSTLEGFNISSQGSNDFQGQLLESSYFEKIESNALRAFIKICHKAIPPFGRSAGYNKTLDCIEVENKDL
FLISNKDSLNDINLSEEKIKPETTVFFKDKLPPQDITLSNYDFTEANSIPSISQQNILERNEELYEPIRNSLFEI
KTIYVDKLTTFHFLEAQNIDESIDSSKIRVELTDSVDEALSNPNKVYSPFKNMSNTINSIETGITSTYIFYQWLR
SIVKDFSDETGKIDVIDKSSDTLAIVPYIGPLLNIGNDIRHGDFVGAIELAGITALLEYVPEFTIPILVGLEVIG
GELAREQVEAIVNNALDKRDQKWAEVYNITKAQWWGTIHLQINTRLAHTYKALSRQANAIKMNMEFQLANYKGNI
DDKAKIKNAISETEILLNKSVEQAMKNTEKFMIKLSNSYLTKEMIPKVQDNLKNFDLETKKTLDKFIKEKEDILG
TNLSSSLRRKVSIRLNKNIAFDINDIPFSEFDDLINQYKNE
LC/X is italicised
Exogenous activation loop of SEQ ID NO: 63 is bold and underlined
H_{N}X is C-terminal to the activation loop and is neither underlined nor italicised
Exemplary engineered LC/A1- modified Cloop(LTPRGVRL) of SEQ ID
NO: 57-HN/A1
SEQ ID NO: 166:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDINCINVIQPDGSYRSEELN
LVIIGPSADIIQFECKSFGHEVLNLTRNGYGSTQYIRFSPDFTFGFEESLEVDINPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCHKALTPRGVRLKTLDCIKVN
NWDLFFSPSEDNFTNDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVS
YIANKVLTVQTIDNALSKRNEKWDEVYKYIVTNWLAKVNTQIDLIRKKMKEALENQAEATKAIINYQYNQYTEEE
KNNINFNIDDLSSKLNESINKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKN
LC/A1 is italicised
Exogenous activation loop of SEQ ID NO: 57 is bold and underlined
H_{NA1} is C-terminal to the activation loop and is neither underlined nor italicised
Exemplary engineered LC/A1- modified Cloop(PPFGRSAG) of SEQ ID
NO: 63-H_{N}/A1
SEQ ID NO: 167:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDINCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCHKAIPPFGRSAGYNKTLDCI
KVNNWDLFFSPSEDNFTNDLNKGEEITSDTNIEAAEENISLDLIQQYYLTFNFDNEPENISIENLSSDIIGQLEL
MPNIERFPNGKKYELDKYTMFHYLRAQEFEHGKSRIALTNSVNEALLNPSRVYTFFSSDYVKKVNKATEAAMFLG
WVEQLVYDFTDETSEVSTTDKIADITIIIPYIGPALNIGNMLYKDDFVGALIFSGAVILLEFIPEIAIPVLGTFA
LVSYIANKVLTVQTIDNALSKRNEKWDEVYKYIVTNWLAKVNTQIDLIRKKMKEALENQAEATKAIINYQYNQYT
EEEKNNINFNIDDLSSKLNESINKAMININKFLNQCSVSYLMNSMIPYGVKRLEDFDASLKDALLKYIYDNRGTL
IGQVDRLKDKVNNTLSTDIPFQLSKYVDNQRLLSTFTEYIKN
LC/A1 is italicised
Exogenous activation loop of SEQ ID NO: 63 is bold and underlined
H_{NA1} is C-terminal to the activation loop and is neither underlined nor italicised
Exemplary engineered LC/A1- modified Cloop(SGRSA) of SEQ ID NO:
65-H_{N}/A1
SEQ ID NO: 168:
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDINCINVIQPDGSYRSEELN
LVIIGPSADIIQFECKSFGHEVLNLTRNGYGSTQYIRFSPDFTFGFEESLEVDINPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGONTEINNMNFTKLKNFTGLFEFYKLLCHKAISGRSAYNKTLDCIKVN
NWDLFFSPSEDNFTNDLNKGEEITSDTNIEAAEENISLDLIQQYYLTFNFDNEPENISIENLSSDIIGQLELMPN
IERFPNGKKYELDKYTMFHYLRAQEFEHGKSRIALTNSVNEALLNPSRVYTFFSSDYVKKVNKATEAAMFLGWVE
QLVYDFTDETSEVSTTDKIADITIIIPYIGPALNIGNMLYKDDFVGALIFSGAVILLEFIPEIAIPVLGTFALVS
YIANKVLTVQTIDNALSKRNEKWDEVYKYIVTNWLAKVNTQIDLIRKKMKEALENQAEATKAIINYQYNQYTEEE
KNNINFNIDDLSSKLNESINKAMININKFLNQCSVSYLMNSMIPYGVKRLEDFDASLKDALLKYIYDNRGTLIGQ
VDRLKDKVNNTLSTDIPFQLSKYVDNQRLLSTFTEYIKN
LC/A1 is italicised
Exogenous activation loop of SEQ ID NO: 65 is bold and underlined
H_{NA1} is C-terminal to the activation loop and is neither underlined nor italicised
SEQ ID NO: 169: Preferred thrombin consensus sequence
(AL)X(PLAG)(RK)|(SAG)(AVL)(GL)X
wherein each X may be independently selected from any amino acid, the amino acid
residues shown in parentheses are alternative possible amino acid residues at each
position, wherein the bold and underlined residues are preferred. The “|” shows the site of
cleavage (i.e. the peptide bond that is hydrolysed).
SEQ ID NO: 170: Preferred thrombin consensus sequence
(AL)XPR|G(AVL)(GL)X
wherein each X may be independently selected from any amino acid, the amino acid
residues shown in parentheses are alternative possible amino acid residues at each
position, wherein the bold and underlined residues are preferred. The “|” shows the site of
cleavage (i.e. the peptide bond that is hydrolysed).
Preferred motif within a thrombin cleavage site
SEQ ID NO: 171:
PRG
Preferred uPA and/or t-PA consensus sequence
SEQ ID NO: 172:
P(GPAL)(SGF)(GS)(RK)|(SGV)(AVLR)
wherein each X may be independently selected from any amino acid, the amino acid
residues shown in parentheses are alternative possible amino acid residues at each
position, wherein the bold and underlined residues are preferred. The “|” shows the site of
cleavage (i.e. the peptide bond that is hydrolysed).
Preferred uPA and/or t-PA consensus sequence
SEQ ID NO: 173:
P(GPAL)(SGF)GR|SA
wherein each X may be independently selected from any amino acid, the amino acid
residues shown in parentheses are alternative possible amino acid residues at each
position, wherein the bold and underlined residues are preferred. The “|” shows the site of
cleavage (i.e. the peptide bond that is hydrolysed).
Preferred motif within a uPA and/or t-PA cleavage site
SEQ ID NO: 174:
GRSA
SEQ ID NO: 175
Exemplary nucleic acid encoding LC/X-Cloop-H_{N}/X
atgaagctggaaatcaacaagttcaactataacgatccgatcgatggcattaacgttattaccatgcgtccgcct
cgtcatagcgataaaatcaataaaggtaaaggtccgttcaaagcctttcaggtgattaaaaacatttggattgtg
ccggaacgctacaactttaccaataataccaacgatctgaacattccgagcgaaccgattatggaagcagatgcc
atttataacccgaactatctgaataccccgagcgaaaaagatgaatttctgcagggtgttatcaaagtgctggaa
cgcattaaaagcaaaccggaaggtgaaaaactgctggaactgattagcagcagcattccgctgccgctggttagc
aatggtgcactgaccctgagcgataatgaaaccattgcatatcaagagaacaacaacattgtgagcaatctgcag
gcaaacctggttatttatggtccgggtcctgatattgcaaataatgcaacctatggtctgtatagcaccccgatt
agtaatggtgaaggtacactgagcgaagttagctttagcccgttttatctgaaaccgtttgatgaaagctatggc
aattatcgtagcctggtgaatatcgtgaacaaattcgtgaaacgtgaatttgcacctgatccggcaagcaccctg
atgcacgagctggttcatgttacccataatctgtatggcattagcaaccgcaacttctactataactttgacacc
ggcaaaattgaaaccagccgtcagcagaatagcctgatttttgaagaactgctgacctttggtggcattgatagc
aaagcaattagcagcctgatcatcaagaaaattatcgaaaccgccaagaacaactataccacgctgattagcgaa
cgcctgaataccgttaccgttgaaaatgatctgctgaaatatatcaaaaacaaaatcccggttcagggtcgtctg
ggtaactttaaactggataccgcagaattcgagaaaaagctgaataccattctgtttgtgctgaacgaaagcaat
ctggcacagcgttttagcattctggttcgtaaacattacctgaaagaacgtccgattgatccgatttatgtgaac
attctggatgacaatagctacagcaccctggaaggttttaacattagcagtcagggtagcaatgattttcagggc
cagctgctggaaagcagctattttgaaaaaattgaatccaatgcgctgcgtgcctttatcaaaatttgtcataaa
gccattgatggtcgcagcctgtataacaaaaccctggattgtattgaggtggaaaacaaagacctgtttctgatc
agcaataaagatagcctgaacgatattaacctgagcgaggaaaaaatcaaacctgaaaccaccgtgttcttcaag
gataaactgcctccgcaggatattaccctgtccaattatgattttaccgaagccaatagcatcccgagcattagc
cagcaaaatattctggaacgtaacgaggaactgtatgaaccgattcgtaatagcctgtttgagatcaaaaccatc
tatgtggacaaactgaccacctttcattttctggaagcgcagaatattgatgagagcatcgatagcagcaaaatt
cgtgttgaactgaccgatagcgttgatgaagcactgagcaatccgaataaagtttatagcccgttcaagaacatg
agcaacaccattaatagcattgaaaccggtattaccagcacctacatcttttatcagtggctgcgtagcatcgtg
aaagattttagtgatgaaacgggcaaaatcgacgtgattgataaaagcagcgataccctggcaattgttccgtat
attggtccgctgctgaatattggtaatgatattcgtcatggcgattttgtgggtgcaattgaactggcaggcatt
acagccctgctggaatatgttccggaatttaccattccgattctggttggtctggaagttattggtggcgaactg
gcacgtgaacaggttgaagcaattgttaataatgccctggataaacgcgatcagaaatgggcagaagtttacaat
attaccaaagcacagtggtggggcaccattcatttacagattaatacccgtctggcccatacctataaagccctg
agccgtcaggcaaatgccattaaaatgaatatggaatttcagctggccaactacaaaggcaacattgatgataaa
gccaagatcaaaaacgccatcagcgaaaccgaaatcctgctgaacaaaagtgttgaacaggccatgaaaaacacc
gagaagtttatgattaaactgagcaacagctacctgaccaaagaaatgattccgaaagttcaggacaacctgaaa
aactttgatctggaaaccaaaaagaccctggacaagttcatcaaagagaaagaagatattctgggcaccaatctg
agcagcagcctgcgtcgtaaagttagcattcgtctgaataagaacattgccttcgatatcaacgatatcccgttt
agcgaattcgatgatctgatcaaccagtacaaaaatgaa
nucleic acid encoding the Cloop is bold and underlined
nucleic acid encoding 2xAP linker is dash underlined
Exemplary nucleic acid Cloop
SEQ ID NO: 176:
tgtcataaagccattgatggtcgcagcctgtataacaaaaccctggat
```

### EXAMPLES

The invention will be further illustrated by the following examples, which are intended to be purely exemplary of the invention and are in no way limiting. In addition to the data presented below, the contents of PCT/GB2022/050756, and in particular the examples section, is incorporated herein by reference. Although that case concerns engineered clostridial neurotoxins comprising furin cleavage sites, rather than exogenous protease cleavage sites, it nevertheless contains data obtained from engineered clostridial neurotoxins with features in common with those of the present invention, as well as relevant synthetic methods.

### Example 1—Design and Production of Engineered BoNT Comprising Exogenous Protease Cleavage Sites

Engineered BoNT were produced by determining the desired amino acid sequence, back-translating this to the corresponding nucleic acid sequence followed by codon optimisation for recombinant expression in bacteria.

The resulting gene sequences were checked to ensure no commonly-used restriction sites (Ndel, XhoI, BamHI, HindIII, NcoI, and EcoRI) were present within the sequence. A start codon was added at the 5′ end, a stop codon at the 3′ end, and appropriate terminal restriction sites (e.g. an Ndel restriction site to the 5′ end and BamHI restriction site at the 3′ end) to enable subcloning into expression vectors.

The gene sequences for the engineered BoNT were then subcloned into a pK8 vector (which comprises a kanamycin resistance gene, a T7 promoter, a T7 terminator, a pBR322 origin of replication, and a multiple cloning site). The plasmid for each engineered BoNT was then amplified in E. coli strain DH5a with kanamycin selection, and extracted by miniprep using standard molecular biology techniques.

E. coli expression strain BL21 with ADE3 was then transformed with the plasmid DNA, spread onto agar supplemented with kanamycin, and incubated overnight at 37° C. Colonies were then harvested and used to prepare a glycerol stock. A stab was then used to inoculate 100 mL of modified TB media supplemented kanamycin, followed by incubation at 37° C. overnight with shaking at 225 RPM to provide aeration. 10 mL of this starter culture was used to inoculate several baffled conical flasks, each containing up to 1 L of the same nutrient media and antibiotic. The cultures were grown under the same conditions for a few hours to an optical density (A600) of 20.6 and then set the incubator temperature to 16° C. The cultures were then induced to express the engineered BoNTs by addition of IPTG an hour later. After 20 hours, the cells were harvested by centrifugation and stored at −80° C. before use.

The cells were thawed in 0.25 M NaCl in 50 mM Tris pH 7.4 (5 mL/g cells) and lysed at 4° C. by either two passes through a cell homogenisor at 20k PSI or by ultrasonication (10×30 s on/off). Cell debris was removed by centrifugation and the clarified supernatant loaded onto a nickel affinity column pre-equilibrated with 0.5 M NaCl in 50 mM Tris pH 7.4 (“Buffer NA”) using an FPLC system (GE). The column was washed with Buffer NA until a steady baseline at A280 was achieved. The wash and elute proteins were collected off the column with a linear gradient of 0-0.5 M imidazole in Buffer NA in over 25 column volumes (CV) while collecting 3 mL fractions.

All collected material was stored at 4° C. while analysing samples by SDS PAGE with staining (Invitrogen). Fractions showing a protein strong band at the calculated MW of the target molecule based on the protein marker were pooled, and the total protein concentration measured using a Nanodrop (Thermo Fisher).

The pooled fractions were desalted into 50 mM Tris pH 8 (“Buffer QA”) for further purification by anionic exchange chromatography (e.g., Q HP). After washing the column with Buffer QA to a steady baseline, proteins were eluted with a linear gradient of 0-0.5 M NaCl in Buffer QA over 25 CV. Samples were analysed by SDS-PAGE and fractions containing pure target desalted into 150 mM NaCl in 50 mM HEPES pH 7.2 before dividing into aliquots for storage at −80° C. Analysis of a sample of the final product in the presence and absence of DTT by SDS PAGE showed a single band The above method was used to produce X engineered BoNT with the structure LC/A1-ExoLoop-H_{N}/A1-TM: When ExoLoop=modified C loop comprising a KRV cleavage site (A4952), modified C loop comprising a GRI cleavage site (A49523) and modified C loop comprising a PRG cleavage site (A4954). All three of these engineered BoNT comprise the same targeting moiety (TM), BoNT/A1 light chain (LC/A1), and BoNT/A1 translocation domain (H_{N}/A1), but with different modified C loops comprising different exogenous protease cleavage sites. The pre-engineering LC/A1-Cloop-H_{N}/A1 sequence for each of these engineered BoNT is SEQ ID NO: 150, in which the Cloop (SEQ ID NO: 2) has been replaced by a modified C loop. In particular, A4952 comprises the modified C loop of SEQ ID NO: 53, which itself comprises a cleavage site KRV which is specific for u-PA and t-PA. A49523 comprises the modified C loop of SEQ ID NO: 55, which itself comprises a cleavage site GRI which is specific for. FIX and FVII. A49524 comprises the modified C loop of SEQ ID NO: 54, which itself comprises a cleavage site PRG which is specific for thrombin.

### Example 2—BoNT Engineered to Comprise an KRV Modified C-Loop are Effectively Cleaved by u-PA

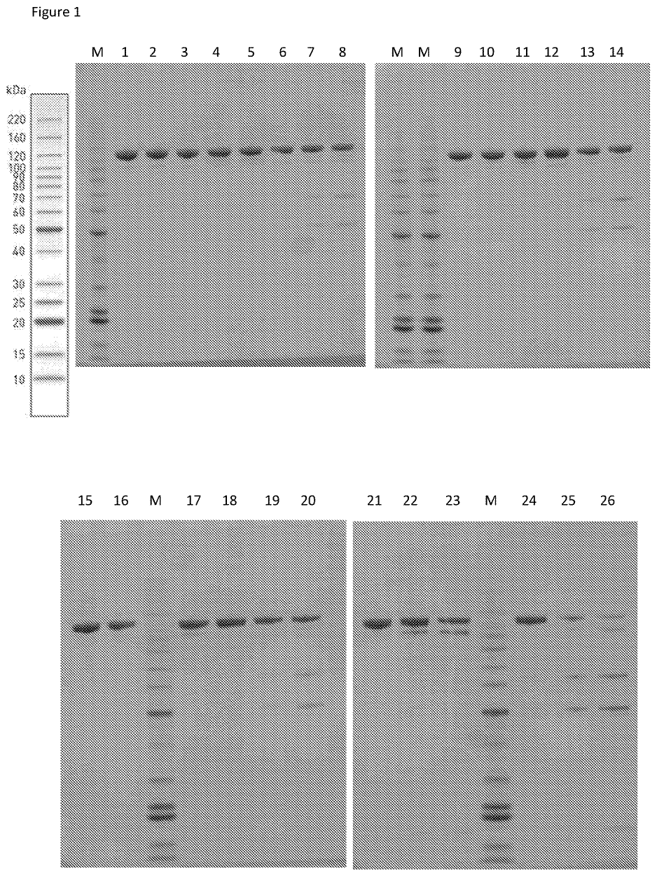

A4952 from Example 1 was incubated with incubated with increasing concentrations of urokinase ((i) 1 μl, giving a final u-PA concentration of 0.85 μg/mL; (ii) 5 μl, giving a final u-PA concentration of 4.22 μg/mL; and (iii) 10 μl, giving a final u-PA concentration of 8.41 μg/mL). Samples taken at time 3 hrs, 4 hrs, 5 hrs and 19 hrs after the reaction start time were analysed by SDS PAGE under reducing and non-reducing conditions. As can be seen in FIG. 1A, at the higher concentrations of uPA (4.22 μg/mL; and 8.41 μg/mL), some cleavage of A4952 was observed as early 3 hrs. By 19 hrs, cleavage was observed at all concentrations of u-PA tested. The table below sets out the lanes of the gels in FIG. 1A:

| | | | Protease | | |
|---|---|---|---|---|---|
| | | | Concentration | | Time |
| Lane | Sample | Protease | μg/ml | DTT | hrs |
| M | Mw | n/a | n/a | n/a | n/a |
| | Marker | | | | |
| 1 | A00004952 | None | n/a | − | 19 |
| 2 | A00004952 | None | n/a | + | 19 |
| 3 | A00004952 | Urokinase | 0.85 | − | 3 |
| 4 | A00004952 | Urokinase | 4.22 | − | 3 |
| 5 | A00004952 | Urokinase | 8.41 | − | 3 |
| 6 | A00004952 | Urokinase | 0.85 | + | 3 |
| 7 | A00004952 | Urokinase | 4.22 | + | 3 |
| 8 | A00004952 | Urokinase | 8.41 | + | 3 |
| 9 | A00004952 | Urokinase | 0.85 | − | 4 |
| 10 | A00004952 | Urokinase | 4.22 | − | 4 |
| 11 | A00004952 | Urokinase | 8.41 | − | 4 |
| 12 | A00004952 | Urokinase | 0.85 | + | 4 |
| 13 | A00004952 | Urokinase | 4.22 | + | 4 |
| 14 | A00004952 | Urokinase | 8.41 | + | 4 |
| 15 | A00004952 | Urokinase | 0.85 | − | 5 |
| 16 | A00004952 | Urokinase | 4.22 | − | 5 |
| 17 | A00004952 | Urokinase | 8.41 | − | 5 |
| 18 | A00004952 | Urokinase | 0.85 | + | 5 |
| 19 | A00004952 | Urokinase | 4.22 | + | 5 |
| 20 | A00004952 | Urokinase | 8.41 | + | 5 |
| 21 | A00004952 | Urokinase | 0.85 | − | 19 |
| 22 | A00004952 | Urokinase | 4.22 | − | 19 |
| 23 | A00004952 | Urokinase | 8.41 | − | 19 |
| 24 | A00004952 | Urokinase | 0.85 | + | 19 |
| 25 | A00004952 | Urokinase | 4.22 | + | 19 |
| 26 | A00004952 | Urokinase | 8.41 | + | 19 |

### Example 3—BoNT Engineered to Comprise a GRI Modified C-Loop were not Effectively Cleaved by FIX

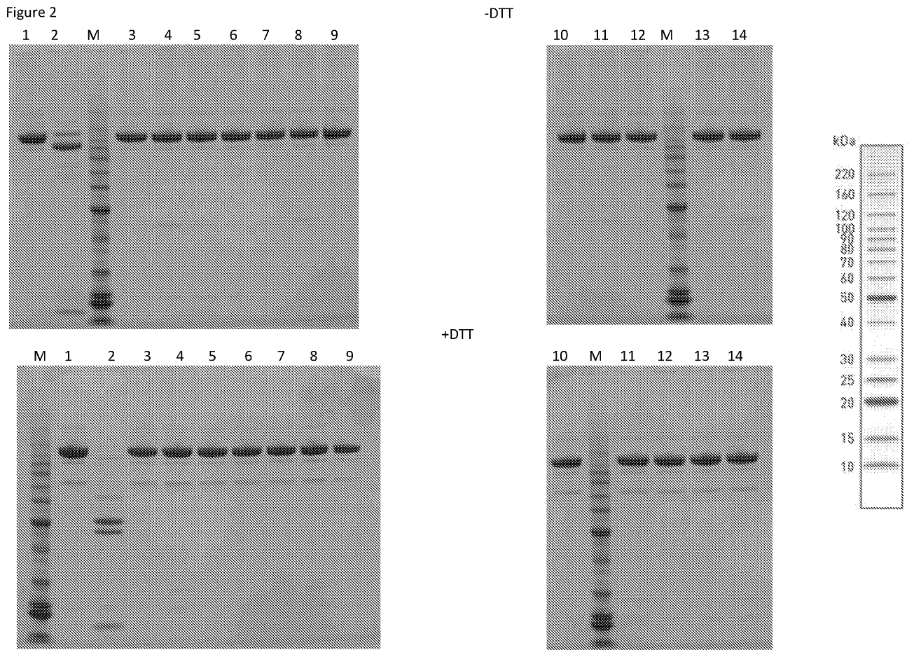

A4953 from Example 1 was incubated with incubated with increasing concentrations of FIX ((i) 1 μl, giving a final FIX concentration of 1 μg/mL; (ii) 5 μl, giving a final FIX concentration of 5 μg/mL; and (iii) 10 μl, giving a final FIX concentration of 10 μg/mL). Samples taken at time 1 hr, 2 hrs, 3 hrs and 19 hrs after the reaction start time were analysed by SDS PAGE under reducing and non-reducing conditions. As can be seen in FIG. 2, no cleavage of A4953 was observed, even at the higher concentrations of FIX and a reaction time of 19 hrs. The table below sets out the lanes of the gels in FIG. 2:

| | | | Protease | | Time/ |
|---|---|---|---|---|---|
| Lane | Sample | Protease | Added | | hrs |
| M | Mw | n/a | n/a | | n/a |
| | Marker | | | | |
| 1 | A00004953 | None | | | 19 |
| 2 | A00004953 | Lys-C | 0.45 | U/mL | 19 |
| 3 | A00004953 | FIX | 1 | μL | 1 |
| 4 | A00004953 | FIX | 5 | μL | 1 |
| 5 | A00004953 | FIX | 10 | μL | 1 |
| 6 | A00004953 | FIX | 1 | μL | 2 |
| 7 | A00004953 | FIX | 5 | μL | 2 |
| 8 | A00004953 | FIX | 10 | μL | 2 |
| 9 | A00004953 | FIX | 1 | μL | 3 |
| 10 | A00004953 | FIX | 5 | μL | 3 |
| 11 | A00004953 | FIX | 10 | μL | 3 |
| 12 | A00004953 | FIX | 1 | μL | 19 |
| 13 | A00004953 | FIX | 5 | μL | 19 |
| 14 | A00004953 | FIX | 10 | μL | 19 |

### Example 4—BoNT Engineered to Comprise an PRG Modified C-Loop are Effectively Cleaved by Thrombin

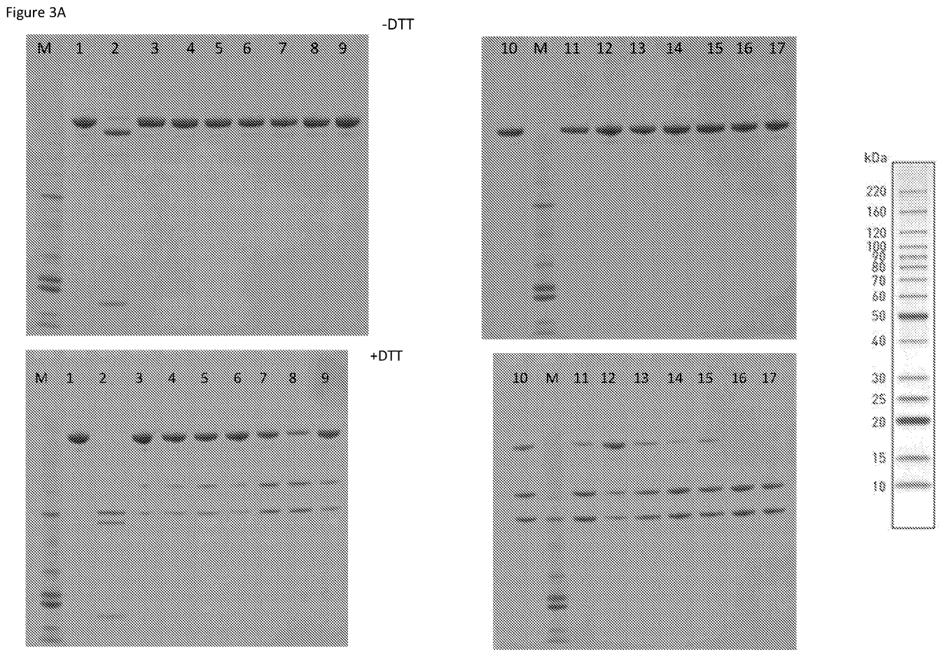

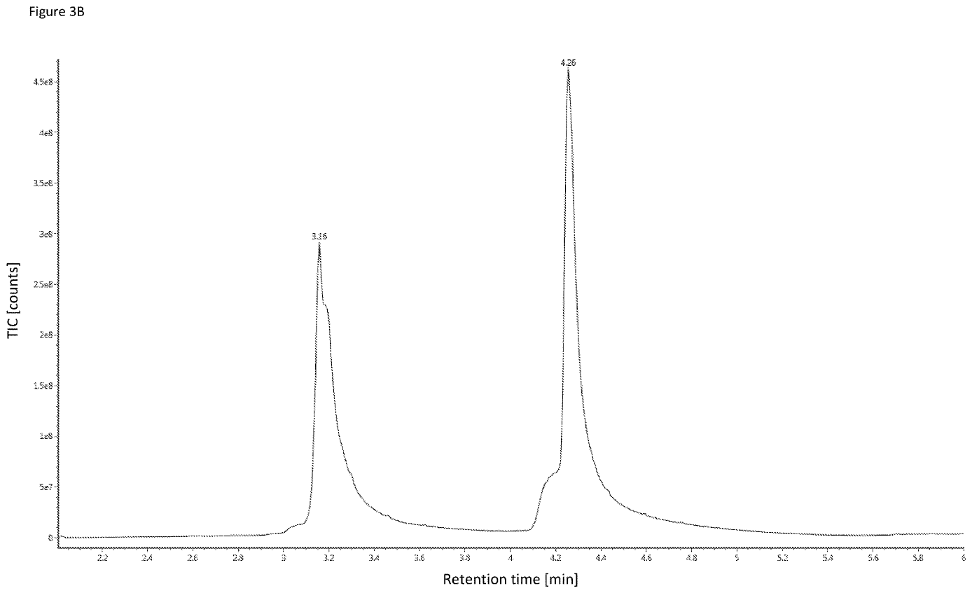

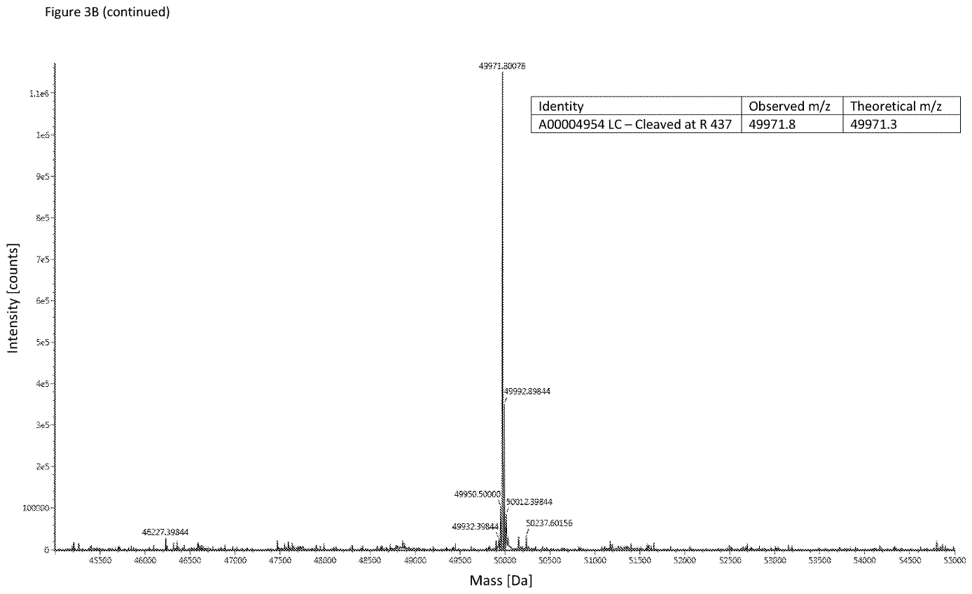

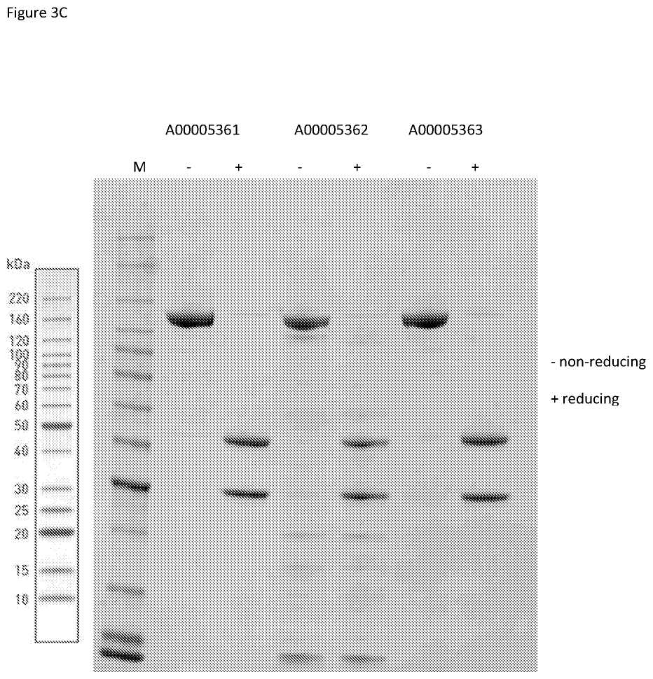

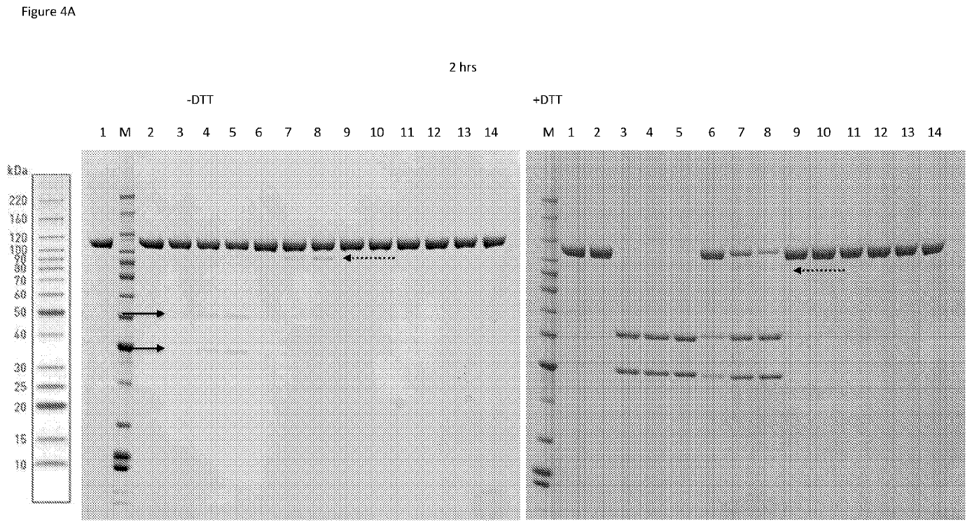

A4954 from Example 1 was incubated with incubated with increasing concentrations of thrombin ((i) 1 μl, giving a final thrombin concentration of 0.46 μg/mL; (ii) 5 μl, giving a final thrombin concentration of 2.3 μg/mL; and (iii) 10 μl, giving a final thrombin concentration of 4.6 g/mL). Samples taken at time 1 hr, 2 hrs, 3 hrs, 4 hrs and 19 hrs after the reaction start time were analysed by SDS PAGE under reducing and non-reducing conditions. As can be seen in FIG. 3A, cleavage was observed after 1 hr even at the lowest concentration of thrombin (0.46 μg/mL), and by 19 hrs complete cleavage of A4954 was observed for the higher thrombin concentrations (2.3 μg/mL and 4.6 g/mL). The table below sets out the lanes of the gels in FIG. 3A:

| | | | Protease | | Time/ |
|---|---|---|---|---|---|
| Lane | Sample | Protease | Concentration | | hrs |
| M | Mw Marker | n/a | n/a | | n/a |
| 1 | A00004954 | None | | | 19 |
| 2 | A00004954 | Lys-C | 0.45 | U/mL | 19 |
| 3 | A00004954 | Thrombin | 0.46 | μg/mL | 1 |
| 4 | A00004954 | Thrombin | 2.3 | μg/mL | 1 |
| 5 | A00004954 | Thrombin | 4.6 | μg/mL | 1 |
| 6 | A00004954 | Thrombin | 0.46 | μg/mL | 2 |
| 7 | A00004954 | Thrombin | 2.3 | μg/mL | 2 |
| 8 | A00004954 | Thrombin | 4.6 | μg/mL | 2 |
| 9 | A00004954 | Thrombin | 0.46 | μg/mL | 3 |
| 10 | A00004954 | Thrombin | 2.3 | μg/mL | 3 |
| 11 | A00004954 | Thrombin | 4.6 | μg/mL | 3 |
| 12 | A00004954 | Thrombin | 0.46 | μg/mL | 4 |
| 13 | A00004954 | Thrombin | 2.3 | μg/ml | 4 |
| 14 | A00004954 | Thrombin | 4.6 | μg/mL | 4 |
| 15 | A00004954 | Thrombin | 0.46 | μg/mL | 19 |
| 16 | A00004954 | Thrombin | 2.3 | μg/mL | 19 |
| 17 | A00004954 | Thrombin | 4.6 | μg/mL | 19 |

The resulting cleaved A4954 products were analysed by mass spectrometry. Samples were desalted using thermo ZEBA 0.5 m 7K MWCO spin columns. Columns were prepared by 3× washes with 300 μl 100 mM tris prior to thawing of samples. After desalting protein concentration was measured on nanodrop and the concentration was corrected to a concentration of 0.1 AU and 1 μl DTT was added per 99 μl of sample and was incubated for 30 minutes at 37° C. Samples were then analysed by LC/MS. The HPLC column used was a BioResolve, RP mAb Polyphenyl 450 Å 2.7 μm 2.1×150 mm, S/N 01153907216925. One microlitre of sample was injected. Samples were analysed using the method SXNA004472 Bioresolve sensitivity 6 mins segmented gradient as described in NBK88-86. As shown in FIG. 3B, a single cleavage site was identified within the activation loop, at residue R437. In addition, no off-site cleavage of A4954 by thrombin was observed.

Further, the same modified C loop comprising a PRG cleavage site as present in A4954 was used to replace the BoNT/A activation loop in a further three retargeted BoNTs, each with the LC/A1-ExoLoop-H_{N}/A1-TM architecture, but with different targeting moieties (A00005361, A00005362 and A00005363). As shown in FIG. 30, in each case thrombin was able to cleave the PGR cleavage site within the modified C loop, with no truncation products being visible. The table below sets out the lanes of the gels in FIG. 30

| Construct | Full Protein Name |
|---|---|
| A00005361 | pK8-LC/A1-PRG loop-Hn/A1-3xG4S-TM |
| A00005362 | pK8-LC/A1-PRG loop-Hn/A1-3xG4S-TM |
| A00005363 | pK8-LC/A1-PRG loop-Hn/A1-3xG4S-TM |

### Example 5—Design of Additional Exogenous Activation Loops and Modified BoNT/C1 Activation Loops

The methodology of Example 1 was used to generate engineered BoNTs with the same LC/A1-ExoLoop-H_{N}/A1-TM architecture as Example 1, but with exogenous activation loops and modified BoNT/C1 activation loops as shown in the table below.

| Loop | Sequence | Length | Suitable Proteases |
|---|---|---|---|
| C-loop Extended | CHKAENKSLVPR▾GSYNKTLDC | 21 | thrombin |
| Thrombin | | | |
| C-loop Alternate | CHKALTPR▾GVRLKTLDC | 17 | thrombin |
| Thrombin | | | |
| C-loop with Optimal | CHKAISGR▾SAYNKTLDC | 17 | Urokinase |
| Urokinase | | | |
| C-loop Alternative | CHKAVVPR▾VELVATLDC | 17 | t-PA_{fl}, t-PA_{frag} |
| t-PA | | | |
| Native | CPGR▾VVGGC | 9 | Urokinase, t-PA_{frag}, |
| Plasminogen | | | t-PA_{fl} |
| Plasminogen with | CGGGGSPGR▾VVGGGGSC | 16 | Urokinase, t-PA_{frag}, |
| spacers | | | t-PA_{fl} |
| Plasminogen loop | CSGR▾SAGGC | 9 | Urokinase |
| with Optimal | | | |
| Urokinase | | | |
| Optimal Urokinase | CGGGGSGR▾SAGGGGSC | 16 | Urokinase |
| and Spacers | | | |

▾ = cleavage site

### Example 6—BoNTs Engineered to Comprise an ENKSLVPRGS Modified C-Loop or an SGRSA Modified C-Loop are Effectively Cleaved by Thrombin and Urokinase Respectively

200 μg of engineered BoNT with an ENKSLVPRGS modified C-loop was treated with increasing amounts of (i) thrombin (0.39 μg, 1.95 μg and 3.90 μg) at 20° C. and samples taken at 2, 4 and 20 hours post-activation.

200 μg of engineered BoNT with an SGRSA modified C-loop was treated with increasing amounts of (i) urokinase; (ii) a functional t-PA fragment (t-PA_{frag}); or (iii) full-length t-PA (t-PA_{fl}) at increasing concentrations (0.39 μg, 1.95 μg and 3.90 μg) at 20° C. and samples taken at 2, 4 and 20 hours post-activation.

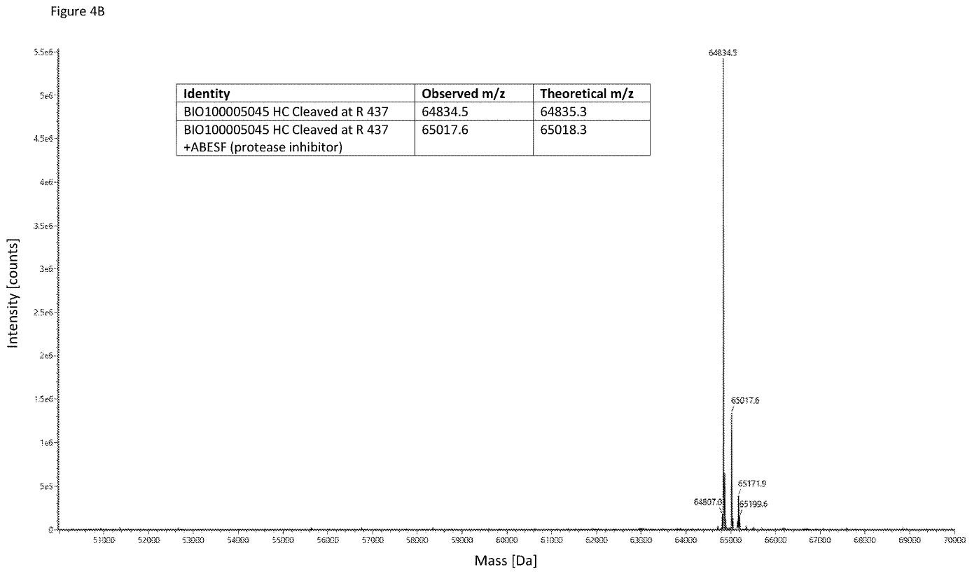

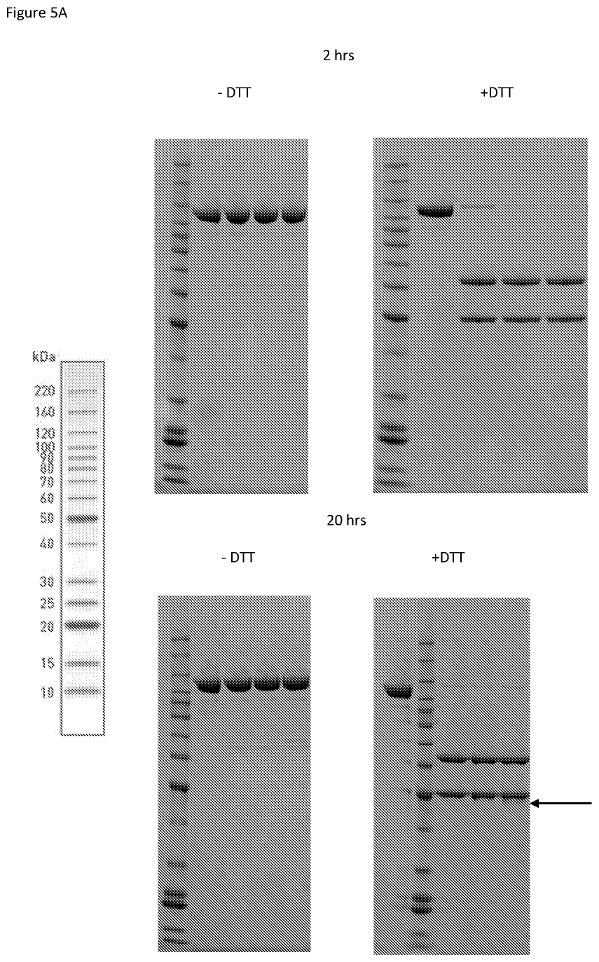

As shown in FIG. 4A, at 2 hours, cleavage of the engineered BoNT with an ENKSLVPRGS modified C-loop with thrombin was nearly complete, even at the lowest concentration of thrombin tested. The heavy and light chains are visible (black arrows) on the non-reducing gel. Cleavage of the engineered BoNT with an SGRSA modified C-loop had begun at 2 hours with urokinase at 0.39 μg, with trace amounts of a possible truncation product (dotted arrow). However, there was no observable cleavage of the engineered BoNT with an SGRSA modified C-loop with either t-PA. The table below sets out the lanes of the gels in FIG. 4A:

| | | | Amount |
|---|---|---|---|
| Lane | Loop | Protease | ug |
| M | n/a | Mw marker | n/a |
| 1 | ENKSLVPR▾GS | -ve control | 0 |
| 2 | SGR▾SA | -ve control | 0 |
| 3 | ENKSLVPR▾GS | thrombin | 0.39 |
| 4 | ENKSLVPR▾GS | thrombin | 1.95 |
| 5 | ENKSLVPR▾GS | thrombin | 3.9 |
| 6 | SGR▾SA | Urokinase | 0.39 |
| 7 | SGR▾SA | Urokinase | 1.95 |
| 8 | SGR▾SA | Urokinase | 3.9 |
| 9 | SGR▾SA | t-PA_{fl} | 0.39 |
| 10 | SGR▾SA | t-PA_{fl} | 1.95 |
| 11 | SGR▾SA | t-PA_{fl} | 3.9 |
| 12 | SGR▾SA | t-PA_{frag} | 0.39 |
| 13 | SGR▾SA | t-PA_{frag} | 1.95 |
| 14 | SGR▾SA | t-PA_{frag} | 3.9 |

Cleavage progressed over the time course, and by 4 hours thrombin cleavage of the engineered BoNT with an ENKSLVPRGS modified C-loop was nearly complete at all concentrations of thrombin tested (not shown). Cleavage of the engineered BoNT with an SGRSA modified C-loop was almost complete with urokinase at 0.39 μg, with trace amounts of a possible truncation product (not shown).

By 20 hours, cleavage of the engineered BoNT with an ENKSLVPRGS modified C-loop with thrombin nearly complete at all concentrations of thrombin tested. Cleavage of the engineered BoNT with an SGRSA modified C-loop was almost complete with urokinase at 0.39 μg. However, there was only trace cleavage of the engineered BoNT with an SGRSA modified C-loop with either t-PA.

The results of cleaving the engineered BoNT with an SGRSA modified C-loop with urokinase were compared with the urokinase cleavage of A4952 from Example 2. Compared with the KVR cleavage site, inclusion of a SGRSA cleavage site in a modified C-loop resulted in improved cleavage and specificity, and less off target activity. Thus, these results suggest that an SGRSA modified C-loop may be advantageous when engineering BoNTs for urokinase activation, compared with a KVR modified C-loop.

The results of cleaving the engineered BoNT with an ENKSLVPRGS modified C-loop with thrombin were compared with the thrombin cleavage of A4954 from Example 4.

Compared with the PRG cleavage site, inclusion of a ENKSLVPRGS cleavage site in a modified C-loop increased the rate of cleavage, with no observable off target activity. Thus, these results suggest that an ENKSLVPRGS modified C-loop may be advantageous when engineering BoNTs for thrombin activation, compared with a PRG modified C-loop.

Furthermore, even 72 hrs after thrombin activation (at 20° C., pH7.2, 150 mM NaCl), there was no observable truncation of the engineered BoNT with an ENKSLVPRGS modified C-loop, even when the concentration of thrombin was increased 10-fold (19.5 μg) (data not shown). This suggests that the ENKSLVPRGS modified C-loop would advantageously facilitate engineered BoNT stability, e.g. during manufacturing delays.

The resulting products from cleaving the engineered BoNT with an SGRSA modified C-loop (A5045) were analysed by mass spectrometry. Samples were desalted using thermo ZEBA 0.5 m 7K MWCO spin columns. Columns were prepared by 3× washes with 300 μl 100 mM tris prior to thawing of samples. After desalting protein concentration was measured on nanodrop and the concentration was corrected to a concentration of 0.1 AU and 1 μl DTT was added per 99 μl of sample and was incubated for 30 minutes at 37° C. Samples were then analysed by LC/MS. The HPLC column used was a BioResolve, RP mAb Polyphenyl 450 Å 2.7 μm 2.1×150 mm, S/N 01153907216925. One microlitre of sample was injected. Samples were analysed using the method SXNA004472 Bioresolve sensitivity 6 mins segmented gradient as described in NBK88-86. As shown in FIG. 4B, a single cleavage site was identified within the activation loop, at residue R437. Two off-site cleavages of A5045 were observed, at residues F167 and K683, both of which are potentially amenable to removal by genetic engineering.

### Example 7—Design of Further Exogenous Activation Loops and Modified BoNT/C1 Activation Loops

Based on the experimental results observed in Example 6, the methodology of Example 1 was used to generate further engineered BoNTs with the same LC/A1-ExoLoop-H_{N}/A1-TM architecture as Example 1, but with further exogenous protease cleavage sites as shown in the table below introduced into a C-loop.

| Activation loop | Recognition sequence |
|---|---|
| Thrombin | LTPR▾GVRL |
| Urokinase | PGSGR▾SAG |
| | PGSGR▾SASGTTGTG |
| t-PA_{frag} t-PA_{fl} | PPFGR▾SAG |

▾ = cleavage site

### Example 8—BoNTs Engineered to Comprise an LTPRGVRL Modified C-Loop, a PGSGRSAG Modified C-Loop or a PPFGRSAG Modified C-Loop are Effectively Cleaved by Thrombin, Urokinase and/or t-PA Respectively

200 μg of engineered BoNT with an LTPRGVRL modified C-loop was treated with increasing amounts of (i) thrombin (0.39 μg, 1.95 μg and 3.90 μg) at 20° C. and pH7.2 and samples taken at 2, 4 and 20 hours post-activation.

200 μg of engineered BoNT with an PGSGRSAG modified C-loop or an extended PGSGRSAG modified C-loop (comprising PGSGRSASGTTGTG) was treated with increasing amounts of (i) urokinase; (ii) a functional fragment of t-PA (t-PA_{frag}); or (iii) full-length t-PA (t-PA_{fl}) at increasing concentrations (0.39 μg, 1.95 μg and 3.90 μg) at 20° C. and pH7.2 and samples taken at 2, 4 and 20 hours post-activation.

200 μg of engineered BoNT with an PPFGRSAG modified C-loop was treated with increasing amounts of (i) urokinase; (ii) t-PA_{frag}; or (iii) t-PAi at increasing concentrations (0.39 μg, 1.95 μg and 3.90 μg) at 20° cand pH7.2 and samples taken at 2, 4 and 20 hours post-activation.

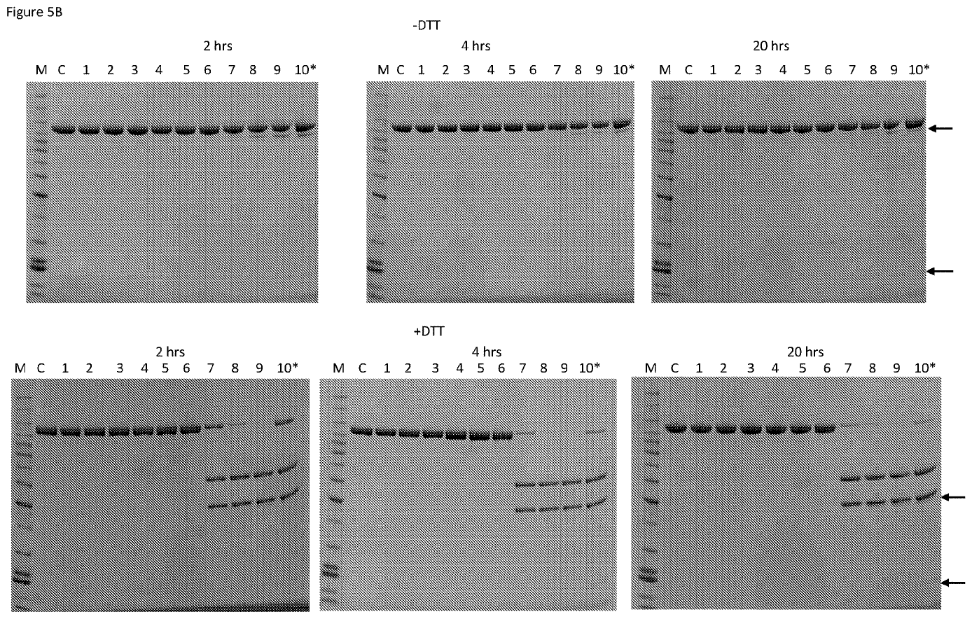

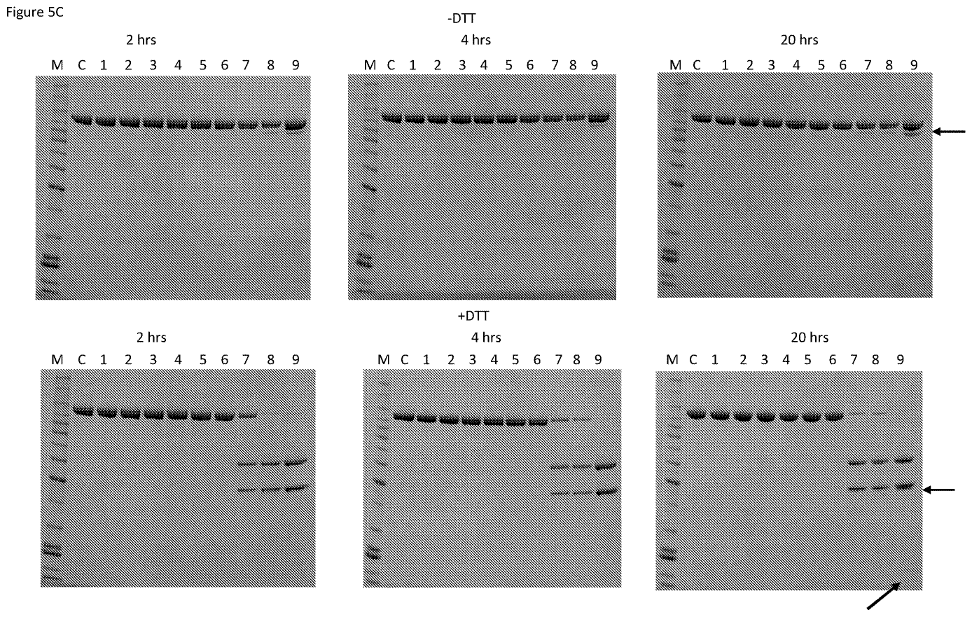

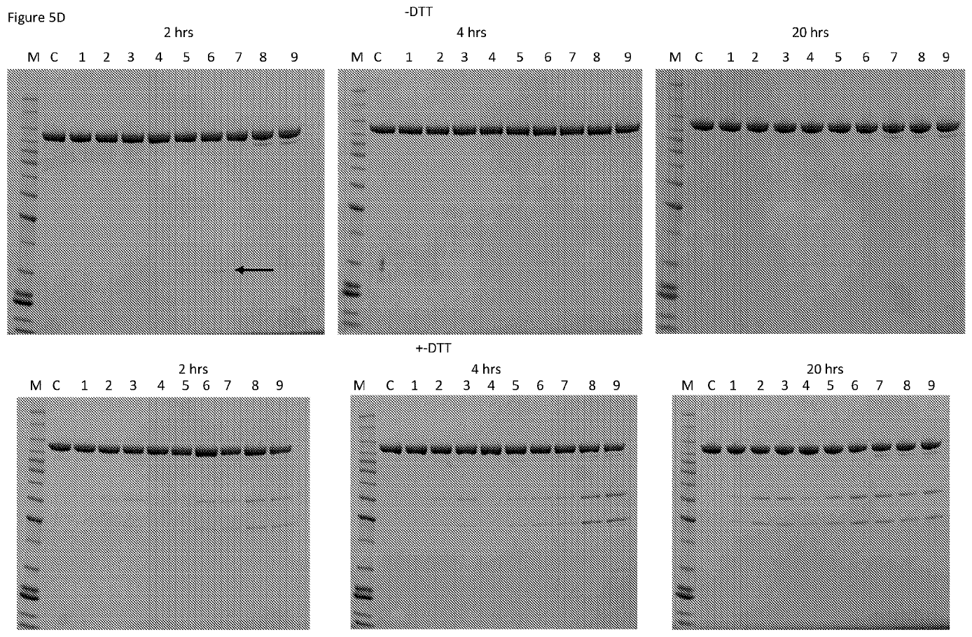

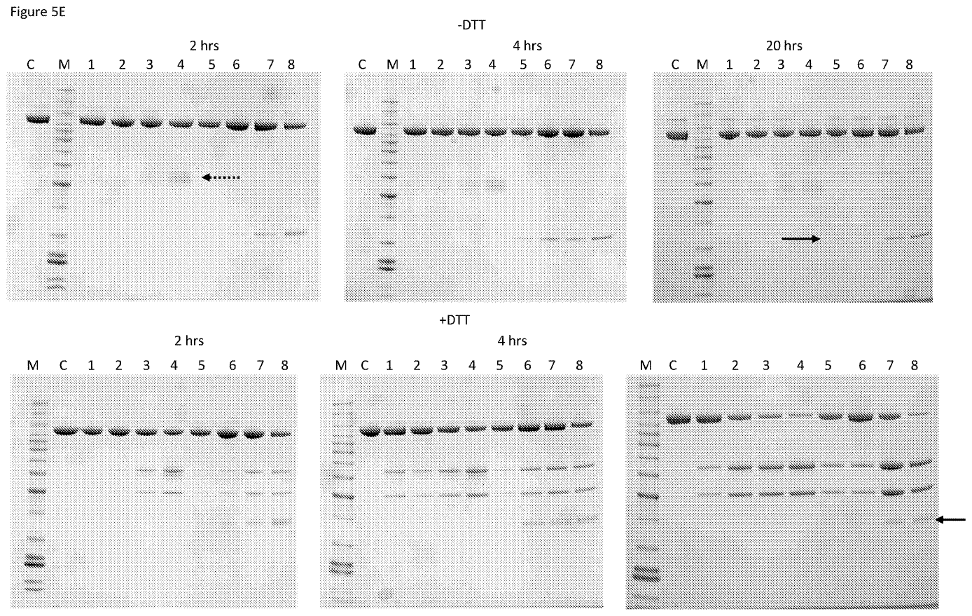

As shown in FIG. 5A, at 2 hours, cleavage of the engineered BoNT with an LTPRGVRL modified C-loop with thrombin was nearly complete, even at the lowest concentration of thrombin tested. Cleavage was essentially complete by 4 hours (data not shown). By 20 hours some truncation was observed, following extended incubation after activation had been completed. The table below sets out the lanes of the gels in FIG. 5A:

| M | Benchmark Mw Marker |
|---|---|
| C | No Protease Control |
| 1 | 0.39 μg thrombin |
| 2 | 1.95 μg thrombin |
| 3 | 3.9 μg thrombin |

As shown in FIG. 5B, at 2 hours, cleavage of the engineered BoNT with a PGSGRSAG modified C-loop with urokinase was underway, even at the lowest concentration of urokinase tested, with significant activation already achieved when 1.95 μg or 3.90 μg urokinase was used. Compared with the engineered BoNT with a SGRSA modified C-loop, the rate of activation by urokinase of the PGSGRSAG modified C-loop engineered BoNT was increased. Cleavage of the PGSGRSAG modified C-loop engineered BoNTs was essentially complete by 4 hours when 1.95 μg or 3.90 μg urokinase was used. Trace amounts of a possible urokinase-produced truncation product (black arrow) were observed at all time points.

However, there was no observable cleavage of the engineered BoNT with a PGSGRSAG modified C-loop with t-PA_{frag} or t-PA_{fl}. The table below sets out the lanes of the gels in FIG. 5B:

| M | Benchmark Mw Marker |
|---|---|
| C | No Protease Control |
| 1 | 0.39 μg t-PA_{fl} |
| 2 | 1.95 μg t-PA_{fl} |
| 3 | 3.9 μg t-PA_{fl} |
| 4 | 0.39 μg t-PA_{frag} |
| 5 | 1.95 μg t-PA_{frag} |
| 6 | 3.9 μg t-PA_{frag} |
| 7 | 0.39 μg Urokinase |
| 8 | 1.95 μg Urokinase |
| 9 | 3.9 μg Urokinase |
| 10* | 3.9 μg Urokinase |

As shown in FIG. 5C, at 2 hours, cleavage of the engineered BoNT with a PGSGRSASGTTGTG modified C-loop with urokinase was underway, even at the lowest concentration of urokinase tested, with significant activation already achieved when 1.95 μg or 3.90 μg urokinase was used. Cleavage of the PGSGRSASGTTGTG modified C-loop engineered BoNTs was essentially complete by 4 hours when 3.90 μg urokinase was used. Trace amounts of a possible urokinase-produced truncation product (black arrow) were observed at all time points. However, there was no observable cleavage of the engineered BoNT with a PGSGRSASGTTGTG modified C-loop with t-PA_{frag} or t-PA_{fl}. The table below sets out the lanes of the gels in FIG. 5C:

| M | Benchmark Mw Marker |
|---|---|
| C | No Protease Control |
| 1 | 0.39 μg t-PA_{fl} |
| 2 | 1.95 μg t-PA_{fl} |
| 3 | 3.9 μg t-PA_{fl} |
| 4 | 0.39 μg t-PA_{frag} |
| 5 | 1.95 μg t-PA_{frag} |
| 6 | 3.9 μg t-PA_{frag} |
| 7 | 0.39 μg Urokinase |
| 8 | 1.95 μg Urokinase |
| 9 | 3.9 μg Urokinase |

As shown in FIG. 5D, at 2 hours, cleavage of the engineered BoNT with a PPFGRSAG modified C-loop with was underway with each of urokinase, t-PA_{frag} and t-PA_{fl}. With urokinase, some activation was observed even at the lowest concentration of urokinase tested. For with t-PA_{frag} and t-PA_{fl} some activation was achieved when 1.95 μg or 3.90 μg t-PA_{frag} or t-PA_{fl} was used. Cleavage of the PPFGRSAG modified C-loop engineered BoNTs progressed over time, such that by 20 hours significant cleavage was observed for all of urokinase, t-PA_{frag} and t-PA_{fl} at each concentration tested. No truncation products produced using t-PA_{frag} or t-PA_{fl} were observed at any time points. The table below sets out the lanes of the gels in FIG. 5D:

| M | Benchmark Mw Marker |
|---|---|
| C | No Protease Control |
| 1 | 0.39 μg t-PA_{fl} |
| 2 | 1.95 μg t-PA_{fl} |
| 3 | 3.9 μg t-PA_{fl} |
| 4 | 0.39 μg t-PA_{frag} |
| 5 | 1.95 μg t-PA_{frag} |
| 6 | 3.9 μg t-PA_{frag} |
| 7 | 0.39 μg Urokinase |
| 8 | 1.95 μg Urokinase |
| 9 | 3.9 μg Urokinase |

The cleavage experiment was repeats with engineered BoNT with an PPFGRSAG modified C-loop and increasing concentrations of t-PA_{frag} and t-PA_{fl}. 200 μg of engineered BoNT with an PPFGRSAG modified C-loop was treated with t-PA_{frag} or t-PA_{fl} at increasing concentrations (3.9 μg, 7.8 μg, 15.6 μg and 31.2 μg) at 20° Cand pH7.2 and samples taken at 2, 4 and 20 hours post-activation. As shown in FIG. 5E, increasing concentrations t-PA_{frag} and t-PA_{fl} increased cleavage of the engineered BoNT with a PPFGRSAG modified C-loop at each time point, with almost complete activation being observed with higher concentrations. Even with these increased concentrations of t-PA_{frag} and t-PA_{fl}, no truncation products produced using t-PA_{frag} and t-PA_{fl} were observed at any time points. The table below sets out the lanes of the gels in FIG. 5E:

| M | Benchmark Mw Marker |
|---|---|
| C | No Protease Control |
| 1 | 3.9 μg t-PA_{fl} |
| 2 | 7.8 μg t-PA_{fl} |
| 3 | 15.6 μg t-PA_{fl} |
| 4 | 31.2 μg t-PA_{fl} |
| 5 | 3.9 μg t-PA_{frag} |
| 6 | 7.8 μg t-PA_{frag} |
| 7 | 15.6 μg t-PA_{frag} |
| 8 | 31.2 μg t-PA_{frag} |

### DISCUSSION

The ENKSLVPRGS and LTPRGVRL modified C-loops were highly effective, indicating that they have potential to be used to increase activation and/or reduce the amount of protease required for activation. Further, minimal truncation was observed in the range of engineered BoNTs tested, with trace truncation being observed only at 20 hours, when activation was complete by 4 hours. Furthermore, thrombin appears to be compatible with BoNT/X based engineered molecules (data not shown) Therefore, use of thrombin, particularly with ENKSLVPRGS and LTPRGVRL modified C-loops, and most preferably a LTPRGVRL modified C-loop is a commercially preferred option.

For activation with urokinase, the SGRSA and PGSGRSAG modified C-loops show improved cleavage with urokinase. The PGSGRSASGTTGTG modified C-loop also shows improvement over the KRV modified C-loop for urokinase. Whilst some truncation products were observed, they could potentially be managed during the production and purification process, and/or the susceptible sites within BoNT backbones could potentially be removed by further engineering.

For activation with t-PA, the VVPRVELVA modified C-loop shows no evidence of cleavage by any of the proteases tested. The SGRSA modified C-loop and extended PGSGRSASGTTGTG modified C-loop show some cleavage with t-PA variants (t-PA_{frag} & t-PA_{fl}), and the PPFGRSAG modified C-loop shows reasonable activation at higher concentrations with the t-PA variants. Significantly, no truncation of the SGRSA modified C-loop, extended PGSGRSASGTTGTG modified C-loop or PPFGRSAG modified C-loop was observed, even at high concentrations of the t-PA variants.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. An engineered clostridial neurotoxin, wherein the endogenous activation loop is replaced by a modified BoNT/C activation loop, in which the endogenous activation site of the BoNT/C activation loop, or part thereof, has been replaced by an exogenous protease cleavage site,
wherein cleavage at said cleavage site results in the production of a di-chain form of the engineered clostridial neurotoxin.

2. The engineered clostridial neurotoxin according to claim 1, wherein the exogenous protease cleavage site is a cleavage site specific for an exogenous protease selected from:
(a) thrombin;
(b) tissue plasminogen activator (t-PA); and/or
(c) urokinase (u-PA).

3. (canceled)

4. The engineered clostridial neurotoxin of claim 1, wherein the exogenous protease cleavage site comprises one or more of a thrombin consensus sequence of SEQ ID NO: 169 or SEQ ID NO: 170, a u-PA or t-PA consensus sequence of SEQ ID NO: 172 or SEQ ID NO: 173, LTPRGVRL (SEQ ID NO: 15), LVPRGS (SEQ ID NO: 16), ENKSLVPRGS (SEQ ID NO: 17), SGRSA (SEQ ID NO: 25), PPFGRSAG (SEQ ID NO: 33), PGSGRSAG (SEQ ID NO: 26), PGSGRSASGTTGTG (SEQ ID NO: 27), KRV (SEQ ID NO: 24), GRI (SEQ ID NO: 44) or PGRVVGG (SEQ ID NO: 50).

5. The engineered clostridial neurotoxin according to claim 1, wherein the exogenous protease cleavage site is between 3 and 10 amino acids in length.

6.-8. (canceled)

9. The engineered clostridial neurotoxin according to claim 1, wherein the endogenous neurotoxin activation loop is one or more selected from SEQ ID NO: 67 to 90.

10. The engineered clostridial neurotoxin according to claim 1, wherein:
(a) the exogenous protease cleavage site within the modified BoNT/C activation loop is the only cleavage site for the exogenous protease within the engineered clostridial neurotoxin;
(b) no off-site cleavage occurs on contacting the engineered clostridial neurotoxin with the exogenous protease; and/or
(c) no light chain truncation of the engineered clostridial neurotoxin occurs on contacting the engineered clostridial neurotoxin with the exogenous protease;
(d) an endogenous H_{C} or H_{CC} of a clostridial neurotoxin is replaced by an exogenous targeting moiety (TM).

11.-13. (canceled)

14. The engineered clostridial neurotoxin according to claim 1, wherein the engineered clostridial neurotoxin is a single-chain clostridial neurotoxin:
(a) (i) encoded by a nucleotide sequence having at least 70% sequence identity to SEQ ID NO: 154, 156, 158, 160, 162 or 164; or
(ii) encoded by a nucleotide sequence having at least 70% sequence identity to SEQ ID NO: 175, wherein SEQ ID NO: 176 within SEQ ID NO: 175 is replaced by a nucleotide sequence encoding at least one exogenous protease consensus sequence, cleavage site or a modified BoNT/C activation loop as defined in any one of claim 3; and/or
(b) comprising a polypeptide sequence having at least 70% sequence identity to one or more of SEQ ID NOs: 155, 157, 159, 161_163, 165, 166, 167_or 168; and/or
(c) comprising a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as defined in any one of claim 3, wherein the one or more exogenous protease site is selected from one or more SEQ ID NOs: 15, 33, or 25, and/or the modified BoNT/C activation loop is selected from one or more SEQ ID NOs: 57, 63, and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as defined in any one of claim 3, wherein the one or more exogenous protease site is selected from one or more SEQ ID NOs: 15, 33, or 25, and/or the modified BoNT/C activation loop is prefer-ably selected from one or more SEQ ID NOs: 57, 63, and/or 65.

15. A method for proteolytically processing an engineered clostridial neurotoxin according to claim 1 into a corresponding di-chain clostridial neurotoxin, the method comprising contacting the engineered clostridial neurotoxin with an exogenous protease specific for the exogenous protease cleavage site, thereby producing a di-chain clostridial neurotoxin.

16. A di-chain clostridial neurotoxin obtainable by the method of claim 15, wherein
(a) the C-terminus of the clostridial neurotoxin light chain ends with the amino acid sequence LVPR and the N-terminus of the clostridial neurotoxin heavy chain begins with the amino acid sequence GSK or GSY;
(b) the C-terminus of the clostridial neurotoxin light chain ends with the amino acid sequence SLVPR, and the N-terminus of the clostridial neurotoxin heavy chain begins with the amino acid sequence GSY;
(c) the C-terminus of the clostridial neurotoxin light chain ends with the amino acid sequence LTPR, and the N-terminus of the clostridial neurotoxin heavy chain begins with the amino acid sequence GVR;
(d) the C-terminus of the clostridial neurotoxin light chain ends with the amino acid sequence PGR, and the N-terminus of the clostridial neurotoxin heavy chain begins with the amino acid sequence VVG;
(e) the C-terminus of the clostridial neurotoxin light chain ends with the amino acid sequence DKR, and the N-terminus of the clostridial neurotoxin heavy chain begins with the amino acid sequence VLY;
(f) the C-terminus of the clostridial neurotoxin light chain ends with the amino acid sequence SGR, and the N-terminus of the clostridial neurotoxin heavy chain begins with the amino acid sequence SA;
(g) the C-terminus of the cleaved engineered clostridial neurotoxin light chain may end with the amino acid sequence SGR, and the N-terminus of the cleaved engineered clostridial neurotoxin heavy chain may begin with the amino acid sequence TL; or
(h) the C-terminus of the clostridial neurotoxin light chain ends with the amino acid sequence FGR, and the N-terminus of the clostridial neurotoxin heavy chain begins with the amino acid sequence SA.

17. A polynucleotide encoding an engineered clostridial neurotoxin as defined in claim 1.

18. An expression vector comprising a polynucleotide as defined in claim 17, which is operably linked to a promoter.

19. (canceled)

20. A method of producing an engineered clostridial neurotoxin as defined in claim 1, the method comprising the step of expressing a polynucleotide or an expression vector in a cell, and recovering the expressed engineered clostridial neurotoxin.

21. (canceled)

22. A cell expressing an engineered clostridial neurotoxin as defined in claim 1.

23. (canceled)

24. A pharmaceutical composition comprising an engineered clostridial neurotoxin as defined in claim 1, and a pharmaceutically acceptable carrier, excipient, diluent, adjuvant, propellant and/or salt.

25. A method of preventing or treating a disease or disorder for which a therapy with a botulinum neurotoxin is indicated, wherein the clostridial neurotoxin is administered to a subject in need the composition of claim 24.

26.-32. (canceled)

33. A cosmetic composition comprising an engineered clostridial neurotoxin as defined in claim 1, and a cosmetically acceptable carrier, excipient, diluent, adjuvant, propellant, and/or salt.

34. A method for preventing or alleviating a cosmetic indication using the cosmetic composition of claim 33, wherein the application of a botulinum neurotoxin is indicated for preventing or alleviating the cosmetic indication.

35. The cosmetic composition according to claim 34, wherein the clostridial neurotoxin is for administration to a subject in single-chain or di-chain form.

36.-40. (canceled)

41. A method for proteolytically processing a single-chain clostridial neurotoxin into a corresponding di-chain clostridial neurotoxin, the method comprising:
(a) providing a single-chain clostridial neurotoxin; and
(b) contacting the single-chain clostridial neurotoxin with an exogenous protease;
wherein the single-chain clostridial neurotoxin has an exogenous protease cleavage site and/or modified BoNT/C activation loop as defined in claim 3,
wherein the exogenous protease hydrolyses a peptide bond of the exogenous protease cleavage site and/or modified BoNT/C activation loop, thereby producing a di-chain clostridial neurotoxin.

42. The method according to claim 41, wherein the single-chain clostridial neurotoxin:
(a) is an engineered clostridial neurotoxin as defined in claim 1;
(b) is encoded by a nucleic acid sequence comprising or consisting of a nucleotide sequence having at least 70% sequence identity to SEQ ID NO: 154, 156, 158, 160, 162, or 164; or (ii) at least 70% sequence identity to SEQ ID NO: 175, wherein SEQ ID NO: 176 within SEQ ID NO: 175 is replaced by a nucleotide sequence encoding at least one exogenous protease consensus sequence, cleavage site or a modified BoNT/C activation loop as defined in claim 3;
(c) comprises or consists of a polypeptide sequence having at least 70% sequence identity to one or more of SEQ ID NOs: 155, 157, 159, 161, 163, 165, 166, 167, or 168; and/or
(d) comprises or consists of a polypeptide sequence having (i) at least 70% sequence identity to SEQ ID NO: 150, wherein SEQ ID NO: 2 within SEQ ID NO: 150 has been replaced by one or more exogenous protease cleavage site and/or modified BoNT/C activation loop as defined in claim 3, wherein the one or more exogenous protease site is selected from one or more SEQ ID NOs: 15, 33, or 25, and/or the modified BoNT/C activation loop is selected from one or more SEQ ID NOs: 57, 63, and/or 65; or (ii) at least 70% identity to SEQ ID NO: 152, wherein SEQ ID NO: 2 within SEQ ID NO: 152 has been replaced by one or more exogenous protease cleavage site and/or modified BoNT/C activation loop as defined in claim 3, wherein the one or more exogenous protease site is selected from one or more SEQ ID NOs: 15, 33, or 25, and/or the modified BoNT/C activation loop is selected from one or more SEQ ID NOs: 57, 63, and/or 65.
